(19) United States
(12) Patent Application Publication
(10) Pub. No.: US 2018/0327806 A1
(43) Pub. Date: Nov. 15, 2018

(54) MULTIPLEX HIGH-RESOLUTION DETECTION OF MICRO-ORGANISM STRAINS, RELATED KITS, DIAGNOSTICS METHODS AND SCREENING ASSAYS

(71) Applicants: THE BROAD INSTITUTE, INC., Cambridge, MA (US); THE GENERAL HOSPITAL CORPORATION, Boston, MA (US)

(72) Inventors: Deborah HUNG, Cambridge, MA (US); Eachan JOHNSON, Boston, MA (US); Brad POULSEN, Boston, MA (US)

(21) Appl. No.: 15/773,895

(22) PCT Filed: Nov. 4, 2016
(86) PCT No.: PCT/US16/60730

§ 371 Date: May 4, 2018

Related U.S. Application Data
(60) Provisional application No. 62/250,610, filed on Nov. 4, 2015.

(51) Int. Cl. C12Q 1/18; C07K 14/35; C07K 14/21

(52) U.S. Cl. CPC C12Q 1/18; C12N 15/70; C07K 14/21; C07K 14/35

(57) ABSTRACT

The present invention relates to multiplex high-resolution detection of micro-organism strains. It provides kits, diagnostics methods and screening assays.

### CROSS-REFERENCE TO RELATED APPLICATIONS

This application is the U.S. National Stage of International Application No. PCT/US2016/060730, filed Nov. 4, 2016, which claims the benefit of U.S. Provisional Application No. 62/250,610, filed Nov. 4, 2015. The entire contents of the above-identified priority applications are hereby fully incorporated herein by reference.

### FEDERAL FUNDING LEGEND

This invention was made with government support under grant numbers 1R21AI098705-01 and 5R33AI098705-04 awarded by the National Institutes of Health. The government has certain rights in the invention.

### FIELD OF THE INVENTION

The present invention relates to the field of micro-organism strain detection and identification. It pertains to sets of primers, collection of double-stranded nucleic acid molecules, sets of probes and kits for such detection and identification, in particular for multiplex high-resolution detection of micro-organism strains amongst a strain collection and for multiplex identification of given growth conditions of said micro-organism strains. The present invention also relates to the field of diagnostics and screening assays, in particular assays for the identification of compounds with antibacterial properties.

### BACKGROUND OF THE INVENTION

The National Institute of Health estimates that 70% of pathogenic bacteria have developed resistance to antibiotics and of the 1.7 million hospital-acquired infections in the United States per year, 99,000 cases result in death [Klevens, R. M., et al., Estimating health care-associated infections and deaths in U.S. hospitals, 2002. Public Health Rep, 2007. 122(2): p. 160-6]. Pseudomonas aeruginosa is among one of the most challenging of these pathogens with significant resistance, and is particularly prevalent in immunocompromised individuals such as patients with cystic fibrosis. By age 20, 60-70% of cystic fibrosis patients develop a P. aeruginosa infection that often persists resulting in chronic infections until eventually succumbing to the infection (Folkesson, A., et al., Adaptation of Pseudomonas aeruginosa to the cystic fibrosis airway: an evolutionary perspective. Nat Rev Microbiol, 2012. 10(12): p. 841-51). Due to its ability to evade current antibiotics or develop resistance, P. aeruginosa clinical strains are increasingly resistant to all current clinically relevant antibiotics (Hancock, R. E., Resistance mechanisms in Pseudomonas aeruginosa and other nonfermentative gram-negative bacteria. Clin Infect Dis, 1998. 27 Suppl 1: p. S93-9., Strateva, T. and D. Yordanov, Pseudomonas aeruginosa—a phenomenon of bacterial resistance. J Med Microbiol, 2009. 58(Pt 9): p. 1133-48). New approaches for treating pseudomonal infections are paramount to overcoming antibiotic resistance thereby allowing cystic fibrosis patients longer and more comfortable lives. Unfortunately, the current pipeline of antibiotics in general, but Gram-negative bacteria in particular, is alarmingly empty. Much of this failure is due to the incredible challenge of finding lead compounds against organisms such as P. aeruginosa for further development because of its intrinsic barriers and resistance to small molecules.

P. aeruginosa is inherently resistant to antibiotics due to many different factors (Nikaido, H., Multidrug resistance in bacteria. Annu Rev Biochem, 2009. 78: p. 119-46). Many isolates have acquired antibiotic resistance conferring elements through horizontal gene transfer of plasmids or chromosomally integrated transposons. Such acquired resistance mechanisms include inactivation of the antibiotic (e.g. β-lactams, aminoglycosides), modification of the molecular target (e.g. quinolones, streptomycin), and changes in intracellular drug concentration due to increased transport out of the cell by multidrug efflux pumps [Walsh, C., Antibiotics: actions, origins, resistance 2003]. While each of these antibiotic resistance mechanisms contributes to P. aeruginosa drug-resistance, its intrinsic cell impermeability, which is on the order of 100 times less permeable than that of another Gram negative organism such as E. coli (Nakae, T., Role of membrane permeability in determining antibiotic resistance in Pseudomonas aeruginosa. Microbiol Immunol, 1995. 39(4): p. 221-9.), is a major barrier in achieving bacterial death. This impermeability, coupled with numerous efflux systems, results in low intracellular drug concentrations that are insufficient to kill the cell. The P. aeruginosa genome contains 5570 open reading frames, 71 of which (by homology) are outer membrane proteins (OMPs) that regulate transport of small molecules in and out of the cell. Importantly, the outer cell membrane structure can be exploited as a target for effective bacterial killing. Natural innate defense mechanisms such as antimicrobial peptides target the outer membrane of the cell and have been reported to interact with OMPs [Lin, Y. M., et al., Outer membrane protein I of Pseudomonas aeruginosa is a target of cationic antimicrobial peptide/protein. J Biol Chem, 2010. 285(12): p. 8985-94]. Furthermore, numerous antibiotics target enzymes involved in cell wall biosynthesis. Finally, a study recently reported the effective targeting of the essential OMP OstA by a peptidomimetic antibiotic in P. aeruginosa [9]. Thus, in order to address the significant hurdle created by the inability to find lead small molecule candidates against P. aeruginosa for antibiotic development, it is desirable to identify novel small molecule leads that combat the intrinsic resistance properties of P. aeruginosa by selectively targeting essential OMPs, thus bypassing the need for molecules to penetrate the cell wall and accumulate to sufficient concentrations for effective killing.

Further, Mycobacterium tuberculosis is a 9,000 year old plague and tuberculosis (TB) is the most deadly disease caused by a bacterium (Hershkovitz et al., PLoS ONE, 2008).

It would be desirable to identify new mechanism of actions for candidate antibacterial agents. This would be advantageous, because new drugs must be effective against resistant strains. Anti-bacterial agents that are effective according to new mechanisms minimize the overlap with resistance currently observed with known therapies. In order to do so, it would be desirable to be able to assay such novel mechanisms of action in order to screen for new targets.

Conventional target-based screening is advantageous in that the mechanism of action is known, activity assays are already available, and the lead development is well-informed. However, there are drawbacks, namely whole-cell activity remains unknown, and the target must remain stable (Kumar et al, PLoS ONE, 2012).

On the other hand, conventional whole-cell screening is advantageous in that it reflects whole-cell activity, and is easy to set up. However, disadvantages thereof include the fact that the mechanism of action is unknown, and lead development is conducted in a blind fashion (Stanley et al, ACS Chem Bio, 2012).

Finally, target-based whole-cell screening offer the advantages of pertaining to whole-cell activity combined with provided clues as to the mechanism of action (see, e.g., DeVito et al., Nature Biotechnology, 2002). However, there still are disadvantages, as the molecular biology might be difficult, there is still a requirement for an investigational follow up on the mechanism, and there may be off-target confounding effect.

Citation or identification of any document in this application is not an admission that such document is available as prior art to the present invention.

### SUMMARY OF THE INVENTION

The availability of multiple whole-cell target-based screens would be desirable, as this could improve knowledge on mechanism of action, and facilitate screening, in that the requirements for labor, time, and hence costs, increase linearly with the number of screens.

In certain example embodiments, a recombinant hypomorph microbial cell is provided that is recombinantly engineered to have reduced expression of one or more essential genes and further modified to comprise a strain specific nucleic acid identifier that identifies the hypomorph microbial cell. In certain example embodiments, the strain specific nucleic acid identifier is a non-naturally occurring nucleotide sequence. In certain example embodiments, the strain specific nucleic acid identifier is incorporated into the genome of the hypomorph microbial cell. The strain specific nucleic acid identifier may comprise, in a 5′ to 3′ direction, a first primer binding sight, a strain specific nucleic acid sequence, and a second primer binding site, wherein the hypomorph specific nucleic acid sequence identifies the one or more essential genes having reduced expression.

The recombinant hypomorph cell may be a bacterial cell, a fungal cell, a mycological cell, a protozoal cell, a nematode cell, a trematode cell, or a cestode cell. In certain example embodiments, the recombinant hypomorph is a bacterial cell. The bacterial cell may be an Eschericia, a Klebsiella, a Psuedomonas, a Staphylococcus, an Acinetobacter, a Candida, an Enterobacter, an Enterococcus, a Proteus, a Streptococcus, or a Stenotrophomonas bacteria. In certain example embodiments, the cell is selected from the group consisting of Eschericia coli, Klebsiella pneumoniae, Pseudomonas aeruginosa, Staphylococcus aureus, Acinetobacter baumannii, Candida albicans, Enterobacter cloacae, Enterococcus faecalis, Enterococcus faecium, Proteus mirabalis, Streptococcus agalactiae, and Stenotrophomonas maltophila. In certain example embodiments, the cell is P. aeruginosa. In certain other example embodiments, the cell is a Mycobacterium. In certain example embodiments, the Mycobacterium is M. tuberculosis, M. avium-intracellulare, M. kansasii, M. fortuitum, M. chelonae, M. leprae, M. africanum, M. microti, M. avium paratuberculosis, M. intracellulare, M. scrofulaceum, M. xenopi, M. marinum, or M. ulcerns.

In certain example embodiments, reduced expression of the one or more essential genes is achieved by recombinantly engineering the microbial cell so that one or more essential genes is under the control of a weak promoter. In certain example embodiments, the weak promoter may comprise a spacer sequence between the promoter and the RNA polymerase binding site. In certain other example embodiments, reduced expression of the one or more essential genes may be achieved by recombinantly engineering the cell such that the one or more essential genes further encodes a protein degradation signal that is appended to the expressed protein upon translation and that targets the protein expression product for degradation. In certain example embodiments, the protein degradation tag targets the protein for degradation by a clp-protease. In certain example embodiments, targeted protein degradation may be further enhanced by engineering the cell to further express a protease adapter protein. The protease adapter protein may be operatively linked to an inducible promoter.

In certain example embodiments, the one or more essential genes are genes whose expression products are localized to the cytoplasam, cytoplasmic membrane, periplasm, outer membrane, or extracellular space. In certain example embodiments, the one or more essential proteins are localized to the outer membrane. In certain example embodiments, the function of the essential gene expression product is outer membrane protein assembly, cell structure/outer membrane integrity, outer membrane protein chaperone/assembly, LPS biosynthesis, rod-shape structural protein, endonuclease, folate synthesis, cell wall synthesis, or leucyl-tRNA synthesis. In certain example embodiments, the one or more essential genes are selected from the group consisting of ostA, opr86, oprL, lol B, omlA, lppL, surA, lolA, tolB, tolA, mreC, lptA, lptD, lptE, dhfR, folP, murA, gyrA, lpcX, leuS and gcp. In certain other example embodiments, the one or more essential proteins are selected from the group consisting of ccsX, ctaC, eno, fba, folB, glcB, marP, mdh, mshC, murG, nadE, pstP, sucD, topA, efpA, tpi, dlat, and mesa

In certain example embodiments, a set of hypomorph recombinant cells for use in various multiplex screening assays described further herein comprises a collection of the hypomorph recombinant cells described herein. In certain other example embodiments, a set of nucleic acid primer pairs for detecting and amplifying the hypomorph's strain specific nucleic acid identifier comprises a first primer that binds to the first primer binding site of the strain specific nucleic acid identifier and a second primer that binds to the second primer binding site of the strain specific nucleic acid identifier. One or both of the primers may further comprise an origin-specific nucleic acid identifier specific to the individual discrete volume to which a given primer pair is delivered. One or both of the primers may also further comprise an experimental condition specific nucleic acid identifier sequence identifying the type of experimental conditions present in a given discrete volume. In certain example embodiments, the primers may further comprise a first and second sequencing primer binding site and/or a first and second sequencing adapter.

In certain example embodiments, a multiplex method for whole-cell target-based screening of microbes comprises culturing each hypomorph microbial cell of a given set in different individual discrete volumes and under differing experimental conditions, then detecting the hypomorph microbial cells from the individual discrete volumes, where the failure to detect one or more hypomorph cells, or the detection of a decreased amount of one or more hypomorph cells relative to other hypomorph cells or a control, indicates susceptibility of the one or more hypomorph cells to the experimental condition. In certain example embodiments, detecting the hypomorph cells comprises amplifying the strain specific nucleic acid identifier using the nucleic acid primer pairs disclosed herein, sequencing the resulting amplicons, and determining an exact or relative number of reads where the sequencing reads can be deconvoluted based on the type of hypomorph cell the read originated from, the individual discrete volume the sequencing read originated from, and the experimental conditions present in that individual discrete volume. The absence of reduced amounts of a given hypomorph cell under a given set of experimental conditions indicates that susceptibility of the hypomorph to those experimental conditions. Further, the type of hypomorph, and the one or more essential genes whose expression was reduced therein, may further indicate a mechanism of action by which a given set of experimental conditions acts to render the hypomorph cell susceptible to those experimental conditions. Thus, the methods disclosed herein may be used to screen for novel target agents. In certain example embodiments, the target agents may be chemical agents. In certain other example embodiments, the chemical agents may be antibiotics.

The present invention also relates to a collection of double-stranded nucleic acid molecules for multiplex high-resolution detection of micro-organism strains amongst a strain collection and for multiplex identification of given growth conditions of said micro-organism strains, wherein each molecule may comprise an experimental conditions sequence; and a unique polynucleotide identifier.

The present invention also relates to a set of probes for multiplex high-resolution detection of micro-organism strains amongst a strain collection and for multiplex identification of given growth conditions of said micro-organism strains, wherein each probe may be a single stranded nucleic acid molecule as herein described.

The present invention also relates to a method for the diagnostic of a pathogenic infection, by multiplex high-resolution detection of micro-organism strains from a strain collection, wherein said method may comprise: providing a test sample from a patient; extracting exogenous nucleic acids from said test sample; and hybridizing said exogenous nucleic acids with a set of primers as herein described or a set of probes as herein described.

The present invention also relates to a method of generating and selecting a collection of hypomorph strains of a micro-organism population, which may comprise: generating a collection of strains of micro-organisms, wherein for each strain the level of expression of a unique gene is controlled by an exogenous promoter, whereby the level of expression of the unique gene is altered compared with the level of expression of the unique gene under its endogenous promoter, each strain of micro-organism having a unique polynucleotide identifier, whereby each unique polynucleotide identifier is configured for multiplex high-resolution detection of the corresponding strain amongst said collection of strains; outgrowing the generated strains of micro-organisms; and selecting the hypomorph strains of micro-organisms based on growth kinetics and the expression level of the unique gene, the expression level of the unique gene being indicative of the promoter strength.

The present invention also relates to a method of screening assay of a set of experimental conditions on a collection of strains of a micro-organism, which may comprise, for each strain: providing a collection of hypomorph micro-organism strains; preparing a pool of strains from said collection; subjecting said pool of strains to a set of experimental conditions; and performing multiplex high-resolution detection of the strains amongst said collection of strains.

The present invention also relates to a method for identifying a pathogenic micro-organism with a set of primers as herein described or detection of double-stranded nucleic acid molecules as herein described or a collection of probes as herein described.

The present invention also relates to a kit for multiplex high-resolution detection of micro-organism strains amongst a strain collection and for multiplex identification of given growth conditions of said micro-organism strain.

The present invention also relates to a diagnostic kit for multiplex high-resolution detection of micro-organism strains amongst a strain collection and for multiplex identification of given growth conditions of said micro-organism strain.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other aspects, objects, features, and advantages of the example embodiments will become apparent to those having ordinary skill in the art upon consideration of the following detailed description and illustrated example embodiments.

### BRIEF DESCRIPTION OF THE DRAWINGS

FIG. 1 shows an illustrative protocol for Multiplexed Growth and Quantitation Using Illumina® Sequencing.

FIG. 2 depicts outline for a Tn-seq based strategy for identifying essential genes in P. aeruginosa.

FIG. 3 illustrates a strategy for creating knockdown strains and developing variable promoters for use in P. aeruginosa.

FIG. 4 shows the results of PA14 strains of chromosomally-integrated GFP driven by constitutive variable promoters.

FIG. 5 illustrates the use of variable promoters for generating and selecting hypomorph strains.

FIG. 6A shows that PA14 strain with DhfR knockdown (hypomorph) is hypersensitive to trimethoprim. FIG. 6B shows that PA14 strain with MurA knockdown (hypomorph) is hypersensitive to fosfomycin.

FIG. 7 show that DhfR knockdown PA14 strain displays dose-response to trimethoprim.

FIG. 8A illustrates PA14 hypomorph screen reproducibility of chlorhexidine. FIG. 8B illustrates PA14 hypomorph screen reproducibility of broxyquinoline.

FIG. 9 depicts a strategy for the generation of hypomorph strains of M. tuberculosis.

FIG. 10A shows that the strain obtained is hypersensitive to methotrexate targeting dfrA (dose response curve). FIG. 10B shows that the strain obtained is hypersensitive to 4592 targeting trpA (dose response curve).

FIG. 11 shows principle for multiplex detection of the invention.

FIG. 12 illustrates that the method of the invention allows to reliably detect and count micro-organism cells.

FIG. 13 illustrates a screening method of the invention.

FIG. 14 shows part I of the screening: hypomorph strains are outgrown in presence of anhydrotetracycline (atc) so as to obtain a hypomorph phenotype.

FIG. 15 shows part II of the screening method of using multiplex PCR to generate the collections of ds DNA molecules of the invention.

FIG. 16 shows a part III of the screening method comprising data processing.

FIG. 17 illustrates the high reproducibility obtained.

FIG. 18 shows results that validate the method with respect to positive controls with compounds trimethoprim and rifampin.

FIG. 19 illustrates that the on-board controls show robust statistical performance of the assay.

FIG. 20 illustrates that pilot screen demonstrated clear differential inhibition.

FIG. 21 shows differential inhibition demonstrated by OD₆₀₀ dose response.

FIG. 22 shows that the screening assay has a high validation rate.

FIG. 23 shows that the scaled-up screen was highly reproducible.

FIG. 24 shows multiplex growth curves.

FIG. 25 shows screen performance across strains.

FIG. 26 shows the relationship between Z′-factors and growth rate.

FIG. 27 provides a schematic of an example multiplex screening method for screening a chemical agent library in accordance with certain example embodiments.

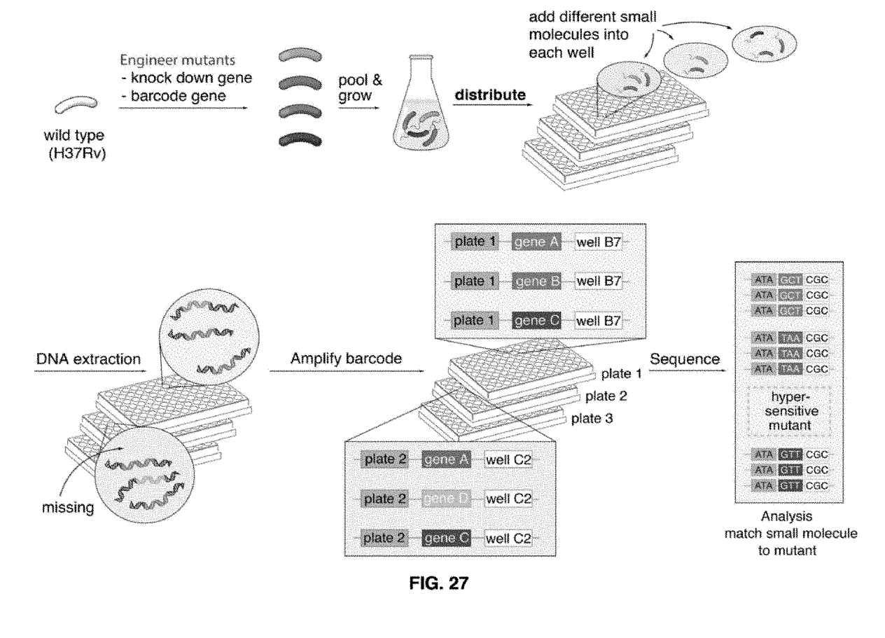

FIG. 28 provides a schematic of a multiplex assay for screening a chemical agent library using hypomorphs with DAS+4 mediated knockdown of essential gene products in accordance with certain example embodiments.

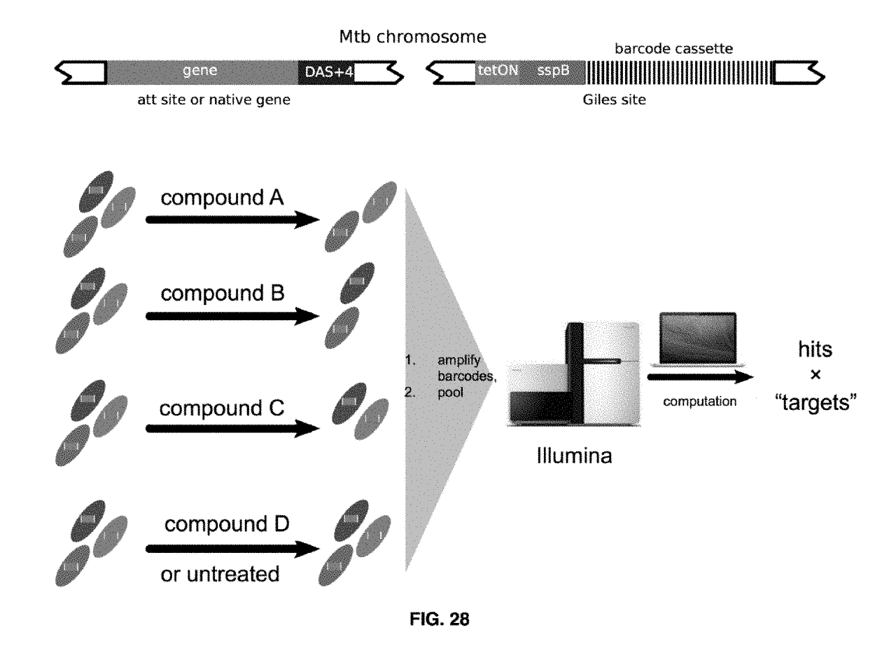

FIG. 29 provides a more detailed view of the BSL-3 assay component of the overall assay depicted in FIG. 28.

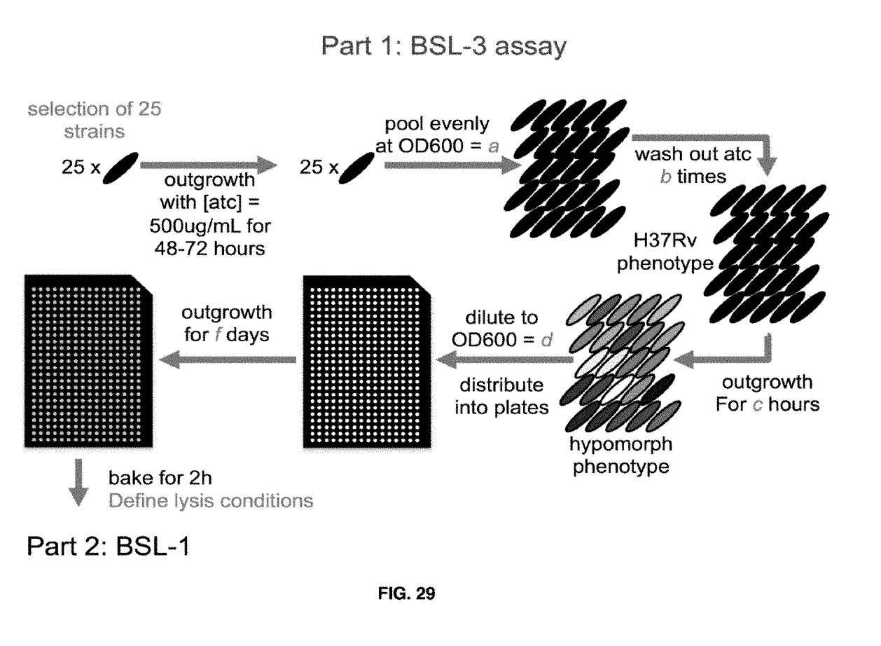

FIG. 30 provides a more detailed view of the BSL-1 readout component of the overall assay depicted in FIG. 28.

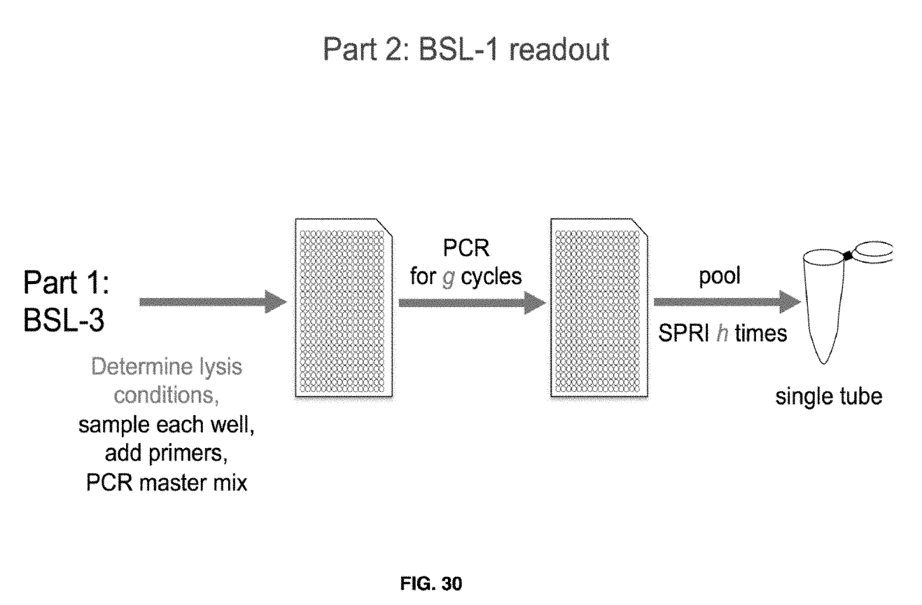

FIG. 31 lists a set of example screening parameters to be optimized in the methods disclosed herein.

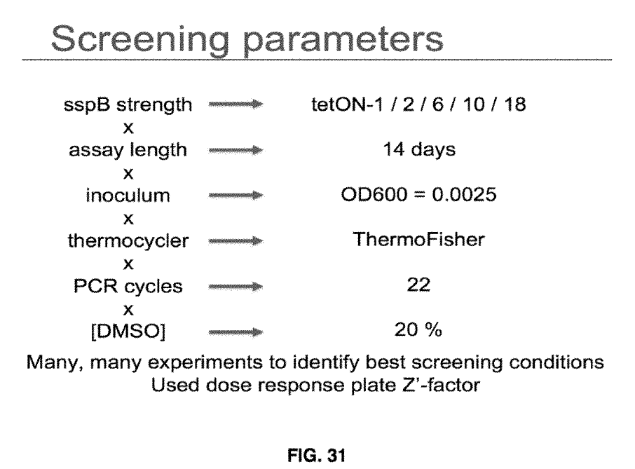

FIG. 32 provides a schematic of an example assay design in accordance with certain example embodiments.

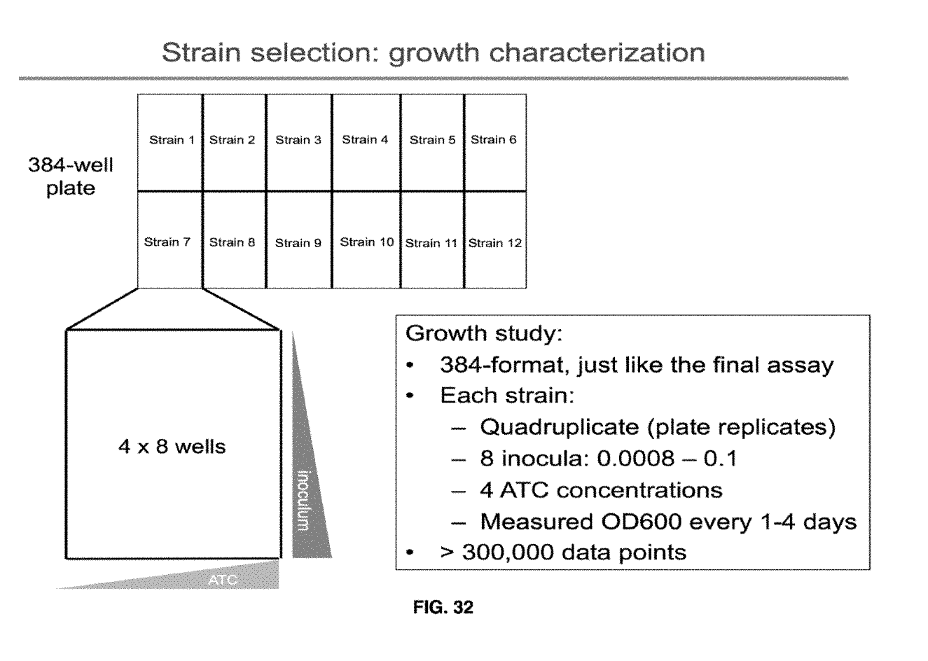

FIG. 33 is a graph showing H37Rv growth in a 384-well format.

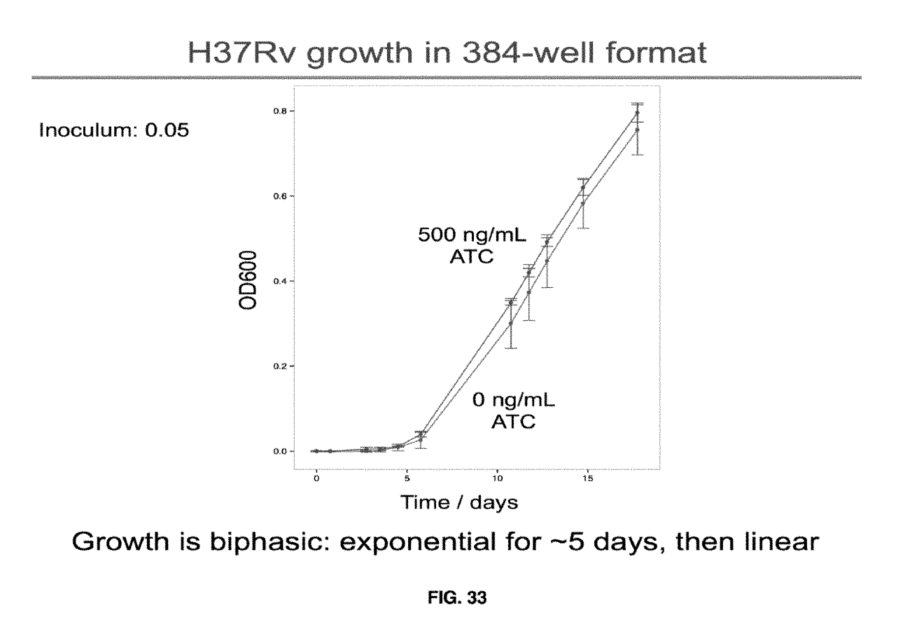

FIG. 34A is a graph showing strong gene promoter growth phenotype. FIG. 34B is a graph showing weak gene promoter growth phenotype.

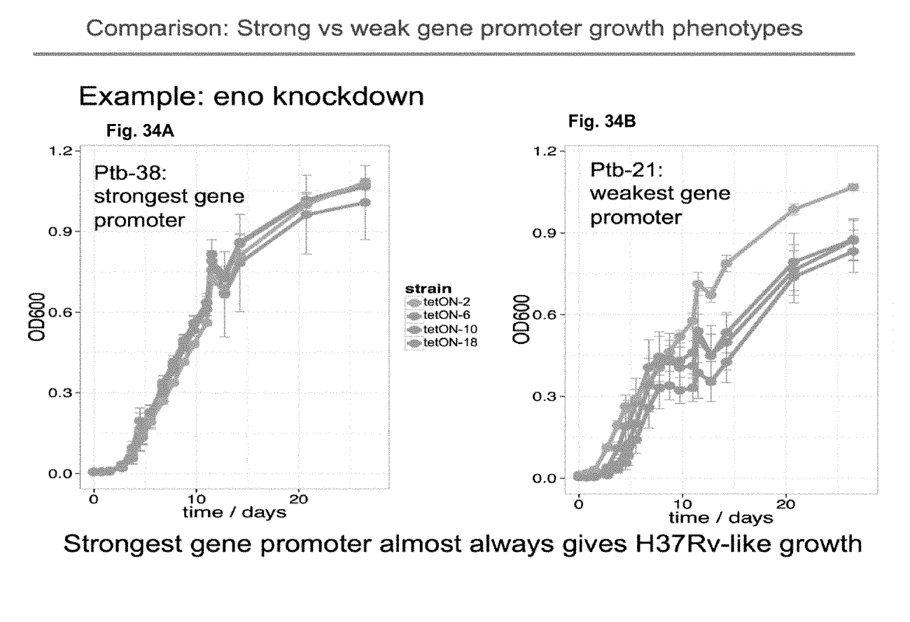

FIG. 35A shows positive control strain growth of alr knockdown. FIG. 35B shows positive control strain growth of dfrA knockdown.

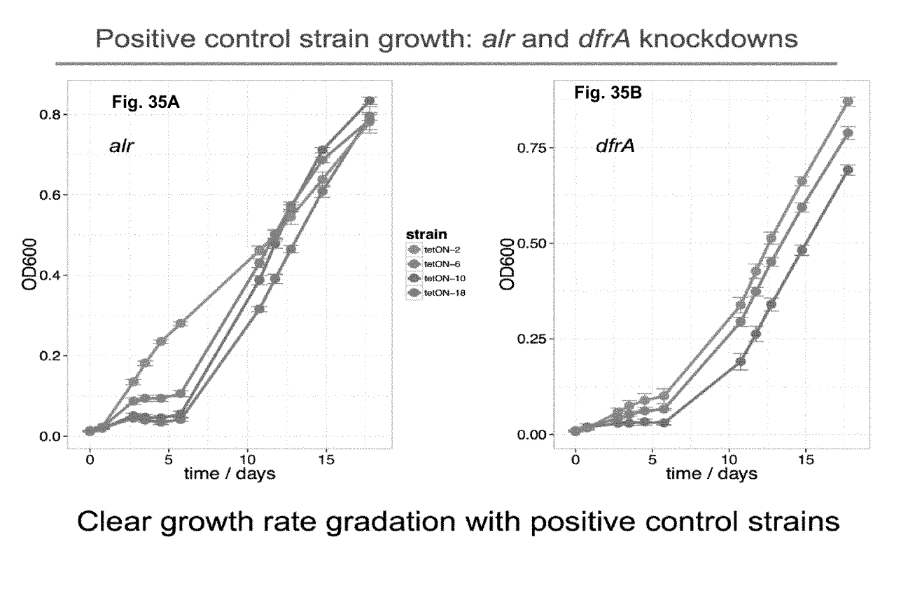

FIG. 36A shows type I H37Rv-like growth phenotype. FIG. 36B shows type II (significantly slowed) growth phenotype. FIG. 37C shows type III (no growth, then recovery) growth phenotype.

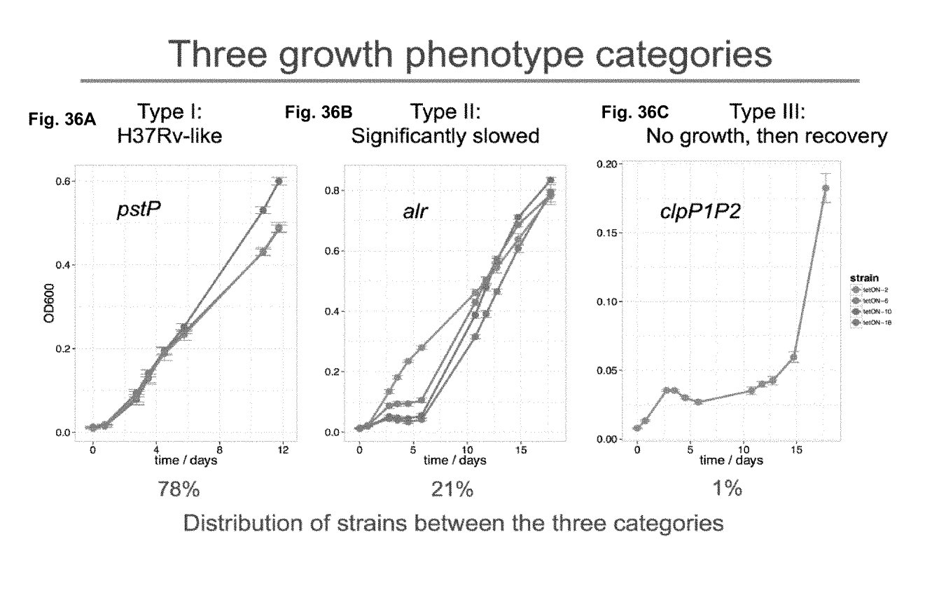

FIG. 37A shows dose response curve of cycloserine. FIG. 37B shows dose response curve of trimethoprim.

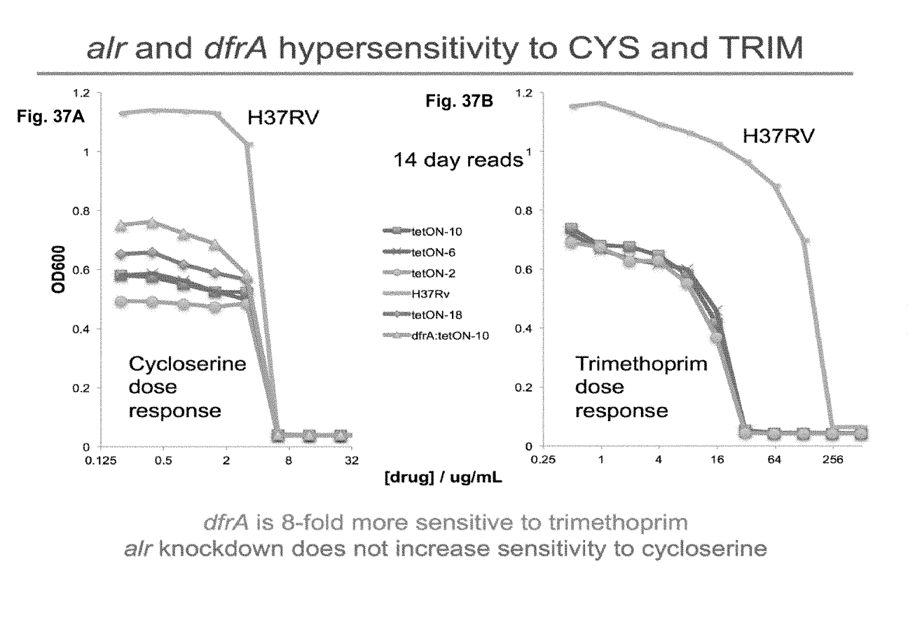

FIG. 38A shows trimethoprim dose-response of dfrA control strains of 0h after ATC removal. FIG. 38B shows trimethoprim dose-response of dfrA control strains of 22h after ATC removal.

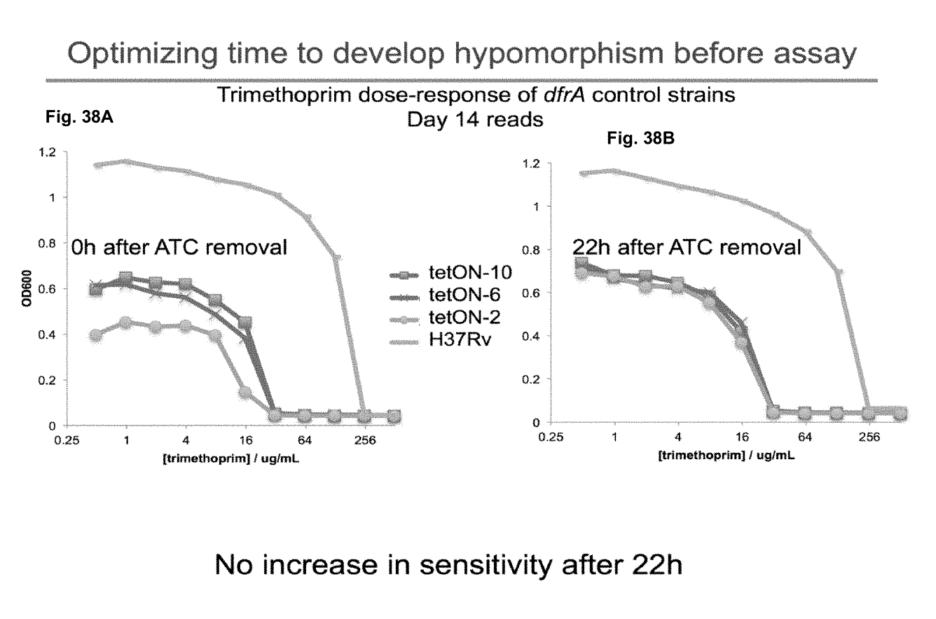

FIG. 39A shows trimethoprim dose-response of dfrA control strains at day 7 reads. FIG. 39B shows trimethoprim dose-response of dfrA control strains at day 14 reads. FIG. 39C shows trimethoprim dose-response of dfrA control strains at day 21 reads.

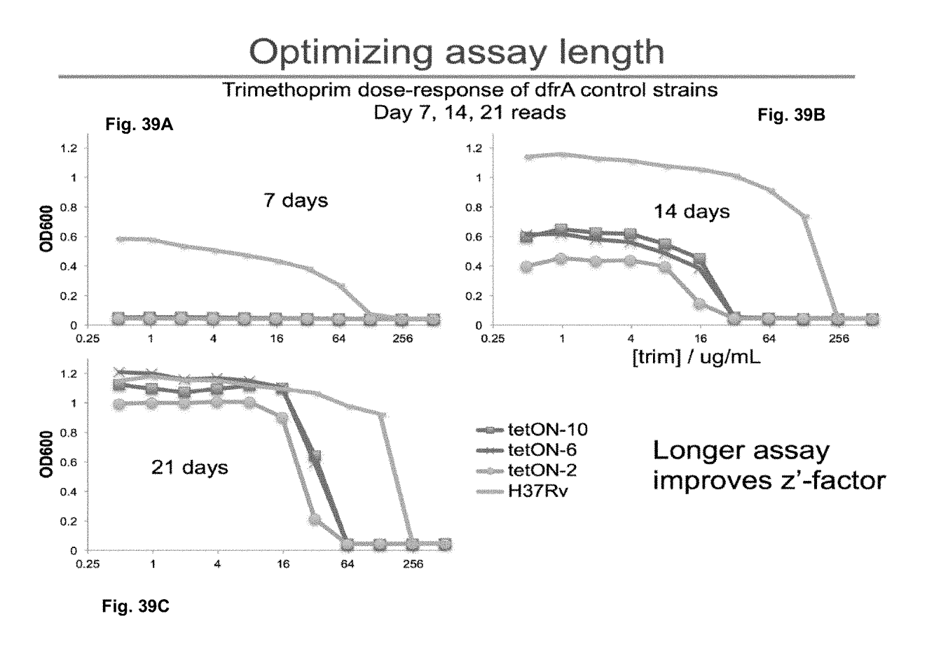

FIG. 40 provides a schematic of an example library construction in accordance with certain example embodiments.

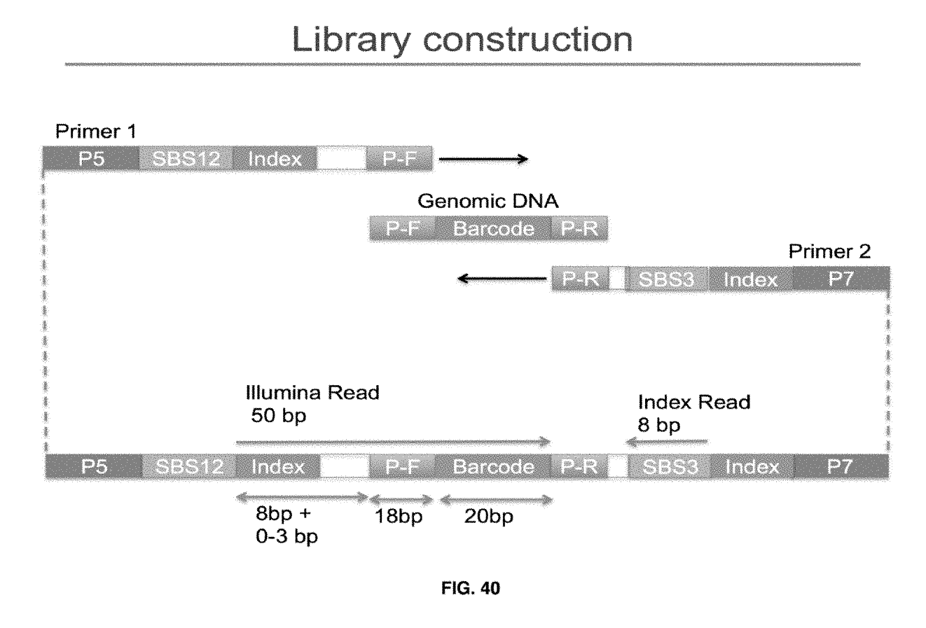

FIG. 41 provides a schematic of an example analysis of raw Illumina reads in accordance with certain example embodiments.

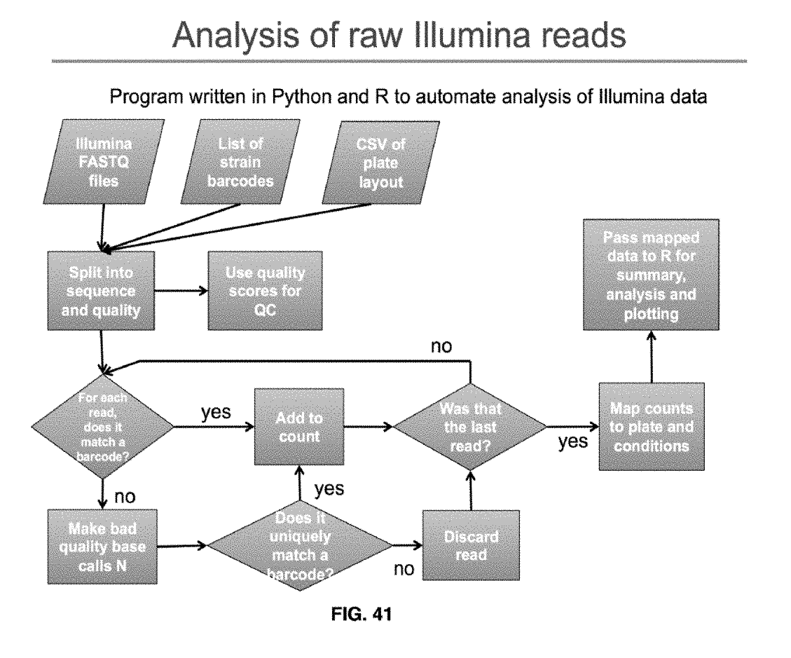

FIG. 42 is a graph showing the relationship between OD₆₀₀ readings and Illumina read counts.

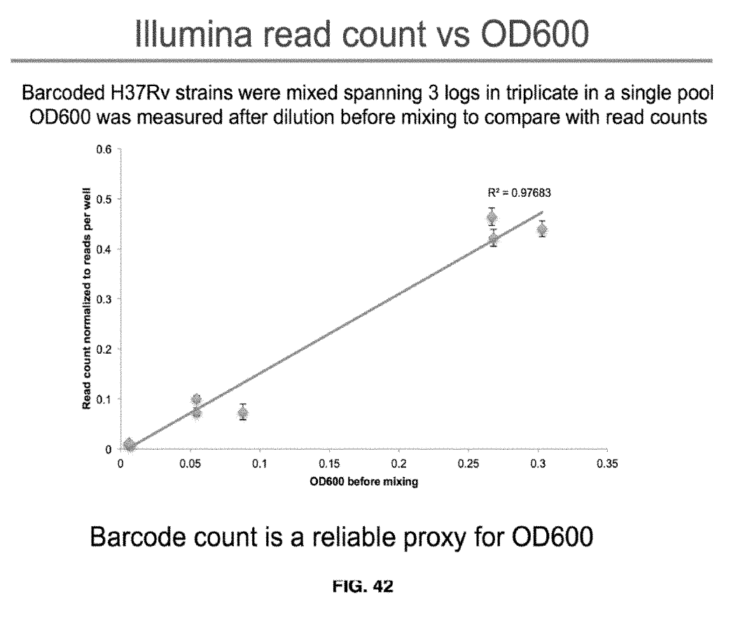

FIG. 43 shows that dfrA⁻ is hypersensitive to methotrexate. FIG. 43B shows that trpA⁻ is hypersensitive to 4592.

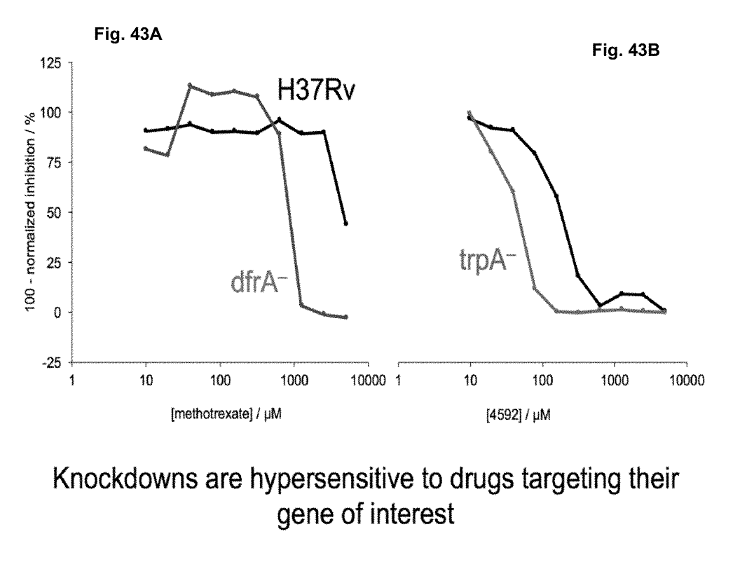

FIG. 44A shows log reads of dhfR. FIG. 44B shows log reads of folP.

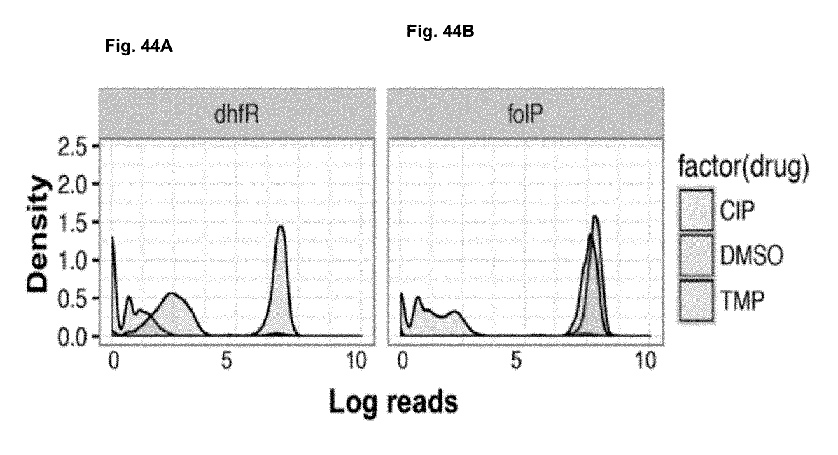

FIG. 45 is process flow chart of an example analysis method for analyzing sequencing reads.

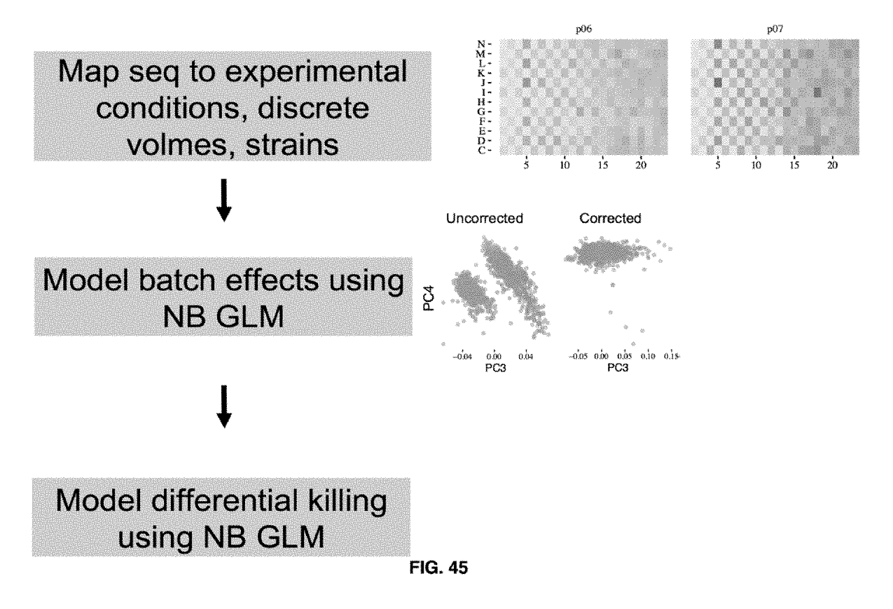

FIG. 46 is an example process low for identifying and developing new anti-microbial leads based on screening date obtain using the methods disclosed herein.

### DETAILED DESCRIPTION OF THE INVENTION

### Definitions

For purpose of this invention, “amplification” means any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. In particular, the isolated RNA can be subjected to a reverse transcription assay that is coupled with a quantitative polymerase chain reaction (RT-PCR) in order to quantify the expression level of a sequence associated with a signaling biochemical pathway.

As used herein, a “collection” of strains comprises a plurality of strains. The collection may comprise one or more strains from one or more genera. It may also comprise one or more strains from one or more species. It may also comprise one or more strains from one or more genera, and one or more strains from one or more species. It may also comprise strains from a single genus or it may also comprise strains from a single species. Micro-organisms are as described above. The collection of strains may comprise about at least 5, 10, 15, 20, 25, 30, 35, 40, 45, 50, 55, 60, 65, 75, 80, 90 or 100 strains.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, a “double-stranded nucleic acid molecule” comprises a nucleic acid molecule comprises two strands that are at least partially or fully complementary. The two strands may be the same length, they may be hybridized or in a denatured state. Examples include ds-DNA (double-stranded DNA). Said double-stranded molecule may be obtained as an amplification product, such as a PCR amplification product.

As used herein, a “discrete volume” refers to a defined volume or space that can be defined by properties that prevent and/or inhibit migration of microbial cells, for example a volume or space defined by physical properties such as walls, for example the walls of a well, tube, or a surface of a droplet, which may be permeable or semipermeable. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells on plates (such as wells in 6, 12, 24, 96, 384, 1536-well format), pipettes, or pipette tips among others.

As used herein, “expression of a genomic locus” or “gene expression” is the process by which information from a gene is used in the synthesis of a functional gene product. The products of gene expression are often proteins, but in non-protein coding genes such as rRNA genes or tRNA genes, the product is functional RNA. The process of gene expression is used by all known life—eukaryotes (including multicellular organisms), prokaryotes (bacteria and archaea) and viruses to generate functional products to survive. As used herein “expression” of a gene or nucleic acid encompasses not only cellular gene expression, but also the transcription and translation of nucleic acid(s) in cloning systems and in any other context. As used herein, “expression” also refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, the term “genomic locus” or “locus” (plural loci) is the specific location of a gene or DNA sequence on a chromosome. A “gene” refers to stretches of DNA or RNA that encode a polypeptide or an RNA chain that has functional role to play in an organism and hence is the molecular unit of heredity in living organisms. For the purpose of this invention it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“High-throughput screening” (HTS) refers to a process that uses a combination of modern robotics, data processing and control software, liquid handling devices, and/or sensitive detectors, to efficiently process a large amount of (e.g., thousands, hundreds of thousands, or millions of) samples in biochemical, genetic or pharmacological experiments, either in parallel or in sequence, within a reasonably short period of time (e.g., days). Preferably, the process is amenable to automation, such as robotic simultaneous handling of 96 samples, 384 samples, 1536 samples or more. A typical HTS robot tests up to 100,000 to a few hundred thousand compounds per day. The samples are often in small volumes, such as no more than 1 mL, 500 μl, 200 μl, 100 μl, 50 μl or less. Through this process, one can rapidly identify active compounds, small molecules, antibodies, proteins or polynucleotides which modulate a particular biomolecular/genetic pathway. The results of these experiments provide starting points for further drug design and for understanding the interaction or role of a particular biochemical process in biology. Thus “high-throughput screening” as used herein does not include handling large quantities of radioactive materials, slow and complicated operator-dependent screening steps, and/or prohibitively expensive reagent costs, etc.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

As used herein, “multiplex” refers to experimental conditions that allow parallel processing of samples, for example in partially or fully pooled formats. Multiplex processing may include pooled processing. Multiplex PCR may refer to multiple PCR reactions within the same reactor (e.g. a tube or a well). Multiplex PCR may refer to the use of multiple possible primer pairs, and/or multiple probes, and/or to the amplification of multiple targets within the same reaction. Multiplex may also refer to cell culture conditions, namely that a plurality of microorganism strains can be processed in co-culture. For example, it is possible to grow a collection of strains within the same well or plate. Multiplex may also refer to detection method, wherein detection may be carried out in pooled format, such as for example, detection from pooled PCR-amplified samples. Thus, according to embodiments of the invention, it is possible to pool the strains for growth (multiplex growth), lyse cells and PCR in plate (possible multiplex PCR), then pool the wells, then process for quantification (multiplex detection by sequencing).

As used herein, a “primer” refers to a single-stranded nucleic acid molecule. It generally comprises a stretch of nucleotides, such deoxyribonucleotides. Part of all of the primer sequence may be used for the purpose of nucleic acid amplification, such as by PCR (polymerase china reaction). This means that said primer comprises or consists of a sequence that may be used for ‘priming’ (target hybridization) for subsequent elongation with a polymerase enzyme. Total length of the primer may vary. Examples of total length include about 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80 nt. The part of the primer that may be used for priming in a PCR reaction may comprise or consist of a nucleotide stretch of about 15, 16, 17, 18, 19, 20, 21, 22, 23, 24, 25, 26, 27, 28, 29 or 30 nt.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, “probe” refers to any molecule capable of attaching and/or binding and/or hybridizing to a nucleic acid (i.e., for example, a barcode nucleic acid). For example, a capture probe may be an oligonucleotide or a primer. A probe may be a nucleic acid sequence, the nucleic acid being, for example, deoxyribonucleic acid (DNA), ribonucleic acid (RNA), peptide nucleic acid (PNA) or other non-naturally occurring nucleic acid. A collection of probes may comprise about at least 5, 10, 15, 20, 25, 30, 35, 40, 45, 50, 55, 60, 65, 75, 80, 90 or 100 probes.

As used herein, a “set” of items comprises a plurality of items. For example, a set of primers of the invention may comprise at least about 96, 192, 384, n×96 (with n being an integer) primers. The set of primers may include control primers such as positive and negative control primers. The set of primers may be configured for use with a given format for cell culture or cell growth, such as well plate formats, for example configured for use with 96 well-plates or 384-well plates.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y.

As used herein the term “variant” should be taken to mean the exhibition of qualities that differ, such as, but not limited to, genetic variations including SNPs, insertion deletion events, and the like.

### Overview

The present invention provides multiple whole-cell target-based screens. Labor, time and costs are advantageously reduced by performing the screens in multiplex. The invention generally relies on the generation of a collection of hypomorph strains, namely a series of cells that are knocked down for an essential gene. An “essential gene” may be determined using the techniques described further herein, and is a gene for which loss of function is not tolerated within a given microbial cell. Thus, microbial cells that are modified to exhibit reduced expression of such genes (hypomorphs) exhibit increased sensitivity to agents that target the essential genes. Thus, use of such hypomorphs may be used to screen agents for anti-microbial activity, while at the same time providing insight into the mechanism of action of such agents. In some embodiments, the hypomorphs strains may be genetically barcoded (unique polynucleotide strain identifier), so as to allow individual cell detection and counting by sequencing. In some embodiments, genetic strain barcode is engineered, while in other embodiments, the strain barcode is endogenous (e.g. 16S gene).

Essential genes may be identified using genome-wide negative selection technology, for example, one that combines transposon mutagenesis with massively parallel sequencing (Tn-seq (Gallagher, L. A., J. Shendure, and C. Manoil, Genome-Scale Identification of Resistance Functions in Pseudomonas aeruginosa Using Tn-seq. MBio, 2011. 2(1)) may be used to identify such genes. Importantly, in contrast to previous efforts which have largely identified essential genes in a single strain under lab growth conditions, the present invention defines essential genes across a set of different strains of P. aeruginosa (e.g. set of 20 strains) under a number of different growth conditions (e.g. 4) including urine, blood, rich media (LB), and minimal media (M9) to clearly define a core set of essential genes that represent possible gene targets across all clinical isolates under clinically relevant growth conditions. After generating and selecting for a transposon library on a particular growth condition, sequencing of transposon/chromosome junctions in surviving mutants leads to the identification of genes in which insertions are tolerated, while absent genes may be characterized as essential [Sassetti, C. M., D. H. Boyd, and E. J. Rubin, Comprehensive identification of conditionally essential genes in mycobacteria. Proc Natl Acad Sci USA, 2001. 98(22): p. 12712-7].

In certain example embodiments, the one or more essential genes are genes whose expression products are localized to the cytoplasam, cytoplasmic membrane, periplasm, outer membrane, or extracellular space. In certain example embodiments, the one or more essential proteins are localized to the outer membrane. In certain example embodiments, the function of the essential gene expression product is outer membrane protein assembly, cell structure/outer membrane integrity, outer membrane protein chaperone/assembly, LPS biosynthesis, rod-shape structural protein, endonuclease, folate synthesis, cell wall synthesis, or leucyl-tRNA synthesis. In certain example embodiments, the one or more essential genes are selected from the group consisting of ostA, opr86, oprL, lolB, omlA, lppL, surA, lolA, tolB, tolA, mreC, lptA, lptD, lptE, dhfR, folP, murA, gyrA, lpcX, leuS and gcp. In certain other example embodiments, the one or more essential proteins are selected from the group consisting of ccsX, ctaC, eno, fba, folB, glcB, marP, mdh, mshC, murG, nadE, pstP, sucD, topA, efpA, tpi, dlat, and mesa

Once identified, hypomorph strains may be generated by recombinantly modifying a microbial cell to exhibit reduced expression of the essential gene. A different hypomorph strain may have reduced expression of a unique essential gene or a unique combination of essential genes. As such, a collection of hypomorph stains may be produced that can be screened in multiplex to identify agents with anti-microbial activity and to identify the target of said agents.

In one example embodiment, the hypomorph cell is generated by recombinantly modifying a microbial cell such that the one or more essential genes are under the control of a weak promoter. The term “hypomorph strain” may be used interchangeably herein with “hypomorph cell,” and refers to a cell modified to have reduced expression of one or more essential genes. The hypomorph strain or cell may also be referred to a herein as “knock down.” As used herein a “weak promoter” refers to a promoter that results in lowered expression of a gene product compared to expression of the gene product under the control of an endogenous promoter of the modified cell. In certain example embodiments, the endogenous promoter may reduce expression by 5%, 6%, 7%, 8%, 9% 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98%, or 99% as compared to the endogenous promoter. Multiple hypomorph cells or strains may be generated encoding the same knock downed essential gene under the control of different promoters of differing strengths. In certain example embodiments, it may be useful to generate a promoter library with promoters of varying strengths, for example by varying the spacing between the promoter and the RNA polymerase binding site, in order to screen and select optimal assay conditions. In certain example embodiments, the weak promoters may be based on the promoters used to drive varying levels of GFP expression in E. coli and as described in Sauer et al.(Nucleic Acids Res, 2011. 39(3): p. 1131-41). Alternatively, other promoters may be generated by modifying the spacing between the RNA polymerase binding site of the promoters.

Example weak promoters are disclosed in the following table.

| | | Promoter | | |
|---|---|---|---|---|
| | | strength | Relative | |
| | | based on GFP | Strength to | |
| | New | synthesis rate | Consensus | Sequence (underlined is the RNA |
| Old Name | Name | per cell (au) | Promoter | Polymerase −35 and −10 binding sites |
| Pro1-15 | P1 | 0.242097537 | 0.3 | TTCTAGAGCACAGCTAACACCACGTCGTCCCTATCTGCTGCCCTAGG |
| | | | | TCTATGAGTGGTTGCTGGATAACTTTACGCATGCATAAGGCTCGGTA |
| | | | | TCTATATTCAGGGAGACCACAACGGTTTCCCTCTACAAATAATTTTG |
| | | | | TTTAACTTTTACTAGAGTCACACAGGAAAGTACTAG (SEQ ID |
| | | | | NO: 1048) |
| Pro1-14 | P2 | 3.545360341 | 4.2 | TTCTAGAGCACAGCTAACACCACGTCGTCCCTATCTGCTGCCCTAGG |
| | | | | TCTATGAGTGGTTGCTGGATAACTTTACGGTGCATAAGGCTCGGTAT |
| | | | | CTATATTCAGGGAGACCACAACGGTTTCCCTCTACAAATAATTTTGT |
| | | | | TTAACTTTTACTAGAGTCACACAGGAAAGTACTAG (SEQ ID NO: |
| | | | | 1049) |
| Pro1-16 | P3 | 4.923570091 | 5.8 | |
| Pro1-20 | | 4.988749061 | 5.9 | |
| ProD-14 | | 5.083296133 | 6.0 | |
| Pro1-19 | | 5.481493157 | 6.5 | |
| ProD-20 | | 5.569721063 | 6.6 | |
| Pro1-18 | P4 | 5.869609966 | 7.0 | |
| ProD-19 | P5 | 8.122773684 | 9.6 | |
| Pro2* | P6 | 11.56994591 | 13.7 | TTCTAGAGCACAGCTAACACCACGTCGTCCCTATCTGCTGCCCTAGG |
| | | | | TCTATGAGTGGTTGCTGGATAACGCGGTGGGCATGCATAAGGCTCGT |
| | | | | ATAATATATTCAGGGAGACCACAACGGTTTCCCTCTACAAATAATTT |
| | | | | TGTTTAACTTTTACTAGAGTCACACAGGAAAGTACTAG (SEQ ID |
| | | | | NO: 1050) |
| Pro1* | P7 | 19.95581074 | 23.7 | TTCTAGAGCACAGCTAACACCACGTCGTCCCTATCTGCTGCCCTAGG |
| | | | | TCTATGAGTGGTTGCTGGATAACTTTACGGGCATGCATAAGGCTCGG |
| | | | | TATCTATATTCAGGGAGACCACAACGGTTTCCCTCTACAAATAATTT |
| | | | | TGTTTAACTTTTACTAGAGTCACACAGGAAAGTACTAG (SEQ ID |
| | | | | NO: 1051) |
| Pro5* | P8 | 26.66074905 | 31.6 | TTCTAGAGCACAGCTAACACCACGTCGTCCCTATCTGCTGCCCTAGG |
| | | | | TCTATGAGTGGTTGCTGGATAACTTTACGGGCATGCATAAGGCTCGT |
| | | | | AGGATATATTCAGGGAGACCACAACGGTTTCCCTCTACAAATAATTT |
| | | | | TGTTTAACTTTTACTAGAGTCACACAGGAAAGTACTAG (SEQ ID |
| | | | | NO: 1052) |
| ProB* | P9 | 32.80908782 | 38.9 | TTCTAGAGCACAGCTAACACCACGTCGTCCCTATCTGCTGCCCTAGG |
| | | | | TCTATGAGTGGTTGCTGGATAACTTTACGGGCATGCATAAGGCTCGT |
| | | | | AATATATATTCAGGGAGACCACAACGGTTTCCCTCTACAAATAATTT |
| | | | | TGTTTAACTTTTACTAGAGTCACACAGGAAAGTACTAG (SEQ ID |
| | | | | NO: 1053) |
| ProD-16 | | 32.99877981 | 39.1 | |
| ProA* | P10 | 34.35395685 | 40.7 | |
| ProD-15 | | 36.75954452 | 43.6 | |
| ProD-18 | | 37.17760884 | 44.1 | |
| Pro6* | P11 | 44.0145159 | 52.2 | TTCTAGAGCACAGCTAACACCACGTCGTCCCTATCTGCTGCCCTAGG |
| | | | | TCTATGAGTGGTTGCTGGATAACTTTACGGGCATGCATAAGGCTCGT |
| | | | | AAAATATATTCAGGGAGACCACAACGGTTTCCCTCTACAAATAATTT |
| | | | | TGTTTAACTTTTACTAGAGTCACACAGGAAAGTACTAG (SEQ ID |
| | | | | NO: 1054) |
| ProC* | P12 | 54.91594599 | 65.1 | TTCTAGAGCACAGCTAACACCACGTCGTCCCTATCTGCTGCCCTAGG |
| | | | | TCTATGAGTGGTTGCTGGATAACTTTACGGGCATGCATAAGGCTCGT |
| | | | | ATGATATATTCAGGGAGACCACAACGGTTTCCCTCTACAAATAATTT |
| | | | | TGTTTAACTTTTACTAGAGTCACACAGGAAAGTACTAG (SEQ ID |
| | | | | NO: 1055) |
| ProD* | Pconsensus | 84.36853934 | 100.0 | TTCTAGAGCACAGCTAACACCACGTCGTCCCTATCTGCTGCCCTAGG |
| | | | | TCTATGAGTGGTTGCTGGATAACTTTACGGGCATGCATAAGGCTCGT |
| | | | | ATAATATATTCAGGGAGACCACAACGGTTTCCCTCTACAAATAATTT |
| | | | | TGTTTAACTTTTACTAGAGTCACACAGGAAAGTACTAG (SEQ ID |
| | | | | NO: 1056) |

*Sauer nomenclature.(Nucleic Acids Res, 2011. 39(3): p. 1131-41).

In certain other example embodiments, the hypomorph cell is generated by modifying one or more essential genes to encode a protein degradation tag that is appended to the expressed protein product, thus marking the protein for degradation by an endogenous degradation protein or system. The degradation tag may be any tag that marks the expressed protein and may depend on the species of microbial cell and the type of endogenous protein degradation system expressed in said microbial cell. In certain example embodiments, the degradation tag is a clp-protease tag. In certain example embodiments, the clp-protease tag is a DAS4+ tag. In certain example embodiments, the hypomorph may be further modified to express a protease adapter protein that facilitates recognition of degradation tags by a protease or protease complex, shuttles proteins expressing the degradation tag to a protease or protease complex, or activates a protease or protease complex. The shuttle protein may be under the control of a second promoter. The second promoter may be inducible. In certain example embodiments, the inducible promoter is a tetOn on tetOff promoter. In certain example embodiments, the protease adapter protein gene is sspB.

The hypomorph cells disclosed herein are further modified to include a strain specific nucleic aid identifier or barcode. A nucleic acid identifier or barcode may be an artificial sequence have a length of at least 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. Each hypomorph is assigned a unique barcode that identifies the hypomorph from other hypomorph strains and provides information on the species and the essential gene or combination of essential genes that are knocked down in a given strain. The strain specific nucleic acid identifier may further comprise a first primer binding site and a second primer binding site. The first and second primer binding sites provide two regions that hybridize to a corresponding set of amplification primers that may be used to amplify the strain specific nucleic acid identifier. The resulting amplicons may then be sequenced. The number of reads of a given hypomorph's strain specific nucleic acid identifier is tied to the amount of a that hypomorph in a given sample. As demonstrated further below, sequencing reads function as a proxy for OD₆₀₀ values and provide a measure of the abundance of a given hypomorph in a sample. Thus, the relative amounts of a given hypomorph in a sample or volume may be determined in the methods further disclosed herein via sequencing.

In certain aspects, the embodiments disclosed herein are directed to the nucleic acid primers used to amplify the above strain specific nucleic acid identifiers. In certain example embodiments, the first primer and second primer binding site used in the strain specific nucleic acid identifiers are the same. Thus, the target binding site for the first and second primers may be the same for all hypomorph strains. The first and second primers, however, may further include additional sequences that are incorporated into amplicons during amplification reactions using the first and second primers. In certain example embodiments, one of the primers may include an origin specific barcode. The origin specific barcode is used to identify a discrete volume from which a given hypomorph sequencing read originated. Thus, all primer pairs delivered to a given sample or discrete volume will have the same origin specific barcode. In this way, all sequencing reads originating from the same sample or discrete volume may be identified. The origin specific barcode may be included on the first primer or the second primer. In certain example embodiments, the first or second primer may further include a experimental condition specific barcode. This barcode is uniquely assigned to the experimental conditions being tested in a given sample or discrete volume. Samples may be tested in multiplicate so each sample receiving the same experimental conditions will receive primers encoding different origin specific barcodes but the same experimental condition barcodes. Collectively, the strain specific barcodes, origin specific barcodes, and experimental condition barcodes can be used to identify, via the sequencing of amplicons, to determine the identity and relative amounts of all hypomorphs originating from the same sample or discrete volume, and the experimental conditions tested in that particular sample or discrete volume. In certain example embodiments, the first primer and second primer may further comprise a first primer sequencing primer binding site and/or first sequencing adapter and a second primer sequencing binding site and/or second sequencing adapter respectively. Accordingly, the resulting amplicons will incorporate sequencing primer binding sites and sequencing adapters. In certain other example embodiments, the sequencing primer binding sites and sequencing adapter may be appended to the amplicons via ligation after amplification.

Microbial cells that may be used to generate hypomorphs include bacterial cells, fungal cells, mycological cells, protozoal cells, nematode cells, trematode cells, or cestode cells. In certain example embodiments, the microbial cells are bacterial cells. The bacterial cells may include, but are not limited to, Bordetella, Bacillis, Borrelia, Brucella, Campylobacter, Chlamydia, Clamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, and Yersinia. In certain example embodiments, the bacterial cells are Eschericia coli, Klebsiella pneumoniae, Pseudomonas aeruginosa, Staphylococcus aureus, Acinetobacter baumannii, Candida albicans, Enterobacter cloacae, Enterococcus faecalis, Enterococcus faecium, Proteus mirabalis, Streptococcus agalactiae, and Stenotrophomonas maltophila. In certain other example embodiments, the bacterial cell is Pseudomonas aeruginosa. In certain other example embodiments, the bacterial cell is a Mycobacterium. The Mycobacterium may include, but is not limited to, M. tuberculosis, M. avium-intracellulare, M. kansasii, M. fortuitum, M. chelonae, M. leprae, M. africanum, M. microti, M. avium paratuberculosis, M. intracellulare, M. scrofulaceum, M. xenopi, M. marinum, and M. ulcerans. In one example embodiment, the microbial cell is M. tuberculosis.

In certain example embodiments, the microbial cell is a fungal cell. The fungal cells used may include, but are not limited to, Candida, Aspergillus, Cryptococcus, Histoplasma, Pneumocystis, and Stachybotrys. In certain example embodiments, the microbial cell may be a protozoa including, but not limited to, Entamoeba histolytica, Dientamoeba fragilis, Giardia lamblia, Trichomonas vaginalis, Balantidium coli, Naegleria fowleri, Acanthamoeba, Plasmodium falciparium, P. malariae, P. ovale, P. vivax, Isospora belli, Cryptosporidium parvum, Cyclospora cayetanensis, Enterocytozoon nieneusi, Babesia microti, Toxoplasma gondii, L. donovani, L. tropica, L. braziliensis, Trypanosoma gambiense, T rhodesiense, T cruzi, and Penumocystis jiroveci. In certain example embodiments, the microbial cell may be a nematode including, but not limited to, Enterobius vermicularis, Ascaris lumbricoides, Toxocara canis, Toxocara cati, Baylisascaris procyonis, Ancylostoma duodenale, Necator americnaus, Strongyloides stercoralis, Ancylostoma braziliense, Trichuris trichiura, Trichinella spiralis, Wuchereria bancrofti, Brugia malaya, Loa loa, Onchocerca volvulus, Dracunculus medinensis, Capillaria phihppinensis. In certain example embodiments, the microbial cell may be a trematode including, but not limited to, Fasciolopsis buski, Fasciola hepatica, Opisthorchis sinensis, Paragonimus westermani, P. kellicotti, Schistosoma mansoni, S. japonicum, and S. haematobium. In certain example embodiments, the microbial cell may be a cestode including, but not limited to, Taenia solium, T saginata, Diphyllobothrium latum, Dipylidium caninum, Echinococcus granulosis, E. multilocularis, and Hymenolepis nana.

The hypomorph cells disclosed herein may be used to screen a series of experimental conditions. As described above, a hypomorph strain will exhibit hypersensitivity to a set of experimental conditions that target the essential genes or combination of essential genes knocked down in that hypomorph. Therefore, assessing the amount of multiple hypomorph strains exposed to the same experimental conditions can help identify potential targets for further validation, for example, as anti-microbial agents.

Each hypomorph strain is cultured in an individual discrete volume. In certain example embodiments, the discrete volume is the well of a microplate. Each well is then exposed to a different set of experimental conditions. The experimental conditions may comprise exposure to different test agents, combinations of test agents, or different concentrations of test agents or combinations of test agents. For example, the methods disclosed herein may be used to screen a chemical library for anti-microbial activity. The experimental conditions may further comprise assessment under different physical growth conditions such as different growth media, different pH, different temperatures, different atmospheric pressures, different atmospheric 02 concentrations, different atmospheric CO₂ concentrations, or a combination thereof.

After a sufficient time period, and as dictated by the experimental conditions to be assessed, the cells are lysed and the strain specific barcodes are amplified using the primers disclosed herein. As noted above, the primer pairs delivered to each volume will comprise the appropriate origin specific and experimental condition specific conditions barcodes for each discrete volume. The resulting amplicons are then sequenced, for example, using next generation sequencing.

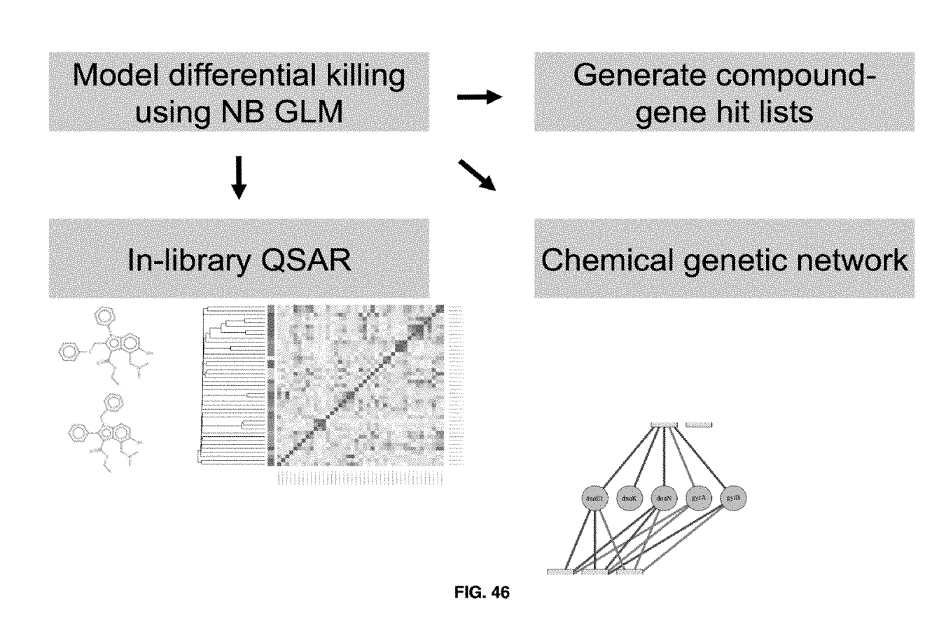

The sequencing reads are then mapped to the corresponding experimental conditions, discrete volumes, and hypomorph strains. Analysis may be conducted on the resulting sequencing read data to determine the amount of different hypomorphs in each discrete volume. If a hypomorph is missing or demonstrates less abundance than other hypomorph strains or a control condition, this then indicates both potential anti-microbial activity as well as identifying the knockdown essential genes as the potential target for exhibiting the anti-microbial effect. An example process flow for analyzing the sequencing read data is shown in FIG. 46. In certain example embodiments, the sequencing count data may be analyzed as if a negative binomial marginal distribution (NB) and a generalized linear model (GLM).

The present application also may be utilized in conjunction with other assays that detect and identify bacteria and fungi (see, e.g., the LightCycler® SeptiFast Test MGRADE assay kit; and Bravo et al., International Society for Infectious Diseases, May 2011 Volume 15, Issue 5, Pages e326-e331).

Advantageously according to the invention, the detection may be carried out by nucleic acid sequencing, preferably quantitative or semi-quantitative nucleic acid sequencing. This allows to determine the presence (or absence) of a given nucleic acid sequence in a pool of nucleic acids. For example, one may determine the presence of a double-stranded nucleic acid molecule as per the invention, by determining its nucleotide sequence. Within said determined sequence, it is then possible to identify stretches of nucleotides of interest. For example, within a given double-stranded nucleic acid molecule, sequencing allows to identify presence of a given unique polynucleotide identifier (thus allowing the identification of the corresponding micro-organism strain), and/or presence of a given polynucleotide sequence indicative of given growth conditions, such as a first polynucleotide or 5′-polynucleotide sequence identifying a culture plate or a polynucleotide or 5′-polynucleotide sequence identifying a well within a plate (thus allowing the identification of the corresponding growth conditions). As a result, detection may advantageously allow, in a multiplex fashion, to determine the presence or absence of a given micro-organism strain that was cultured in given growth conditions.

Embodiments of the invention include sequences (both polynucleotide or polypeptide) which may comprise homologous substitution (substitution and replacement are both used herein to mean the interchange of an existing amino acid residue or nucleotide, with an alternative residue or nucleotide) that may occur i.e., like-for-like substitution in the case of amino acids such as basic for basic, acidic for acidic, polar for polar, etc. Non-homologous substitution may also occur i.e., from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Hybridization can be performed under conditions of various stringency. Suitable hybridization conditions for the practice of the present invention are such that the recognition interaction between the probe and sequences associated with a signaling biochemical pathway is both sufficiently specific and sufficiently stable. Conditions that increase the stringency of a hybridization reaction are widely known and published in the art. See, for example, (Sambrook, et al., (1989); Nonradioactive In Situ Hybridization Application Manual, Boehringer Mannheim, second edition). The hybridization assay can be formed using probes immobilized on any solid support, including, but are not limited to, nitrocellulose, glass, silicon, and a variety of gene arrays. A preferred hybridization assay is conducted on high-density gene chips as described in U.S. Pat. No. 5,445,934.

Examples of the labeling substance which may be employed include labeling substances known to those skilled in the art, such as fluorescent dyes, enzymes, coenzymes, chemiluminescent substances, and radioactive substances. Specific examples include radioisotopes (e.g., 32P, 14C, 125I, 3H, and 131I), fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a labeling substance, preferably, after addition of a biotin-labeled antibody, streptavidin bound to an enzyme (e.g., peroxidase) is further added.

Advantageously, the label is a fluorescent label. Examples of fluorescent labels include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-i sothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™. Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine.

The fluorescent label may be a fluorescent protein, such as blue fluorescent protein, cyan fluorescent protein, green fluorescent protein, red fluorescent protein, yellow fluorescent protein or any photoconvertible protein. Colormetric labeling, bioluminescent labeling and/or chemiluminescent labeling may further accomplish labeling. Labeling further may include energy transfer between molecules in the hybridization complex by perturbation analysis, quenching, or electron transport between donor and acceptor molecules, the latter of which may be facilitated by double stranded match hybridization complexes. The fluorescent label may be a perylene or a terrylen. In the alternative, the fluorescent label may be a fluorescent bar code.

In an advantageous embodiment, the label may be light sensitive, wherein the label is light-activated and/or light cleaves the one or more linkers to release the molecular cargo. The light-activated molecular cargo may be a major light-harvesting complex (LHCII). In another embodiment, the fluorescent label may induce free radical formation.

In an advantageous embodiment, agents may be uniquely labeled in a dynamic manner (see, e.g., international patent application serial no. PCT/US2013/61182 filed Sep. 23, 2012). The unique labels are, at least in part, nucleic acid in nature, and may be generated by sequentially attaching two or more detectable oligonucleotide tags to each other and each unique label may be associated with a separate agent. A detectable oligonucleotide tag may be an oligonucleotide that may be detected by sequencing of its nucleotide sequence and/or by detecting non-nucleic acid detectable moieties to which it may be attached.

The oligonucleotide tags may be detectable by virtue of their nucleotide sequence, or by virtue of a non-nucleic acid detectable moiety that is attached to the oligonucleotide such as, but not limited to, a fluorophore, or by virtue of a combination of their nucleotide sequence and the nonnucleic acid detectable moiety.

In some embodiments, a detectable oligonucleotide tag may comprise one or more nonoligonucleotide detectable moieties. Examples of detectable moieties may include, but are not limited to, fluorophores, microparticles including quantum dots (Empodocles, et al., Nature 399:126-130, 1999), gold nanoparticles (Reichert et al., Anal. Chem. 72:6025-6029, 2000), biotin, DNP (dinitrophenyl), fucose, digoxigenin, haptens, and other detectable moieties known to those skilled in the art. In some embodiments, the detectable moieties may be quantum dots. Methods for detecting such moieties are described herein and/or are known in the art.

Thus, detectable oligonucleotide tags may be, but are not limited to, oligonucleotides which may comprise unique nucleotide sequences, oligonucleotides which may comprise detectable moieties, and oligonucleotides which may comprise both unique nucleotide sequences and detectable moieties.

A unique label may be produced by sequentially attaching two or more detectable oligonucleotide tags to each other. The detectable tags may be present or provided in a plurality of detectable tags. The same or a different plurality of tags may be used as the source of each detectable tag may be part of a unique label. In other words, a plurality of tags may be subdivided into subsets and single subsets may be used as the source for each tag.

In some embodiments, a detectable oligonucleotide tag may comprise one or more non-oligonucleotide detectable moieties. Examples of detectable moieties include, but are not limited to, fluorophores, microparticles including quantum dots (Empodocles, et al., Nature 399:126-130, 1999), gold nanoparticles (Reichert et al., Anal. Chem. 72:6025-6029, 2000), biotin, DNP (dinitrophenyl), fucose, digoxigenin, haptens, and other detectable moieties known to those skilled in the art. In some embodiments, the detectable moieties are quantum dots. Methods for detecting such moieties are described herein and/or are known in the art.

A unique nucleotide sequence may be a nucleotide sequence that is different (and thus distinguishable) from the sequence of each detectable oligonucleotide tag in a plurality of detectable oligonucleotide tags. A unique nucleotide sequence may also be a nucleotide sequence that is different (and thus distinguishable) from the sequence of each detectable oligonucleotide tag in a first plurality of detectable oligonucleotide tags but identical to the sequence of at least one detectable oligonucleotide tag in a second plurality of detectable oligonucleotide tags. A unique sequence may differ from other sequences by multiple bases (or base pairs). The multiple bases may be contiguous or non-contiguous. Methods for obtaining nucleotide sequences (e.g., sequencing methods) are described herein and/or are known in the art.

In some embodiments, detectable oligonucleotide tags comprise one or more of a ligation sequence, a priming sequence, a capture sequence, and a unique sequence (optionally referred to herein as an index sequence). A ligation sequence is a sequence complementary to a second nucleotide sequence which allows for ligation of the detectable oligonucleotide tag to another entity which may comprise the second nucleotide sequence, e.g., another detectable oligonucleotide tag or an oligonucleotide adapter. A priming sequence is a sequence complementary to a primer, e.g., an oligonucleotide primer used for an amplification reaction such as, but not limited to, PCR. A capture sequence is a sequence capable of being bound by a capture entity. A capture entity may be an oligonucleotide which may comprise a nucleotide sequence complementary to a capture sequence, e.g. a second detectable oligonucleotide tag. A capture entity may also be any other entity capable of binding to the capture sequence, e.g. an antibody, hapten or peptide. An index sequence is a sequence which may comprise a unique nucleotide sequence and/or a detectable moiety as described above.

The present invention is particularly useful for discovery methods. For example, growth conditions may include the presence of a given candidate compound, such as a candidate agent in a screen for antibacterial agents. The methods of the invention allow to determine the presence of a given strain in given growth conditions, for a multiplicity of strains and a multiplicity of growth conditions. The invention thus makes it possible to screen a multiplicity of candidate compounds, at varying concentrations, on a plurality of micro-organism strains. The method is multiplexed, so that throughput is high: it is made possible to screen a high number of strains, e.g. more than 20, 50, 75, 100, 200, 300, 400 or 500 strains. Said strains may be tested against a high number of candidate compounds, such as more than 1,000, 2,000, 5,000, 10,000, 15,000, 20,000, 25,000, 30,000, 40,000 or 50,000 candidate compounds. Compounds may be tested at carrying concentrations. For example, it is possible to establish dose-response profiles for a given compound. The screens may be validated using known antibacterial agents (positive controls) and/or unmutated strains. Controls may be used for inhibition or specificity (e.g. respectively rifampin and trimethoprim for P. aeruginosa). The invention also allows the identification of candidate compounds that are either specific or with broader spectrum activity.

The methods of the inventions may be conducted in duplicate, triplicate or multi-plicate, etc. This may increase robustness of the methods or confirm reproducibility, for example by detecting experimental errors, etc.

Detection of the gene expression level can be conducted in real time in an amplification assay. In one aspect, the amplified products can be directly visualized with fluorescent DNA-binding agents including, but not limited to, DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dye suitable for this application include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

In another aspect, other fluorescent labels, such as sequence specific probes, can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan® probes) resulting in increased specificity and sensitivity. Methods for performing probe-based quantitative amplification are well established in the art and are taught in U.S. Pat. No. 5,210,015.

Sequencing may be performed on any high-throughput platform with read-length (either single- or paired-end) sufficient to cover both template and cross-linking event UIDs. Methods of sequencing oligonucleotides and nucleic acids are well known in the art (see, e.g., WO93/23564, WO98/28440 and WO98/13523; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem.136:192 (1984); and Elgen and Rigler, Proc. Natl. Acad. Sci. USA 91(13):5740 (1994), all of which are expressly incorporated by reference).

The sample may be a biological sample, for example a blood, buccal, cell, cerebrospinal fluid, mucus, saliva, semen, tissue, tumor, feces, urine, or vaginal sample. It may be obtained from an animal, a plant or a fungus. The animal may be a mammal. The mammal may be a primate. The primate may be a human. In other embodiments, the sample may be an environmental sample, such as water or soil.

The present invention also relates to methods of high throughput screening HTS of a compound diversity oriented synthesis library using MTEP against the mixture of pooled screening strains. Advantageously, the compound libraries of the Broad Institute are contemplated for screening (https://www.broadinstitute.org/scientific-community/science/programs/csoft/therapeutics-platform/compound-libraries). Advantageously, the compounds may have antibacterial properties. The compounds may be or resemble β-Lactam antibiotics: penicillin G, penicillin V, cloxacilliin, dicloxacillin, methicillin, nafcillin, oxacillin, ampicillin, amoxicillin, bacampicillin, azlocillin, carbenicillin, mezlocillin, piperacillin, and ticarcillin; Aminoglycosides: amikacin, gentamicin, kanamycin, neomycin, netilmicin, and streptomycin; Tobramycin Macrolides: azithromycin, clarithromycin erythromycin, lincomycin, and clindamycin; Tetracyclines: demeclocycline, doxycycline, minocycline, oxytetracycline, tetracycline quinolones: cinoxacin, nalidixic acid Fluoroquinolones: ciprofloxacin, enoxacin, grepafloxacin, levofloxacin, lomefloxacin, norfloxacin, ofloxacin, and sparfloxacin; Trovafloxicin polypeptides: bacitracin, colistin, and polymyxin B; Sulfonamides: sulfisoxazole, sulfamethoxazole, sulfadiazine, sulfamethizole, and sulfacetamide; or Miscellaneous Antibacterial Agents: trimethoprim, sulfamethazole, chloramphenicol, vancomycin, metronidazole, quinupristin, dalfopristin, rifampin, spectinomycin, nitrorurantoin.

As used herein, a “kit” refers to one or more elements as described herein, that may be accompanied by instructions or directions for use.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds. (1987)).

The present invention also relates to a computer system involved in carrying out the methods of the invention relating to both computations and sequencing.

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present invention can be transmitted over such networks or connections (or any other suitable means for transmitting information, including, but not limited to, mailing a physical report, such as a print-out) for reception and/or for review by a receiver. The receiver can be, but is not limited to, an individual, or electronic system (e.g. one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the invention. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the invention, the server computer handles all of the database functionality. The client computer can have software that handles all the front-end data management and can also receive data input from users.

A machine readable medium which may comprise computer-executable code may take many forms, including, but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include, for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

Although the present invention and its advantages have been described in detail, it should be understood that various changes, substitutions and alterations can be made herein without departing from the spirit and scope of the invention as defined in the appended claims.

The invention may be further understood with reference to the following set of numbered clauses:

1. A set of primers configured for multiplex high-resolution detection of micro-organism strains amongst a strain collection,
wherein each micro-organism strain comprises a unique polynucleotide identifier,
wherein each primer comprises: a first polynucleotide sequence indicative of experimental conditions, and a second polynucleotide sequence configured for the amplification and subsequent detection of said unique polynucleotide identifier.

2. The set of primers of clause 1, wherein the unique polynucleotide identifier is configured for identification of strain or species.

3. The set of primers of clause 1 or 2, wherein the unique polynucleotide identifier is configured for identification of strain by nucleic acid sequencing.

4. The set of primers of any one of clauses 1-3, wherein the unique polynucleotide identifier is flanked by upstream and downstream respective flanking sequences.

5. The set of primers of any one of clauses 1-4, wherein the multiplex high-resolution detection comprises absolute or relative quantification.

6. The set of primers of any one of clauses 1-5, wherein the first polynucleotide sequence comprises a 5′-polynucleotide sequence.

7. The set of primers of any one of clauses 1-6, wherein the second polynucleotide sequence comprises a 3′-polynucleotide sequence.

8. The set of primers of any one of clauses 1-7, wherein experimental conditions comprise growth conditions.

9. The set of primers of any one of clauses 1-8, wherein the first polynucleotide sequence identifies a culture plate or a well within a culture plate, the culture plate or the well within the culture plate being indicative of predetermined experimental conditions.

10. The set of primers of any one of clauses 1-9, wherein the set of primers comprises: a first subset of primers with a first polynucleotide sequence identifying a culture plate and a second subset of primers with a first polynucleotide sequence identifying a well within a plate.

11. The set of primers of any one of clauses 1-10, wherein the set of primers comprises one or more pairs of primers.

12. The pair of primers of clause 11, wherein each pair comprises: a primer with a first polynucleotide sequence identifying a culture plate adjacent to a second polynucleotide sequence which is the upstream flanking sequence; and a primer with a first polynucleotide sequence identifying a well within a culture plate adjacent to a second polynucleotide sequence which is the downstream flanking sequence.

13. The pair of primers of clause 11, wherein each pair comprises a primer with a first polynucleotide sequence identifying a culture plate adjacent to a second polynucleotide sequence which is the downstream flanking sequence; and a primer with a first polynucleotide sequence identifying a well within a culture plate adjacent to a second polynucleotide sequence which is the upstream flanking sequence.

14. The set of primers of any one of clauses 1-11, wherein the set of primers comprises a first subset of primers with a first polynucleotide sequence identifying a culture plate adjacent to a second polynucleotide sequence which is the downstream flanking sequence; and a second subset of primers with a first polynucleotide sequence identifying a well within a culture plate adjacent to a second polynucleotide sequence which is the upstream flanking sequence.

15. The set of primers of any one of clauses 1-14, wherein the wherein the first polynucleotide sequence is about 4 to about 25 nt long.

16. The set of primers of any one of clauses 1-15, wherein the first polynucleotide sequence is about 8 to about 20 nt long.

17. The set of primers of any one of clauses 1-16, wherein the first polynucleotide sequence comprises any one of the below sequences, or the reverse complement thereof:

| Primer Name | Sequence |
|---|---|
| A1 | ATCGACTG (SEQ. I.D. No. 11) |
| B1 | GCTAGCAG (SEQ. I.D. No. 12) |
| C1 | TACTCTCC (SEQ. I.D. No. 13) |
| D1 | TGACAGCA (SEQ. I.D. No. 14) |
| E1 | GCAGGTTG (SEQ. I.D. No. 15) |
| F1 | TTCCAGCT (SEQ. I.D. No. 16) |
| G1 | TAGTTAGC (SEQ. I.D. No. 17) |
| H1 | AGCGCTAA (SEQ. I.D. No. 18) |
| A2 | CGGTTCTT (SEQ. I.D. No. 19) |
| B2 | TAGCATTG (SEQ. I.D. No. 20) |
| C2 | AATTCAAC (SEQ. I.D. No. 21) |
| D2 | TTCACAGA (SEQ. I.D. No. 22) |
| E2 | GCTCTCTT (SEQ. I.D. No. 23) |
| F2 | TGACTTGG (SEQ. I.D. No. 24) |
| G2 | TATGGTTC (SEQ. I.D. No. 25) |
| H2 | CACTAGCC (SEQ. I.D. No. 26) |
| A3 | AACCTCTT (SEQ. I.D. No. 27) |
| B3 | CTACATTG (SEQ. I.D. No. 28) |
| C3 | GCGATTAC (SEQ. I.D. No. 29) |
| D3 | AATTGGCC (SEQ. I.D. No. 30) |
| E3 | AATTGCTT (SEQ. I.D. No. 31) |
| F3 | TTGGTCTG (SEQ. I.D. No. 32) |
| G3 | CATCCTGG (SEQ. I.D. No. 33) |
| H3 | GGATTAAC (SEQ. I.D. No. 34) |
| A4 | CGCATATT (SEQ. I.D. No. 35) |
| B4 | TCATTCGA (SEQ. I.D. No. 36) |
| C4 | GTCCAATC (SEQ. I.D. No. 37) |
| D4 | CTTGGTCA (SEQ. I.D. No. 38) |
| E4 | CCAACGCT (SEQ. I.D. No. 39) |
| F4 | TCCACTTC (SEQ. I.D. No. 40) |
| G4 | AATCTCCA (SEQ. I.D. No. 41) |
| H4 | GTCTGCAC (SEQ. I.D. No. 42) |
| A5 | CTGCTCCT (SEQ. I.D. No. 43) |
| B5 | TTAGCCAG (SEQ. I.D. No. 44) |
| C5 | GCTGATTC (SEQ. I.D. No. 45) |
| D5 | GAATCGAC (SEQ. I.D. No. 46) |
| E5 | AGTCACCT (SEQ. I.D. No. 47) |
| F5 | CACGATTC (SEQ. I.D. No. 48) |
| G5 | GCTCCGAT (SEQ. I.D. No. 49) |
| H5 | CTTGGCTT (SEQ. I.D. No. 50) |
| A6 | GCTGCACT (SEQ. I.D. No. 51) |
| B6 | GAACTTCG (SEQ. I.D. No. 52) |
| C6 | CTGTATTC (SEQ. I.D. No. 53) |
| D6 | ATATCCGA (SEQ. I.D. No. 54) |
| E6 | TTGTCCAT (SEQ. I.D. No. 55) |
| F6 | AGTAAGTC (SEQ. I.D. No. 56) |
| G6 | GAATATCA (SEQ. I.D. No. 57) |
| H6 | CAACTGAT (SEQ. I.D. No. 58) |
| A7 | CCTGTCAT (SEQ. I.D. No. 59) |
| B7 | GACGGTTA (SEQ. I.D. No. 60) |
| C7 | CTATTAGC (SEQ. I.D. No. 61) |
| D7 | TCCAACCA (SEQ. I.D. No. 62) |
| E7 | CTGGCTAT (SEQ. I.D. No. 63) |
| F7 | GCGGACTT (SEQ. I.D. No. 64) |
| G7 | CCATCACA (SEQ. I.D. No. 65) |
| H7 | GGCAATAC (SEQ. I.D. No. 66) |
| A8 | CACTTCAT (SEQ. I.D. No. 67) |
| B8 | CAAGCTTA (SEQ. I.D. No. 68) |
| C8 | AGGTACCA (SEQ. I.D. No. 69) |
| D8 | TCCATAAC (SEQ. I.D. No. 70) |
| E8 | GTCCTCAT (SEQ. I.D. No. 71) |
| F8 | AGTACTGC (SEQ. I.D. No. 72) |
| G8 | CTTGAATC (SEQ. I.D. No. 73) |
| H8 | CCAACTAA (SEQ. I.D. No. 74) |
| A9 | AATACCAT (SEQ. I.D. No. 75) |
| B9 | GCGATATT (SEQ. I.D. No. 76) |
| C9 | GAACGCTA (SEQ. I.D. No. 77) |
| D9 | CTGACATC (SEQ. I.D. No. 78) |
| E9 | GCCACCAT (SEQ. I.D. No. 79) |
| F9 | CGACTCTC (SEQ. I.D. No. 80) |
| G9 | TGCTATTA (SEQ. I.D. No. 81) |
| H9 | CTTCTGGC (SEQ. I.D. No. 82) |
| A10 | ATGAATTA (SEQ. I.D. No. 83) |
| B10 | TACTCCAG (SEQ. I.D. No. 84) |
| C10 | ATCATACC (SEQ. I.D. No. 85) |
| D10 | CCTCTAAC (SEQ. I.D. No. 86) |
| E10 | ATCTTCTC (SEQ. I.D. No. 87) |
| F10 | CAGCTCAC (SEQ. I.D. No. 88) |
| G10 | GGTTATCT (SEQ. I.D. No. 89) |
| H10 | TCCGCATA (SEQ. I.D. No. 90) |
| A11 | TGCTTCAC (SEQ. I.D. No. 91) |
| B11 | GCTTCCTA (SEQ. I.D. No. 92) |
| C11 | GACCATCT (SEQ. I.D. No. 93) |
| D11 | CTGGTATT (SEQ. I.D. No. 94) |
| E11 | TTAATCAC (SEQ. I.D. No. 95) |
| F11 | CGCGAATA (SEQ. I.D. No. 96) |
| G11 | GCTCACCA (SEQ. I.D. No. 97) |
| H11 | TCATGTCT (SEQ. I.D. No. 98) |
| A12 | ATCCTTAA (SEQ. I.D. No. 99) |
| B12 | TTCTTGGC (SEQ. I.D. No. 100) |
| C12 | CATCACTT (SEQ. I.D. No. 101) |
| D12 | CGAACTTC (SEQ. I.D. No. 102) |
| E12 | GACATTAA (SEQ. I.D. No. 103) |
| F12 | TTCACCTT (SEQ. I.D. No. 104) |
| G12 | CCAATCTG (SEQ. I.D. No. 105) |
| H12 | CGACAGTT (SEQ. I.D. No. 106) |
| Plate1 | AAGTAGAG (SEQ. I.D. No. 107) |
| Plate2 | CATGCTTA (SEQ. I.D. No. 108) |
| Plate3 | GCACATCT (SEQ. I.D. No. 109) |
| Plate4 | TGCTCGAC (SEQ. I.D. No. 110) |
| Plate5 | AGCAATTC (SEQ. I.D. No. 111) |
| Plate6 | AGTTGCTT (SEQ. I.D. No. 112) |
| Plate7 | CCAGTTAG (SEQ. I.D. No. 113) |
| Plate8 | TTGAGCCT (SEQ. I.D. No. 114) |
| Plate9 | ACACGATC (SEQ. I.D. No. 115) |
| Plate10 | GGTCCAGA (SEQ. I.D. No. 116) |
| Plate11 | GTATAACA (SEQ. I.D. No. 117) |
| Plate12 | TTCGCTGA (SEQ. I.D. No. 118) |
| Plate13 | AACTTGAC (SEQ. I.D. No. 119) |
| Plate14 | CACATCCT (SEQ. I.D. No. 120) |
| Plate15 | TCGGAATG (SEQ. I.D. No. 121) |
| Plate16 | AAGGATGT (SEQ. I.D. No. 122) |
| Plate17 | CGCGCGGT (SEQ. I.D. No. 123) |
| Plate18 | TCTGGCGA (SEQ. I.D. No. 124) |
| Plate19 | CATAGCGA (SEQ. I.D. No. 125) |
| Plate20 | CAGGAGCC (SEQ. I.D. No. 126) |
| Plate21 | TGTCGGAT (SEQ. I.D. No. 127) |
| Plate22 | ATTATGTT (SEQ. I.D. No. 128) |
| Plate23 | CCTACCAT (SEQ. I.D. No. 129) |
| Plate24 | TACTTAGC (SEQ. I.D. No. 130) |
| Plate25 | CATGATCG (SEQ. I.D. No. 131) |
| Plate26 | AGGATCTA (SEQ. I.D. No. 132) |
| Plate27 | GACAGTAA (SEQ. I.D. No. 133) |
| Plate28 | CCTATGCC (SEQ. I.D. No. 134) |
| Plate29 | TCGCCTTG (SEQ. I.D. No. 135) |
| Plate30 | ATAGCGTC (SEQ. I.D. No. 136) |
| Plate31 | GAAGAAGT (SEQ. I.D. No. 137) |
| Plate32 | ATTCTAGG (SEQ. I.D. No. 138) |
| Plate33 | CGTTACCA (SEQ. I.D. No. 139) |
| Plate34 | GTCTGATG (SEQ. I.D. No. 140) |
| Plate35 | TTACGCAC (SEQ. I.D. No. 141) |
| Plate36 | TTGAATAG (SEQ. I.D. No. 142) |
| Plate37 | AAGACACT (SEQ. I.D. No. 143) |
| Plate38 | CAGCAAGG (SEQ. I.D. No. 144) |
| Plate39 | TCCAGCAA (SEQ. I.D. No. 145) |
| Plate40 | CCAGAGCT (SEQ. I.D. No. 146) |
| Plate41 | TCCTTGGT (SEQ. I.D. No. 147) |
| Plate42 | AGGTTATC (SEQ. I.D. No. 148) |
| Plate43 | GTCATCTA (SEQ. I.D. No. 149) |
| Plate44 | CCTTCGCA (SEQ. I.D. No. 150) |
| Plate45 | TCTCGGTC (SEQ. I.D. No. 151) |
| Plate46 | ATTGTCTG (SEQ. I.D. No. 152) |
| Plate47 | GAACCTAG (SEQ. I.D. No. 153) |
| Plate92 | TTAATCAG (SEQ. I.D. No. 198) |
| Plate93 | AGGTGCGA (SEQ. I.D. No. 199) |
| Plate94 | CTGTGGCG (SEQ. I.D. No. 200) |
| Plate95 | GCCGCAAC (SEQ. I.D. No. 201) |
| Plate96 | TTATATCT (SEQ. I.D. No. 202) |

18. The set of primers of any one of clauses 1-17, wherein first polynucleotide sequence further comprises a 5′-GC-sequence.

19. The set of primers of any one of clauses 1-18, wherein the second polynucleotide sequence is at least about 15 or about 20 nt long.

20. The set of primers of any one of clauses 1-19, wherein the second polynucleotide sequence is at least about 25 nt long.

21. The set of primers of any one of clauses 1-20, wherein the unique polynucleotide identifier is an exogenous polynucleotide identifier, flanked by upstream and downstream respective flanking sequences common for all strains of the strain collection;
wherein the set of primers comprises a first subset of primers, the second polynucleotide sequence of which is the upstream flanking sequence; and
wherein the set of primers comprises a second subset of primers, the second polynucleotide sequence of which is the downstream flanking sequence.

22. The set of primers of any one of clauses 1-21, wherein the second polynucleotide sequence comprises any one of the below sequences, or the reverse complement thereof:

5′ Flank

(SEQ. I.D. No. 203)

TATTTATGCAGAGGCCGAGG
3′ Flank Sequence

(SEQ. I.D. No. 204)

GGATTATTCATACCGTCCCA.

23. The set of primers of any one of clauses 1-22, wherein the each primer comprises any one of the below sequences, or the reverse complement thereof:

5′ Primer Sequence (GC + Well
BC + 5′Flank)

SEQ. I.D. NO. 205

GCATCGACTGTATTTATGCAGAGGCCGAGG

SEQ. I.D. NO. 206

GCGCTAGCAGTATTTATGCAGAGGCCGAGG

SEQ. I.D. NO. 207

GCTACTCTCCTATTTATGCAGAGGCCGAGG

SEQ. I.D. NO. 208

GCTGACAGCATATTTATGCAGAGGCCGAGG

SEQ. I.D. NO. 209

GCGCAGGTTGTATTTATGCAGAGGCCGAGG

SEQ. I.D. NO. 210

GCTTCCAGCTTATTTATGCAGAGGCCGAGG

SEQ. I.D. NO. 211

GCTAGTTAGCTATTTATGCAGAGGCCGAGG

SEQ. I.D. NO. 212

GCAGCGCTAATATTTATGCAGAGGCCGAGG

SEQ. I.D. NO. 213

GCCGGTTCTTTATTTATGCAGAGGCCGAGG

SEQ. I.D. NO. 214

GCTAGCATTGTATTTATGCAGAGGCCGAGG

SEQ. I.D. NO. 215

GCAATTCAACTATTTATGCAGAGGCCGAGG

SEQ. I.D. NO. 216

GCTTCACAGATATTTATGCAGAGGCCGAGG

SEQ. I.D. NO. 217

GCGCTCTCTTTATTTATGCAGAGGCCGAGG

SEQ. I.D. NO. 218

GCTGACTTGGTATTTATGCAGAGGCCGAGG

SEQ. I.D. NO. 219

GCTATGGTTCTATTTATGCAGAGGCCGAGG

SEQ. I.D. NO. 220

GCCACTAGCCTATTTATGCAGAGGCCGAGG

SEQ. I.D. NO. 221

GCAACCTCTTTATTTATGCAGAGGCCGAGG

SEQ. I.D. NO. 222

GCCTACATTGTATTTATGCAGAGGCCGAGG

SEQ. I.D. NO. 223

GCGCGATTACTATTTATGCAGAGGCCGAGG

SEQ. I.D. NO. 224

GCAATTGGCCTATTTATGCAGAGGCCGAGG

SEQ. I.D. NO. 225

GCAATTGCTTTATTTATGCAGAGGCCGAGG

SEQ. I.D. NO. 226

GCTTGGTCTGTATTTATGCAGAGGCCGAGG

SEQ. I.D. NO. 227

GCCATCCTGGTATTTATGCAGAGGCCGAGG

SEQ. I.D. NO. 228

GCGGATTAACTATTTATGCAGAGGCCGAGG

SEQ. I.D. NO. 229

GCCGCATATTTATTTATGCAGAGGCCGAGG

SEQ. I.D. NO. 230

GCTCATTCGATATTTATGCAGAGGCCGAGG

SEQ. I.D. NO. 231

GCGTCCAATCTATTTATGCAGAGGCCGAGG

SEQ. I.D. NO. 232

GCCTTGGTCATATTTATGCAGAGGCCGAGG

SEQ. I.D. NO. 233

GCCCAACGCTTATTTATGCAGAGGCCGAGG

SEQ. I.D. NO. 234

GCTCCACTTCTATTTATGCAGAGGCCGAGG

SEQ. I.D. NO. 235

GCAATCTCCATATTTATGCAGAGGCCGAGG

SEQ. I.D. NO. 236

GCGTCTGCACTATTTATGCAGAGGCCGAGG

SEQ. I.D. NO. 237

GCCTGCTCCTTATTTATGCAGAGGCCGAGG

SEQ. I.D. NO. 238

GCTTAGCCAGTATTTATGCAGAGGCCGAGG

SEQ. I.D. NO. 239

GCGCTGATTCTATTTATGCAGAGGCCGAGG

SEQ. I.D. NO. 240

GCGAATCGACTATTTATGCAGAGGCCGAGG

SEQ. I.D. NO. 241

GCAGTCACCTTATTTATGCAGAGGCCGAGG

SEQ. I.D. NO. 242

GCCACGATTCTATTTATGCAGAGGCCGAGG

SEQ. I.D. NO. 243

GCGCTCCGATTATTTATGCAGAGGCCGAGG

SEQ. I.D. NO. 244

GCCTTGGCTTTATTTATGCAGAGGCCGAGG

SEQ. I.D. NO. 245

GCGCTGCACTTATTTATGCAGAGGCCGAGG

SEQ. I.D. NO. 246

GCGAACTTCGTATTTATGCAGAGGCCGAGG

SEQ. I.D. NO. 247

GCCTGTATTCTATTTATGCAGAGGCCGAGG

SEQ. I.D. NO. 248

GCATATCCGATATTTATGCAGAGGCCGAGG

SEQ. I.D. NO. 249

GCTTGTCCATTATTTATGCAGAGGCCGAGG

SEQ. I.D. NO. 250

GCAGTAAGTCTATTTATGCAGAGGCCGAGG

SEQ. I.D. NO. 251

GCGAATATCATATTTATGCAGAGGCCGAGG

SEQ. I.D. NO. 252

GCCAACTGATTATTTATGCAGAGGCCGAGG

SEQ. I.D. NO. 253

GCCCTGTCATTATTTATGCAGAGGCCGAGG

SEQ. I.D. NO. 254

GCGACGGTTATATTTATGCAGAGGCCGAGG

SEQ. I.D. NO. 255

GCCTATTAGCTATTTATGCAGAGGCCGAGG

SEQ. I.D. NO. 256

GCTCCAACCATATTTATGCAGAGGCCGAGG

SEQ. I.D. NO. 257

GCCTGGCTATTATTTATGCAGAGGCCGAGG

SEQ. I.D. NO. 258

GCGCGGACTTTATTTATGCAGAGGCCGAGG

SEQ. I.D. NO. 259

GCCCATCACATATTTATGCAGAGGCCGAGG

SEQ. I.D. NO. 260

GCGGCAATACTATTTATGCAGAGGCCGAGG

SEQ. I.D. NO. 261

GCCACTTCATTATTTATGCAGAGGCCGAGG

SEQ. I.D. NO. 262

GCCAAGCTTATATTTATGCAGAGGCCGAGG

SEQ. I.D. NO. 263

GCAGGTACCATATTTATGCAGAGGCCGAGG

SEQ. I.D. NO. 264

GCTCCATAACTATTTATGCAGAGGCCGAGG

SEQ. I.D. NO. 265

GCGTCCTCATTATTTATGCAGAGGCCGAGG

SEQ. I.D. NO. 266

GCAGTACTGCTATTTATGCAGAGGCCGAGG

SEQ. I.D. NO. 267

GCCTTGAATCTATTTATGCAGAGGCCGAGG

SEQ. I.D. NO. 268

GCCCAACTAATATTTATGCAGAGGCCGAGG

SEQ. I.D. NO. 269

GCAATACCATTATTTATGCAGAGGCCGAGG

SEQ. I.D. NO. 270

GCGCGATATTTATTTATGCAGAGGCCGAGG

SEQ. I.D. NO. 271

GCGAACGCTATATTTATGCAGAGGCCGAGG

SEQ. I.D. NO. 272

GCCTGACATCTATTTATGCAGAGGCCGAGG

SEQ. I.D. NO. 273

GCGCCACCATTATTTATGCAGAGGCCGAGG

SEQ. I.D. NO. 274

GCCGACTCTCTATTTATGCAGAGGCCGAGG

SEQ. I.D. NO. 275

GCTGCTATTATATTTATGCAGAGGCCGAGG

SEQ. I.D. NO. 276

GCCTTCTGGCTATTTATGCAGAGGCCGAGG

SEQ. I.D. NO. 277

GCATGAATTATATTTATGCAGAGGCCGAGG

SEQ. I.D. NO. 278

GCTACTCCAGTATTTATGCAGAGGCCGAGG

SEQ. I.D. NO. 279

GCATCATACCTATTTATGCAGAGGCCGAGG

SEQ. I.D. NO. 280

GCCCTCTAACTATTTATGCAGAGGCCGAGG

SEQ. I.D. NO. 281

GCATCTTCTCTATTTATGCAGAGGCCGAGG

SEQ. I.D. NO. 282

GCCAGCTCACTATTTATGCAGAGGCCGAGG

SEQ. I.D. NO. 283

GCGGTTATCTTATTTATGCAGAGGCCGAGG

SEQ. I.D. NO. 284

GCTCCGCATATATTTATGCAGAGGCCGAGG

SEQ. I.D. NO. 285

GCTGCTTCACTATTTATGCAGAGGCCGAGG

SEQ. I.D. NO. 286

GCGCTTCCTATATTTATGCAGAGGCCGAGG

SEQ. I.D. NO. 287

GCGACCATCTTATTTATGCAGAGGCCGAGG

SEQ. I.D. NO. 288

GCCTGGTATTTATTTATGCAGAGGCCGAGG

SEQ. I.D. NO. 289

GCTTAATCACTATTTATGCAGAGGCCGAGG

SEQ. I.D. NO. 290

GCCGCGAATATATTTATGCAGAGGCCGAGG

SEQ. I.D. NO. 291

GCGCTCACCATATTTATGCAGAGGCCGAGG

SEQ. I.D. NO. 292

GCTCATGTCTTATTTATGCAGAGGCCGAGG

SEQ. I.D. NO. 293

GCATCCTTAATATTTATGCAGAGGCCGAGG

SEQ. I.D. NO. 294

GCTTCTTGGCTATTTATGCAGAGGCCGAGG

SEQ. I.D. NO. 295

GCCATCACTTTATTTATGCAGAGGCCGAGG

SEQ. I.D. NO. 296

GCCGAACTTCTATTTATGCAGAGGCCGAGG

SEQ. I.D. NO. 297

GCGACATTAATATTTATGCAGAGGCCGAGG

SEQ. I.D. NO. 298

GCTTCACCTTTATTTATGCAGAGGCCGAGG

SEQ. I.D. NO. 299

GCCCAATCTGTATTTATGCAGAGGCCGAGG

SEQ. I.D. NO. 300

GCCGACAGTTTATTTATGCAGAGGCCGAGG
3′ Primer Sequence (GC + Plate
BC + Rev. comp. of 3′ Flank)

SEQ. I.D. NO. 301

GCCTCTACTTTGGGACGGTATGAATAATCC

SEQ. I.D. NO. 302

GCTAAGCATGTGGGACGGTATGAATAATCC

SEQ. I.D. NO. 303

GCAGATGTGCTGGGACGGTATGAATAATCC

SEQ. I.D. NO. 304

GCGTCGAGCATGGGACGGTATGAATAATCC

SEQ. I.D. NO. 305

GCGAATTGCTTGGGACGGTATGAATAATCC

SEQ. I.D. NO. 306

GCAAGCAACTTGGGACGGTATGAATAATCC

SEQ. I.D. NO. 307

GCCTAACTGGTGGGACGGTATGAATAATCC

SEQ. I.D. NO. 308

GCAGGCTCAATGGGACGGTATGAATAATCC

SEQ. I.D. NO. 309

GCGATCGTGTTGGGACGGTATGAATAATCC

SEQ. I.D. NO. 310

GCTCTGGACCTGGGACGGTATGAATAATCC

SEQ. I.D. NO. 311

GCTGTTATACTGGGACGGTATGAATAATCC

SEQ. I.D. NO. 312

GCTCAGCGAATGGGACGGTATGAATAATCC

SEQ. I.D. NO. 313

GCGTCAAGTTTGGGACGGTATGAATAATCC

SEQ. I.D. NO. 314

GCAGGATGTGTGGGACGGTATGAATAATCC

SEQ. I.D. NO. 315

GCCATTCCGATGGGACGGTATGAATAATCC

SEQ. I.D. NO. 316

GCACATCCTTTGGGACGGTATGAATAATCC

SEQ. I.D. NO. 317

GCACCGCGCGTGGGACGGTATGAATAATCC

SEQ. I.D. NO. 318

GCTCGCCAGATGGGACGGTATGAATAATCC

SEQ. I.D. NO. 319

GCTCGCTATGTGGGACGGTATGAATAATCC

SEQ. I.D. NO. 320

GCGGCTCCTGTGGGACGGTATGAATAATCC

SEQ. I.D. NO. 321

GCATCCGACATGGGACGGTATGAATAATCC

SEQ. I.D. NO. 322

GCAACATAATTGGGACGGTATGAATAATCC

SEQ. I.D. NO. 323

GCATGGTAGGTGGGACGGTATGAATAATCC

SEQ. I.D. NO. 324

GCGCTAAGTATGGGACGGTATGAATAATCC

SEQ. I.D. NO. 325

GCCGATCATGTGGGACGGTATGAATAATCC

SEQ. I.D. NO. 326

GCTAGATCCTTGGGACGGTATGAATAATCC

SEQ. I.D. NO. 327

GCTTACTGTCTGGGACGGTATGAATAATCC

SEQ. I.D. NO. 328

GCGGCATAGGTGGGACGGTATGAATAATCC

SEQ. I.D. NO. 329

GCCAAGGCGATGGGACGGTATGAATAATCC

SEQ. I.D. NO. 330

GCGACGCTATTGGGACGGTATGAATAATCC

SEQ. I.D. NO. 331

GCACTTCTTCTGGGACGGTATGAATAATCC

SEQ. I.D. NO. 332

GCCCTAGAATTGGGACGGTATGAATAATCC

SEQ. I.D. NO. 333

GCTGGTAACGTGGGACGGTATGAATAATCC

SEQ. I.D. NO. 334

GCCATCAGACTGGGACGGTATGAATAATCC

SEQ. I.D. NO. 335

GCGTGCGTAATGGGACGGTATGAATAATCC

SEQ. I.D. NO. 336

GCCTATTCAATGGGACGGTATGAATAATCC

SEQ. I.D. NO. 337

GCAGTGTCTTTGGGACGGTATGAATAATCC

SEQ. I.D. NO. 338

GCCCTTGCTGTGGGACGGTATGAATAATCC

SEQ. I.D. NO. 339

GCTTGCTGGATGGGACGGTATGAATAATCC

SEQ. I.D. NO. 340

GCAGCTCTGGTGGGACGGTATGAATAATCC

SEQ. I.D. NO. 341

GCACCAAGGATGGGACGGTATGAATAATCC

SEQ. I.D. NO. 342

GCGATAACCTTGGGACGGTATGAATAATCC

SEQ. I.D. NO. 343

GCTAGATGACTGGGACGGTATGAATAATCC

SEQ. I.D. NO. 344

GCTGCGAAGGTGGGACGGTATGAATAATCC

SEQ. I.D. NO. 345

GCGACCGAGATGGGACGGTATGAATAATCC

SEQ. I.D. NO. 346

GCCAGACAATTGGGACGGTATGAATAATCC

SEQ. I.D. NO. 347

GCCTAGGTTCTGGGACGGTATGAATAATCC

SEQ. I.D. NO. 348

GCGTTCATTATGGGACGGTATGAATAATCC

SEQ. I.D. NO. 349

GCAATGCGTTTGGGACGGTATGAATAATCC

SEQ. I.D. NO. 350

GCGAGAGTTGTGGGACGGTATGAATAATCC

SEQ. I.D. NO. 351

GCGATTACAGTGGGACGGTATGAATAATCC

SEQ. I.D. NO. 352

GCTGTGCTTATGGGACGGTATGAATAATCC

SEQ. I.D. NO. 353

GCAGAACATTTGGGACGGTATGAATAATCC

SEQ. I.D. NO. 354

GCTACCGCTGTGGGACGGTATGAATAATCC

SEQ. I.D. NO. 355

GCTCCTGGTCTGGGACGGTATGAATAATCC

SEQ. I.D. NO. 356

GCCCTGGATATGGGACGGTATGAATAATCC

SEQ. I.D. NO. 357

GCATACCTGTTGGGACGGTATGAATAATCC

SEQ. I.D. NO. 358

GCAATGTTGGTGGGACGGTATGAATAATCC

SEQ. I.D. NO. 359

GCTCGACGGCTGGGACGGTATGAATAATCC

SEQ. I.D. NO. 360

GCGGCAGATATGGGACGGTATGAATAATCC

SEQ. I.D. NO. 361

GCGTCTTAGTTGGGACGGTATGAATAATCC

SEQ. I.D. NO. 362

GCGGAAGGCGTGGGACGGTATGAATAATCC

SEQ. I.D. NO. 363

GCGGCTAGGCTGGGACGGTATGAATAATCC

SEQ. I.D. NO. 364

GCCAGCAGCATGGGACGGTATGAATAATCC

SEQ. I.D. NO. 365

GCCCTTACCTTGGGACGGTATGAATAATCC

SEQ. I.D. NO. 366

GCCGAGTTAGTGGGACGGTATGAATAATCC

SEQ. I.D. NO. 367

GCGATGTTACTGGGACGGTATGAATAATCC

SEQ. I.D. NO. 368

GCTGATTACATGGGACGGTATGAATAATCC

SEQ. I.D. NO. 369

GCTTGATAATTGGGACGGTATGAATAATCC

SEQ. I.D. NO. 370

GCACGCATAGTGGGACGGTATGAATAATCC

SEQ. I.D. NO. 371

GCCTGTGGACTGGGACGGTATGAATAATCC

SEQ. I.D. NO. 372

GCATAGACAATGGGACGGTATGAATAATCC

SEQ. I.D. NO. 373

GCCCATTGTTTGGGACGGTATGAATAATCC

SEQ. I.D. NO. 374

GCAGAGGAATTGGGACGGTATGAATAATCC

SEQ. I.D. NO. 375

GCCTTCCTTCTGGGACGGTATGAATAATCC

SEQ. I.D. NO. 376

GCTCTAGCGATGGGACGGTATGAATAATCC

SEQ. I.D. NO. 377

GCTCAACTGTTGGGACGGTATGAATAATCC

SEQ. I.D. NO. 378

GCGACTATTGTGGGACGGTATGAATAATCC

SEQ. I.D. NO. 379

GCCAACGGTCTGGGACGGTATGAATAATCC

SEQ. I.D. NO. 380

GCCTTGCAGATGGGACGGTATGAATAATCC

SEQ. I.D. NO. 381

GCGATACAGTTGGGACGGTATGAATAATCC

SEQ. I.D. NO. 382

GCCCTGGTAGTGGGACGGTATGAATAATCC

SEQ. I.D. NO. 383

GCGTTAGGTCTGGGACGGTATGAATAATCC

SEQ. I.D. NO. 384

GCTACTTGCATGGGACGGTATGAATAATCC

SEQ. I.D. NO. 385

GCTCCATGCTTGGGACGGTATGAATAATCC

SEQ. I.D. NO. 386

GCACATAGCGTGGGACGGTATGAATAATCC

SEQ. I.D. NO. 387

GCTGGATATCTGGGACGGTATGAATAATCC

SEQ. I.D. NO. 388

GCGAGTTACATGGGACGGTATGAATAATCC

SEQ. I.D. NO. 389

GCTGCGACCTTGGGACGGTATGAATAATCC

SEQ. I.D. NO. 390

GCATCCGCAGTGGGACGGTATGAATAATCC

SEQ. I.D. NO. 391

GCCAGTTGGTTGGGACGGTATGAATAATCC

SEQ. I.D. NO. 392

GCCTGATTAATGGGACGGTATGAATAATCC

SEQ. I.D. NO. 393

GCTCGCACCTTGGGACGGTATGAATAATCC

SEQ. I.D. NO. 394

GCCGCCACAGTGGGACGGTATGAATAATCC

SEQ. I.D. NO. 395

GCGTTGCGGCTGGGACGGTATGAATAATCC

SEQ. I.D. NO. 396

GCAGATATAATGGGACGGTATGAATAATCC
5′ Primer Sequence (no more
GC + Well BC + 5′Flank)

SEQ. I.D. NO. 397

ATCGACTGTATTTATGCAGAGGCCGAGG

SEQ. I.D. NO. 398

GCTAGCAGTATTTATGCAGAGGCCGAGG

SEQ. I.D. NO. 399

TACTCTCCTATTTATGCAGAGGCCGAGG

SEQ. I.D. NO. 400

TGACAGCATATTTATGCAGAGGCCGAGG

SEQ. I.D. NO. 401

GCAGGTTGTATTTATGCAGAGGCCGAGG

SEQ. I.D. NO. 402

TTCCAGCTTATTTATGCAGAGGCCGAGG

SEQ. I.D. NO. 403

TAGTTAGCTATTTATGCAGAGGCCGAGG

SEQ. I.D. NO. 404

AGCGCTAATATTTATGCAGAGGCCGAGG

SEQ. I.D. NO. 405

CGGTTCTTTATTTATGCAGAGGCCGAGG

SEQ. I.D. NO. 406

TAGCATTGTATTTATGCAGAGGCCGAGG

SEQ. I.D. NO. 407

AATTCAACTATTTATGCAGAGGCCGAGG

SEQ. I.D. NO. 408

TTCACAGATATTTATGCAGAGGCCGAGG

SEQ. I.D. NO. 409

GCTCTCTTTATTTATGCAGAGGCCGAGG

SEQ. I.D. NO. 410

TGACTTGGTATTTATGCAGAGGCCGAGG

SEQ. I.D. NO. 411

TATGGTTCTATTTATGCAGAGGCCGAGG

SEQ. I.D. NO. 412

CACTAGCCTATTTATGCAGAGGCCGAGG

SEQ. I.D. NO. 413

AACCTCTTTATTTATGCAGAGGCCGAGG

SEQ. I.D. NO. 414

CTACATTGTATTTATGCAGAGGCCGAGG

SEQ. I.D. NO. 415

GCGATTACTATTTATGCAGAGGCCGAGG

SEQ. I.D. NO. 416

AATTGGCCTATTTATGCAGAGGCCGAGG

SEQ. I.D. NO. 417

AATTGCTTTATTTATGCAGAGGCCGAGG

SEQ. I.D. NO. 418

TTGGTCTGTATTTATGCAGAGGCCGAGG

SEQ. I.D. NO. 419

CATCCTGGTATTTATGCAGAGGCCGAGG

SEQ. I.D. NO. 420

GGATTAACTATTTATGCAGAGGCCGAGG

SEQ. I.D. NO. 421

CGCATATTTATTTATGCAGAGGCCGAGG

SEQ. I.D. NO. 422

TCATTCGATATTTATGCAGAGGCCGAGG

SEQ. I.D. NO. 423

GTCCAATCTATTTATGCAGAGGCCGAGG

SEQ. I.D. NO. 424

CTTGGTCATATTTATGCAGAGGCCGAGG

SEQ. I.D. NO. 425

CCAACGCTTATTTATGCAGAGGCCGAGG

SEQ. I.D. NO. 426

TCCACTTCTATTTATGCAGAGGCCGAGG

SEQ. I.D. NO. 427

AATCTCCATATTTATGCAGAGGCCGAGG

SEQ. I.D. NO. 428

GTCTGCACTATTTATGCAGAGGCCGAGG

SEQ. I.D. NO. 429

CTGCTCCTTATTTATGCAGAGGCCGAGG

SEQ. I.D. NO. 430

TTAGCCAGTATTTATGCAGAGGCCGAGG

SEQ. I.D. NO. 431

GCTGATTCTATTTATGCAGAGGCCGAGG

SEQ. I.D. NO. 432

GAATCGACTATTTATGCAGAGGCCGAGG

SEQ. I.D. NO. 433

AGTCACCTTATTTATGCAGAGGCCGAGG

SEQ. I.D. NO. 434

CACGATTCTATTTATGCAGAGGCCGAGG

SEQ. I.D. NO. 435

GCTCCGATTATTTATGCAGAGGCCGAGG

SEQ. I.D. NO. 436

CTTGGCTTTATTTATGCAGAGGCCGAGG

SEQ. I.D. NO. 437

GCTGCACTTATTTATGCAGAGGCCGAGG

SEQ. I.D. NO. 438

GAACTTCGTATTTATGCAGAGGCCGAGG

SEQ. I.D. NO. 439

CTGTATTCTATTTATGCAGAGGCCGAGG

SEQ. I.D. NO. 440

ATATCCGATATTTATGCAGAGGCCGAGG

SEQ. I.D. NO. 441

TTGTCCATTATTTATGCAGAGGCCGAGG

SEQ. I.D. NO. 442

AGTAAGTCTATTTATGCAGAGGCCGAGG

SEQ. I.D. NO. 443

GAATATCATATTTATGCAGAGGCCGAGG

SEQ. I.D. NO. 444

CAACTGATTATTTATGCAGAGGCCGAGG

SEQ. I.D. NO. 445

CCTGTCATTATTTATGCAGAGGCCGAGG

SEQ. I.D. NO. 446

GACGGTTATATTTATGCAGAGGCCGAGG

SEQ. I.D. NO. 447

CTATTAGCTATTTATGCAGAGGCCGAGG

SEQ. I.D. NO. 448

TCCAACCATATTTATGCAGAGGCCGAGG

SEQ. I.D. NO. 449

CTGGCTATTATTTATGCAGAGGCCGAGG

SEQ. I.D. NO. 450

GCGGACTTTATTTATGCAGAGGCCGAGG

SEQ. I.D. NO. 451

CCATCACATATTTATGCAGAGGCCGAGG

SEQ. I.D. NO. 452

GGCAATACTATTTATGCAGAGGCCGAGG

SEQ. I.D. NO. 453

CACTTCATTATTTATGCAGAGGCCGAGG

SEQ. I.D. NO. 454

CAAGCTTATATTTATGCAGAGGCCGAGG

SEQ. I.D. NO. 455

AGGTACCATATTTATGCAGAGGCCGAGG

SEQ. I.D. NO. 456

TCCATAACTATTTATGCAGAGGCCGAGG

SEQ. I.D. NO. 457

GTCCTCATTATTTATGCAGAGGCCGAGG

SEQ. I.D. NO. 458

AGTACTGCTATTTATGCAGAGGCCGAGG

SEQ. I.D. NO. 459

CTTGAATCTATTTATGCAGAGGCCGAGG

SEQ. I.D. NO. 460

CCAACTAATATTTATGCAGAGGCCGAGG

SEQ. I.D. NO. 461

AATACCATTATTTATGCAGAGGCCGAGG

SEQ. I.D. NO. 462

GCGATATTTATTTATGCAGAGGCCGAGG

SEQ. I.D. NO. 463

GAACGCTATATTTATGCAGAGGCCGAGG

SEQ. I.D. NO. 464

CTGACATCTATTTATGCAGAGGCCGAGG

SEQ. I.D. NO. 465

GCCACCATTATTTATGCAGAGGCCGAGG

SEQ. I.D. NO. 466

CGACTCTCTATTTATGCAGAGGCCGAGG

SEQ. I.D. NO. 467

TGCTATTATATTTATGCAGAGGCCGAGG

SEQ. I.D. NO. 468

CTTCTGGCTATTTATGCAGAGGCCGAGG

SEQ. I.D. NO. 469

ATGAATTATATTTATGCAGAGGCCGAGG

SEQ. I.D. NO. 470

TACTCCAGTATTTATGCAGAGGCCGAGG

SEQ. I.D. NO. 471

ATCATACCTATTTATGCAGAGGCCGAGG

SEQ. I.D. NO. 472

CCTCTAACTATTTATGCAGAGGCCGAGG

SEQ. I.D. NO. 473

ATCTTCTCTATTTATGCAGAGGCCGAGG

SEQ. I.D. NO. 474

CAGCTCACTATTTATGCAGAGGCCGAGG

SEQ. I.D. NO. 475

GGTTATCTTATTTATGCAGAGGCCGAGG

SEQ. I.D. NO. 476

TCCGCATATATTTATGCAGAGGCCGAGG

SEQ. I.D. NO. 477

TGCTTCACTATTTATGCAGAGGCCGAGG

SEQ. I.D. NO. 478

GCTTCCTATATTTATGCAGAGGCCGAGG

SEQ. I.D. NO. 479

GACCATCTTATTTATGCAGAGGCCGAGG

SEQ. I.D. NO. 480

CTGGTATTTATTTATGCAGAGGCCGAGG

SEQ. I.D. NO. 481

TTAATCACTATTTATGCAGAGGCCGAGG

SEQ. I.D. NO. 482

CGCGAATATATTTATGCAGAGGCCGAGG

SEQ. I.D. NO. 483

GCTCACCATATTTATGCAGAGGCCGAGG

SEQ. I.D. NO. 484

TCATGTCTTATTTATGCAGAGGCCGAGG

SEQ. I.D. NO. 485

ATCCTTAATATTTATGCAGAGGCCGAGG

SEQ. I.D. NO. 486

TTCTTGGCTATTTATGCAGAGGCCGAGG

SEQ. I.D. NO. 487

CATCACTTTATTTATGCAGAGGCCGAGG

SEQ. I.D. NO. 488

CGAACTTCTATTTATGCAGAGGCCGAGG

SEQ. I.D. NO. 489

GACATTAATATTTATGCAGAGGCCGAGG

SEQ. I.D. NO. 490

TTCACCTTTATTTATGCAGAGGCCGAGG

SEQ. I.D. NO. 491

CCAATCTGTATTTATGCAGAGGCCGAGG

SEQ. I.D. NO. 492

CGACAGTTTATTTATGCAGAGGCCGAGG
3′ Primer Sequence (GC + Plate
BC + Rev. comp. of 3′ Flank)

SEQ. I.D. NO. 493

CTCTACTTTGGGACGGTATGAATAATCC

SEQ. I.D. NO. 494

TAAGCATGTGGGACGGTATGAATAATCC

SEQ. I.D. NO. 495

AGATGTGCTGGGACGGTATGAATAATCC

SEQ. I.D. NO. 496

GTCGAGCATGGGACGGTATGAATAATCC

SEQ. I.D. NO. 497

GAATTGCTTGGGACGGTATGAATAATCC

SEQ. I.D. NO. 498

AAGCAACTTGGGACGGTATGAATAATCC

SEQ. I.D. NO. 499

CTAACTGGTGGGACGGTATGAATAATCC

SEQ. I.D. NO. 500

AGGCTCAATGGGACGGTATGAATAATCC

SEQ. I.D. NO. 501

GATCGTGTTGGGACGGTATGAATAATCC

SEQ. I.D. NO. 502

TCTGGACCTGGGACGGTATGAATAATCC

SEQ. I.D. NO. 503

TGTTATACTGGGACGGTATGAATAATCC

SEQ. I.D. NO. 504

TCAGCGAATGGGACGGTATGAATAATCC

SEQ. I.D. NO. 505

GTCAAGTTTGGGACGGTATGAATAATCC

SEQ. I.D. NO. 506

AGGATGTGTGGGACGGTATGAATAATCC

SEQ. I.D. NO. 507

CATTCCGATGGGACGGTATGAATAATCC

SEQ. I.D. NO. 508

ACATCCTTTGGGACGGTATGAATAATCC

SEQ. I.D. NO. 509

ACCGCGCGTGGGACGGTATGAATAATCC

SEQ. I.D. NO. 510

TCGCCAGATGGGACGGTATGAATAATCC

SEQ. I.D. NO. 511

TCGCTATGTGGGACGGTATGAATAATCC

SEQ. I.D. NO. 512

GGCTCCTGTGGGACGGTATGAATAATCC

SEQ. I.D. NO. 513

ATCCGACATGGGACGGTATGAATAATCC

SEQ. I.D. NO. 514

AACATAATTGGGACGGTATGAATAATCC

SEQ. I.D. NO. 515

ATGGTAGGTGGGACGGTATGAATAATCC

SEQ. I.D. NO. 516

GCTAAGTATGGGACGGTATGAATAATCC

SEQ. I.D. NO. 517

CGATCATGTGGGACGGTATGAATAATCC

SEQ. I.D. NO. 518

TAGATCCTTGGGACGGTATGAATAATCC

SEQ. I.D. NO. 519

TTACTGTCTGGGACGGTATGAATAATCC

SEQ. I.D. NO. 520

GGCATAGGTGGGACGGTATGAATAATCC

SEQ. I.D. NO. 521

CAAGGCGATGGGACGGTATGAATAATCC

SEQ. I.D. NO. 522

GACGCTATTGGGACGGTATGAATAATCC

SEQ. I.D. NO. 523

ACTTCTTCTGGGACGGTATGAATAATCC

SEQ. I.D. NO. 524

CCTAGAATTGGGACGGTATGAATAATCC

SEQ. I.D. NO. 525

TGGTAACGTGGGACGGTATGAATAATCC

SEQ. I.D. NO. 526

CATCAGACTGGGACGGTATGAATAATCC

SEQ. I.D. NO. 527

GTGCGTAATGGGACGGTATGAATAATCC

SEQ. I.D. NO. 528

CTATTCAATGGGACGGTATGAATAATCC

SEQ. I.D. NO. 529

AGTGTCTTTGGGACGGTATGAATAATCC

SEQ. I.D. NO. 530

CCTTGCTGTGGGACGGTATGAATAATCC

SEQ. I.D. NO. 531

TTGCTGGATGGGACGGTATGAATAATCC

SEQ. I.D. NO. 532

AGCTCTGGTGGGACGGTATGAATAATCC

SEQ. I.D. NO. 533

ACCAAGGATGGGACGGTATGAATAATCC

SEQ. I.D. NO. 534

GATAACCTTGGGACGGTATGAATAATCC

SEQ. I.D. NO. 535

TAGATGACTGGGACGGTATGAATAATCC

SEQ. I.D. NO. 536

TGCGAAGGTGGGACGGTATGAATAATCC

SEQ. I.D. NO. 537

GACCGAGATGGGACGGTATGAATAATCC

SEQ. I.D. NO. 538

CAGACAATTGGGACGGTATGAATAATCC

SEQ. I.D. NO. 539

CTAGGTTCTGGGACGGTATGAATAATCC

SEQ. I.D. NO. 540

GTTCATTATGGGACGGTATGAATAATCC

SEQ. I.D. NO. 541

AATGCGTTTGGGACGGTATGAATAATCC

SEQ. I.D. NO. 542

GAGAGTTGTGGGACGGTATGAATAATCC

SEQ. I.D. NO. 543

GATTACAGTGGGACGGTATGAATAATCC

SEQ. I.D. NO. 544

TGTGCTTATGGGACGGTATGAATAATCC

SEQ. I.D. NO. 545

AGAACATTTGGGACGGTATGAATAATCC

SEQ. I.D. NO. 546

TACCGCTGTGGGACGGTATGAATAATCC

SEQ. I.D. NO. 547

TCCTGGTCTGGGACGGTATGAATAATCC

SEQ. I.D. NO. 548

CCTGGATATGGGACGGTATGAATAATCC

SEQ. I.D. NO. 549

ATACCTGTTGGGACGGTATGAATAATCC

SEQ. I.D. NO. 550

AATGTTGGTGGGACGGTATGAATAATCC

SEQ. I.D. NO. 551

TCGACGGCTGGGACGGTATGAATAATCC

SEQ. I.D. NO. 552

GGCAGATATGGGACGGTATGAATAATCC

SEQ. I.D. NO. 553

GTCTTAGTTGGGACGGTATGAATAATCC

SEQ. I.D. NO. 554

GGAAGGCGTGGGACGGTATGAATAATCC

SEQ. I.D. NO. 555

GGCTAGGCTGGGACGGTATGAATAATCC

SEQ. I.D. NO. 556

CAGCAGCATGGGACGGTATGAATAATCC

SEQ. I.D. NO. 557

CCTTACCTTGGGACGGTATGAATAATCC

SEQ. I.D. NO. 558

CGAGTTAGTGGGACGGTATGAATAATCC

SEQ. I.D. NO. 559

GATGTTACTGGGACGGTATGAATAATCC

SEQ. I.D. NO. 560

TGATTACATGGGACGGTATGAATAATCC

SEQ. I.D. NO. 561

TTGATAATTGGGACGGTATGAATAATCC

SEQ. I.D. NO. 562

ACGCATAGTGGGACGGTATGAATAATCC

SEQ. I.D. NO. 563

CTGTGGACTGGGACGGTATGAATAATCC

SEQ. I.D. NO. 564

ATAGACAATGGGACGGTATGAATAATCC

SEQ. I.D. NO. 565

CCATTGTTTGGGACGGTATGAATAATCC

SEQ. I.D. NO. 566

AGAGGAATTGGGACGGTATGAATAATCC

SEQ. I.D. NO. 567

CTTCCTTCTGGGACGGTATGAATAATCC

SEQ. I.D. NO. 568

TCTAGCGATGGGACGGTATGAATAATCC

SEQ. I.D. NO. 569

TCAACTGTTGGGACGGTATGAATAATCC

SEQ. I.D. NO. 570

GACTATTGTGGGACGGTATGAATAATCC

SEQ. I.D. NO. 571

CAACGGTCTGGGACGGTATGAATAATCC

SEQ. I.D. NO. 572

CTTGCAGATGGGACGGTATGAATAATCC

SEQ. I.D. NO. 573

GATACAGTTGGGACGGTATGAATAATCC

SEQ. I.D. NO. 574

CCTGGTAGTGGGACGGTATGAATAATCC

SEQ. I.D. NO. 575

GTTAGGTCTGGGACGGTATGAATAATCC

SEQ. I.D. NO. 576

TACTTGCATGGGACGGTATGAATAATCC

SEQ. I.D. NO. 577

TCCATGCTTGGGACGGTATGAATAATCC

SEQ. I.D. NO. 578

ACATAGCGTGGGACGGTATGAATAATCC

SEQ. I.D. NO. 579

TGGATATCTGGGACGGTATGAATAATCC

SEQ. I.D. NO. 580

GAGTTACATGGGACGGTATGAATAATCC

SEQ. I.D. NO. 581

TGCGACCTTGGGACGGTATGAATAATCC

SEQ. I.D. NO. 582

ATCCGCAGTGGGACGGTATGAATAATCC

SEQ. I.D. NO. 583

CAGTTGGTTGGGACGGTATGAATAATCC

SEQ. I.D. NO. 584

CTGATTAATGGGACGGTATGAATAATCC

SEQ. I.D. NO. 585

TCGCACCTTGGGACGGTATGAATAATCC

SEQ. I.D. NO. 586

CGCCACAGTGGGACGGTATGAATAATCC

SEQ. I.D. NO. 587

GTTGCGGCTGGGACGGTATGAATAATCC

SEQ. I.D. NO. 588

AGATATAATGGGACGGTATGAATAATCC

24. The set of primers of any one of clauses 1-23, wherein the unique polynucleotide identifier comprises an endogenous polynucleotide identifier.

25. The set of primers of any one of clauses 1-24, wherein the unique polynucleotide identifier comprises a 16S sequence.

26. The set of primers of any one of clauses 1-25, wherein the set of primers comprises primers for detection of a 16S sequence.

27. The set of primers of clause 26, wherein the set of primers is a pair of primers and wherein each pair of primers comprises a second polynucleotide sequence configured for strain-specific 16S detection.

28. The set of primers of any one of clauses 1-27, wherein the second polynucleotide sequence comprises any one of the below sequences, or the reverse complement thereof:

| Primer* | Sequence (5′-3′) | Target Group | Reference |
|---|---|---|---|
| 8F | AGAGTTTGATCCTGGCT | Universal | Turner et |
| | CAG | | al. 1999 |
| | SEQ. I.D. NO. 589 | | |
| 27F | AGAGTTTGATCMTGGC | Universal | Lane et al. |
| | TCAG | | 1991 |
| | SEQ. I.D. NO. 590 | | |
| CYA106F | CGGACGGGTGAGTAACGCGTGA | Cyanobacteria | Nübel et |
| | | | al. 1997 |
| | SEQ. I.D. NO. 591 | | |
| CC [F] | CCAGACTCCTACGGGAGGCAGC | Universal | Rudi et al. |
| | | | 1997 |
| | SEQ. I.D. NO. 592 | | |
| 357F | CTCCTACGGGAGGCAG | Universal | Turner et |
| | CAG | | al. 1999 |
| | SEQ. I.D. NO. 593 | | |
| CYA359F | GGGGAATYTTCCGCAA | Cyanobacteria | Nübel et |
| | TGGG | | al. 1997 |
| | SEQ. I.D. NO. 594 | | |
| 515F | GTGCCAGCMGCCGCGG | Universal | Turner et |
| | TAA | | al. 1999 |
| | SEQ. I.D. NO. 595 | | |
| 533F | GTGCCAGCAGCCGCGG | Universal | Weisburg |
| | TAA | | et al. 1991 |
| | SEQ. I.D. NO. 596 | | |
| 895F | CRCCTGGGGAGTRCRG | Bacteria exc. | Hodkinson |
| | SEQ. I.D. NO. 597 | plastids & | & Lutzoni |
| | | Cyanobacteria | 2009 |
| 16S.1100.F16 | CAACGAGCGCAACCCT | Universal | Turner et |
| | SEQ. I.D. NO. 598 | | al. 1999 |
| 1237F | GGGCTACACACGYGCW | Universal | Turner et |
| | AC | | al. 1999 |
| | SEQ. I.D. NO. 599 | | |
| 519R | GWATTACCGCGGCKGC | Universal | Turner et |
| | TG | | al. 1999 |
| | SEQ. I.D. NO. 600 | | |
| CYA781R | GACTACWGGGGTATCT | Cyanobacteria | Nübel et |
| | AATCCCWTT | | al. 1997 |
| | SEQ. I.D. NO. 601 | | |
| CD [R] | CTTGTGCGGGCCCCCGT | Universal | Rudi et al. |
| | CAATTC | | 1997 |
| | SEQ. I.D. NO. 602 | | |
| 902R | GTCAATTCITTTGAGTTT | Bacteria exc. | Hodkinson |
| | YARYC | plastids & | & Lutzoni |
| | SEQ. I.D. NO. 603 | Cyanobacteria | 2009 |
| 904R | CCCCGTCAATTCITTTGA | Bacteria exc. | Hodkinson |
| | GTTTYAR | plastids & | & Lutzoni |
| | SEQ. I.D. NO. 604 | Cyanobacteria | 2009 |
| 907R | CCGTCAATTCMTTTRAG | Universal | Lane et al. |
| | TTT | | 1991 |
| | SEQ. I.D. NO. 605 | | |
| 1100R | AGGGTTGCGCTCGTTG | Bacteria | Turner et |
| | SEQ. I.D. NO. 606 | | al. 1999 |
| 1185mR | GAYTTGACGTCATCCM | Bacteria exc. | Hodkinson |
| | SEQ. I.D. NO. 607 | plastids & | & Lutzoni |
| | | Cyanobacteria | 2009 |
| 1185aR | GAYTTGACGTCATCCA | Lichen- | Hodkinson |
| | SEQ. I.D. NO. 608 | associated | & Lutzoni |
| | | Rhizobiales | 2009 |
| 1381R | CGGTGTGTACAAGRCC | Bacteria exc. | Hodkinson |
| | YGRGA | Asterochloris | & Lutzoni |
| | SEQ. I.D. NO. 609 | sp. plastids | 2009 |
| 1381bR | CGGGCGGTGTGTACAA | Bacteria exc. | Hodkinson |
| | GRCCYGRGA | Asterochloris | & Lutzoni |
| | SEQ. I.D. NO. 610 | sp. plastids | 2009 |
| 1391R | GACGGGCGGTGTGTRC | Universal | Turner et |
| | A | | al. 1999 |
| | SEQ. I.D. NO. 611 | | |
| 1492R (l) | GGTTACCTTGTTACGAC | Universal | Turner et |
| | TT | | al. 1999 |
| | SEQ. I.D. NO. 612 | | |
| 1492R (s) | ACCTTGTTACGACTT | Universal | Lane et al. |
| | SEQ. I.D. NO. 613 | | 1991 |

29. The set of primers of any one of clauses 1-28, wherein the second polynucleotide sequence comprises any one of the below sequences, or the reverse complement thereof:

F:
5′-AAGGGGCATGATGACTTGAC-3′
R:
5′-GAGATGTCGGTTCCCTTGTG-3′
F:
5′-TCCTACGGGAGGCAGCAGT-3′
R:
5′-GGACTACCAGGGTATCTAATCCTGTT-3′.

30. The set of primers of any one of clauses 1-29, wherein the growth conditions comprise temperature, exposure to one or more chemical or biological agent, time duration of each exposure, concentration of each chemical or biological agent, or any combination thereof.

31. A collection of double-stranded nucleic acid molecules for multiplex high-resolution detection of micro-organism strains amongst a strain collection and for multiplex identification of given growth conditions of said micro-organism strains, wherein each molecule comprises an experimental conditions sequence; and a unique polynucleotide identifier.

32. The collection of double-stranded nucleic acid molecules of clause 31, wherein detection comprises absolute or relative quantification.

33. The collection of double-stranded nucleic acid molecules of any one of clauses 31-32, wherein experimental conditions comprise growth conditions.

34. The collection of double-stranded nucleic acid molecules of any one of clauses 31-33, wherein the unique polynucleotide identifier comprises an exogenous or endogenous polynucleotide sequence.

35. The collection of double-stranded nucleic acid molecules of any one of clauses 31-34 wherein the unique polynucleotide identifier comprises an exogenous polynucleotide identifier flanked by upstream and downstream respective flanking sequences common for all strains of the strain collection.

36. The collection of double-stranded nucleic acid molecules of any one of clauses 31-35, wherein the double-stranded nucleic acid molecules comprises any one of the below sequences or the reverse complement thereof:

| Primer Name | Sequence (SEQ ID NOs. 11-202) |
|---|---|
| A1 | ATCGACTG |
| B1 | GCTAGCAG |
| C1 | TACTCTCC |
| D1 | TGACAGCA |
| E1 | GCAGGTTG |
| F1 | TTCCAGCT |
| G1 | TAGTTAGC |
| H1 | AGCGCTAA |
| A2 | CGGTTCTT |
| B2 | TAGCATTG |
| C2 | AATTCAAC |
| D2 | TTCACAGA |
| E2 | GCTCTCTT |
| F2 | TGACTTGG |
| G2 | TATGGTTC |
| H2 | CACTAGCC |
| A3 | AACCTCTT |
| B3 | CTACATTG |
| C3 | GCGATTAC |
| D3 | AATTGGCC |
| E3 | AATTGCTT |
| F3 | TTGGTCTG |
| G3 | CATCCTGG |
| H3 | GGATTAAC |
| A4 | CGCATATT |
| B4 | TCATTCGA |
| C4 | GTCCAATC |
| D4 | CTTGGTCA |
| E4 | CCAACGCT |
| F4 | TCCACTTC |
| G4 | AATCTCCA |
| H4 | GTCTGCAC |
| A5 | CTGCTCCT |
| B5 | TTAGCCAG |
| C5 | GCTGATTC |
| D5 | GAATCGAC |
| E5 | AGTCACCT |
| F5 | CACGATTC |
| G5 | GCTCCGAT |
| H5 | CTTGGCTT |
| A6 | GCTGCACT |
| B6 | GAACTTCG |
| C6 | CTGTATTC |
| D6 | ATATCCGA |
| E6 | TTGTCCAT |
| F6 | AGTAAGTC |
| G6 | GAATATCA |
| H6 | CAACTGAT |
| A7 | CCTGTCAT |
| B7 | GACGGTTA |
| C7 | CTATTAGC |
| D7 | TCCAACCA |
| E7 | CTGGCTAT |
| F7 | GCGGACTT |
| G7 | CCATCACA |
| H7 | GGCAATAC |
| A8 | CACTTCAT |
| B8 | CAAGCTTA |
| C8 | AGGTACCA |
| D8 | TCCATAAC |
| E8 | GTCCTCAT |
| F8 | AGTACTGC |
| G8 | CTTGAATC |
| H8 | CCAACTAA |
| A9 | AATACCAT |
| B9 | GCGATATT |
| C9 | GAACGCTA |
| D9 | CTGACATC |
| E9 | GCCACCAT |
| F9 | CGACTCTC |
| G9 | TGCTATTA |
| H9 | CTTCTGGC |
| A10 | ATGAATTA |
| B10 | TACTCCAG |
| C10 | ATCATACC |
| D10 | CCTCTAAC |
| E10 | ATCTTCTC |
| F10 | CAGCTCAC |
| G10 | GGTTATCT |
| H10 | TCCGCATA |
| A11 | TGCTTCAC |
| B11 | GCTTCCTA |
| C11 | GACCATCT |
| D11 | CTGGTATT |
| E11 | TTAATCAC |
| F11 | CGCGAATA |
| G11 | GCTCACCA |
| H11 | TCATGTCT |
| A12 | ATCCTTAA |
| B12 | TTCTTGGC |
| C12 | CATCACTT |
| D12 | CGAACTTC |
| E12 | GACATTAA |
| F12 | TTCACCTT |
| G12 | CCAATCTG |
| H12 | CGACAGTT |
| Plate1 | AAGTAGAG |
| Plate2 | CATGCTTA |
| Plate3 | GCACATCT |
| Plate4 | TGCTCGAC |
| Plate5 | AGCAATTC |
| Plate6 | AGTTGCTT |
| Plate7 | CCAGTTAG |
| Plate8 | TTGAGCCT |
| Plate9 | ACACGATC |
| Plate10 | GGTCCAGA |
| Plate11 | GTATAACA |
| Plate12 | TTCGCTGA |
| Plate13 | AACTTGAC |
| Plate14 | CACATCCT |
| Plate15 | TCGGAATG |
| Plate16 | AAGGATGT |
| Plate17 | CGCGCGGT |
| Plate18 | TCTGGCGA |
| Plate19 | CATAGCGA |
| Plate20 | CAGGAGCC |
| Plate21 | TGTCGGAT |
| Plate22 | ATTATGTT |
| Plate23 | CCTACCAT |
| Plate24 | TACTTAGC |
| Plate25 | CATGATCG |
| Plate26 | AGGATCTA |
| Plate27 | GACAGTAA |
| Plate28 | CCTATGCC |
| Plate29 | TCGCCTTG |
| Plate30 | ATAGCGTC |
| Plate31 | GAAGAAGT |
| Plate32 | ATTCTAGG |
| Plate33 | CGTTACCA |
| Plate34 | GTCTGATG |
| Plate35 | TTACGCAC |
| Plate36 | TTGAATAG |
| Plate37 | AAGACACT |
| Plate38 | CAGCAAGG |
| Plate39 | TCCAGCAA |
| Plate40 | CCAGAGCT |
| Plate41 | TCCTTGGT |
| Plate42 | AGGTTATC |
| Plate43 | GTCATCTA |
| Plate44 | CCTTCGCA |
| Plate45 | TCTCGGTC |
| Plate46 | ATTGTCTG |
| Plate47 | GAACCTAG |
| Plate48 | TAATGAAC |
| Plate49 | AACGCATT |
| Plate50 | CAACTCTC |
| Plate51 | CTGTAATC |
| Plate52 | TAAGCACA |
| Plate53 | AATGTTCT |
| Plate54 | CAGCGGTA |
| Plate55 | GACCAGGA |
| Plate56 | TATCCAGG |
| Plate57 | ACAGGTAT |
| Plate58 | CCAACATT |
| Plate59 | GCCGTCGA |
| Plate60 | TATCTGCC |
| Plate61 | ACTAAGAC |
| Plate62 | CGCCTTCC |
| Plate63 | GCCTAGCC |
| Plate64 | TGCTGCTG |
| Plate65 | AGGTAAGG |
| Plate66 | CTAACTCG |
| Plate67 | GTAACATC |
| Plate68 | TGTAATCA |
| Plate69 | ATTATCAA |
| Plate70 | CTATGCGT |
| Plate71 | GTCCACAG |
| Plate72 | TTGTCTAT |
| Plate73 | AACAATGG |
| Plate74 | ATTCCTCT |
| Plate75 | GAAGGAAG |
| Plate76 | TCGCTAGA |
| Plate77 | ACAGTTGA |
| Plate78 | CAATAGTC |
| Plate79 | GACCGTTG |
| Plate80 | TCTGCAAG |
| Plate81 | ACTGTATC |
| Plate82 | CTACCAGG |
| Plate83 | GACCTAAC |
| Plate84 | TGCAAGTA |
| Plate85 | AGCATGGA |
| Plate86 | CGCTATGT |
| Plate87 | GATATCCA |
| Plate88 | TGTAACTC |
| Plate89 | AGGTCGCA |
| Plate90 | CTGCGGAT |
| Plate91 | ACCAACTG |
| Plate92 | TTAATCAG |
| Plate93 | AGGTGCGA |
| Plate94 | CTGTGGCG |
| Plate95 | GCCGCAAC |
| Plate96 | TTATATCT |

5′ Flank
TATTTATGCAGAGGCCGAGG
3′ Flank Sequence SEQ ID NO: 203
GGATTATTCATACCGTCCCA.
5′ Primer Sequence (GC + Well
BC + 5′Flank) SEQ ID NO. 204
(SEQ ID NOs. 205-300)
GCATCGACTGTATTTATGCAGAGGCCGAGG
GCGCTAGCAGTATTTATGCAGAGGCCGAGG
GCTACTCTCCTATTTATGCAGAGGCCGAGG
GCTGACAGCATATTTATGCAGAGGCCGAGG
GCGCAGGTTGTATTTATGCAGAGGCCGAGG
GCTTCCAGCTTATTTATGCAGAGGCCGAGG
GCTAGTTAGCTATTTATGCAGAGGCCGAGG
GCAGCGCTAATATTTATGCAGAGGCCGAGG
GCCGGTTCTTTATTTATGCAGAGGCCGAGG
GCTAGCATTGTATTTATGCAGAGGCCGAGG
GCAATTCAACTATTTATGCAGAGGCCGAGG
GCTTCACAGATATTTATGCAGAGGCCGAGG
GCGCTCTCTTTATTTATGCAGAGGCCGAGG
GCTGACTTGGTATTTATGCAGAGGCCGAGG
GCTATGGTTCTATTTATGCAGAGGCCGAGG
GCCACTAGCCTATTTATGCAGAGGCCGAGG
GCAACCTCTTTATTTATGCAGAGGCCGAGG
GCCTACATTGTATTTATGCAGAGGCCGAGG
GCGCGATTACTATTTATGCAGAGGCCGAGG
GCAATTGGCCTATTTATGCAGAGGCCGAGG
GCAATTGCTTTATTTATGCAGAGGCCGAGG
GCTTGGTCTGTATTTATGCAGAGGCCGAGG
GCCATCCTGGTATTTATGCAGAGGCCGAGG
GCGGATTAACTATTTATGCAGAGGCCGAGG
GCCGCATATTTATTTATGCAGAGGCCGAGG
GCTCATTCGATATTTATGCAGAGGCCGAGG
GCGTCCAATCTATTTATGCAGAGGCCGAGG
GCCTTGGTCATATTTATGCAGAGGCCGAGG
GCCCAACGCTTATTTATGCAGAGGCCGAGG
GCTCCACTTCTATTTATGCAGAGGCCGAGG
GCAATCTCCATATTTATGCAGAGGCCGAGG
GCGTCTGCACTATTTATGCAGAGGCCGAGG
GCCTGCTCCTTATTTATGCAGAGGCCGAGG
GCTTAGCCAGTATTTATGCAGAGGCCGAGG
GCGCTGATTCTATTTATGCAGAGGCCGAGG
GCGAATCGACTATTTATGCAGAGGCCGAGG
GCAGTCACCTTATTTATGCAGAGGCCGAGG
GCCACGATTCTATTTATGCAGAGGCCGAGG
GCGCTCCGATTATTTATGCAGAGGCCGAGG
GCCTTGGCTTTATTTATGCAGAGGCCGAGG
GCGCTGCACTTATTTATGCAGAGGCCGAGG
GCGAACTTCGTATTTATGCAGAGGCCGAGG
GCCTGTATTCTATTTATGCAGAGGCCGAGG
GCATATCCGATATTTATGCAGAGGCCGAGG
GCTTGTCCATTATTTATGCAGAGGCCGAGG
GCAGTAAGTCTATTTATGCAGAGGCCGAGG
GCGAATATCATATTTATGCAGAGGCCGAGG
GCCAACTGATTATTTATGCAGAGGCCGAGG
GCCCTGTCATTATTTATGCAGAGGCCGAGG
GCGACGGTTATATTTATGCAGAGGCCGAGG
GCCTATTAGCTATTTATGCAGAGGCCGAGG
GCTCCAACCATATTTATGCAGAGGCCGAGG
GCCTGGCTATTATTTATGCAGAGGCCGAGG
GCGCGGACTTTATTTATGCAGAGGCCGAGG
GCCCATCACATATTTATGCAGAGGCCGAGG
GCGGCAATACTATTTATGCAGAGGCCGAGG
GCCACTTCATTATTTATGCAGAGGCCGAGG
GCCAAGCTTATATTTATGCAGAGGCCGAGG
GCAGGTACCATATTTATGCAGAGGCCGAGG
GCTCCATAACTATTTATGCAGAGGCCGAGG
GCGTCCTCATTATTTATGCAGAGGCCGAGG
GCAGTACTGCTATTTATGCAGAGGCCGAGG
GCCTTGAATCTATTTATGCAGAGGCCGAGG
GCCCAACTAATATTTATGCAGAGGCCGAGG
GCAATACCATTATTTATGCAGAGGCCGAGG
GCGCGATATTTATTTATGCAGAGGCCGAGG
GCGAACGCTATATTTATGCAGAGGCCGAGG
GCCTGACATCTATTTATGCAGAGGCCGAGG
GCGCCACCATTATTTATGCAGAGGCCGAGG
GCCGACTCTCTATTTATGCAGAGGCCGAGG
GCTGCTATTATATTTATGCAGAGGCCGAGG
GCCTTCTGGCTATTTATGCAGAGGCCGAGG
GCATGAATTATATTTATGCAGAGGCCGAGG
GCTACTCCAGTATTTATGCAGAGGCCGAGG
GCATCATACCTATTTATGCAGAGGCCGAGG
GCCCTCTAACTATTTATGCAGAGGCCGAGG
GCATCTTCTCTATTTATGCAGAGGCCGAGG
GCCAGCTCACTATTTATGCAGAGGCCGAGG
GCGGTTATCTTATTTATGCAGAGGCCGAGG
GCTCCGCATATATTTATGCAGAGGCCGAGG
GCTGCTTCACTATTTATGCAGAGGCCGAGG
GCGCTTCCTATATTTATGCAGAGGCCGAGG
GCGACCATCTTATTTATGCAGAGGCCGAGG
GCCTGGTATTTATTTATGCAGAGGCCGAGG
GCTTAATCACTATTTATGCAGAGGCCGAGG
GCCGCGAATATATTTATGCAGAGGCCGAGG
GCGCTCACCATATTTATGCAGAGGCCGAGG
GCTCATGTCTTATTTATGCAGAGGCCGAGG
GCATCCTTAATATTTATGCAGAGGCCGAGG
GCTTCTTGGCTATTTATGCAGAGGCCGAGG
GCCATCACTTTATTTATGCAGAGGCCGAGG
GCCGAACTTCTATTTATGCAGAGGCCGAGG
GCGACATTAATATTTATGCAGAGGCCGAGG
GCTTCACCTTTATTTATGCAGAGGCCGAGG
GCCCAATCTGTATTTATGCAGAGGCCGAGG
GCCGACAGTTTATTTATGCAGAGGCCGAGG
3′ Primer Sequence
(GC + Plate BC + Rev. comp.
of 3′ Flank) (SEQ ID NOs. 301-396)
GCCTCTACTTTGGGACGGTATGAATAATCC
GCTAAGCATGTGGGACGGTATGAATAATCC
GCAGATGTGCTGGGACGGTATGAATAATCC
GCGTCGAGCATGGGACGGTATGAATAATCC
GCGAATTGCTTGGGACGGTATGAATAATCC
GCAAGCAACTTGGGACGGTATGAATAATCC
GCCTAACTGGTGGGACGGTATGAATAATCC
GCAGGCTCAATGGGACGGTATGAATAATCC
GCGATCGTGTTGGGACGGTATGAATAATCC
GCTCTGGACCTGGGACGGTATGAATAATCC
GCTGTTATACTGGGACGGTATGAATAATCC
GCTCAGCGAATGGGACGGTATGAATAATCC
GCGTCAAGTTTGGGACGGTATGAATAATCC
GCAGGATGTGTGGGACGGTATGAATAATCC
GCCATTCCGATGGGACGGTATGAATAATCC
GCACATCCTTTGGGACGGTATGAATAATCC
GCACCGCGCGTGGGACGGTATGAATAATCC
GCTCGCCAGATGGGACGGTATGAATAATCC
GCTCGCTATGTGGGACGGTATGAATAATCC
GCGGCTCCTGTGGGACGGTATGAATAATCC
GCATCCGACATGGGACGGTATGAATAATCC
GCAACATAATTGGGACGGTATGAATAATCC
GCATGGTAGGTGGGACGGTATGAATAATCC
GCGCTAAGTATGGGACGGTATGAATAATCC
GCCGATCATGTGGGACGGTATGAATAATCC
GCTAGATCCTTGGGACGGTATGAATAATCC
GCTTACTGTCTGGGACGGTATGAATAATCC
GCGGCATAGGTGGGACGGTATGAATAATCC
GCCAAGGCGATGGGACGGTATGAATAATCC
GCGACGCTATTGGGACGGTATGAATAATCC
GCACTTCTTCTGGGACGGTATGAATAATCC
GCCCTAGAATTGGGACGGTATGAATAATCC
GCTGGTAACGTGGGACGGTATGAATAATCC
GCCATCAGACTGGGACGGTATGAATAATCC
GCGTGCGTAATGGGACGGTATGAATAATCC
GCCTATTCAATGGGACGGTATGAATAATCC
GCAGTGTCTTTGGGACGGTATGAATAATCC
GCCCTTGCTGTGGGACGGTATGAATAATCC
GCTTGCTGGATGGGACGGTATGAATAATCC
GCAGCTCTGGTGGGACGGTATGAATAATCC
GCACCAAGGATGGGACGGTATGAATAATCC
GCGATAACCTTGGGACGGTATGAATAATCC
GCTAGATGACTGGGACGGTATGAATAATCC
GCTGCGAAGGTGGGACGGTATGAATAATCC
GCGACCGAGATGGGACGGTATGAATAATCC
GCCAGACAATTGGGACGGTATGAATAATCC
GCCTAGGTTCTGGGACGGTATGAATAATCC
GCGTTCATTATGGGACGGTATGAATAATCC
GCAATGCGTTTGGGACGGTATGAATAATCC
GCGAGAGTTGTGGGACGGTATGAATAATCC
GCGATTACAGTGGGACGGTATGAATAATCC
GCTGTGCTTATGGGACGGTATGAATAATCC
GCAGAACATTTGGGACGGTATGAATAATCC
GCTACCGCTGTGGGACGGTATGAATAATCC
GCTCCTGGTCTGGGACGGTATGAATAATCC
GCCCTGGATATGGGACGGTATGAATAATCC
GCATACCTGTTGGGACGGTATGAATAATCC
GCAATGTTGGTGGGACGGTATGAATAATCC
GCTCGACGGCTGGGACGGTATGAATAATCC
GCGGCAGATATGGGACGGTATGAATAATCC
GCGTCTTAGTTGGGACGGTATGAATAATCC
GCGGAAGGCGTGGGACGGTATGAATAATCC
GCGGCTAGGCTGGGACGGTATGAATAATCC
GCCAGCAGCATGGGACGGTATGAATAATCC
GCCCTTACCTTGGGACGGTATGAATAATCC
GCCGAGTTAGTGGGACGGTATGAATAATCC
GCGATGTTACTGGGACGGTATGAATAATCC
GCTGATTACATGGGACGGTATGAATAATCC
GCTTGATAATTGGGACGGTATGAATAATCC
GCACGCATAGTGGGACGGTATGAATAATCC
GCCTGTGGACTGGGACGGTATGAATAATCC
GCATAGACAATGGGACGGTATGAATAATCC
GCCCATTGTTTGGGACGGTATGAATAATCC
GCAGAGGAATTGGGACGGTATGAATAATCC
GCCTTCCTTCTGGGACGGTATGAATAATCC
GCTCTAGCGATGGGACGGTATGAATAATCC
GCTCAACTGTTGGGACGGTATGAATAATCC
GCGACTATTGTGGGACGGTATGAATAATCC
GCCAACGGTCTGGGACGGTATGAATAATCC
GCCTTGCAGATGGGACGGTATGAATAATCC
GCGATACAGTTGGGACGGTATGAATAATCC
GCCCTGGTAGTGGGACGGTATGAATAATCC
GCGTTAGGTCTGGGACGGTATGAATAATCC
GCTACTTGCATGGGACGGTATGAATAATCC
GCTCCATGCTTGGGACGGTATGAATAATCC
GCACATAGCGTGGGACGGTATGAATAATCC
GCTGGATATCTGGGACGGTATGAATAATCC
GCGAGTTACATGGGACGGTATGAATAATCC
GCTGCGACCTTGGGACGGTATGAATAATCC
GCATCCGCAGTGGGACGGTATGAATAATCC
GCCAGTTGGTTGGGACGGTATGAATAATCC
GCCTGATTAATGGGACGGTATGAATAATCC
GCTCGCACCTTGGGACGGTATGAATAATCC
GCCGCCACAGTGGGACGGTATGAATAATCC
GCGTTGCGGCTGGGACGGTATGAATAATCC
GCAGATATAATGGGACGGTATGAATAATCC
5′ Primer Sequence
(No more GC + Well BC +
5′Flank) (SEQ ID NOs. 397-492)
ATCGACTGTATTTATGCAGAGGCCGAGG
GCTAGCAGTATTTATGCAGAGGCCGAGG
TACTCTCCTATTTATGCAGAGGCCGAGG
TGACAGCATATTTATGCAGAGGCCGAGG
GCAGGTTGTATTTATGCAGAGGCCGAGG
TTCCAGCTTATTTATGCAGAGGCCGAGG
TAGTTAGCTATTTATGCAGAGGCCGAGG
AGCGCTAATATTTATGCAGAGGCCGAGG
CGGTTCTTTATTTATGCAGAGGCCGAGG
TAGCATTGTATTTATGCAGAGGCCGAGG
AATTCAACTATTTATGCAGAGGCCGAGG
TTCACAGATATTTATGCAGAGGCCGAGG
GCTCTCTTTATTTATGCAGAGGCCGAGG
TGACTTGGTATTTATGCAGAGGCCGAGG
TATGGTTCTATTTATGCAGAGGCCGAGG
CACTAGCCTATTTATGCAGAGGCCGAGG
AACCTCTTTATTTATGCAGAGGCCGAGG
CTACATTGTATTTATGCAGAGGCCGAGG
GCGATTACTATTTATGCAGAGGCCGAGG
AATTGGCCTATTTATGCAGAGGCCGAGG
AATTGCTTTATTTATGCAGAGGCCGAGG
TTGGTCTGTATTTATGCAGAGGCCGAGG
CATCCTGGTATTTATGCAGAGGCCGAGG
GGATTAACTATTTATGCAGAGGCCGAGG
CGCATATTTATTTATGCAGAGGCCGAGG
TCATTCGATATTTATGCAGAGGCCGAGG
GTCCAATCTATTTATGCAGAGGCCGAGG
CTTGGTCATATTTATGCAGAGGCCGAGG
CCAACGCTTATTTATGCAGAGGCCGAGG
TCCACTTCTATTTATGCAGAGGCCGAGG
AATCTCCATATTTATGCAGAGGCCGAGG
GTCTGCACTATTTATGCAGAGGCCGAGG
CTGCTCCTTATTTATGCAGAGGCCGAGG
TTAGCCAGTATTTATGCAGAGGCCGAGG
GCTGATTCTATTTATGCAGAGGCCGAGG
GAATCGACTATTTATGCAGAGGCCGAGG
AGTCACCTTATTTATGCAGAGGCCGAGG
CACGATTCTATTTATGCAGAGGCCGAGG
GCTCCGATTATTTATGCAGAGGCCGAGG
CTTGGCTTTATTTATGCAGAGGCCGAGG
GCTGCACTTATTTATGCAGAGGCCGAGG
GAACTTCGTATTTATGCAGAGGCCGAGG
CTGTATTCTATTTATGCAGAGGCCGAGG
ATATCCGATATTTATGCAGAGGCCGAGG
TTGTCCATTATTTATGCAGAGGCCGAGG
AGTAAGTCTATTTATGCAGAGGCCGAGG
GAATATCATATTTATGCAGAGGCCGAGG
CAACTGATTATTTATGCAGAGGCCGAGG
CCTGTCATTATTTATGCAGAGGCCGAGG
GACGGTTATATTTATGCAGAGGCCGAGG
CTATTAGCTATTTATGCAGAGGCCGAGG
TCCAACCATATTTATGCAGAGGCCGAGG
CTGGCTATTATTTATGCAGAGGCCGAGG
GCGGACTTTATTTATGCAGAGGCCGAGG
CCATCACATATTTATGCAGAGGCCGAGG
GGCAATACTATTTATGCAGAGGCCGAGG
CACTTCATTATTTATGCAGAGGCCGAGG
CAAGCTTATATTTATGCAGAGGCCGAGG
AGGTACCATATTTATGCAGAGGCCGAGG
TCCATAACTATTTATGCAGAGGCCGAGG
GTCCTCATTATTTATGCAGAGGCCGAGG
AGTACTGCTATTTATGCAGAGGCCGAGG
CTTGAATCTATTTATGCAGAGGCCGAGG
CCAACTAATATTTATGCAGAGGCCGAGG
AATACCATTATTTATGCAGAGGCCGAGG
GCGATATTTATTTATGCAGAGGCCGAGG
GAACGCTATATTTATGCAGAGGCCGAGG
CTGACATCTATTTATGCAGAGGCCGAGG
GCCACCATTATTTATGCAGAGGCCGAGG
CGACTCTCTATTTATGCAGAGGCCGAGG
TGCTATTATATTTATGCAGAGGCCGAGG
CTTCTGGCTATTTATGCAGAGGCCGAGG
ATGAATTATATTTATGCAGAGGCCGAGG
TACTCCAGTATTTATGCAGAGGCCGAGG
ATCATACCTATTTATGCAGAGGCCGAGG
CCTCTAACTATTTATGCAGAGGCCGAGG
ATCTTCTCTATTTATGCAGAGGCCGAGG
CAGCTCACTATTTATGCAGAGGCCGAGG
GGTTATCTTATTTATGCAGAGGCCGAGG
TCCGCATATATTTATGCAGAGGCCGAGG
TGCTTCACTATTTATGCAGAGGCCGAGG
GCTTCCTATATTTATGCAGAGGCCGAGG
GACCATCTTATTTATGCAGAGGCCGAGG
CTGGTATTTATTTATGCAGAGGCCGAGG
TTAATCACTATTTATGCAGAGGCCGAGG
CGCGAATATATTTATGCAGAGGCCGAGG
GCTCACCATATTTATGCAGAGGCCGAGG
TCATGTCTTATTTATGCAGAGGCCGAGG
ATCCTTAATATTTATGCAGAGGCCGAGG
TTCTTGGCTATTTATGCAGAGGCCGAGG
CATCACTTTATTTATGCAGAGGCCGAGG
CGAACTTCTATTTATGCAGAGGCCGAGG
GACATTAATATTTATGCAGAGGCCGAGG
TTCACCTTTATTTATGCAGAGGCCGAGG
CCAATCTGTATTTATGCAGAGGCCGAGG
CGACAGTTTATTTATGCAGAGGCCGAGG
3′ Primer Sequence
(GC + Plate BC + Rev. comp. of
3′ Flank) (SEQ ID NOs. 493-588)
CTCTACTTTGGGACGGTATGAATAATCC
TAAGCATGTGGGACGGTATGAATAATCC
AGATGTGCTGGGACGGTATGAATAATCC
GTCGAGCATGGGACGGTATGAATAATCC
GAATTGCTTGGGACGGTATGAATAATCC
AAGCAACTTGGGACGGTATGAATAATCC
CTAACTGGTGGGACGGTATGAATAATCC
AGGCTCAATGGGACGGTATGAATAATCC
GATCGTGTTGGGACGGTATGAATAATCC
TCTGGACCTGGGACGGTATGAATAATCC
TGTTATACTGGGACGGTATGAATAATCC
TCAGCGAATGGGACGGTATGAATAATCC
GTCAAGTTTGGGACGGTATGAATAATCC
AGGATGTGTGGGACGGTATGAATAATCC
CATTCCGATGGGACGGTATGAATAATCC
ACATCCTTTGGGACGGTATGAATAATCC
ACCGCGCGTGGGACGGTATGAATAATCC
TCGCCAGATGGGACGGTATGAATAATCC
TCGCTATGTGGGACGGTATGAATAATCC
GGCTCCTGTGGGACGGTATGAATAATCC
ATCCGACATGGGACGGTATGAATAATCC
AACATAATTGGGACGGTATGAATAATCC
ATGGTAGGTGGGACGGTATGAATAATCC
GCTAAGTATGGGACGGTATGAATAATCC
CGATCATGTGGGACGGTATGAATAATCC
TAGATCCTTGGGACGGTATGAATAATCC
TTACTGTCTGGGACGGTATGAATAATCC
GGCATAGGTGGGACGGTATGAATAATCC
CAAGGCGATGGGACGGTATGAATAATCC
GACGCTATTGGGACGGTATGAATAATCC
ACTTCTTCTGGGACGGTATGAATAATCC
CCTAGAATTGGGACGGTATGAATAATCC
TGGTAACGTGGGACGGTATGAATAATCC
CATCAGACTGGGACGGTATGAATAATCC
GTGCGTAATGGGACGGTATGAATAATCC
CTATTCAATGGGACGGTATGAATAATCC
AGTGTCTTTGGGACGGTATGAATAATCC
CCTTGCTGTGGGACGGTATGAATAATCC
TTGCTGGATGGGACGGTATGAATAATCC
AGCTCTGGTGGGACGGTATGAATAATCC
ACCAAGGATGGGACGGTATGAATAATCC
GATAACCTTGGGACGGTATGAATAATCC
TAGATGACTGGGACGGTATGAATAATCC
TGCGAAGGTGGGACGGTATGAATAATCC
GACCGAGATGGGACGGTATGAATAATCC
CAGACAATTGGGACGGTATGAATAATCC
CTAGGTTCTGGGACGGTATGAATAATCC
GTTCATTATGGGACGGTATGAATAATCC
AATGCGTTTGGGACGGTATGAATAATCC
GAGAGTTGTGGGACGGTATGAATAATCC
GATTACAGTGGGACGGTATGAATAATCC
TGTGCTTATGGGACGGTATGAATAATCC
AGAACATTTGGGACGGTATGAATAATCC
TACCGCTGTGGGACGGTATGAATAATCC
TCCTGGTCTGGGACGGTATGAATAATCC
CCTGGATATGGGACGGTATGAATAATCC
ATACCTGTTGGGACGGTATGAATAATCC
AATGTTGGTGGGACGGTATGAATAATCC
TCGACGGCTGGGACGGTATGAATAATCC
GGCAGATATGGGACGGTATGAATAATCC
GTCTTAGTTGGGACGGTATGAATAATCC
GGAAGGCGTGGGACGGTATGAATAATCC
GGCTAGGCTGGGACGGTATGAATAATCC
CAGCAGCATGGGACGGTATGAATAATCC
CCTTACCTTGGGACGGTATGAATAATCC
CGAGTTAGTGGGACGGTATGAATAATCC
GATGTTACTGGGACGGTATGAATAATCC
TGATTACATGGGACGGTATGAATAATCC
TTGATAATTGGGACGGTATGAATAATCC
ACGCATAGTGGGACGGTATGAATAATCC
CTGTGGACTGGGACGGTATGAATAATCC
ATAGACAATGGGACGGTATGAATAATCC
CCATTGTTTGGGACGGTATGAATAATCC
AGAGGAATTGGGACGGTATGAATAATCC
CTTCCTTCTGGGACGGTATGAATAATCC
TCTAGCGATGGGACGGTATGAATAATCC
TCAACTGTTGGGACGGTATGAATAATCC
GACTATTGTGGGACGGTATGAATAATCC
CAACGGTCTGGGACGGTATGAATAATCC
CTTGCAGATGGGACGGTATGAATAATCC
GATACAGTTGGGACGGTATGAATAATCC
CCTGGTAGTGGGACGGTATGAATAATCC
GTTAGGTCTGGGACGGTATGAATAATCC
TACTTGCATGGGACGGTATGAATAATCC
TCCATGCTTGGGACGGTATGAATAATCC
ACATAGCGTGGGACGGTATGAATAATCC
TGGATATCTGGGACGGTATGAATAATCC
GAGTTACATGGGACGGTATGAATAATCC
TGCGACCTTGGGACGGTATGAATAATCC
ATCCGCAGTGGGACGGTATGAATAATCC
CAGTTGGTTGGGACGGTATGAATAATCC
CTGATTAATGGGACGGTATGAATAATCC
TCGCACCTTGGGACGGTATGAATAATCC
CGCCACAGTGGGACGGTATGAATAATCC
GTTGCGGCTGGGACGGTATGAATAATCC
AGATATAATGGGACGGTATGAATAATCC

37. The collection of double-stranded nucleic acid molecules of any one of clauses 31-36, wherein the unique polynucleotide identifier comprises any one of the below nucleotide sequences:

| Barcode | | |
|---|---|---|
| Name | Barcode Sequence | |
| 1 | ACATATCCAACCTTATATAACATT | SEQ. I.D. NO. 614 |
| 2 | TCTAACATACACTCATAATAATAC | SEQ. I.D. NO. 615 |
| 3 | TATATAATTCCTCATACCACATAA | SEQ. I.D. NO. 616 |
| 4 | TCAATTACACTCTATAATACCTTA | SEQ. I.D. NO. 617 |
| 5 | TAATTATACATCTCATCTTCTACA | SEQ. I.D. NO. 618 |
| 6 | CTACTATACATCTTACTATACTTT | SEQ. I.D. NO. 619 |
| 7 | AACATCTATCTTTCTAACTTTCAA | SEQ. I.D. NO. 620 |
| 8 | AACCTATTATTCTCTACCTATAAT | SEQ. I.D. NO. 621 |
| 9 | CTACATCTAATCATTACTATAACA | SEQ. I.D. NO. 622 |
| 10 | CATTCAATACACAAATACTCAAAT | SEQ. I.D. NO. 623 |
| 11 | CTTCTATCTATCTTTCATTTCTAT | SEQ. I.D. NO. 624 |
| 12 | TTAATCTTCAATATACCTTACCAA | SEQ. I.D. NO. 625 |
| 13 | CAACTACACTTATCATTACATAAA | SEQ. I.D. NO. 626 |
| 14 | TTAATCTTCAATATACCTTACCAA | SEQ. I.D. NO. 627 |
| 15 | TAATACATAACTACTAACTCTAAC | SEQ. I.D. NO. 628 |
| 16 | TTCACTTATCTACTATTTCTTAAC | SEQ. I.D. NO. 629 |
| 17 | TCTATAACTCCACTTAATAACATA | SEQ. I.D. NO. 630 |
| 18 | AACTTAATCTCTTATAACTACCTT | SEQ. I.D. NO. 631 |
| 19 | ATTAATTCCACTTACCTTACAATA | SEQ. I.D. NO. 632 |
| 20 | ATTATTATCATTCCTATCTAACCA | SEQ. I.D. NO. 633 |
| 21 | TTACCTTAACTATATTCTACAACA | SEQ. I.D. NO. 634 |
| 22 | ATTTACACTACTTACACACAATAA | SEQ. I.D. NO. 635 |
| 23 | TACTTAAACATACAAACTTACTCA | SEQ. I.D. NO. 636 |
| 24 | TCATATACTACTCTTTAAACACTA | SEQ. I.D. NO. 637 |
| 25 | TCTTTCAAACAATACTTCTCTAAA | SEQ. I.D. NO. 638 |
| 26 | TTATTACACTCTATACTCTAATTC | SEQ. I.D. NO. 639 |
| 27 | CTACACTATATATTCTACACAATT | SEQ. I.D. NO. 640 |
| 28 | AATTCAACTACTCTCAATTTACTA | SEQ. I.D. NO. 641 |
| 29 | ACATAATTCTACTCTAACTCATTT | SEQ. I.D. NO. 642 |
| 30 | TAATTATACATCTCATCTTCTACA | SEQ. I.D. NO. 643 |
| 31 | TCACTAATTAATCACCTACATATT | SEQ. I.D. NO. 644 |
| 32 | CTACTATACATCTTACTATACTTT | SEQ. I.D. NO. 645 |
| 33 | AACATCTATCTTTCTAACTTTCAA | SEQ. I.D. NO. 646 |
| 34 | ATATCTATCATCCTACTACATATA | SEQ. I.D. NO. 647 |

38. The collection of double-stranded nucleic acid molecules of any one of clauses 31-37, wherein the collection is obtainable by PCR amplification using the set of primers of any one of clauses 1-30.

39. The collection of double-stranded nucleic acid molecules of any one of clauses 31-38, wherein each molecule further comprises any one of the below adapter sequences:

P5 Adapter

(SEQ. I.D. No. 5)

AATGATACGGCGACCACCGA

(SEQ. I.D. No. 648)

AATGATACGGCGACCACCGAGATCT

(SEQ. I.D. No. 3)

AATGATACGGCGACCACCGAGATCTACACTCTTTCCCTACACGACGCTCT
TCCGATCT
Illumina Sequencing Primers

(SEQ. I.D. No. 649)

ACACTCTTTCCCTACACGACGCTCTTCCGATCT

(SEQ. I.D. No. 650)

ACACTCTTTCCCTACACGACGCTCTTCCGATCTGC
P7 Adapter

(SEQ. I.D. No. 6)

CAAGCAGAAGACGGCATACGA

(SEQ. I.D. No. 651)

TCGTATGCCGTCTTCTGCTTG

(SEQ. I.D. No. 652)

CAAGCAGAAGACGGCATACGAGCTCTTCCGATC

(SEQ. I.D. No. 653)

GATCGGAAGAGCATCTCGTATGCCGTCTTCTGCTTG

40. The collection of double-stranded nucleic acid molecules of any one of clauses 31-39, wherein each molecule is about 150 to about 500 bp.

41. The collection of double-stranded nucleic acid molecules of any one of clauses 31-40, wherein each molecule is about 150 to about 300 bp.

42. A collection of probes, wherein the probes comprise denatured double-stranded nucleic acid molecules amplified by the set of primers of any one of clauses 1-30.

43. A set of probes for multiplex high-resolution detection of micro-organism strains amongst a strain collection and for multiplex identification of given growth conditions of said micro-organism strains, wherein each probe is a single stranded nucleic acid molecule from a collection of any one of clauses 1-30.

44. A set of primers of any one of clauses 1-30 or set of probes of any one of clauses 42 or 43 for use in diagnostics.

45. A method for the diagnostic of a pathogenic infection, by multiplex high-resolution detection of micro-organism strains from a strain collection, wherein said method comprises:
providing a test sample from a patient;
extracting exogenous nucleic acids from said test sample; and
hybridizing said exogenous nucleic acids with the set of primers of any one of clauses 1-30 or set of probes of any one of clauses 42 or 43.

46. A method of generating and selecting a collection of hypomorph strains of a micro-organism population, comprising:
generating a collection of strains of micro-organisms, wherein for each strain the level of expression of a unique gene is controlled by an exogenous promoter, whereby the level of expression of the unique gene is altered compared with the level of expression of the unique gene under its endogenous promoter, each strain of micro-organism having a unique polynucleotide identifier, whereby each unique polynucleotide identifier is configured for multiplex high-resolution detection of the corresponding strain amongst said collection of strains;
outgrowing the generated strains of micro-organisms; and
selecting the hypomorph strains of micro-organisms based on growth kinetics and the expression level of the unique gene, the expression level of the unique gene being indicative of the promoter strength.

47. The method of clause 46, wherein detection comprises absolute or relative quantification.

48. The method of any one of clauses 46 or 47, wherein the exogenous promoter reduces the level expression of a unique gene by 2-10 times the level expression of the unique gene under its endogenous promoter.

49. The method of any one of clauses 46-48, wherein generating the collection of strains comprises replacing the endogenous promoter of the unique gene.

50. The method of any one of clauses 46-49, wherein generating the collection of strains comprises:
integrating an engineered copy of the unique gene into the genome of the organism population, the engineered copy comprising the unique gene and an exogenous promoter and
deleting the endogenous copy of the unique gene from the genome of the organism population.

51. The method of any one of clauses 46-50, further comprising generating and selecting a set of promoters and selecting the exogenous promoters.

52. The method of any one of clauses 46-51, wherein generating the set of promoters comprises:
generating a set of candidate promoters;
generating a collection of tested strains of a micro-organism population, wherein for each tested strain a marker-coding polynucleotide sequence and one of the candidate promoters operatively linked to the marker-coding polynucleotide sequence are integrated into the genome of the micro-organism population;
measuring expression of the marker of each tested strains; and
selecting the promoters based on marker expression.

53. The method of clause 52, wherein the marker is a color marker. 54. The method of clause 53, wherein the color marker is GFP. 55. The method of any one of clauses 52-54, wherein the marker-coding polynucleotide sequence is integrated at the attTn7 site.

56. The method of clause 55, wherein integration is by a mini-Tn7 suicide vector. 57. The method of any one of clauses 46-56, wherein generating a set of candidate promoters comprises selecting a first set of variable promoters based on their ability to promote marker expression in one model micro-organism, wherein the variable promoters are obtained through random mutation on common nucleic sequences.

58. The method of clause 57, wherein the common nucleic sequences comprise the −35 and −10 RNA Pol binding sequences.

59. The method of any one of clauses 57-58, wherein the other nucleic sequences are the nucleic sequence between −35 and −10 RNA Pol binding sequences.

60. The method of any one of clauses 57-59, wherein generating the set of candidate promoters further comprises generating a second set of variable promoters from the first set by altering other nucleic sequences.

61. The method of any one of clauses 46-60, wherein the micro-organism population comprises a pathogenic micro-organism population.

62. The method of clause 61, wherein the pathogenic micro-organism population is or was derived from a bacterial cell, or a fungus cell.

63. The method of clause 62, wherein the bacterial cell is a Gram negative or Gram positive bacterial cell.

64. The method of clause 62, wherein the pathogenic micro-organism population is or was derived from Acinetabacter baumanii, Klebsiella pneumonaie, Enterobacteriaceae spp., Pseudomonas aeruginosa, Staphylococcus aureus or Mycobacteriium tuberculosis.

65. The method of any one of clauses 46-64, wherein the unique polynucleotide identifier comprises an exogenous or endogenous polynucleotide sequence.

66. The method of any one of clauses 46-65, wherein the unique polynucleotide identifier comprises an exogenous polynucleotide identifier flanked by upstream and downstream respective flanking sequences common for all strains of the strain collection.

67. The method of any one of clauses 46-66, wherein the unique polynucleotide identifier comprises an endogenous polynucleotide identifier.

68. The method of any one of clauses 46-67, wherein the unique polynucleotide identifier comprises a 16S sequence.

69. The method of clause 68, wherein the 16S sequence comprises any one of the below sequences, or the reverse complement thereof:

| | | Target | |
|---|---|---|---|
| Primer* | Sequence (5′-3′) | Group | Reference |
| 8F | AGAGTTTGATCCTGGCTCAG | Universal | Turner et |
| | | | al. 1999 |
| 27F | AGAGTTTGATCMTGGCTCAG | Universal | Lane et al. |
| | | | 1991 |
| CYA106F | CGGACGGGTGAGTAACGCGTGA | Cyanobacteria | Nubel et |
| | | | al. 1997 |
| CC [F] | CCAGACTCCTACGGGAGGCAGC | Universal | Rudi et al. |
| | | | 1997 |
| 357F | CTCCTACGGGAGGCAGCAG | Universal | Turner et |
| | | | al. 1999 |
| CYA359F | GGGGAATYTTCCGCAATGGG | Cyanobacteria | Nubel et |
| | | | al. 1997 |
| 515F | GTGCCAGCMGCCGCGGTAA | Universal | Turner et |
| | | | al. 1999 |
| 533F | GTGCCAGCAGCCGCGGTAA | Universal | Weisburg |
| | | | et al. 1991 |
| 895F | CRCCTGGGGAGTRCRG | Bacteria exc. | Hodkinson & |
| | | plastids & | Lutzoni |
| | | Cyanobacteria | 2009 |
| 16S.1100.F16 | CAACGAGCGCAACCCT | Universal | Turner et |
| | | | al. 1999 |
| 1237F | GGGCTACACACGYGCWAC | Universal | Turner et |
| | | | al. 1999 |
| 519R | GWATTACCGCGGCKGCTG | Universal | Turner et |
| | | | al. 1999 |
| CYA781R | GACTACWGGGGTATCTAATCCCWTT | Cyanobacteria | Nubel et |
| | | | al. 1997 |
| CD [R] | CTTGTGCGGGCCCCCGTCAATTC | Universal | Rudi et al. |
| | | | 1997 |
| 902R | GTCAATTCITTTGAGTTTYARYC | Bacteria exc. | Hodkinson & |
| | | plastids & | Lutzoni |
| | | Cyanobacteria | 2009 |
| 904R | CCCCGTCAATTCITTTGAGTTTYAR | Bacteria exc. | Hodkinson & |
| | | plastids & | Lutzoni |
| | | Cyanobacteria | 2009 |
| 907R | CCGTCAATTCMTTTRAGTTT | Universal | Lane et al. |
| | | | 1991 |
| 1100R | AGGGTTGCGCTCGTTG | Bacteria | Turner et |
| | | | al. 1999 |
| 1185mR | GAYTTGACGTCATCCM | Bacteria exc. | Hodkinson & |
| | | plastids & | Lutzoni |
| | | Cyanobacteria | 2009 |
| 1185aR | GAYTTGACGTCATCCA | Lichen- | Hodkinson & |
| | | associated | Lutzoni |
| | | Rhizobiales | 2009 |
| 1381R | CGGTGTGTACAAGRCCYGRGA | Bacteria exc. | Hodkinson & |
| | | Asterochloris | Lutzoni |
| | | sp. plastids | 2009 |
| 1381bR | CGGGCGGTGTGTACAAGRCCYGRGA | Bacteria exc. | Hodkinson & |
| | | Asterochloris | Lutzoni |
| | | sp. plastids | 2009 |
| 1391R | GACGGGCGGTGTGTRCA | Universal | Turner et |
| | | | al. 1999 |
| 1492R (l) | GGTTACCTTGTTACGACTT | Universal | Turner et |
| | | | al. 1999 |
| 1492R (s) | ACCTTGTTACGACTT | Universal | Lane et al. |
| | | | 1991 |

70. A collection of hypomorph strains of a micro-organism population obtainable by the method of any one of clauses 46-69.

71. A method of screening assay of a set of experimental conditions on a collection of strains of a micro-organism, comprising, for each strain:
providing a collection of hypomorph micro-organism strains;
preparing a pool of strains from said collection;
subjecting said pool of strains to a set of experimental conditions; and
performing multiplex high-resolution detection of the strains amongst said collection of strains.

72. The method of clause 71, wherein experimental conditions comprise growth conditions.

73. The method of any one of clauses 71-72, wherein the method further comprises PCR-detection or sequencing.

74. The method of any one of clauses 71-73, wherein detection comprises absolute or relative quantification.

75. The method of any one of clauses 71-74, wherein the collection of hypomorph strains comprises the collection of clause 70.

76. The method of any one of clauses 71-75, wherein the detection is performed with the set of primers of any one of clauses 1-30 or detection of double-stranded nucleic acid molecules of any one of clauses 31-41 or collection of probes of any one of clauses 42-43.

77. The method of any one of clauses 71-76, wherein the experimental or growth conditions comprise temperature, exposure to a chemical or biological agent, time duration of each exposure, concentration of each chemical or biological agent, or any combination thereof.

78. The method of any one of clauses 71-77, further comprising pooling all hypomorph genotypes of the strain before subjecting them to a set of experimental conditions.

79. The method of any one of clauses 71-78, further comprising prior to pooling the hypomorph genotypes of the strain, outgrowing the hypomorph genotypes of the strain under conditions that repress hypomorph phenotype expression so that phenotype close to that of the wild type of the strain is obtained for all hypomorph genotypes of the strain.

80. The method of any one of clauses 71-79, wherein the exogenous promoter comprises a Tet-on promoter and wherein the method further comprises prior to pooling all hypomorph genotypes strain, outgrowing the hypomorph genotypes of the strain with tetracycline, a tetracycline derivative, doxycycline or anhydrotetracycline.

81. The method of clause 80, wherein the strain is outgrown with anhydrotetracycline at a concentration of about 300 to about 700 μg/mL.

82. The method of clause 81, wherein the strain is outgrown with anhydrotetracycline at a concentration of about 400 to about 600 μg/mL.

83. The method of clause 81, wherein the strain is outgrown with anhydrotetracycline at a concentration of about 450 to about 550 μg/mL.

84. The method of clause 81, wherein the strain is outgrown with anhydrotetracycline at a concentration of about 500 μg/mL.

85. The method of any one of clauses 80-84, wherein the strain is outgrown with anhydrotetracycline for 18 to 78 hours.

86. The method of any one of clauses 80-84, wherein the strain is outgrown with anhydrotetracycline for 48 to 72 hours.

87. The method of any one of clauses 71-86, wherein the experimental or growth condition comprises exposure to a chemical or biological agent at an effective concentration, wherein the micro-organism is a pathogen, and wherein analyzing all hypomorph genotypes of all strains comprises determining the effectiveness of the chemical or biological agent to control or stop proliferation of the hypomorph genotype.

88. The method of any one of clauses 71-87, wherein the experimental or growth condition comprises exposure to a chemical or biological agent at a range of values of concentration, wherein the micro-organism is a pathogen, and wherein analyzing all hypomorph genotypes of all strains comprises determining a value of effective concentration of the chemical or biological agent to control or stop proliferation of the hypomorph genotype.

89. The method of any one of clauses 87-88, wherein determining the effectiveness of the chemical or biological agent to control or stop proliferation of the hypomorph genotype comprises determining at least one of IC50 value of the chemical or biological agent and MIC90 value of the chemical or biological agent for each hypomorph genotype, the IC50 or MIC90 value being indicative of the effectiveness of the chemical or biological agent to control or stop proliferation of the hypomorph genotype.

90. The method of any one of clauses 71-89, wherein analyzing all hypomorph genotypes of all strains further comprises
determining the specificity of the chemical or biological agent to the strains and identifying a chemical or biological agent specific to a group of hypomorph genotypes or to only one hypomorph genotype.

91. The method of any one of clauses 71-90, further comprising PCR amplifying the unique polynucleotide identifier using a set of primers of any one of clauses 1-30.

92. The method of clause 91, wherein PCR amplification comprises about 15 to about 30 cycles.

93. The method of clause 91, wherein PCR amplification comprises about 17 to about 25 cycles.

94. The method of clause 91, wherein PCR amplification comprises about 22 cycles.

95. A method for identifying a compound or compound structure with anti-bacterial property, comprising the method of assay of any one of clauses 71-94.

96. The method of clause 95, wherein the antibacterial compound comprises a chemical or biological agent.

97. The method of clause 95, wherein the antibacterial compound comprises a bactericidal or bacteriostatic agent.

98. A method for identifying a pathogenic micro-organism with the set of primers of any one of clauses 1-30 or detection of double-stranded nucleic acid molecules of any one of clauses 31-41 or collection of probes of any one of clauses 42-43.

99. A kit for multiplex high-resolution detection of micro-organism strains amongst a strain collection and for multiplex identification of given growth conditions of said micro-organism strain.

100. A diagnostic kit for multiplex high-resolution detection of micro-organism strains amongst a strain collection and for multiplex identification of given growth conditions of said micro-organism strain.

101. The kit of any one of clause 99-100, wherein detection comprises absolute or relative quantification.

102. The kit of any one of clauses 99-101, wherein said kit comprises the set of primers of any one of clauses 1-30, the double-stranded nucleic acid molecules of any one of clauses 31-41 or the collection of probes of any one of clauses 42-43.

The present invention will be further illustrated in the following Examples which are given for illustration purposes only and are not intended to limit the invention in any way.

### EXAMPLES

### Example 1

### Outline and Principle of Identification of Essential Proteins in Pseudomonas aeruginosa

The present inventors have performed Tn-seq on 20 different strains including 5 strains from cystic fibrosis patients isolated at Children's Hospital Boston, as well as strains isolated from urine, blood, ocular infections, ventilator-associated pneumonia, and the environment. The present inventors have constructed Illumina Tn-seq libraries from each transposon library, which are sequenced in collaboration with the Broad Institute Genome Sequencing Center for Infectious Diseases (GSCID) [Gallagher, L. A., J. Shendure, and C. Manoil, Genome-Scale Identification of Resistance Functions in Pseudomonas aeruginosa Using Tn-seq. MBio, 2011. 2(1); Gawronski, J. D., et al., Tracking insertion mutants within libraries by deep sequencing and a genome-wide screen for Haemophilus genes required in the lung. Proc Natl Acad Sci USA, 2009. 106(38): p. 16422-7.]. Illumina TnSeq sequence data for P. aeruginosa PAO1 and PA14 can be compared with the published genome sequences of these strains. In addition, whole genome sequencing and assembly on the 18 strains for which genomes do not currently exist are performed. Thus, Tn-seq libraries for every strain may be compared with the reference genome of the parent strain to determine essentiality. It is then expected to define the common essential genes across all 80 strain and growth condition combinations; these common essential genes should represent the highest probability targets for effective novel antimicrobials. Previous studies have estimated 335 essential gene candidates in LB media alone in strain PA14, which is consistent with our findings for growth of strain PA14 on LB media [Liberati, N. T., et al., Comparing insertion libraries in two Pseudomonas aeruginosa strains to assess gene essentiality. Methods Mol Biol, 2008. 416: p. 153-69.]. From preliminary studies, inventors found that the number of essential genes required for growth under all 4 conditions, reduces the number candidates down to 265 essential genes:

PA14 essential genes in LB, M9, Blood, and Urine

| Cellular Compartment | # of Essential Genes |
|---|---|
| Cytoplasm | 140 |
| Cytoplasmic Membrane | 48 |
| Periplasm | 4 |
| Outer Membrane | 5 |
| Extracellular | 3 |
| Unknown | 65 |
| Total | 265 |

Putative essential genes are as follows

| PA14 Identifier | Gene | Localization | Function |
|---|---|---|---|
| PA14_07770 | ostA | OM | membrane impermeability |
| PA14_12210 | Hypothetical | OM | Unknown; membrane/LPS |
| | | | biogenesis? |
| PA14_17150 | opr86 | OM | outer membrane protein |
| | | | assembly |
| PA14_51710 | oprL | OM | outer membrane integrity |
| PA14_57920 | Hypothetical | OM | Unknown |
| PA14_61740 | lolB | OM | outer membrane protein |
| | | | assembly/chaperone |
| PA14_63030 | omlA | OM | outer membrane protein |
| | | | assembly |
| PA14_69660 | lppL | OM | LPS biosynthesis |
| PA14_07760 | surA | Peri | outer membrane protein |
| | | | assembly |
| PA14_30310 | lolA | Peri | outer membrane protein |
| | | | assembly/chaperone |
| PA14_51720 | tolB | Peri | outer membrane integrity |
| PA14_51730 | tolA | Peri/IM | outer membrane integrity |
| PA14_58130 | mreC | Peri | rod-shape structural protein |
| PA14_07570 | gcp | Extra | endonuclease; cell wall |
| | | | biosynthesis? |

Within the set of 265 genes there are five that have been shown to be outer membrane localized. This list includes ostA, tolA, oprL, omlA, and lppL.

### Example 2

### Outline and Principle of a High-Throughput Chemical Screen for Multiplexed Targeting of Essential Proteins (MTEP) in Pseudomonas aeruginosa

Engineering hypersusceptible strains (hypomorph strains): Strain PA14 is engineered so that the expression of selected essential genes may be lowered using a ‘weaker’ promoter. For each essential gene, one may create a strain using published methods by chromosomally integrating a new gene copy into the attTn7 site using mini-Tn7 (Choi, K. H. and H. P. Schweizer, mini-Tn7 insertion in bacteria with single attTn7 sites: example Pseudomonas aeruginosa. Nat Protoc, 2006. 1(1): p. 153-61) driven by the weak promoter followed by two-step homologous recombination with sacB counter selection to delete the endogenous gene copy (Choi, K. H. and H. P. Schweizer, An improved method for rapid generation of unmarked Pseudomonas aeruginosa deletion mutants. BMC Microbiol, 2005. 5: p. 30). It is possible to use a promoter library of varying strengths that was developed to drive GFP expression in E. coli (Davis, J. H., A. J. Rubin, and R. T. Sauer, Design, construction and characterization of a set of insulated bacterial promoters. Nucleic Acids Res, 2011. 39(3): p. 1131-41). Using these promoters, along with additional ones that were created by modifying the spacing between the RNA polymerase binding sites of the promoters, inventors have tested their efficacy by chromosomally integrating GFP into P. aeruginosa PA14. The weakest promoter that provides the lowest tolerable level of the protein that still yields a viable bacterium may be used for each essential gene to create a hypersensitive strain. It is also proposed to construct control strains by knocking out dihydrofolate reductase dhfr (which is the target of trimethoprim), dihydropteroate synthetase dhps (which is the target of sulfamethoxazole), murA (which is the target of fosfomycin) and ostA (which is the OMP target of POL7001 [Srinivas, N., et al., Peptidomimetic antibiotics target outer-membrane biogenesis in Pseudomonas aeruginosa. Science, 2010. 327(5968): p. 1010-3.]). Then, it is possible to measure the MIC for each of these compounds against their respective strains compared to wild-type PA14. It is expected that the engineered strains be sensitized to the corresponding antibiotic that targets the respective gene product. Inventors have successfully created a dhfr knockdown using this method, which is more sensitive to trimethoprim than the wild-type PA14 strain.

Multiplexed screening assay: a method is proposed where all strains are screened simultaneously in multiplex by pooling them for growth. To accomplish this, inventors genetically barcode each pooled strain by inserting a 76 bp sequence encoding a unique 24 bp barcode with two PCR primer-flanking regions (26 bp each) into each mutant. This allows to amplify the barcoded region and use next-generation Illumina sequencing to identify and quantitate the barcode/strain within the pooled population. Inventors also barcode wild-type strains of PA14 and other organisms (E. coli, S. aureus, K pneumoniae, A. baumannii and the fungus C. albicans) that may also serve as controls within the screen to determine the spectrum of activity of any hit. Molecules which kill both bacterial and fungal strains are likely to be non-specific, perhaps membrane disrupting, toxic compounds which are of little interest. These 10 constructed control strains, including their known antibiotics, may be used for assay development. The general method may involve seeding the control strains into a well with compound or DMSO control (in LB media), allowing growth to occur for a determined amount of time, lysing the cells to release their DNA, PCR amplification of barcodes from lysates using plate and well barcodes for pooling, ligation of Illumina sequencing adapters, and finally demultiplexing and counting the number of reads of each strain following Illumina sequencing.

### Example 3

### Multiplexed Targeting of Essential Proteins (MTEP) Screen for Essential Outer Membrane Proteins (OMPs)

Having optimized the assay for control screening strains, inventors engineer screening strains targeting the candidate list from Example 1 and optimize the assay against the total collection of screening strains for MTEP. First, inventors use the methods of Example 2 to engineer and barcode screening strains for the knockdown of the genes encoding essential OMPs identified in Example 1. This forms the screening population, which may include barcoded wild-type PA14, E. coli, S. aureus, K. pneumoniae, Acinetobacter, C. albicans, and one control engineered strain (dhfr, dhps, or murA) and essential OMP engineered knockdown screening strains (hypomorph strains, including lptD). Inventors confirm the MTEP method and that Illumina sequencing can clearly measure the census of each mutant in a pooled population and detect reduction in a subset of targeted screening strains. Initially, inventors pilot the screen on a 2,000 compound library from the Broad Institute chemical library collection. One may then screen the library in duplicate, using controls used in Example 2 to determine the robustness of the assay and its readiness for large-scale screening. Given the low number of compounds, inventors anticipate that this pilot is predominantly to assess the performance of the screen and do not necessarily anticipate obtaining any specific hits. Once pilot screen is optimized, inventors perform chemical HTS of a unique 40,000 compound diversity oriented synthesis library from the Broad Institute using MTEP against the mixture of pooled screening strains engineered in Example 2. The screen is performed in duplicate in 384-well format to identify hits that can be classified as described above. Assuming a hit rate of ˜1%, inventors pick 400 hits for target confirmation, dose-response testing, and toxicity to eukaryotic cells. In collaboration with synthetic chemists, inventors chemically optimize these compounds with the goal of initially generating at least 60-80 analogues in order to increase both the solubility and the potency against multiple clinical strains of P. aeruginosa. Furthermore, inventors identify the exact mechanism of action and protein-binding sites by the compounds using various biochemical and biophysical techniques, depending on the target identity.

### Example 4

### Exemplary Primers, Double-stranded Nucleic Acid Molecules and Probes

An example of primer has one of the following structures:
5′-[sequencing sequence]-[A/T]-[first polynucleotide sequence]-[second polynucleotide sequence]-3′
5′-[Illumina P5+Primer sequence]-[A/T]-[Well barcode]-[5′(upstream) flanking region]-3′
5′-[Illumina P7+Primer sequence]-[A/T]-[Plate barcode]-[3′(downstream) flanking region]-3′
5′-[Illumina P5+Primer sequence]-[A/T]-[Plate barcode]-[5′(upstream) flanking region]-3′
5′-[Illumina P7+Primer sequence]-[A/T]-[well barcode]-[3′(downstream) flanking region]-3′
5′-[Illumina P7+Primer sequence]-[A/T]-[Well barcode]-[5′(upstream) flanking region]-3′
5′-[Illumina P5+Primer sequence]-[A/T]-[Plate barcode]-[3′(downstream) flanking region]-3′
5′-[Illumina P7+Primer sequence]-[A/T]-[Plate barcode]-[5′(upstream) flanking region]-3′
5′-[Illumina P+Primer sequence]-[A/T]-[well barcode]-[3′(downstream) flanking region]-3′

Primer pairs may be as follows:
5′-[Illumina P5+Primer sequence]-[A/T]-[Well barcode]-[5′(upstream) flanking region]-3′ and
5′-[Illumina P7+Primer sequence]-[A/T]-[Plate barcode]-[3′(downstream) flanking region]-3′
5′-[Illumina P5+Primer sequence]-[A/T]-[Plate barcode]-[5′(upstream) flanking region]-3′ and
5′-[Illumina P7+Primer sequence]-[A/T]-[well barcode]-[3′(downstream) flanking region]-3′
5′-[Illumina P7+Primer sequence]-[A/T]-[Well barcode]-[5′(upstream) flanking region]-3′ and
5′-[Illumina P5+Primer sequence]-[A/T]-[Plate barcode]-[3′(downstream) flanking region]-3′
5′-[Illumina P7+Primer sequence]-[A/T]-[Plate barcode]-[5′(upstream) flanking region]-3′ and
5′-[Illumina P+Primer sequence]-[A/T]-[well barcode]-[3′(downstream) flanking region]-3′.

Double stranded nucleic acid and probes may have the following structure:
5′-[sequencing sequence]-[A/T]-[first polynucleotide sequence]-[second polynucleotide sequence]-[strain unique polynucleotide identifier]-[second polynucleotide sequence]-[first polynucleotide sequence]-[T/A]-[sequencing sequence]-3′
5′-[sequencing sequence]-[A/T]-[well barcode]-[second polynucleotide sequence]-[strain unique polynucleotide identifier]-[second polynucleotide sequence]-[plate barcode]-[T/A]-[sequencing sequence]-3′
5′-[sequencing sequence]-[A/T]-[plate barcode]-[second polynucleotide sequence]-[strain unique polynucleotide identifier]-[second polynucleotide sequence]-[well barcode]-[T/A]-[sequencing sequence]-3′
5′-[Illumina P5+Primer sequence]-[A/T]-[first polynucleotide sequence]-[second polynucleotide sequence]-[strain unique polynucleotide identifier]-[second polynucleotide sequence]-[first polynucleotide sequence]-[T/A]-[Illumina P7+Primer sequence]-3′
5′-[Illumina P7+Primer sequence]-[A/T]-[first polynucleotide sequence]-[second polynucleotide sequence]-[strain unique polynucleotide identifier]-[second polynucleotide sequence]-[first polynucleotide sequence]-[T/A]-[Illumina P5+Primer sequence]-3′
5′-[Illumina P5+Primer sequence]-[A/T]-[well barcode]-[second polynucleotide sequence]-[strain unique polynucleotide identifier]-[second polynucleotide sequence]-[plate barcode]-[T/A]-[Illumina P7+Primer sequence]-3′
5′-[Illumina P5+Primer sequence]-[A/T]-[plate barcode]-[second polynucleotide sequence]-[strain unique polynucleotide identifier]-[second polynucleotide sequence]-[well barcode]-[T/A]-[Illumina P7+Primer sequence]-3′
5′-[Illumina P7+Primer sequence]-[A/T]-[well barcode]-[second polynucleotide sequence]-[strain unique polynucleotide identifier]-[second polynucleotide sequence]-[plate barcode]-[T/A]-[Illumina P5+Primer sequence]-3′
5′-[Illumina P7+Primer sequence]-[A/T]-[plate barcode]-[second polynucleotide sequence]-[strain unique polynucleotide identifier]-[second polynucleotide sequence]-[well barcode]-[T/A]-[Illumina P5+Primer sequence]-3′
5′-[sequencing sequence]-[A/T]-[first polynucleotide sequence]-[5′(upstream) flanking region]-[strain unique polynucleotide identifier]-[3′(downstream) flanking region]-[first polynucleotide sequence]-[T/A]-[sequencing sequence]-3′
5′-[sequencing sequence]-[A/T]-[well barcode]-[5′(upstream) flanking region]-[strain unique polynucleotide identifier]-[3′(downstream) flanking region]-[plate barcode]-[T/A]-[sequencing sequence]-3′
5′-[sequencing sequence]-[A/T]-[plate barcode]-[5′(upstream) flanking region]-[strain unique polynucleotide identifier]-[3′(downstream) flanking region]-[well barcode]-[T/A]-[sequencing sequence]-3′
5′-[Illumina P5+Primer sequence]-[A/T]-[first polynucleotide sequence]-[5′(upstream) flanking region]-[strain unique polynucleotide identifier]-[3′(downstream) flanking region]-[first polynucleotide sequence]-[T/A]-[Illumina P7+Primer sequence]-3′
5′-[Illumina P7+Primer sequence]-[A/T]-[first polynucleotide sequence]-[5′(upstream) flanking region]-[strain unique polynucleotide identifier]-[3′(downstream) flanking region]-[first polynucleotide sequence]-[T/A]-[Illumina P5+Primer sequence]-3′
5′-[Illumina P5+Primer sequence]-[A/T]-[well barcode]-[5′(upstream) flanking region]-[strain unique polynucleotide identifier]-[3′(downstream) flanking region]-[plate barcode]-[T/A]-[Illumina P7+Primer sequence]-3′
5′-[Illumina P5+Primer sequence]-[A/T]-[plate barcode]-[5′(upstream) flanking region]-[strain unique polynucleotide identifier]-[3′(downstream) flanking region]-[well barcode]-[T/A]-[Illumina P7+Primer sequence]-3′
5′-[Illumina P7+Primer sequence]-[A/T]-[well barcode]-[5′(upstream) flanking region]-[strain unique polynucleotide identifier]-[3′(downstream) flanking region]-[plate barcode]-[T/A]-[Illumina P5+Primer sequence]-3′
5′-[Illumina P7+Primer sequence]-[A/T]-[plate barcode]-[5′(upstream) flanking region]-[3′(downstream) flanking region]-[well barcode]-[T/A]-[Illumina P5+Primer sequence]-3′

### Example 5

### Exemplary Protocol for Multiplexed Growth and Quantitation Using Illumina Sequencing

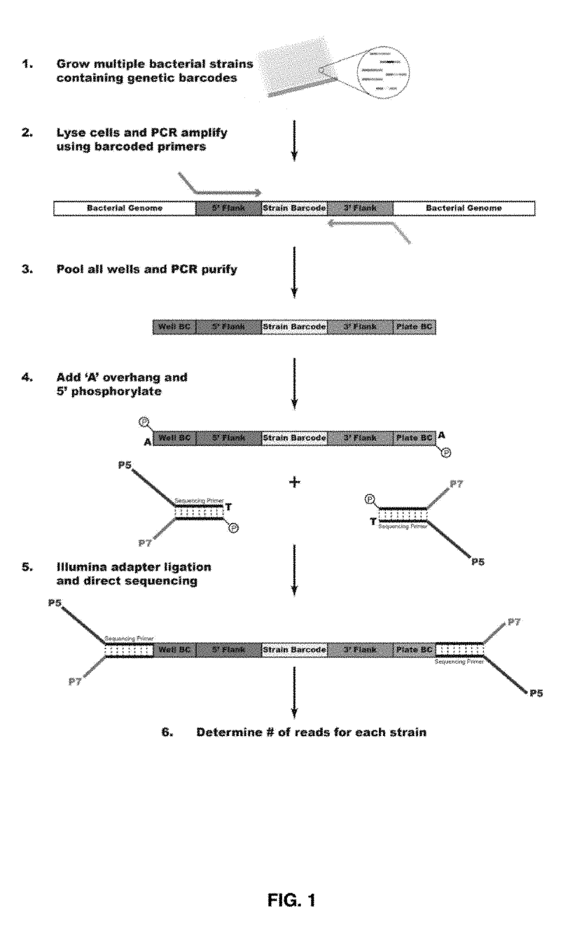

The protocol is illustrated at FIG. 1 in the form of a flow chart. In green, Well BC is a well bar code (identifier) that is in overhang before the first PCR cycle. Plate BC is a plate bar code (identifier) that is in overhang before the first PCR cycle. The strain bar code is the strain unique polynucleotide identifier. The darkened regions are identified for the PCR amplification of the unique strain identifier. These regions may be common to a subset or the entire set of strains (e.g. when the strain BC is non-endogenous, i.e. engineered, its flanking regions may be selected so as to be common to several strains, thereby being advantageous for the PCR amplification of the strain bar codes in the pool). Alternatively, these regions may correspond to an endogenous strain locus, such as 16S.

### Multiplexed Growth

### Day 1

a. Start cultures in LB, grow overnight

### Day 2

a. Subculture all cultures in LB at varying levels to cover the range of quickly vs. slow growing strains (i.e. if all strains are at stationary phase and grow at standard rates, 1/100, 1/200, and 1/500 for 3-6 hours should be sufficient)
b. Measure OD600 once cultures are in mid log phase by removing 200 μl and placing in 96-well plate (Conversion: 1.56×200 ul OD from plate=1 ml OD cuvette)
c. Aim to seed 200 CFU/well of each strain, therefore 6,666 CFU/mL. Slow growers may be seeded at higher concentrations. Do not exceed 500,000 CFU/mL.
d. Make 1× of this by pooling each strain into 1.5 L LB medium for 96 plates.
e. Add 30 μl to all wells containing compound at bottom of plate using ThermoCombi liquid dispenser
f. Pulse spin whole plate to 200 g for 1 second
g. Grow at 37° C. for 12 hours in large Tupperware with wet paper towels at the bottom with plastic lid but no sealing tape. If possible, do not stack plates. If inevitable, 4 plates per stack is the recommended maximum.

### Notes

OD to CFU/ml conversions. OD600=1 is equivalent to:
   1×10⁹ CFU/ml for Gram(−) bacteria
   6×10⁸ CFU/ml for S. aureus
   3×10⁷ CFU/ml for C. albicans

### Cell Lysis

### Day 3

a. Seal plate with Bio-Rad plate sealer B, heat at 65° C. in preheated incubator for 30 minutes
b. Let plate cool to room temperature (approx. 10 minutes; this prevents moisture buildup on seal)
c. Freeze plate at −80° C. for >15 minutes to indefinitely.

### Day 4

a. Thaw plate at room temperature on bench; be sure it is completely thawed (thawed wells are more clear from underneath). It is advised, not to spin plates at this point.
b. Optional: use plate shaker for 30 seconds and measure OD600. Z′-factors should be >0.7.
c. Add 30 μl of 2× lysis buffer to each well, incubate in Tupperware humidity chamber at 37° C. for 1 hour
d. Add 10 μl of ProK solution, incubate in humidity chamber at 37° C. for 1 hour
e. Potential pause point: seal and freeze at −80° C. Upon thawing, continue to f.
f. Spin plate at 1000 g for 5 minutes
g. Remove 20 μl lysate add to 384 well PCR plate; be sure to not allow tips to touch the bottom of the plate.
h. Seal both plates with a Bio-Rad microseal B seal and store remaining lysate at −80° C.
i. Heat denature the 20 μl lysate that has been transferred to the PCR plate in a thermocycler at 95° C. for 2 minutes, cool to 4° C. This denatures the proK. This is the template ready for PCR, and should be frozen at −20° C. with a seal.
   qPCR of Lysate and Controls to Estimate PCR Control Spike-in
Day 5
a. Prepare a template 96-well plate of PCR spike-in standards (control vector or annealed oligos) serially diluted 10-fold with a range of 0.000001-1 ng/μl for vector and 0.0001-100 pM for oligos.
b. Using Bio-Rad CFX384, perform qPCR in 13 μl reactions as follows:
   4.5 μl H₂O
   6.5 μl of 2X Mastermix (Bio-Rad iTaq SYBR)
   1 μl of 6.5 uM Primer Well A1 and Primer Plate 1 Mix (500 nM final)
   1 μl of heat-killed template (in all but 48 wells) OR 1 μl of PCR spike-in standards from a.
c. PCR cycling conditions:
   98° C. for 5 mins
   98° C. for 15 s
   60° C. for 60 s, measure signal
   Cycle 35 times
d. Make a standard curve for the spike-in controls
e. Average all heat-killed template wells and determine the amount of spike-in control relates to standard curves
f. Divide this number by # strains in pool; adding this amount per PCR reaction shall give equal number of reads as each strain
g. Multiply this amount by number of PCR reactions to be completed for the mastermix below (Section 4)
h. Use 0.5×, 1×, 2×, 5× to encompass multiple scenarios

### PCR of Lysate

### Day 6

a. Create a mastermix for 13 μl reactions as follows:
   2.6 μl 5× Q5 Reaction Buffer
   0.26 μl 10mM dNTPs
   0.13 μl Q5-Hotstart Polymerase
   X μl control #1, 2, 3, 4
   X μl H₂O
   1 μl of 6.5 uM Primer Mix (500 nM final)
   1 μl of heat-killed template (in all but 48 wells)
b. Aliquot 11 μl of the PCR Mastermix before adding 1 μl primers followed by 1 μl template
c. PCR cycling conditions:
   Initial: 98° C. for 2.5 mins
   10-20 Cycles: 98° C. for 10 s
   60° C. for 20 s
   72° C. for 20 s
   Final extension: 72° C. for 2 minutes

### PCR Cleanup

a. Pool all samples and PCR cleanup using Qiagen MinElute PCR Purification Kit (this allows for >70bp fragments, according to the invention, it is typically expected 92 bp at this point
b. Depending on # of samples, split into multiple columns. 1 column handles 5 μg; 1 per 384 wells is generous.
c. Follow Qiagen's protocol with an added PE wash
d. Elute in 10 μl EB (NOT H2O), repeat to maximize DNA (20 μl total per column)
e. Pause point: Store at −20° C. Keep 1 μl for bioanalyzer (dilute in 9 μl EB). DNA should also be visible by Nanodrop at this point at a concentration of 10 ng/μl if you have 200ng. Note: genomic DNA is expected to be heavily present.

### 5′ Phosphorylation

### Day 7

a. Performed in a thermocycler
b. Use T4 Polynucleotide Kinase from NEB
   With loss of volume assume 16 μl left per column
   Heat at 70° C. for 10 mins, then ice quickly
   Add 2 μl 10× T4 ligase buffer (not kinase buffer because the ligase buffer has the required 10 mM ATP)
   Add 2 μl T4PNK enzyme (10 U)
   Vortex briefly and spin
   37° C. for 30 minutes
c. PCR purify using the Qiagen MinElute PCR Purification Kit as in Step 3 and according to the Qiagen protocol with the following modifications: include a extra PB wash after binding; elute in 10 μl EB twice.

### 3′ A Overhang Addition

a. Performed in a thermocycler
b. Use Klenow from NEB (Taq is also an option but is performed at 72° C.)
   Assume 18 μl left
   Add 5 μl NEBNext dA-Tailing Buffer 10×, 3 ul Klenow Fragment (3′-5′ exo⁻) and 24 μl H₂O (50 μl total volume). Incubate 30mins at 37° C.
c. PCR purify using the Qiagen MinElute PCR Purification Kit as in Step 3 and according to the Qiagen protocol with the following modifications: include a extra PB wash after binding; elute in 10 μl EB twice.

### Illumina Y-Adapter Ligation

a. Construct stocks of Illumina Y-adapters
   Mix equal volumes of 100 μM P5 Adapter and 100 μM of the 5′phosphorylated P7 Adapter
   Heat to 95° C. for 2 minutes, and decrease temperature to 25° C. at a rate of 1° C./minute.
b. Ligate adapters to PCR product using the Blunt/TA Ligation Master Mix. This is the NEB preferred TA ligase and is deemed NGS compatible.
   Assume 18 μl left
   Add 4 μl of 50 μM Y-adapter (probably overkill but it works), 22 ul Blunt/TA Master Mix (44 μl total volume)
   15 minutes RT then ice
c. Perform 0.45× then 1.2× SPRI cleanup (2-step SPRI removes gDNA)
   Bring volume up to 200 uL with H₂O (ie add 156 μl)
   Add 0.45× of this volume of AMPureXP SPRI beads to the sample (90 μl), mix well by pipetting >10 times, incubate 10 minutes at RT
   Magnetize for 5 minutes, remove supernatant and place in new tube
   Add 1.2× AMPureXP SPRI beads to the sample minus the 0.45× SPRI you already added. Based on original volume of 200 μl, 1.2× would be 240 μl−90 μl=150 μl new beads. Mix well by pipetting >10 times, incubate 10 minutes at RT
   Magnetize for 5 minutes, discard supernatant
   Add 80% fresh EtOH to cover beads, incubate 1 minute, repeat
   Dry in hood until beads are cracked
   Elute in 204, EB
d. Quantify sample using Bioanalyzer, should be 188 bp (runs at 205-280 bp if Y-ends with broad peaks)
e. Quantify using KAPA Library Quantification Kit (need 4 nM minimum for Illumina)
f. Sequence on Illumina platform of your choice with custom primer and SR100 or continue to step 9 below.

### PCR Cleanup of Ends—Optional

a. This cleans the ends (not Y-shaped) and also allows you to increase concentration if required
b. Use NEBNext 2× MasterMix and your template to perform 2-10 PCR cycles, depending on required amount (possibly assume to lose at least half during cleanup, so do 2 cycles more than you think you need)
c. Setup a single 50 μl reaction per library as follows:
   2.5 μl 10 uM P5 Amplification primer
   2.5 μl 10 uM P7 Amplification primer
   25 μl 2× Master Mix
   15 μl Library
   5 μl H₂O
   Split this into 4×12.5 μl aliquots to prevent jackpotting
   Initial: 98° C. 60 s
   2-10 Cycles: 98° C. 10 s, 65° C. 20 s, 72° C. 20 s
   Final Extension: 72° C. 2 mins
   d. Pool 12.5 μl reactions together, raise volume to 100 μl (add 50 μL H₂O)
e. Add 120 μl SPRI beads for 1.2× SPRI cleanup
f. Magnetize for 5 minutes, discard supernatant
g. Add 80% fresh EtOH to cover beads, incubate 1 minute, repeat
h. Dry in hood until beads are cracked
i. Elute in 20 μL EB
j. Quantify sample using Bioanalyzer, should be 188 bp
k. Quantify sample using KAPA kit
l. Sequence on Illumina platform of your choice with custom primer and SR100

### Buffers and Primer Sequences

2× Lysis Buffer (250 ml):

| | |
|---|---|
| 237.75 ml | H₂O |
| 6.25 ml | Triton X-100 (1.25% at working concentration) |
| 5 mL | 1M Tris pH8 (10 mM at working concentration) |
| 1 ml | 0.5M EDTA (1 mM at working concentration) |

0.22 μm filter sterilize

Right before use, add the following enzymes (per ml of 2× Lysis Buffer):
If S. aureus is present: 1.7 μl of Lysostaphin (from 0.1 U/ml stock)
For Gram(−) and many Gram(+): 10 μl of Lysozyme (from 50 mg/ml stock)
If C. albicans is present: 50 μl of 1M DTT and 5 μl of Zymolyase (from 2 mg/ml stock)

### ProK Buffer:

| | |
|---|---|
| 4.337 mL | H₂O |
| 45 μl | 1M Tris pH 8 (filter sterilized) |
| 118 μl | 800 U/mL ProK |

Vortex well since ProK is in glycerol. Makes 4.5 ml, enough for a single 384 well plate. This makes a 21 U/ml solution to be added. 3 U/mL working solution.

### Primers:

| Name | Sequence |
|---|---|
| Primer ‘Well Al’ | GCXXXXXXXXTATTTATGCAGAGGCCGAGG |
| | (X's are unique barcode - strain identifier) (SEQ. I.D. No. 1) |
| Primer ‘Plate 1’ | GCXXXXXXXXTGGGACGGTATGAATAATCC |
| | (X's are unique barcode - strain identifier) (SEQ. I.D. No. 2) |
| P5 Adapter | AATGATACGGCGACCACCGAGATCTACACTCTTTCCCTACACGACGCTCTTCCGATCT |
| | (Synthesize phosphorothioate bond between 3'C and T; HPLC purify; underlined |
| | anneals with P7) (SEQ. I.D. No. 3) |
| P7 Adapter | GATCGGAAGAGCTCGTATGCCGTCTTCTGCTTG |
| | (Synthesize with 5′ phosphorylation; HPLC purify; underlined anneals |
| | with P5) (SEQ. I.D. No. 4) |
| P5 Amp | AATGATACGGCGACCACCGA (SEQ. I.D. No. 5) |
| P7 Amp | CAAGCAGAAGACGGCATACGA (SEQ. I.D. No. 6) |

### Example 6

### Creating Hypomorphic P. aeruginosa Strains and Uses Thereof for Screening

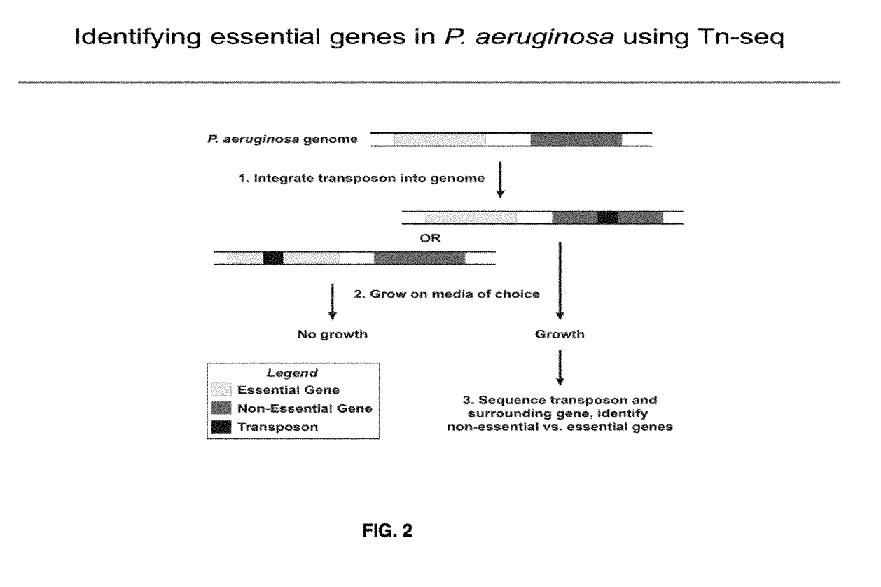

FIG. 2 depicts outline for a Tn-seq based strategy for identifying essential genes in P. aeruginosa. A comparison between P. aeruginosa strains PA14 and PAO1 identified 334 common essential proteins (Liberati, N. T. et al. PNAS (2006)). Common essential genes were identified across 13 P. aeruginosa strains and 4 different solid media: LB, Blood, M9, Urine.

By using 3 independent matings, it is possible to generate >300,000 insertions on each media.

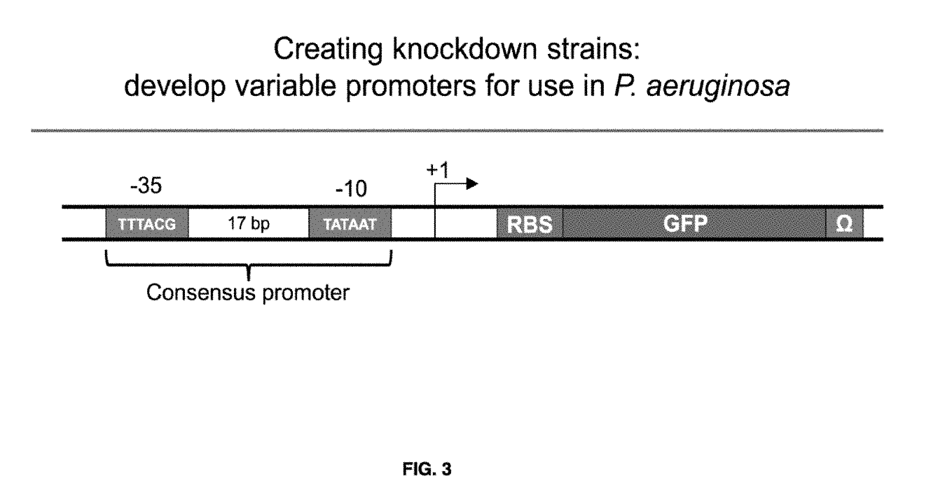

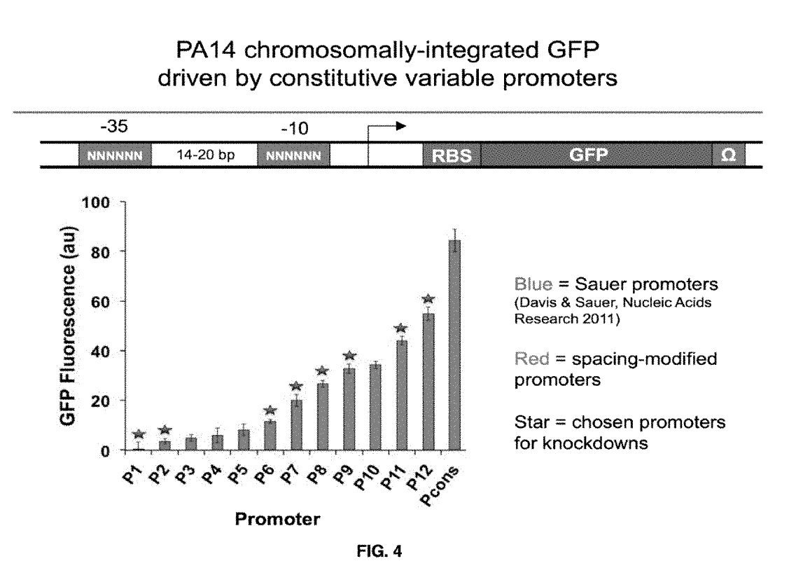

FIG. 3 illustrates a strategy for creating knockdown strains and developing variable promoters for use in P. aeruginosa. Inventors obtained a library of 8 variable promoters that were selected based on GFP expression in E. coli after randomly mutating the −35 and −10 RNA Pol binding regions (Davis & Sauer, Nucleic Acids Research 2011) PA14-GFP strains were created by integrating each promoter driving GFP at the attTn7 site using the mini-Tn7 suicide vector (Choi & Schweizer, Nature Protocols 2006). The library of 8 promoters was expanded by altering the 17bp region. Results are shown on FIG. 4 (levels of GFP fluorescence).

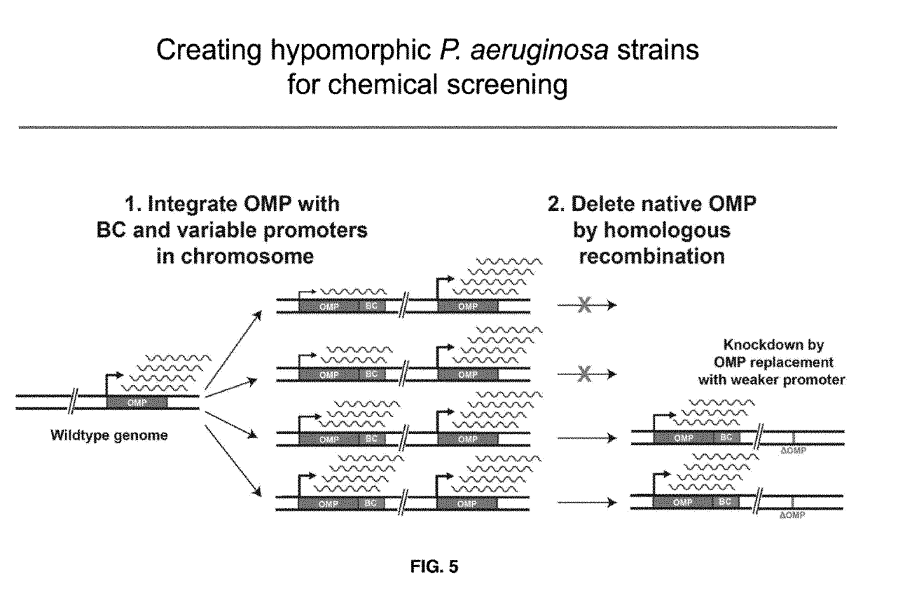

FIG. 5 illustrates the use of variable promoters for generating and selecting hypomorph strains. The waves indicate the strength of the promoters: low strength at the top, with increasing strength going down the figure. Advantageously according to the invention, the OMP under the control of the test promoters is also coupled to a strain barcode (unique strain identifier, noted BC in green). In this example, the endogenous copy of the OMP was knocked down, leaving the version under the test promoters. Survival of the strain indicates replacement with a weak promoter, with sufficient level of expression.

This leads to the generation of the following strains:

3 cytosolic control genes are targeted:
dhfR (dihydrofolate reductase), target of trimethoprim
dhpS (dihydropteroate synthase), target of sulfamethoxazole
murA (UDP-N-acetylglucosamine-3-enolpyruvyltransferase) target of fosfomycin
+17 essential OM and periplasmic proteins

8 different promoters are used, leading to a total of 160 PA14 strains.

Examples of such strains are as follows:

| Gene | Localization | Promoter |
|---|---|---|
| oprL | OM | P2 |
| lppL | OM | P12 |
| lolB | OM | P8 |
| gcp | Extra | P7 |
| CONTROLS | | |
| dhfR | Cytosol | P9 |
| murA | Cytosol | P7 |

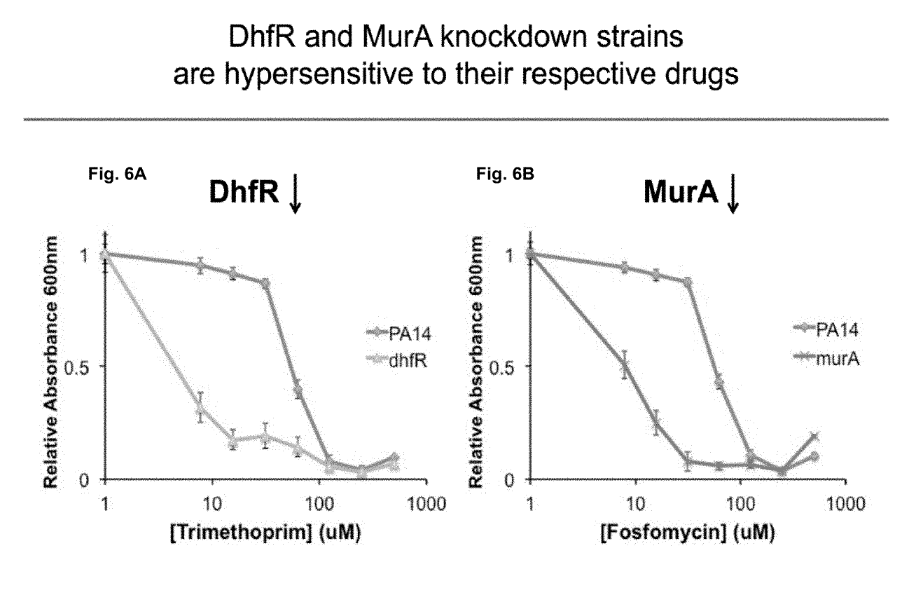

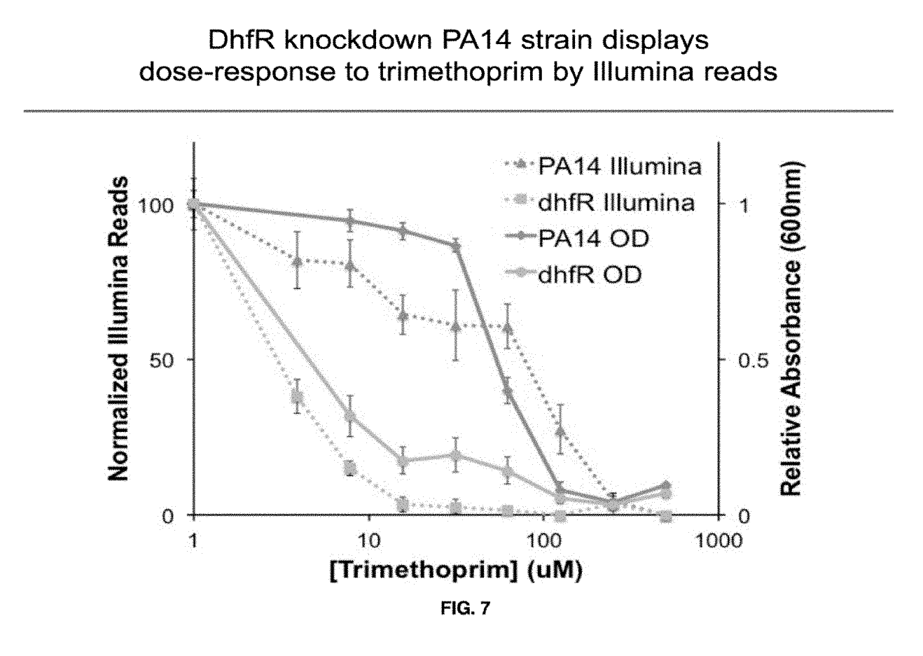

Results show that DhfR and MurA knockdown strains (hypomorphs) are hypersensitive to their respective drugs, as illustrated by FIGS. 6A and 6B. FIG. 7 show that DhfR knockdown PA14 strain displays dose-response to trimethoprim. This validates the hypomorph-based approach for a screen.

The strains can then be used in a screen for anti-bacterial compounds. A pilot screen was performed against 2240 compounds:
Screened 2240 compounds in duplicate using the SPECTRUM collection of known drug components, natural products, and bioactive agents;
Final screening concentration of 23.5 μM
Sixteen 384-well plates grown for 12 hours, cells were lysed and barcodes were amplified
Libraries of the barcodes were sequenced on Illumina HiSeq 2500 v3 Rapid Mode
221,600,000 reads (an average of 1800 reads per strain per well)
Data was deconvoluted using Fastx-toolkit to separate plate, well, and strain barcodes and reads were counted

Results from this pilot screen were as follows

$$Z \text{-} factor = 1 - \frac{3 ( {σ}_{p} + {σ}_{n} )}{\vert {μ}_{p} - {μ}_{n} \vert} , ideally > 0.5$$

$${c}_{v} = \frac{σ}{μ} , ideally < 0.15$$

TABLE
Summary of multiplexed pilot screen of 2240 compounds

| Species/ | | | | | Specific |
|---|---|---|---|---|---|
| Strain | Z′-factor^{a} | CV^{a} | # of Hits^{b} | Hit Rate (%) | Hits^{c} |
| A. baumannii | 0.67 | 0.08 | 18 | 0.80 | |
| E. coli | 0.72 | 0.10 | 27 | 1.21 | |
| P. aeruginosa | 0.68 | 0.04 | 16 | 0.71 | |
| PA14 | | | | | |
| PA14 | 0.49 | 0.10 | 19 | 0.85 | 1 |
| dhfR | | | | | |
| PA14 | 0.63 | 0.08 | 26 | 1.16 | 6 |
| murA | | | | | |
| PA14 | 0.55 | 0.04 | 16 | 0.71 | |
| gcp | | | | | |
| PA14 | 0.52 | 0.09 | 16 | 0.71 | |
| lolB | | | | | |
| PA14 | 0.54 | 0.12 | 21 | 0.94 | 3 |
| oprL | | | | | |

^{a}Values from an average of three growth plates
^{b}The number of hits as determined by impairing growth <10% relative to DMSO, in duplicate
^{c}The number of hits solely impairing growth of the single PA14 knock-down strain

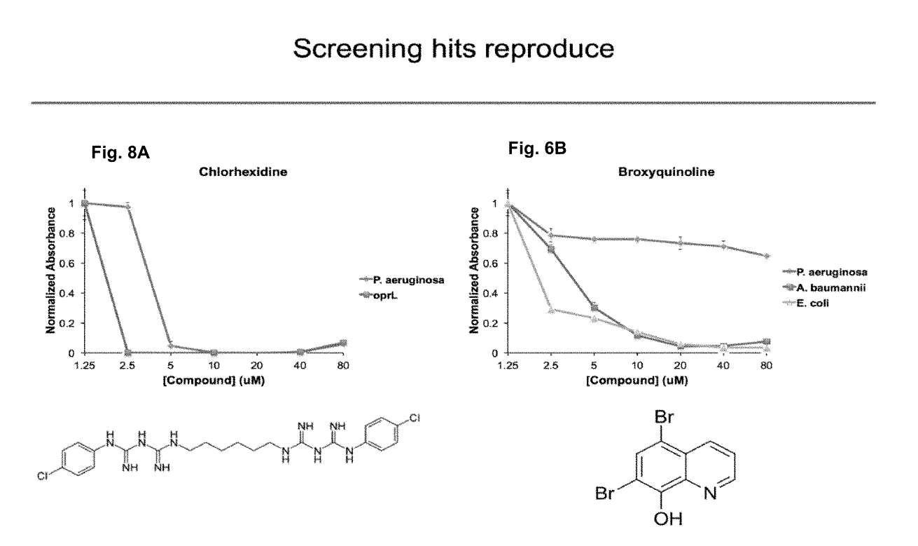

Reproducibility is illustrated by results on FIGS. 8A and 8B.

As a summary, in this Example:
Inventors identified 387 essential genes in PA14 in four different media;
Inventors selected 17 genes for knockdown targeting, consisting of outer membrane, periplasmic, and extracellular proteins;
Inventors optimized a library of variable promoters for use in P. aeruginosa;
Inventors constructed 8 essential gene knockdowns (hypomorphs strains), including dhfR and murA cytosolic controls that are hypersensitive to trimethoprim and fosfomycin, respectively;
A multiplexed growth of barcoded strains method was developed using Illumina sequencing as a readout;
A pilot screen of 2,240 compounds in duplicate was performed, and specific hits for ⅝ knockdown strains were obtained.

Pilot screen of the present example is scaled up to 50,000 compounds against the combination of 25 bacterial species and strains.

### Example 7

### Creating Hypomorphic M. tuberculosis Strains and Uses Thereof for Screening

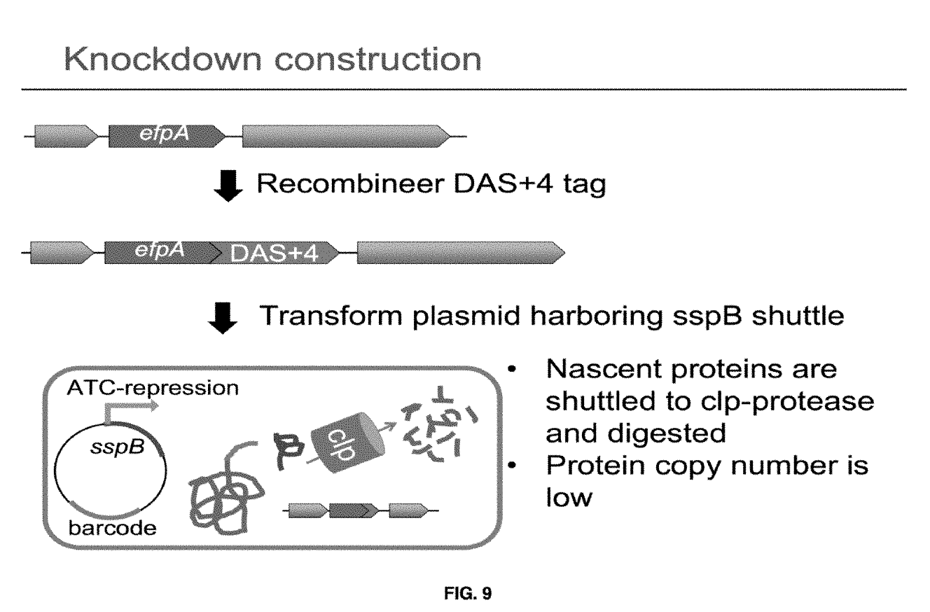

FIG. 9 depicts a strategy for the generation of hypomorph strains of M. tuberculosis.

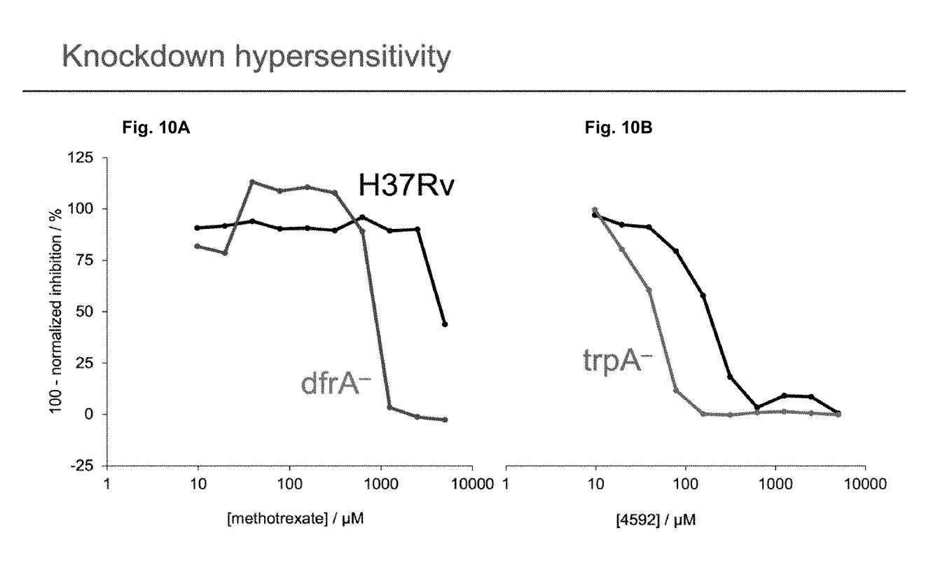

FIGS. 10A and 10B show that the strains obtained are hypersensitive to drugs targeting their gene of interest (dose response curves).

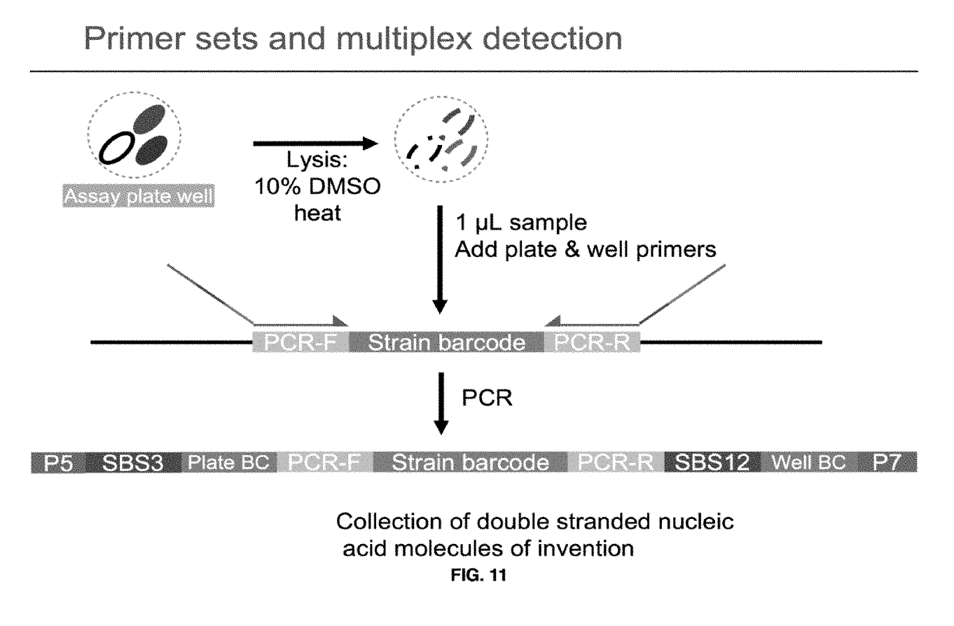

FIG. 11 shows principle for multiplex detection of the invention. Plate well contains several strains. After lysis, PCR can be performed using the primer set of the invention. The strain barcode (unique strain identifier) may be multiplex amplified using primers having amplification sequences PCR-F and PCR-R (such as flanking sequences). The primers also comprise overhang sequences that include polynucleotide sequence indicative of experimental conditions (well barcode=well BC, plate barcode=plate BC), as well as sequences configured for subsequent DNA sequencing (Illumina P5, P7, SBS3, SBS12 for example). This leads to a collection of double stranded nucleic acid molecule of the invention (P5-SBS3-Plate BC-PCRF-Strain BC-PCRR-SBS12-Well BC-P7).

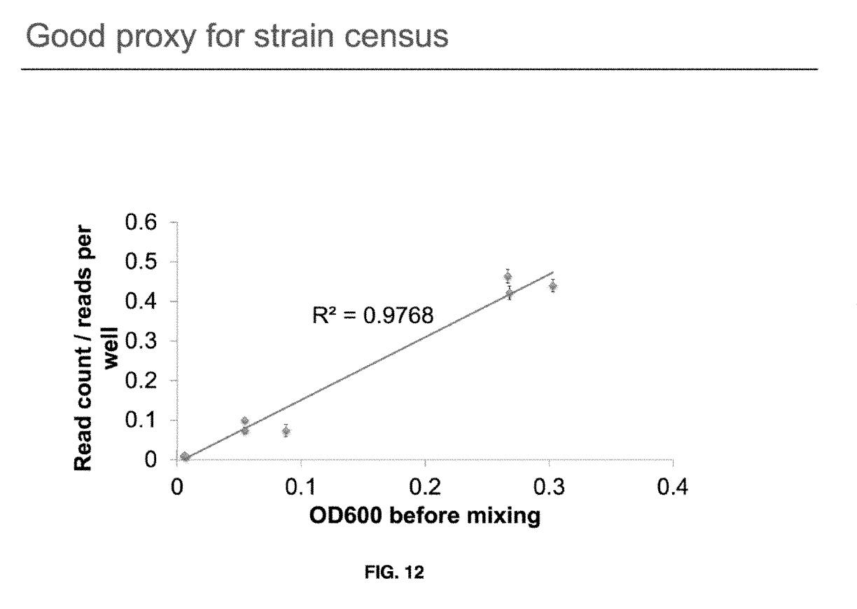

Results shown on FIG. 12 illustrate that the method of the invention allows to reliably detect and count micro-organism cells: the method of the invention provides for a reliable cell ‘census’, barcode (strain identifier) is a reliable indication of OD600 (Barcoded H37Rv strains were mixed spanning 3 logs in triplicate in a single pool OD600 was measured after dilution before mixing to compare with read counts: Barcode count is a reliable proxy for OD600).

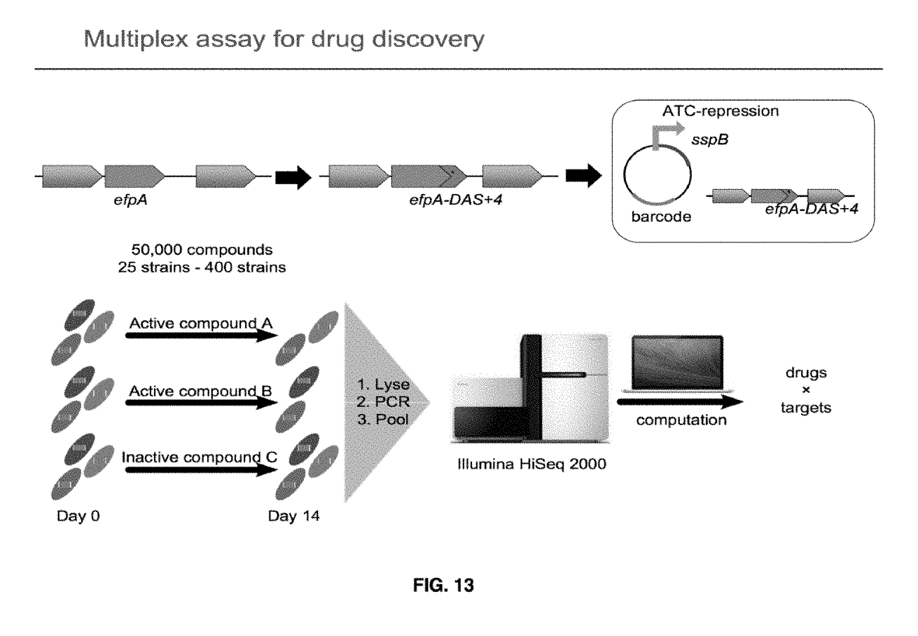

FIG. 13 illustrates a screening method of the invention.

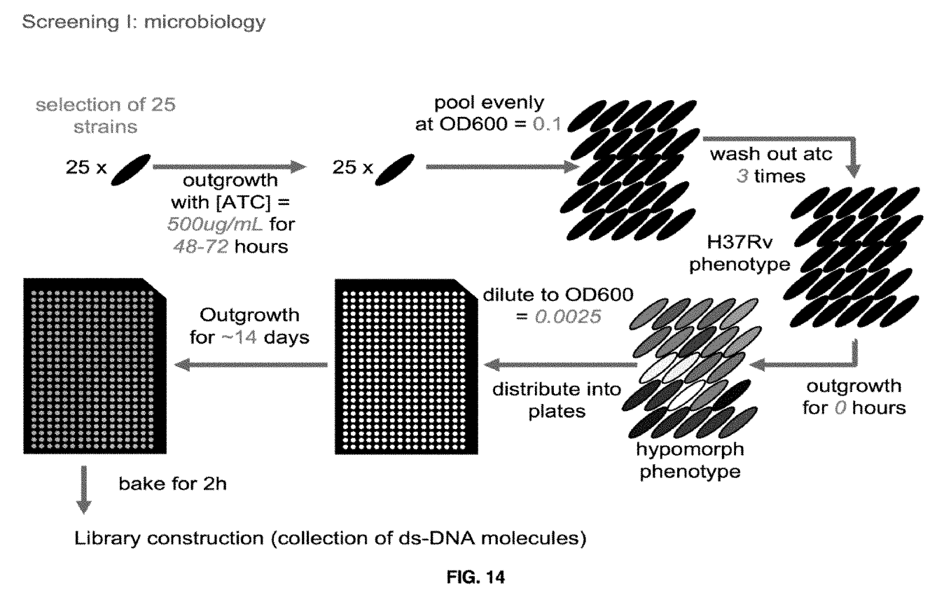

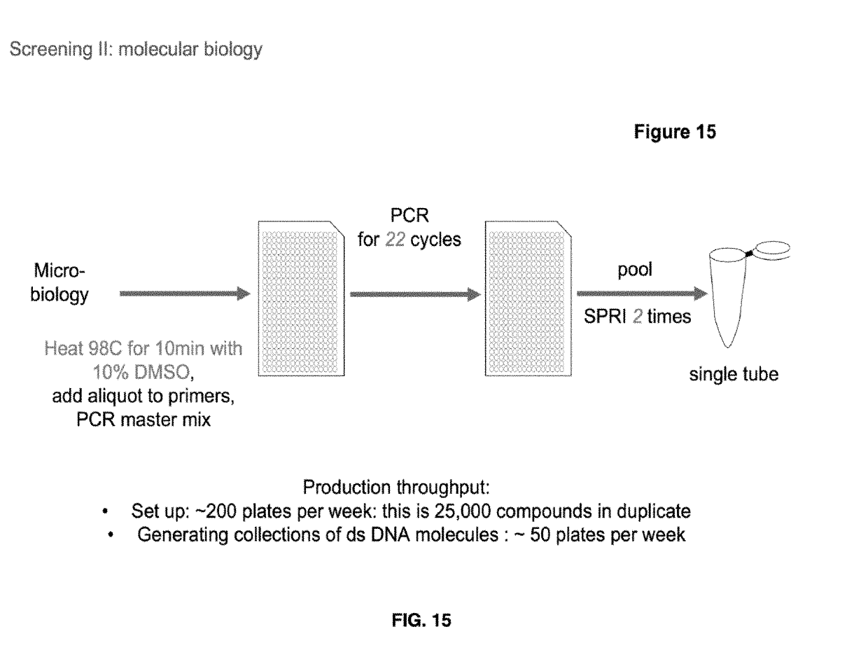

FIG. 14 shows a part I of the screening: hypomorph strains are outgrown in presence of anhydrotetracycline (atc) so as to obtain a hypomorph phenotype. Outgrowth is then performed in well format, before generating by multiplex PCR the collections of ds DNA molecules of the invention as per a part II of the screening method, exemplified on FIG. 15.

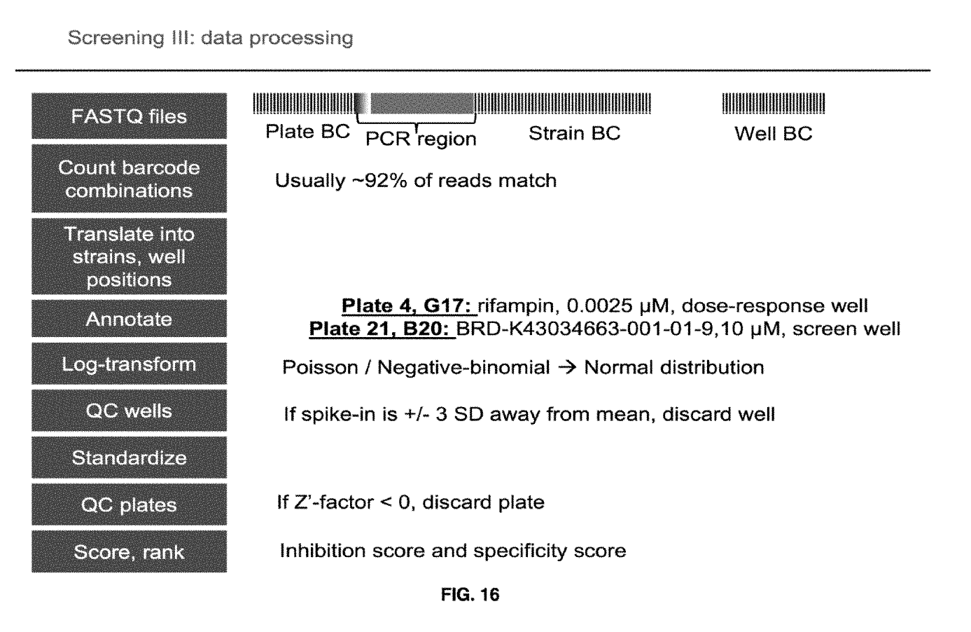

FIG. 16 shows a part III of the screening method comprising data processing.

A pilot screen was performed as described, with
26 strains
   M. tuberculosis H37Rv (a M. tuberculosis wild type strain which is a virulent clinical isolate)
   25 knockdowns strains (hypomorphs)
2000 compounds (candidate for screening)
   Reported in literature as Mtb-inhibitors;
   Confirmed as inhibitors (data not shown);
   4-point dose-response (0.3-10 μM) in duplicate.
Results:
   By-plate-by-strain Z′-factors >0.5;
   Coefficient of variability <10%;
   420,000 data points;
   Hit rate:

| Type | 10 μM | 3 μM | 1 μM | 0.3 μM |
|---|---|---|---|---|
| Inactive | 66% | 87% | 94% | 98% |
| All-killer | 0.9% | 0.06% | 0% | 0% |
| Hit >1 strain selectively | 0.6% | 0.06% | 0.2% | 0% |
| Hit only 1 strain selectively | 2% | 0.4% | 0.3% | 0.06% |

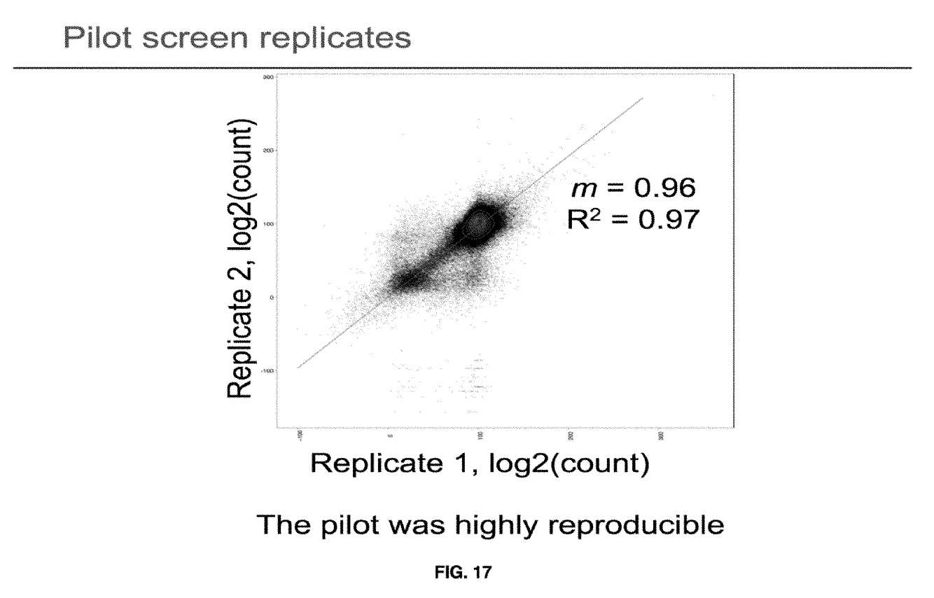

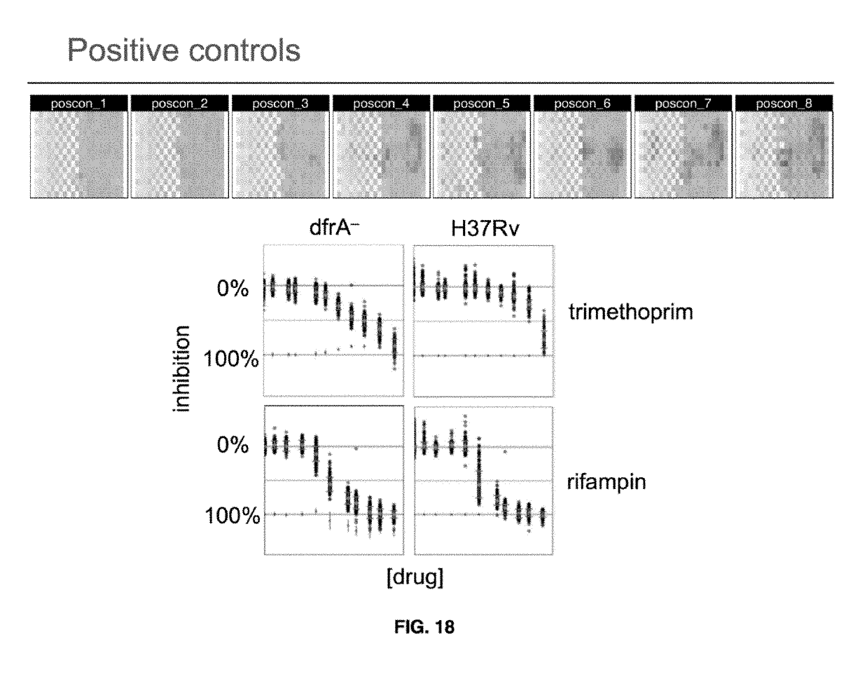

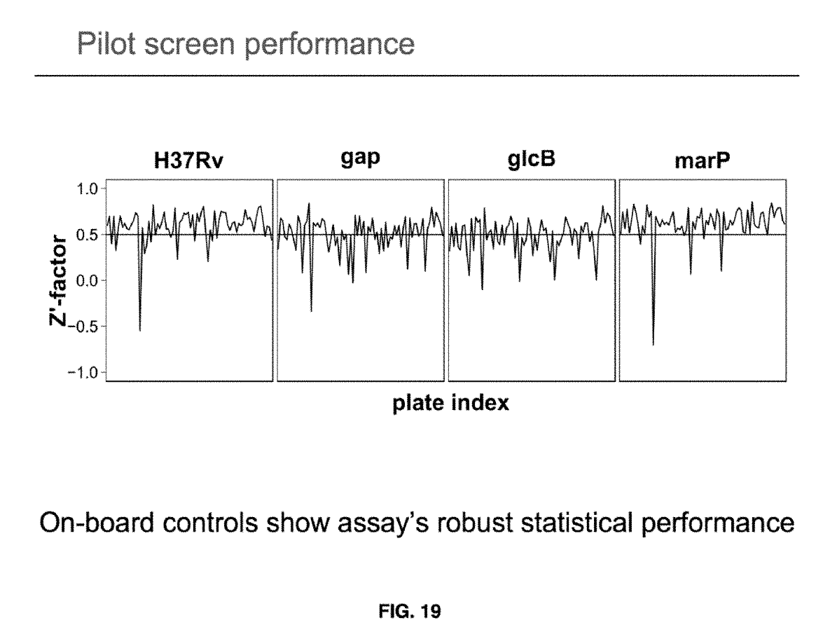

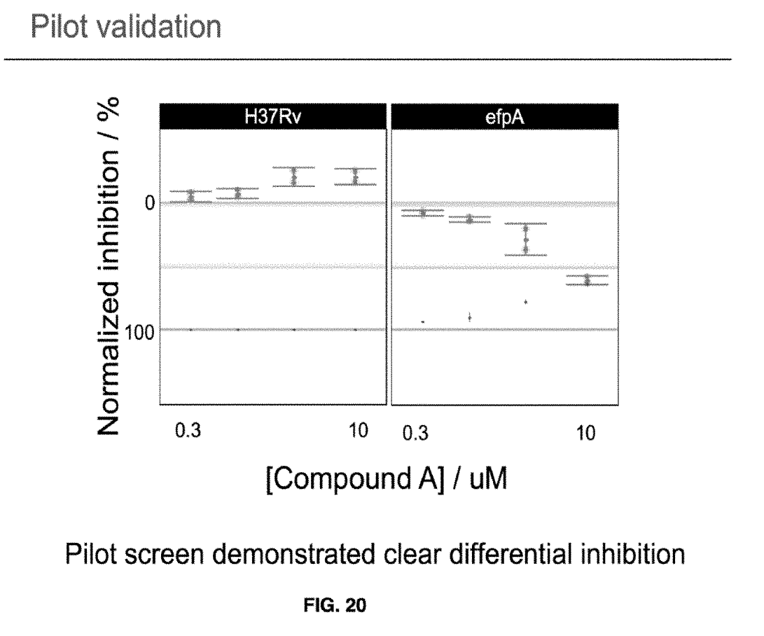

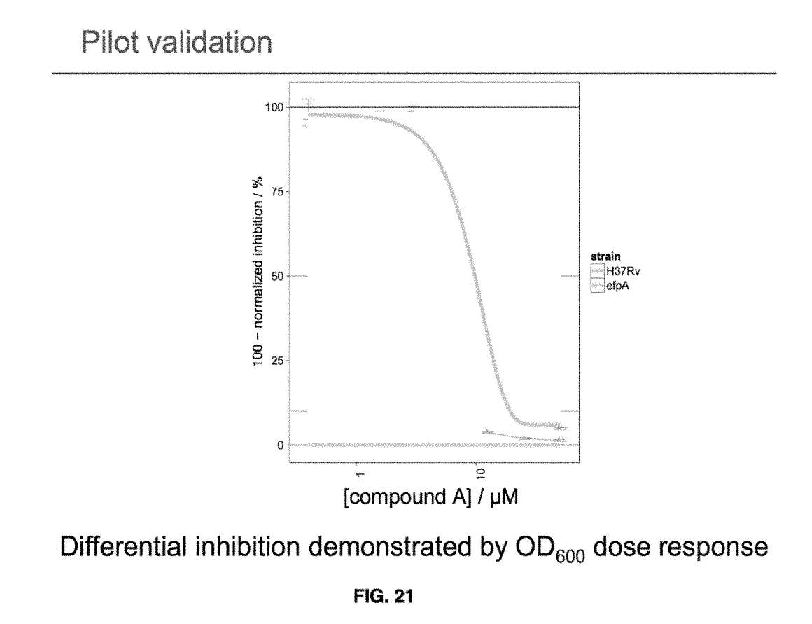

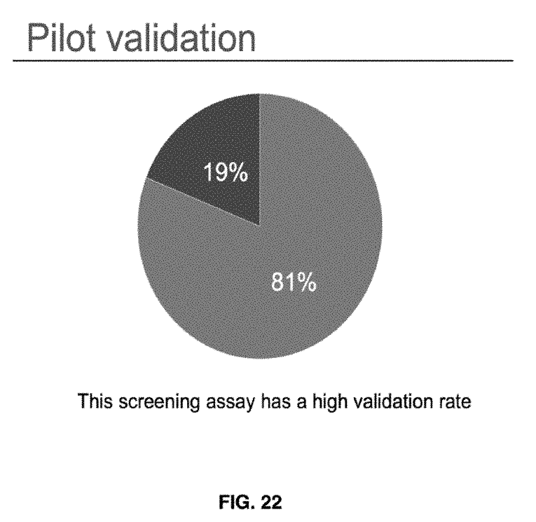

FIGS. 17-22 show results, in particular illustrate the high reproducibility obtained, validates the method with respect to positive controls with compounds trimethoprim and rifampin, highlight robustness of the statistical performance of the method demonstrate detection of differential inhibition, and demonstrate high validation rate.
As a conclusion:
   This illustrates a method to multiplex at least 26 strains;
   Data are very reproducible;
   statistically significant results can be detected;
   Validation rate is very high;
   Resulting data contain mechanism of action information.

A scale up method was performed:
26 strains
   M tuberculosis H37Rv
   25 knockdowns
50,000 compounds
   Library constructed from commercial and in-house collections
   Chosen to be as diverse as possible
   50 μM in duplicate
Results:
   By-plate-by-strain Z′-factors >0.5
   Coefficient of variability <10%
   2,600,000 data points
   Hit rates:

| Type | Rate |
|---|---|
| All-killer | 0.5% |
| H37Rv-killer | 0.3% |
| Single strain killer, not H37Rv | 0.5% |

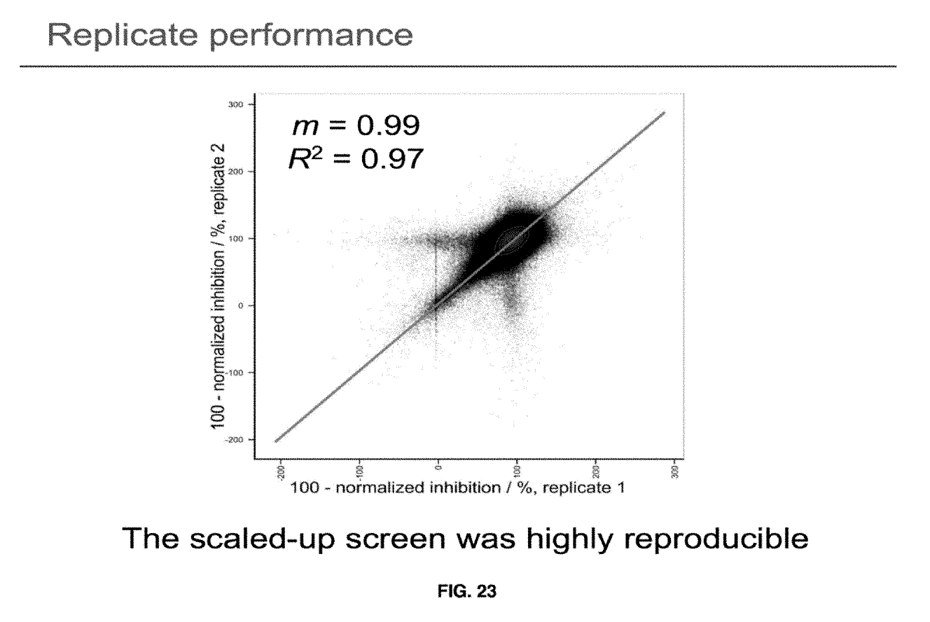

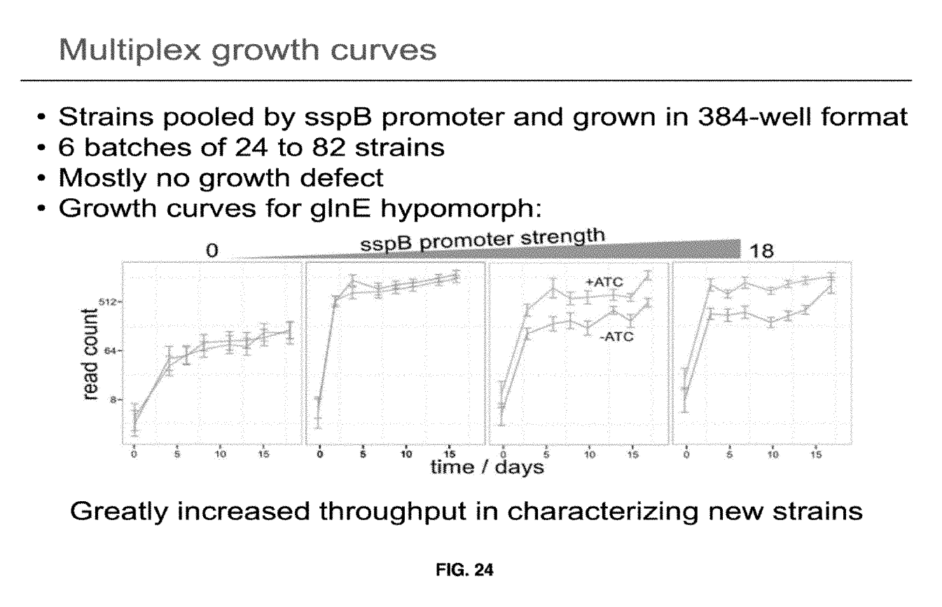

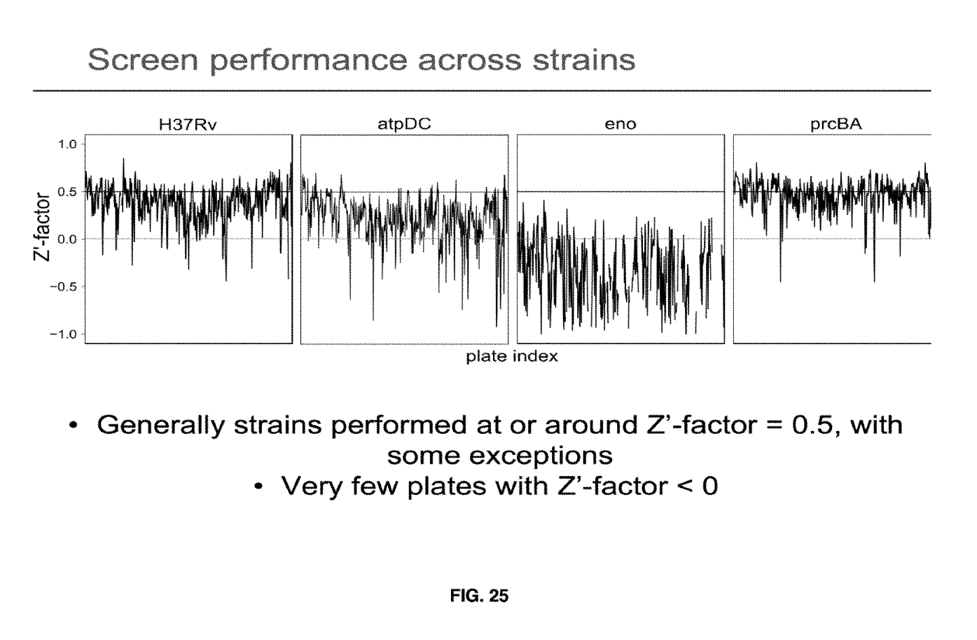

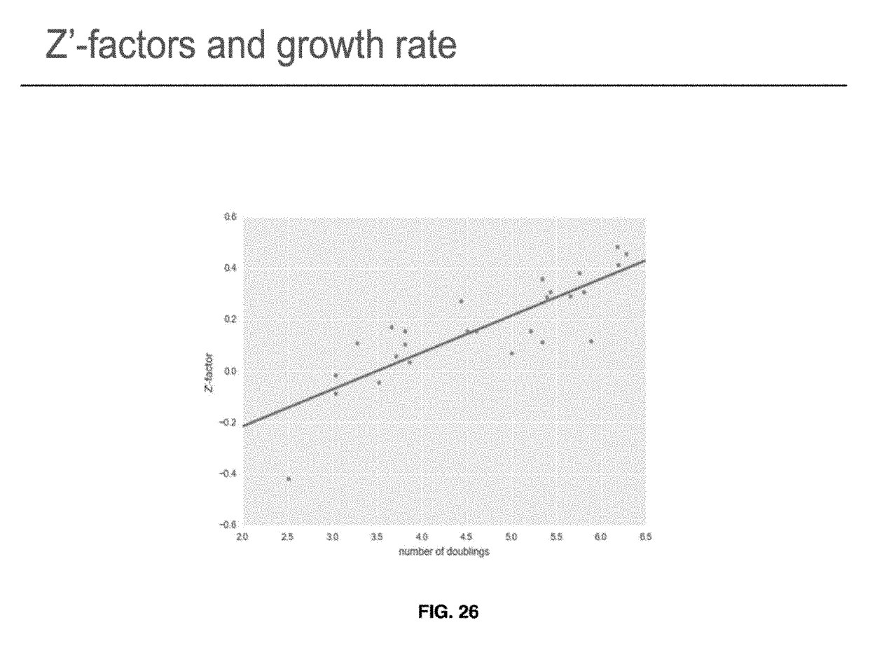

The method allows to identify compounds that would otherwise be missed.
Results are also shown on FIG. 23-26, showing high reproducibility and screen performance.

As a conclusion:
Inventors explored the potential of multiplexing target-based whole-cell screens;
Invention allows to get target information with every hit of a chemical inhibitor screen;
Pilot screen of 2000 “known actives” was shown to be robust;
Scale-up to 50,000 compounds was shown reproducible;
Invention allows to identify new and known chemical and biological insight.

The method of the invention may be further applied to:
Continue scaling up: 100 strains vs 2000 and 50,000 compound screens;
Build reference data with wide range of compounds of known mechanism of action;
Apply supervised machine learning to aid target ID of new hits;
Follow up hits and confirm targets.

### Example 8

### 16S primer sequences

16S primers for Mycobacterium smegmatis:

F:

(SEQ. I.D. No. 7)

5′-AAGGGGCATGATGACTTGAC-3′
R:

(SEQ. I.D. No. 8)

5′-GAGATGTCGGTTCCCTTGTG-3′

primers for Mycobacterium tuberculosis (from Nadkarni 2002 https://www.ncbi.nlm.nih.gov/pubmed/11782518):

F:

(SEQ. I.D. No. 9)

5′-TCCTACGGGAGGCAGCAGT-3′
R:

(SEQ. I.D. No. 10)

5′-GGACTACCAGGGTATCTAATCCTGTT-3′

### Example 9

### Gates Multplex TB assay protocol

### Materials Required

A. Strains
Group 2 strains

| Gene | Gene promoter | sspB promoter | Category | Label | Selection | Comment |
|---|---|---|---|---|---|---|
| — | — | — | control | RvBC02 | strep | barcoded |
| | | | | | | H37Rv |
| clpP1P2 | native | 2 | control | H5 | hyg/zeo/strep | revertant − |
| | | | | | | control |
| mesJ | 38 | 18 | control | H14 | hyg/kan/strep | non- |
| | | | | | | essential + |
| | | | | | | control |
| accD6 | 21 | 2 | control | C60 | hyg/zeo/strep | |
| 1pd | 38 | 10 | control | C66 | hyg/zeo/strep | |
| alr-FLAG | native | 2 | Deg-screen | H4 | hyg/strep | |
| ccsX | 21 | 18 | Deg-screen | C40 | hyg/zeo/strep | |
| ctaC | 38 | 18 | Deg-screen | C4 | hyg/zeo/strep | |
| dfrA-FLAG | native | 2 | Deg-screen | H8 | hyg/strep | sensitivity + |
| | | | | | | control |
| eno | 21 | 2 | Deg-screen | C33 | hyg/zeo/strep | |
| fba | 38 | 2 | Deg-screen | C13 | hyg/zeo/strep | |
| folB | native | 6 | Deg-screen | H12 | hyg/strep | |
| glcB | 38 | 18 | Deg-screen | C12 | hyg/zeo/strep | |
| marP | 21 | 18 | Deg-screen | C44 | hyg/zeo/strep | |
| mdh | 38 | 6 | Deg-screen | U7 | hyg/zeo/strep | |
| mshC | 21 | 18 | Deg-screen | U17 | hyg/zeo/strep | |
| murG | 21 | 18 | Deg-screen | U13 | hyg/zeo/strep | |
| nadE | 38 | 18 | Deg-screen | C8 | hyg/zeo/strep | |
| pstP | 38 | 18 | Deg-screen | U2 | hyg/zeo/strep | |
| sucD | 38 | 2 | Deg-screen | U9 | hyg/zeo/strep | |
| topA | 38 | 18 | Deg-screen | U1 | hyg/zeo/strep | |
| clpP1P2 | native | 6 | Deg-screen | H19 | hyg/zeo/strep | |
| efpA | native | 2 | Deg-screen | H20 | hyg/zeo/strep | |
| tpi | 38 | 6 | Deg-screen | C68 | hyg/zeo/strep | |
| dlat | 38 | 10 | Deg-screen | C73 | hyg/zeo/strep | |
| gap | 38 | 2 | Deg-screen | C63 | hyg/zeo/strep | |
| fum | Phsp60 | 10 | Deg-screen | C80 | hyg/zeo/strep | |
| pth-FLAG | synthetic | 2 | Deg-screen | H21 | hyg/strep | |
| ndhA | synthetic | — | Trans-screen | C83 | hyg/kan/zeo/strep | |
| prcBA | synthetic | — | Trans-screen | C84 | hyg/kan/strep | |
| atpDC | synthetic | — | Trans-screen | C81 | hyg/zeo/strep | |

B. Reagents
Difco Middlebrook 7H9 powder
OADC Enrichment
Acetate
Tween-80
Tyloxapol
Hygromycin
Rifampicin
Trimethoprim
Streptomycin
Kanamycin
Zeocin
Anhydrotetracycline
P5 and P7 primers pre-mixed at 5 uM in 384-well PCR plates
NEB Q5 Hot Start Polymerase
dNTPs
DMSO
Control plasmids: 1=tag_8090, 2=tag_1 150

tag_1180

SEQ. I.D. No. 648

AATGTAACGTCATGTGAGCG
tag_8090

SEQ. I.D. No. 649

ATATTCCTTGACAGGCCGGG

Agilent High Sensitivity DNA Analysis Kit
Vesphene
Bleach
   Selection agents for strains. Selection listed on strain spreadsheet.
      Hyg 50 μg/ml
      Strep 20 μg/ml
      Kan 15 μg/ml
      Zeocin 25 μg/ml
         Library construction/PCR primers
P7 or well index primers 66 unique primers to allow for moat wells (IDT ieHPLC purified)

| Name | Sequence | index | index read |
|---|---|---|---|
| p7_1 | CAAGCAGAAGACGGCATACGAGATAAG | AAGATCGA | TCGATCTT |
| | ATCGAGTGACTGGAGTTCAGACGTGTG | SEQ. I.D. | SEQ. I.D. No. |
| | CTCTTCCGATCTTAAAGCAGCGTATCCA | No. 651 | 652 |
| | CATAGCGT | | |
| | SEQ. I.D. No. 650 | | |
| p7_2 | CAAGCAGAAGACGGCATACGAGATAAT | AATAGCGC | GCGCTATT |
| | AGCGCGTGACTGGAGTTCAGACGTGTG | SEQ. I.D. | SEQ. I.D. No. |
| | CTCTTCCGATCTTAAAGCAGCGTATCCA | No. 654 | 655 |
| | CATAGCGT | | |
| | SEQ. I.D. No. 653 | | |
| p7_3 | CAAGCAGAAGACGGCATACGAGATAAT | AATCTCTT | AAGAGATT |
| | CTCTTGTGACTGGAGTTCAGACGTGTGC | SEQ. I.D. | SEQ. I.D. No. |
| | TCTTCCGATCTTAAAGCAGCGTATCCAC | No. 657 | 658 |
| | ATAGCGT | | |
| | SEQ. I.D. No. 656 | | |
| p7_4 | CAAGCAGAAGACGGCATACGAGATAAT | AATGCACA | TGTGCATT |
| | GCACAGTGACTGGAGTTCAGACGTGTG | SEQ. I.D. | SEQ. I.D. No. |
| | CTCTTCCGATCTTAAAGCAGCGTATCCA | No. 660 | 661 |
| | CATAGCGT | | |
| | SEQ. I.D. No. 659 | | |
| p7_5 | CAAGCAGAAGACGGCATACGAGATACG | ACGCGATC | GATCGCGT |
| | CGATCGTGACTGGAGTTCAGACGTGTG | SEQ. I.D. | SEQ. I.D. No. |
| | CTCTTCCGATCTTAAAGCAGCGTATCCA | No. 663 | 664 |
| | CATAGCGT | | |
| | SEQ. I.D. No. 662 | | |
| p7_6 | CAAGCAGAAGACGGCATACGAGATACT | ACTATCTT | AAGATAGT |
| | ATCTTGTGACTGGAGTTCAGACGTGTGC | SEQ. I.D. | SEQ. I.D. No. |
| | TCTTCCGATCTTAAAGCAGCGTATCCAC | No. 666 | 667 |
| | ATAGCGT | | |
| | SEQ. I.D. No. 665 | | |
| p7_7 | CAAGCAGAAGACGGCATACGAGATACT | ACTGGGAG | CTCCCAGT |
| | GGGAGGTGACTGGAGTTCAGACGTGTG | SEQ. I.D. | SEQ. I.D. No. |
| | CTCTTCCGATCTTAAAGCAGCGTATCCA | No. 669 | 670 |
| | CATAGCGT | | |
| | SEQ. I.D. No. 668 | | |
| p7_8 | CAAGCAGAAGACGGCATACGAGATAGA | AGAATCAC | GTGATTCT |
| | ATCACGTGACTGGAGTTCAGACGTGTG | SEQ. I.D. | SEQ. I.D. No. |
| | CTCTTCCGATCTTAAAGCAGCGTATCCA | No. 672 | 673 |
| | CATAGCGT | | |
| | SEQ. I.D. No. 671 | | |
| p7_9 | CAAGCAGAAGACGGCATACGAGATAGG | AGGATTTT | AAAATCCT |
| | ATTTTGTGACTGGAGTTCAGACGTGTGC | SEQ. I.D. | SEQ. I.D. No. |
| | TCTTCCGATCTTAAAGCAGCGTATCCAC | No. 675 | 676 |
| | ATAGCGT | | |
| | SEQ. I.D. No. 674 | | |
| p7_10 | CAAGCAGAAGACGGCATACGAGATAGT | AGTTAGTC | GACTAACT |
| | TAGTCGTGACTGGAGTTCAGACGTGTGC | SEQ. I.D. | SEQ. I.D. No. |
| | TCTTCCGATCTTAAAGCAGCGTATCCAC | No. 678 | 679 |
| | ATAGCGT | | |
| | SEQ. I.D. No. 677 | | |
| p7_11 | CAAGCAGAAGACGGCATACGAGATAGT | AGTTGAGG | CCTCAACT |
| | TGAGGGTGACTGGAGTTCAGACGTGTG | SEQ. I.D. | SEQ. I.D. No. |
| | CTCTTCCGATCTTAAAGCAGCGTATCCA | No. 681 | 682 |
| | CATAGCGT | | |
| | SEQ. I.D. No. 680 | | |
| p7_12 | CAAGCAGAAGACGGCATACGAGATATA | ATAACGCG | CGCGTTAT |
| | ACGCGGTGACTGGAGTTCAGACGTGTG | SEQ. I.D. | SEQ. I.D. No. |
| | CTCTTCCGATCTTAAAGCAGCGTATCCA | No. 684 | 685 |
| | CATAGCGT | | |
| | SEQ. I.D. No. 683 | | |
| p7_13 | CAAGCAGAAGACGGCATACGAGATATC | ATCAAGGA | TCCTTGAT |
| | AAGGAGTGACTGGAGTTCAGACGTGTG | SEQ. I.D. | SEQ. I.D. No. |
| | CTCTTCCGATCTTAAAGCAGCGTATCCA | No. 687 | 688 |
| | CATAGCGT | | |
| | SEQ. I.D. No. 686 | | |
| p7_14 | CAAGCAGAAGACGGCATACGAGATATC | ATCGTTGG | CCAACGAT |
| | GTTGGGTGACTGGAGTTCAGACGTGTG | SEQ. I.D. | SEQ. I.D. No. |
| | CTCTTCCGATCTTAAAGCAGCGTATCCA | No. 690 | 691 |
| | CATAGCGT | | |
| | SEQ. I.D. No. 689 | | |
| p7_15 | CAAGCAGAAGACGGCATACGAGATATT | ATTGGACT | AGTCCAAT |
| | GGACTGTGACTGGAGTTCAGACGTGTG | SEQ. I.D. | SEQ. I.D. No. |
| | CTCTTCCGATCTTAAAGCAGCGTATCCA | No. 693 | 694 |
| | CATAGCGT | | |
| | SEQ. I.D. No. 692 | | |
| p7_16 | CAAGCAGAAGACGGCATACGAGATCAA | CAAGCGGC | GCCGCTTG |
| | GCGGCGTGACTGGAGTTCAGACGTGTG | SEQ. I.D. | SEQ. I.D. No. |
| | CTCTTCCGATCTTAAAGCAGCGTATCCA | No. 696 | 697 |
| | CATAGCGT | | |
| | SEQ. I.D. No. 695 | | |
| p7_17 | CAAGCAGAAGACGGCATACGAGATCAC | CACGCTCA | TGAGCGTG |
| | GCTCAGTGACTGGAGTTCAGACGTGTG | SEQ. I.D. | SEQ. I.D. No. |
| | CTCTTCCGATCTTAAAGCAGCGTATCCA | No. 699 | 700 |
| | CATAGCGT | | |
| | SEQ. I.D. No. 698 | | |
| p7_18 | CAAGCAGAAGACGGCATACGAGATCAG | CAGTTTGT | ACAAACTG |
| | TTTGTGTGACTGGAGTTCAGACGTGTGC | SEQ. I.D. | SEQ. I.D. No. |
| | TCTTCCGATCTTAAAGCAGCGTATCCAC | No. 702 | 703 |
| | ATAGCGT | | |
| | SEQ. I.D. No. 701 | | |
| p7_19 | CAAGCAGAAGACGGCATACGAGATCAT | CATCGCGA | TCGCGATG |
| | CGCGAGTGACTGGAGTTCAGACGTGTG | SEQ. I.D. | SEQ. I.D. No. |
| | CTCTTCCGATCTTAAAGCAGCGTATCCA | No. 705 | 706 |
| | CATAGCGT | | |
| | SEQ. I.D. No. 704 | | |
| p7_20 | CAAGCAGAAGACGGCATACGAGATCCA | CCACACCG | CGGTGTGG |
| | CACCGGTGACTGGAGTTCAGACGTGTG | SEQ. I.D. | SEQ. I.D. No. |
| | CTCTTCCGATCTTAAAGCAGCGTATCCA | No. 708 | 709 |
| | CATAGCGT | | |
| | SEQ. I.D. No. 707 | | |
| p7_21 | CAAGCAGAAGACGGCATACGAGATCCA | CCACTGTC | GACAGTGG |
| | CTGTCGTGACTGGAGTTCAGACGTGTGC | SEQ. I.D. | SEQ. I.D. No. |
| | TCTTCCGATCTTAAAGCAGCGTATCCAC | No. 711 | 712 |
| | ATAGCGT | | |
| | SEQ. I.D. No. 710 | | |
| p7_22 | CAAGCAGAAGACGGCATACGAGATCCC | CCCACAAC | GTTGTGGG |
| | ACAACGTGACTGGAGTTCAGACGTGTG | SEQ. I.D. | SEQ. I.D. No. |
| | CTCTTCCGATCTTAAAGCAGCGTATCCA | No. 714 | 715 |
| | CATAGCGT | | |
| | SEQ. I.D. No. 713 | | |
| p7_23 | CAAGCAGAAGACGGCATACGAGATCCC | CCCGTATA | TATACGGG |
| | GTATAGTGACTGGAGTTCAGACGTGTG | SEQ. I.D. | SEQ. I.D. No. |
| | CTCTTCCGATCTTAAAGCAGCGTATCCA | No. 717 | 718 |
| | CATAGCGT | | |
| | SEQ. I.D. No. 716 | | |
| p7_24 | CAAGCAGAAGACGGCATACGAGATCCC | CCCTAGTC | GACTAGGG |
| | TAGTCGTGACTGGAGTTCAGACGTGTGC | SEQ. I.D. | SEQ. I.D. No. |
| | TCTTCCGATCTTAAAGCAGCGTATCCAC | No. 720 | 721 |
| | ATAGCGT | | |
| | SEQ. I.D. No. 719 | | |
| p7_25 | CAAGCAGAAGACGGCATACGAGATCCG | CCGTACGG | CCGTACGG |
| | TACGGGTGACTGGAGTTCAGACGTGTG | SEQ. I.D. | SEQ. I.D. No. |
| | CTCTTCCGATCTTAAAGCAGCGTATCCA | No. 723 | 724 |
| | CATAGCGT | | |
| | SEQ. I.D. No. 722 | | |
| p7_26 | CAAGCAGAAGACGGCATACGAGATCGA | CGACGAAG | CTTCGTCG |
| | CGAAGGTGACTGGAGTTCAGACGTGTG | SEQ. I.D. | SEQ. I.D. No. |
| | CTCTTCCGATCTTAAAGCAGCGTATCCA | No. 726 | 727 |
| | CATAGCGT | | |
| | SEQ. I.D. No. 725 | | |
| p7_27 | CAAGCAGAAGACGGCATACGAGATCTG | CTGACCGC | GCGGTCAG |
| | ACCGCGTGACTGGAGTTCAGACGTGTG | SEQ. I.D. | SEQ. I.D. No. |
| | CTCTTCCGATCTTAAAGCAGCGTATCCA | No. 729 | 730 |
| | CATAGCGT | | |
| | SEQ. I.D. No. 728 | | |
| p7_28 | CAAGCAGAAGACGGCATACGAGATGCA | GCAGTGCG | CGCACTGC |
| | GTGCGGTGACTGGAGTTCAGACGTGTG | SEQ. I.D. | SEQ. I.D. No. |
| | CTCTTCCGATCTTAAAGCAGCGTATCCA | No. 732 | 733 |
| | CATAGCGT SEQ. I.D. No. 731 | | |
| p7_29 | CAAGCAGAAGACGGCATACGAGATGCT | GCTAGGAT | ATCCTAGC |
| | AGGATGTGACTGGAGTTCAGACGTGTG | SEQ. I.D. | SEQ. I.D. No. |
| | CTCTTCCGATCTTAAAGCAGCGTATCCA | No. 735 | 736 |
| | CATAGCGT | | |
| | SEQ. I.D. No. 734 | | |
| p7_30 | CAAGCAGAAGACGGCATACGAGATGCT | GCTCCAGA | TCTGGAGC |
| | CCAGAGTGACTGGAGTTCAGACGTGTG | SEQ. I.D. | SEQ. I.D. No. |
| | CTCTTCCGATCTTAAAGCAGCGTATCCA | No. 738 | 739 |
| | CATAGCGT | | |
| | SEQ. I.D. No. 737 | | |
| p7_31 | CAAGCAGAAGACGGCATACGAGATGTC | GTCCGTCA | TGACGGAC |
| | CGTCAGTGACTGGAGTTCAGACGTGTG | SEQ. | SEQ. I.D. No. |
| | CTCTTCCGATCTTAAAGCAGCGTATCCA | I.D. No. | 742 |
| | CATAGCGT | 741 | |
| | SEQ. I.D. No. 740 | | |
| p7_32 | CAAGCAGAAGACGGCATACGAGATGTG | GTGGGTTC | GAACCCAC |
| | GGTTCGTGACTGGAGTTCAGACGTGTGC | SEQ. | SEQ. I.D. No. |
| | TCTTCCGATCTTAAAGCAGCGTATCCAC | I.D. No. | 745 |
| | ATAGCGT | 744 | |
| | SEQ. I.D. No. 743 | | |
| p7_33 | CAAGCAGAAGACGGCATACGAGATGTG | GTGTGGAG | CTCCACAC |
| | TGGAGGTGACTGGAGTTCAGACGTGTG | SEQ. | SEQ. I.D. No. |
| | CTCTTCCGATCTTAAAGCAGCGTATCCA | I.D. No. | 748 |
| | CATAGCGT | 747 | |
| | SEQ. I.D. No. 746 | | |
| p7_34 | CAAGCAGAAGACGGCATACGAGATGTT | GTTAAGAG | CTCTTAAC |
| | AAGAGGTGACTGGAGTTCAGACGTGTG | SEQ. | SEQ. I.D. No. |
| | CTCTTCCGATCTTAAAGCAGCGTATCCA | I.D. No. | 751 |
| | CATAGCGT | 750 | |
| | SEQ. I.D. No. 749 | | |
| p7_35 | CAAGCAGAAGACGGCATACGAGATGTT | GTTCCGGG | CCCGGAAC |
| | CCGGGGTGACTGGAGTTCAGACGTGTG | SEQ. | SEQ. I.D. No. |
| | CTCTTCCGATCTTAAAGCAGCGTATCCA | I.D. No. | 754 |
| | CATAGCGT | 753 | |
| | SEQ. I.D. No. 752 | | |
| p7_36 | CAAGCAGAAGACGGCATACGAGATGTT | GTTGGGTT | AACCCAAC |
| | GGGTTGTGACTGGAGTTCAGACGTGTG | SEQ. | SEQ. I.D. No. |
| | CTCTTCCGATCTTAAAGCAGCGTATCCA | I.D. No. | 757 |
| | CATAGCGT | 756 | |
| | SEQ. I.D. No. 755 | | |
| p7_37 | CAAGCAGAAGACGGCATACGAGATTAC | TACCATGT | ACATGGTA |
| | CATGTGTGACTGGAGTTCAGACGTGTGC | SEQ. | SEQ. I.D. No. |
| | TCTTCCGATCTTAAAGCAGCGTATCCAC | I.D. No. | 760 |
| | ATAGCGT | 759 | |
| | SEQ. I.D. No. 758 | | |
| p7_38 | CAAGCAGAAGACGGCATACGAGATTAC | TACGGGCG | CGCCCGTA |
| | GGGCGGTGACTGGAGTTCAGACGTGTG | SEQ. | SEQ. I.D. No. |
| | CTCTTCCGATCTTAAAGCAGCGTATCCA | I.D. No. | 763 |
| | CATAGCGT | 762 | |
| | SEQ. I.D. No. 761 | | |
| p7_39 | CAAGCAGAAGACGGCATACGAGATTCA | TCAATCAC | GTGATTGA |
| | ATCACGTGACTGGAGTTCAGACGTGTG | SEQ. | SEQ. I.D. No. |
| | CTCTTCCGATCTTAAAGCAGCGTATCCA | I.D. No. | 766 |
| | CATAGCGT | 765 | |
| | SEQ. I.D. No. 764 | | |
| p7_40 | CAAGCAGAAGACGGCATACGAGATTCA | TCATACCA | TGGTATGA |
| | TACCAGTGACTGGAGTTCAGACGTGTG | SEQ. I.D. | SEQ. I.D. No. |
| | CTCTTCCGATCTTAAAGCAGCGTATCCA | No. 768 | 769 |
| | CATAGCGT | | |
| | SEQ. I.D. No. 767 | | |
| p7_41 | CAAGCAGAAGACGGCATACGAGATTCC | TCCGGTTG | CAACCGGA |
| | GGTTGGTGACTGGAGTTCAGACGTGTG | SEQ. I.D. | SEQ. I.D. No. |
| | CTCTTCCGATCTTAAAGCAGCGTATCCA | No. 771 | 772 |
| | CATAGCGT | | |
| | SEQ. I.D. No. 770 | | |
| p7_42 | CAAGCAGAAGACGGCATACGAGATTGA | TGACTTGT | ACAAGTCA |
| | CTTGTGTGACTGGAGTTCAGACGTGTGC | SEQ. I.D. | SEQ. I.D. No. |
| | TCTTCCGATCTTAAAGCAGCGTATCCAC | No. 774 | 775 |
| | ATAGCGT | | |
| | SEQ. I.D. No. 773 | | |
| p7_43 | CAAGCAGAAGACGGCATACGAGATTGC | TGCTGCTC | GAGCAGCA |
| | TGCTCGTGACTGGAGTTCAGACGTGTGC | SEQ. I.D. | SEQ. I.D. No. |
| | TCTTCCGATCTTAAAGCAGCGTATCCAC | No. 777 | 778 |
| | ATAGCGT | | |
| | SEQ. I.D. No. 776 | | |
| p7_44 | CAAGCAGAAGACGGCATACGAGATTGT | TGTAGACC | GGTCTACA |
| | AGACCGTGACTGGAGTTCAGACGTGTG | SEQ. I.D. | SEQ. I.D. No. |
| | CTCTTCCGATCTTAAAGCAGCGTATCCA | No. 780 | 781 |
| | CATAGCGT | | |
| | SEQ. I.D. No. 779 | | |
| p7_45 | CAAGCAGAAGACGGCATACGAGATTTA | TTACGTTG | CAACGTAA |
| | CGTTGGTGACTGGAGTTCAGACGTGTGC | SEQ. I.D. | SEQ. I.D. No. |
| | TCTTCCGATCTTAAAGCAGCGTATCCAC | No. 783 | 784 |
| | ATAGCGT | | |
| | SEQ. I.D. No. 782 | | |
| p7_46 | CAAGCAGAAGACGGCATACGAGATTTC | TTCGCGGA | TCCGCGAA |
| | GCGGAGTGACTGGAGTTCAGACGTGTG | SEQ. I.D. | SEQ. I.D. No. |
| | CTCTTCCGATCTTAAAGCAGCGTATCCA | No. 786 | 787 |
| | CATAGCGT | | |
| | SEQ. I.D. No. 785 | | |
| p7_47 | CAAGCAGAAGACGGCATACGAGATTTG | TTGATCGG | CCGATCAA |
| | ATCGGGTGACTGGAGTTCAGACGTGTG | SEQ. I.D. | SEQ. I.D. No. |
| | CTCTTCCGATCTTAAAGCAGCGTATCCA | No. 789 | 790 |
| | CATAGCGT | | |
| | SEQ. I.D. No. 788 | | |
| p7_48 | CAAGCAGAAGACGGCATACGAGATTTT | TTTGCAGT | ACTGCAAA |
| | GCAGTGTGACTGGAGTTCAGACGTGTG | SEQ. I.D. | SEQ. I.D. No. |
| | CTCTTCCGATCTTAAAGCAGCGTATCCA | No. 792 | 793 |
| | CATAGCGT | | |
| | SEQ. I.D. No. 791 | | |
| P7_49 | CAAGCAGAAGACGGCATACGAGATCAG | CAGTCGAT | ATCGACTG |
| | TCGATGTGACTGGAGTTCAGACGTGTGC | SEQ. I.D. | SEQ. I.D. No. |
| | TCTTCCGATCTTAAAGCAGCGTATCCAC | No. 795 | 796 |
| | ATAGCGT | | |
| | SEQ. I.D. No. 794 | | |
| P7_50 | CAAGCAGAAGACGGCATACGAGATCTG | CTGCTAGC | GCTAGCAG |
| | CTAGCGTGACTGGAGTTCAGACGTGTG | SEQ. I.D. | SEQ. I.D. No. |
| | CTCTTCCGATCTTAAAGCAGCGTATCCA | No. 798 | 799 |
| | CATAGCGT | | |
| | SEQ. I.D. No. 797 | | |
| P7_51 | CAAGCAGAAGACGGCATACGAGATGGA | GGAGAGTA | TACTCTCC |
| | GAGTAGTGACTGGAGTTCAGACGTGTG | SEQ. I.D. | SEQ. I.D. No. |
| | CTCTTCCGATCTTAAAGCAGCGTATCCA | No. 801 | 802 |
| | CATAGCGT | | |
| | SEQ. I.D. No. 800 | | |
| P7_52 | CAAGCAGAAGACGGCATACGAGATTGC | TGCTGTCA | TGACAGCA |
| | TGTCAGTGACTGGAGTTCAGACGTGTGC | SEQ. I.D. | SEQ. I.D. No. |
| | TCTTCCGATCTTAAAGCAGCGTATCCAC | No. 804 | 805 |
| | ATAGCGT | | |
| | SEQ. I.D. No. 803 | | |
| P7_53 | CAAGCAGAAGACGGCATACGAGATCAA | CAACCTGC | GCAGGTTG |
| | CCTGCGTGACTGGAGTTCAGACGTGTGC | SEQ. I.D. | SEQ. I.D. No. |
| | TCTTCCGATCTTAAAGCAGCGTATCCAC | No. 807 | 808 |
| | ATAGCGT | | |
| | SEQ. I.D. No. 806 | | |
| P7_54 | CAAGCAGAAGACGGCATACGAGATAGC | AGCTGGAA | TTCCAGCT |
| | TGGAAGTGACTGGAGTTCAGACGTGTG | SEQ. I.D. | SEQ. I.D. No. |
| | CTCTTCCGATCTTAAAGCAGCGTATCCA | No. 810 | 811 |
| | CATAGCGT | | |
| | SEQ. I.D. No. 809 | | |
| P7_55 | CAAGCAGAAGACGGCATACGAGATGCT | GCTAACTA | TAGTTAGC |
| | AACTAGTGACTGGAGTTCAGACGTGTG | SEQ. I.D. | SEQ. I.D. No. |
| | CTCTTCCGATCTTAAAGCAGCGTATCCA | No. 813 | 814 |
| | CATAGCGT | | |
| | SEQ. I.D. No. 812 | | |
| P7_56 | CAAGCAGAAGACGGCATACGAGATTTA | TTAGCGCT | AGCGCTAA |
| | GCGCTGTGACTGGAGTTCAGACGTGTG | SEQ. I.D. | SEQ. I.D. No. |
| | CTCTTCCGATCTTAAAGCAGCGTATCCA | No. 816 | 817 |
| | CATAGCGT | | |
| | SEQ. I.D. No. 815 | | |
| P7_57 | CAAGCAGAAGACGGCATACGAGATAAG | AAGAACCG | CGGTTCTT |
| | AACCGGTGACTGGAGTTCAGAC GTGTG | SEQ. I.D. | SEQ. I.D. No. |
| | CTCTTCCGATCTTAAAGCAGCGTATCCA | No. 820 | 820 |
| | CATAGCGT | | |
| | SEQ. I.D. No. 818 | | |
| P7_58 | CAAGCAGAAGACGGCATACGAGATCAA | CAATGCTA | TAGCATTG |
| | TGCTAGTGACTGGAGTTCAGACGTGTGC | SEQ. I.D. | SEQ. I.D. No. |
| | TCTTCCGATCTTAAAGCAGCGTATCCAC | No. 822 | 823 |
| | ATAGCGT | | |
| | SEQ. I.D. No. 821 | | |
| P7_59 | CAAGCAGAAGACGGCATACGAGATGTT | GTTGAATT | AATTCAAC |
| | GAATTGTGACTGGAGTTCAGACGTGTG | SEQ. I.D. | SEQ. I.D. No. |
| | CTCTTCCGATCTTAAAGCAGCGTATCCA | No. 825 | 826 |
| | CATAGCGT | | |
| | SEQ. I.D. No. 824 | | |
| P7_60 | CAAGCAGAAGACGGCATACGAGATTCT | TCTGTGAA | TTCACAGA |
| | GTGAAGTGACTGGAGTTCAGACGTGTG | SEQ. I.D. | SEQ. I.D. No. |
| | CTCTTCCGATCTTAAAGCAGCGTATCCA | No. 828 | 829 |
| | CATAGCGT | | |
| | SEQ. I.D. No. 827 | | |
| P7_61 | CAAGCAGAAGACGGCATACGAGATAAG | AAGAGA | GCTCTCTT |
| | AGAGCGTGACTGGAGTTCAGACGTGTG | GC | SEQ. I.D. No. |
| | CTCTTCCGATCTTAAAGCAGCGTATCCA | SEQ. I.D. | 832 |
| | CATAGCGT | No. 831 | |
| | SEQ. I.D. No. 830 | | |
| P7_62 | CAAGCAGAAGACGGCATACGAGATCCA | CCAAGTCA | TGACTTGG |
| | AGTCAGTGACTGGAGTTCAGACGTGTG | AAGAGA | AAGAGAGC |
| | CTCTTCCGATCTTAAAGCAGCGTATCCA | GC | SEQ. I.D. No. |
| | CATAGCGT AAGAGAGC | SEQ. I.D. | 835 |
| | SEQ. I.D. No. 833 | No. 834 | |
| P7_63 | CAAGCAGAAGACGGCATACGAGATGAA | GAACCATA | TATGGTTC |
| | CCATAGTGACTGGAGTTCAGACGTGTG | AAGAGA | AAGAGAGC |
| | CTCTTCCGATCTTAAAGCAGCGTATCCA | GC | SEQ. I.D. No. |
| | CATAGCGT AAGAGAGC | SEQ. I.D. | 838 |
| | SEQ. I.D. No. 836 | No. 837 | |
| P7_64 | CAAGCAGAAGACGGCATACGAGATGGC | GGCTAGTG | CACTAGCC |
| | TAGTGGTGACTGGAGTTCAGACGTGTG | SEQ. I.D. | SEQ. I.D. No. |
| | CTCTTCCGATCTTAAAGCAGCGTATCCA | No. 840 | 841 |
| | CATAGCGT | | |
| | SEQ. I.D. No. 839 | | |
| P7_65 | CAAGCAGAAGACGGCATACGAGATAAG | AAGAGGTT | AACCTCTT |
| | AGGTTGTGACTGGAGTTCAGACGTGTG | SEQ. I.D. | SEQ. I.D. No. |
| | CTCTTCCGATCTTAAAGCAGCGTATCCA | No. 843 | 844 |
| | CATAGCGT | | |
| | SEQ. I.D. No. 842 | | |
| P7_66 | CAAGCAGAAGACGGCATACGAGATCAA | CAATGTAG | CTACATTG |
| | TGTAGGTGACTGGAGTTCAGACGTGTG | SEQ. I.D. | SEQ. I.D. No. |
| | CTCTTCCGATCTTAAAGCAGCGTATCCA | No. 846 | 847 |
| | CATAGCGT | | |
| | SEQ. I.D. No. 845 | | |

P5 or plate primers (100 allow for 100 96-well or 25 384-well plates) (IDT ieHPLC purified)

| Name | Sequence | index (direct) |
|---|---|---|
| S1-p5 | AATGATACGGCGACCACCGAGATCTACACTCTTTCC | ATCGTACG |
| | CTACACGACGCTCTTCCGATCTATCGTACGATCTTGT | SEQ. I.D. No. |
| | GGAAAGGACGA | 849 |
| | SEQ. I.D. No. 848 | |
| S2-p5 | AATGATACGGCGACCACCGAGATCTACACTCTTTCC | ACTATCTG |
| | CTACACGACGCTCTTCCGATCTACTATCTGGATCTTG | SEQ. I.D. No. |
| | TGGAAAGGACGA | 851 |
| | SEQ. I.D. No. 850 | |
| S3-p5 | AATGATACGGCGACCACCGAGATCTACACTCTTTCC | TAGCGAGT |
| | CTACACGACGCTCTTCCGATCTTAGCGAGTCGATCTT | SEQ. I.D. No. |
| | GTGGAAAGGACGA | 853 |
| | SEQ. I.D. No. 852 | |
| S4-p5 | AATGATACGGCGACCACCGAGATCTACACTCTTTCC | CTGCGTGT |
| | CTACACGACGCTCTTCCGATCTCTGCGTGTACGATCT | SEQ. I.D. No. |
| | TGTGGAAAGGACGA | 855 |
| | SEQ. I.D. No. 854 | |
| S5-p5 | AATGATACGGCGACCACCGAGATCTACACTCTTTCC | TCATCGAG |
| | CTACACGACGCTCTTCCGATCTTCATCGAGATCTTGT | SEQ. I.D. No. |
| | GGAAAGGACGA | 857 |
| | SEQ. I.D. No. 856 | |
| S6-p5 | AATGATACGGCGACCACCGAGATCTACACTCTTTCC | CGTGAGTG |
| | CTACACGACGCTCTTCCGATCTCGTGAGTGGATCTTG | SEQ. I.D. No. |
| | TGGAAAGGACGA | 859 |
| | SEQ. I.D. No. 858 | |
| S7-p5 | AATGATACGGCGACCACCGAGATCTACACTCTTTCC | GGATATCT |
| | CTACACGACGCTCTTCCGATCTGGATATCTCGATCTT | SEQ. I.D. No. |
| | GTGGAAAGGACGA | 861 |
| | SEQ. I.D. No. 860 | |
| S8-p5 | AATGATACGGCGACCACCGAGATCTACACTCTTTCC | GACACCGT |
| | CTACACGACGCTCTTCCGATCTGACACCGTACGATCT | SEQ. I.D. No. |
| | TGTGGAAAGGACGA | 863 |
| | SEQ. I.D. No. 862 | |
| S9-p5 | AATGATACGGCGACCACCGAGATCTACACTCTTTCC | CTACTATA |
| | CTACACGACGCTCTTCCGATCTCTACTATAATCTTGT | SEQ. I.D. No. |
| | GGAAAGGACGA | 865 |
| | SEQ. I.D. No. 864 | |
| S10-p5 | AATGATACGGCGACCACCGAGATCTACACTCTTTCC | CGTTACTA |
| | CTACACGACGCTCTTCCGATCTCGTTACTAGATCTTG | SEQ. I.D. No. |
| | TGGAAAGGACGA | 867 |
| | SEQ. I.D. No. 866 | |
| S11-p5 | AATGATACGGCGACCACCGAGATCTACACTCTTTCC | AGAGTCAC |
| | CTACACGACGCTCTTCCGATCTAGAGTCACCGATCTT | SEQ. I.D. No. |
| | GTGGAAAGGACGA | 869 |
| | SEQ. I.D. No. 868 | |
| S12-p5 | AATGATACGGCGACCACCGAGATCTACACTCTTTCC | TACGAGAC |
| | CTACACGACGCTCTTCCGATCTTACGAGACACGATC | SEQ. I.D. No. |
| | TTGTGGAAAGGACGA | 871 |
| | SEQ. I.D. No. 870 | |
| S13-p5 | AATGATACGGCGACCACCGAGATCTACACTCTTTCC | ACGTCTCG |
| | CTACACGACGCTCTTCCGATCTACGTCTCGATCTTGT | SEQ. I.D. No. |
| | GGAAAGGACGA | 873 |
| | SEQ. I.D. No. 872 | |
| S14-p5 | AATGATACGGCGACCACCGAGATCTACACTCTTTCC | TCGACGAG |
| | CTACACGACGCTCTTCCGATCTTCGACGAGGATCTTG | SEQ. I.D. No. |
| | TGGAAAGGACGA | 875 |
| | SEQ. I.D. No. 874 | |
| S15-p5 | AATGATACGGCGACCACCGAGATCTACACTCTTTCC | GATCGTGT |
| | CTACACGACGCTCTTCCGATCTGATCGTGTCGATCTT | SEQ. I.D. No. |
| | GTGGAAAGGACGA | 877 |
| | SEQ. I.D. No. 876 | |
| S16-p5 | AATGATACGGCGACCACCGAGATCTACACTCTTTCC | GTCAGATA |
| | CTACACGACGCTCTTCCGATCTGTCAGATAACGATCT | SEQ. I.D. No. |
| | TGTGGAAAGGACGA | 879 |
| | SEQ. I.D. No. 878 | |
| S17-p5 | AATGATACGGCGACCACCGAGATCTACACTCTTTCC | ACGACGTG |
| | CTACACGACGCTCTTCCGATCTACGACGTGATCTTGT | SEQ. I.D. No. |
| | GGAAAGGACGA | 881 |
| | SEQ. I.D. No. 880 | |
| S18-p5 | AATGATACGGCGACCACCGAGATCTACACTCTTTCC | ATATACAC |
| | CTACACGACGCTCTTCCGATCTATATACACGATCTTG | SEQ. I.D. No. |
| | TGGAAAGGACGA | 883 |
| | SEQ. I.D. No. 882 | |
| S19-p5 | AATGATACGGCGACCACCGAGATCTACACTCTTTCC | CGTCGCTA |
| | CTACACGACGCTCTTCCGATCTCGTCGCTACGATCTT | SEQ. I.D. No. |
| | GTGGAAAGGACGA | 885 |
| | SEQ. I.D. No. 884 | |
| S20-p5 | AATGATACGGCGACCACCGAGATCTACACTCTTTCC | CTAGAGCT |
| | CTACACGACGCTCTTCCGATCTCTAGAGCTACGATCT | SEQ. I.D. No. |
| | TGTGGAAAGGACGA | 887 |
| | SEQ. I.D. No. 886 | |
| S21-p5 | AATGATACGGCGACCACCGAGATCTACACTCTTTCC | GCTCTAGT |
| | CTACACGACGCTCTTCCGATCTGCTCTAGTATCTTGT | SEQ. I.D. No. |
| | GGAAAGGACGA | 889 |
| | SEQ. I.D. No. 888 | |
| S22-p5 | AATGATACGGCGACCACCGAGATCTACACTCTTTCC | GACACTGA |
| | CTACACGACGCTCTTCCGATCTGACACTGAGATCTTG | SEQ. I.D. No. |
| | TGGAAAGGACGA | 891 |
| | SEQ. I.D. No. 890 | |
| S23-p5 | AATGATACGGCGACCACCGAGATCTACACTCTTTCC | TGCGTACG |
| | CTACACGACGCTCTTCCGATCTTGCGTACGCGATCTT | SEQ. I.D. No. |
| | GTGGAAAGGACGA | 893 |
| | SEQ. I.D. No. 892 | |
| S24-p5 | AATGATACGGCGACCACCGAGATCTACACTCTTTCC | TAGTGTAG |
| | CTACACGACGCTCTTCCGATCTTAGTGTAGACGATCT | SEQ. I.D. No. |
| | TGTGGAAAGGACGA | 885 |
| | SEQ. I.D. No. 894 | |
| S25-p5 | AATGATACGGCGACCACCGAGATCTACACTCTTTCC | AAGCAGCA |
| | CTACACGACGCTCTTCCGATCTAAGCAGCAATCTTGT | SEQ. I.D. No. |
| | GGAAAGGACGA | 897 |
| | SEQ. I.D. No. 896 | |
| S26-p5 | AATGATACGGCGACCACCGAGATCTACACTCTTTCC | ACGCGTGA |
| | CTACACGACGCTCTTCCGATCTACGCGTGAGATCTTG | SEQ. I.D. No. |
| | TGGAAAGGACGA | 899 |
| | SEQ. I.D. No. 898 | |
| S27-p5 | AATGATACGGCGACCACCGAGATCTACACTCTTTCC | CGATCTAC |
| | CTACACGACGCTCTTCCGATCTCGATCTACCGATCTT | SEQ. I.D. No. |
| | GTGGAAAGGACGA | 901 |
| | SEQ. I.D. No. 900 | |
| S28-p5 | AATGATACGGCGACCACCGAGATCTACACTCTTTCC | TGCGTCAC |
| | CTACACGACGCTCTTCCGATCTTGCGTCACACGATCT | SEQ. I.D. No. |
| | TGTGGAAAGGACGA | 903 |
| | SEQ. I.D. No. 902 | |
| S29-p5 | AATGATACGGCGACCACCGAGATCTACACTCTTTCC | GTCTAGTG |
| | CTACACGACGCTCTTCCGATCTGTCTAGTGATCTTGT | SEQ. I.D. No. |
| | GGAAAGGACGA | 905 |
| | SEQ. I.D. No. 904 | |
| S30-p5 | AATGATACGGCGACCACCGAGATCTACACTCTTTCC | CTAGTATG |
| | CTACACGACGCTCTTCCGATCTCTAGTATGGATCTTG | SEQ. I.D. No. |
| | TGGAAAGGACGA | 907 |
| | SEQ. I.D. No. 906 | |
| S31-p5 | AATGATACGGCGACCACCGAGATCTACACTCTTTCC | GATAGCGT |
| | CTACACGACGCTCTTCCGATCTGATAGCGTCGATCTT | SEQ. I.D. No. |
| | GTGGAAAGGACGA | 909 |
| | SEQ. I.D. No. 908 | |
| S32-p5 | AATGATACGGCGACCACCGAGATCTACACTCTTTCC | TCTACACT |
| | CTACACGACGCTCTTCCGATCTTCTACACTACGATCT | SEQ. I.D. No. |
| | TGTGGAAAGGACGA | 911 |
| | SEQ. I.D. No. 910 | |
| S33-p5 | AATGATACGGCGACCACCGAGATCTACACTCTTTCC | AACTCTCG |
| | CTACACGACGCTCTTCCGATCTAACTCTCGATCTTGT | SEQ. I.D. No. |
| | GGAAAGGACGA | 913 |
| | SEQ. I.D. No. 912 | |
| S34-p5 | AATGATACGGCGACCACCGAGATCTACACTCTTTCC | ACTATGTC |
| | CTACACGACGCTCTTCCGATCTACTATGTCGATCTTG | SEQ. I.D. No. |
| | TGGAAAGGACGA | 915 |
| | SEQ. I.D. No. 914 | |
| S35-p5 | AATGATACGGCGACCACCGAGATCTACACTCTTTCC | AGTAGCGT |
| | CTACACGACGCTCTTCCGATCTAGTAGCGTCGATCTT | SEQ. I.D. No. |
| | GTGGAAAGGACGA | 917 |
| | SEQ. I.D. No. 916 | |
| S36-p5 | AATGATACGGCGACCACCGAGATCTACACTCTTTCC | CAGTGAGT |
| | CTACACGACGCTCTTCCGATCTCAGTGAGTACGATCT | SEQ. I.D. No. |
| | TGTGGAAAGGACGA | 919 |
| | SEQ. I.D. No. 918 | |
| S37-p5 | AATGATACGGCGACCACCGAGATCTACACTCTTTCC | CGTACTCA |
| | CTACACGACGCTCTTCCGATCTCGTACTCAATCTTGT | SEQ. I.D. No. |
| | GGAAAGGACGA | 921 |
| | SEQ. I.D. No. 920 | |
| S38-p5 | AATGATACGGCGACCACCGAGATCTACACTCTTTCC | CTACGCAG |
| | CTACACGACGCTCTTCCGATCTCTACGCAGGATCTTG | SEQ. I.D. No. |
| | TGGAAAGGACGA | 923 |
| | SEQ. I.D. No. 922 | |
| S39-p5 | AATGATACGGCGACCACCGAGATCTACACTCTTTCC | GGAGACTA |
| | CTACACGACGCTCTTCCGATCTGGAGACTACGATCTT | SEQ. I.D. No. |
| | GTGGAAAGGACGA | 925 |
| | SEQ. I.D. No. 924 | |
| S40-p5 | AATGATACGGCGACCACCGAGATCTACACTCTTTCC | GTCGCTCG |
| | CTACACGACGCTCTTCCGATCTGTCGCTCGACGATCT | SEQ. I.D. No. |
| | TGTGGAAAGG | 927 |
| | ACGA | |
| | SEQ. I.D. No. 926 | |
| S41-p5 | AATGATACGGCGACCACCGAGATCTACACTCTTTCC | GTCGTAGT |
| | CTACACGACGCTCTTCCGATCTGTCGTAGTATCTTGT | SEQ. I.D. No. |
| | GGAAAGGACGA | 929 |
| | SEQ. I.D. No. 928 | |
| S42-p5 | AATGATACGGCGACCACCGAGATCTACACTCTTTCC | TAGCAGAC |
| | CTACACGACGCTCTTCCGATCTTAGCAGACGATCTTG | SEQ. I.D. No. |
| | TGGAAAGGACGA | 931 |
| | SEQ. I.D. No. 930 | |
| S43-p5 | AATGATACGGCGACCACCGAGATCTACACTCTTTCC | TCATAGAC |
| | CTACACGACGCTCTTCCGATCTTCATAGACCGATCTT | SEQ. I.D. No. |
| | GTGGAAAGGACGA | 933 |
| | SEQ. I.D. No. 932 | |
| S44-p5 | AATGATACGGCGACCACCGAGATCTACACTCTTTCC | TCGCTATA |
| | CTACACGACGCTCTTCCGATCTTCGCTATAACGATCT | SEQ. I.D. No. |
| | TGTGGAAAGGACGA | 935 |
| | SEQ. I.D. No. 934 | |
| S45-p5 | AATGATACGGCGACCACCGAGATCTACACTCTTTCC | AAGTCGAG |
| | CTACACGACGCTCTTCCGATCTAAGTCGAGATCTTGT | SEQ. I.D. No. |
| | GGAAAGGACGA | 937 |
| | SEQ. I.D. No. 936 | |
| S46-p5 | AATGATACGGCGACCACCGAGATCTACACTCTTTCC | ATACTTCG |
| | CTACACGACGCTCTTCCGATCTATACTTCGGATCTTG | SEQ. I.D. No. |
| | TGGAAAGGACGA | 939 |
| | SEQ. I.D. No. 938 | |
| S47-p5 | AATGATACGGCGACCACCGAGATCTACACTCTTTCC | AGCTGCTA |
| | CTACACGACGCTCTTCCGATCTAGCTGCTACGATCTT | SEQ. I.D. No. |
| | GTGGAAAGGACGA | 941 |
| | SEQ. I.D. No. 940 | |
| S48-p5 | AATGATACGGCGACCACCGAGATCTACACTCTTTCC | CATAGAGA |
| | CTACACGACGCTCTTCCGATCTCATAGAGAACGATC | SEQ. I.D. No. |
| | TTGTGGAAAGGACGA | 943 |
| | SEQ. I.D. No. 942 | |
| S49-p5 | AATGATACGGCGACCACCGAGATCTACACTCTTTCC | GCTAATAG |
| | CTACACGACGCTCTTCCGATCTGCTAATAGATCTTGT | SEQ. I.D. No. |
| | GGAAAGGACGA | 945 |
| | SEQ. I.D. No. 944 | |
| S50-p5 | AATGATACGGCGACCACCGAGATCTACACTCTTTCC | TGGTTGGA |
| | CTACACGACGCTCTTCCGATCTTGGTTGGAGATCTTG | SEQ. I.D. No. |
| | TGGAAAGGACGA | 947 |
| | SEQ. I.D. No. 946 | |
| S51-p5 | AATGATACGGCGACCACCGAGATCTACACTCTTTCC | ATAGCCAG |
| | CTACACGACGCTCTTCCGATCTATAGCCAGCGATCTT | SEQ. I.D. No. |
| | GTGGAAAGGACGA | 949 |
| | SEQ. I.D. No. 948 | |
| S52-p5 | AATGATACGGCGACCACCGAGATCTACACTCTTTCC | GAGCCAGT |
| | CTACACGACGCTCTTCCGATCTGAGCCAGTACGATC | SEQ. I.D. No. |
| | TTGTGGAAAGGACGA | 951 |
| | SEQ. I.D. No. 950 | |
| S53-p5 | AATGATACGGCGACCACCGAGATCTACACTCTTTCC | TGTGATGG |
| | CTACACGACGCTCTTCCGATCTTGTGATGGATCTTGT | SEQ. I.D. No. |
| | GGAAAGGACGA | 953 |
| | SEQ. I.D. No. 952 | |
| S54-p5 | AATGATACGGCGACCACCGAGATCTACACTCTTTCC | GTATTGCC |
| | CTACACGACGCTCTTCCGATCTGTATTGCCGATCTTG | SEQ. I.D. No. |
| | TGGAAAGGACGA | 955 |
| | SEQ. I.D. No. 954 | |
| S55-p5 | AATGATACGGCGACCACCGAGATCTACACTCTTTCC | ATGAAGTG |
| | CTACACGACGCTCTTCCGATCTATGAAGTGCGATCTT | SEQ. I.D. No. |
| | GTGGAAAGGACGA | 957 |
| | SEQ. I.D. No. 956 | |
| S56-p5 | AATGATACGGCGACCACCGAGATCTACACTCTTTCC | TAAGCTTG |
| | CTACACGACGCTCTTCCGATCTTAAGCTTGACGATCT | SEQ. I.D. No. |
| | TGTGGAAAGGACGA | 959 |
| | SEQ. I.D. No. 958 | |
| S57-p5 | AATGATACGGCGACCACCGAGATCTACACTCTTTCC | TGGTACCT |
| | CTACACGACGCTCTTCCGATCTTGGTACCTATCTTGT | SEQ. I.D. No. |
| | GGAAAGGACGA | 961 |
| | SEQ. I.D. No. 960 | |
| S58-p5 | AATGATACGGCGACCACCGAGATCTACACTCTTTCC | GTTATGGA |
| | CTACACGACGCTCTTCCGATCTGTTATGGAGATCTTG | SEQ. I.D. No. |
| | TGGAAAGGACGA | 963 |
| | SEQ. I.D. No. 962 | |
| S59-p5 | AATGATACGGCGACCACCGAGATCTACACTCTTTCC | ATGAGGAC |
| | CTACACGACGCTCTTCCGATCTATGAGGACCGATCTT | SEQ. I.D. No. |
| | GTGGAAAGGACGA | 965 |
| | SEQ. I.D. No. 964 | |
| S60-p5 | AATGATACGGCGACCACCGAGATCTACACTCTTTCC | GCAGTACT |
| | CTACACGACGCTCTTCCGATCTGCAGTACTACGATCT | SEQ. I.D. No. |
| | TGTGGAAAGGACGA | 967 |
| | SEQ. I.D. No. 966 | |
| S61-p5 | AATGATACGGCGACCACCGAGATCTACACTCTTTCC | CTTGAATC |
| | CTACACGACGCTCTTCCGATCTCTTGAATCATCTTGT | SEQ. I.D. No. |
| | GGAAAGGACGA | 969 |
| | SEQ. I.D. No. 968 | |
| S62-p5 | AATGATACGGCGACCACCGAGATCTACACTCTTTCC | CCAACTAA |
| | CTACACGACGCTCTTCCGATCTCCAACTAAGATCTTG | SEQ. I.D. No. |
| | TGGAAAGGACGA | 971 |
| | SEQ. I.D. No. 970 | |
| S63-p5 | AATGATACGGCGACCACCGAGATCTACACTCTTTCC | AATACCAT |
| | CTACACGACGCTCTTCCGATCTAATACCATCGATCTT | SEQ. I.D. No. |
| | GTGGAAAGGACGA | 973 |
| | SEQ. I.D. No. 972 | |
| S64-p5 | AATGATACGGCGACCACCGAGATCTACACTCTTTCC | ACCTATGC |
| | CTACACGACGCTCTTCCGATCTACCTATGCACGATCT | SEQ. I.D. No. |
| | TGTGGAAAGGACGA | 975 |
| | SEQ. I.D. No. 974 | |
| S65-p5 | AATGATACGGCGACCACCGAGATCTACACTCTTTCC | GAACGCTA |
| | CTACACGACGCTCTTCCGATCTGAACGCTAATCTTGT | SEQ. I.D. No. |
| | GGAAAGGACGA | 977 |
| | SEQ. I.D. No. 976 | |
| S66-p5 | AATGATACGGCGACCACCGAGATCTACACTCTTTCC | CTGACATC |
| | CTACACGACGCTCTTCCGATCTCTGACATCGATCTTG | SEQ. I.D. No. |
| | TGGAAAGGACGA | 979 |
| | SEQ. I.D. No. 978 | |
| S67-p5 | AATGATACGGCGACCACCGAGATCTACACTCTTTCC | GCCACCAT |
| | CTACACGACGCTCTTCCGATCTGCCACCATCGATCTT | SEQ. I.D. No. |
| | GTGGAAAGGACGA | 981 |
| | SEQ. I.D. No. 980 | |
| S68-p5 | AATGATACGGCGACCACCGAGATCTACACTCTTTCC | CGACTCTC |
| | CTACACGACGCTCTTCCGATCTCGACTCTCACGATCT | SEQ. I.D. No. |
| | TGTGGAAAGGACGA | 983 |
| | SEQ. I.D. No. 982 | |
| S69-p5 | AATGATACGGCGACCACCGAGATCTACACTCTTTCC | TGCTATTA |
| | CTACACGACGCTCTTCCGATCTTGCTATTAATCTTGT | SEQ. I.D. No. |
| | GGAAAGGACGA | 985 |
| | SEQ. I.D. No. 984 | |
| S70-p5 | AATGATACGGCGACCACCGAGATCTACACTCTTTCC | CTTCTGGC |
| | CTACACGACGCTCTTCCGATCTCTTCTGGCGATCTTG | SEQ. I.D. No. |
| | TGGAAAGGACGA | 987 |
| | SEQ. I.D. No. 986 | |
| S71-p5 | AATGATACGGCGACCACCGAGATCTACACTCTTTCC | ATGAATTA |
| | CTACACGACGCTCTTCCGATCTATGAATTACGATCTT | SEQ. I.D. No. |
| | GTGGAAAGGACGA | 989 |
| | SEQ. I.D. No. 988 | |
| S72-p5 | AATGATACGGCGACCACCGAGATCTACACTCTTTCC | TACTCCAG |
| | CTACACGACGCTCTTCCGATCTTACTCCAGACGATCT | SEQ. I.D. No. |
| | TGTGGAAAGGACGA | 991 |
| | SEQ. I.D. No. 990 | |
| S73-p5 | AATGATACGGCGACCACCGAGATCTACACTCTTTCC | ATCATACC |
| | CTACACGACGCTCTTCCGATCTATCATACCATCTTGT | SEQ. I.D. No. |
| | GGAAAGGACGA | 993 |
| | SEQ. I.D. No. 992 | |
| S74-p5 | AATGATACGGCGACCACCGAGATCTACACTCTTTCC | CCTCTAAC |
| | CTACACGACGCTCTTCCGATCTCCTCTAACGATCTTG | SEQ. I.D. No. |
| | TGGAAAGGACGA | 995 |
| | SEQ. I.D. No. 994 | |
| S75-p5 | AATGATACGGCGACCACCGAGATCTACACTCTTTCC | ATCTTCTC |
| | CTACACGACGCTCTTCCGATCTATCTTCTCCGATCTT | SEQ. I.D. No. |
| | GTGGAAAGGACGA | 997 |
| | SEQ. I.D. No. 996 | |
| S76-p5 | AATGATACGGCGACCACCGAGATCTACACTCTTTCC | CAGCTCAC |
| | CTACACGACGCTCTTCCGATCTCAGCTCACACGATCT | SEQ. I.D. No. |
| | TGTGGAAAGGACGA | 999 |
| | SEQ. I.D. No. 998 | |
| S77-p5 | AATGATACGGCGACCACCGAGATCTACACTCTTTCC | GGTTATCT |
| | CTACACGACGCTCTTCCGATCTGGTTATCTATCTTGT | SEQ. I.D. No. |
| | GGAAAGGACGA | 1001 |
| | SEQ. I.D. No. 1000 | |
| S78-p5 | AATGATACGGCGACCACCGAGATCTACACTCTTTCC | TCCGCATA |
| | CTACACGACGCTCTTCCGATCTTCCGCATAGATCTTG | SEQ. I.D. No. |
| | TGGAAAGGACGA | 1003 |
| | SEQ. I.D. No. 1002 | |
| S79-p5 | AATGATACGGCGACCACCGAGATCTACACTCTTTCC | TGCTTCAC |
| | CTACACGACGCTCTTCCGATCTTGCTTCACCGATCTT | SEQ. I.D. No. |
| | GTGGAAAGGACGA | 1005 |
| | SEQ. I.D. No. 1004 | |
| S80-p5 | AATGATACGGCGACCACCGAGATCTACACTCTTTCC | GCTTCCTA |
| | CTACACGACGCTCTTCCGATCTGCTTCCTAACGATCT | SEQ. I.D. No. |
| | TGTGGAAAGGACGA | 1007 |
| | SEQ. I.D. No. 1006 | |
| S81-p5 | AATGATACGGCGACCACCGAGATCTACACTCTTTCC | GTAATCGC |
| | CTACACGACGCTCTTCCGATCTGTAATCGCATCTTGT | SEQ. I.D. No. |
| | GGAAAGGACGA | 1009 |
| | SEQ. I.D. No. 1008 | |
| S82-p5 | AATGATACGGCGACCACCGAGATCTACACTCTTTCC | GGCCAATT |
| | CTACACGACGCTCTTCCGATCTGGCCAATTGATCTTG | SEQ. I.D. No. |
| | TGGAAAGGACGA | 1011 |
| | SEQ. I.D. No. 1010 | |
| S83-p5 | AATGATACGGCGACCACCGAGATCTACACTCTTTCC | AAGCAATT |
| | CTACACGACGCTCTTCCGATCTAAGCAATTCGATCTT | SEQ. I.D. No. |
| | GTGGAAAGGACGA | 1013 |
| | SEQ. I.D. No. 1012 | |
| S84-p5 | AATGATACGGCGACCACCGAGATCTACACTCTTTCC | CAGACCAA |
| | CTACACGACGCTCTTCCGATCTCAGACCAAACGATC | SEQ. I.D. No. |
| | TTGTGGAAAGGACGA | 1015 |
| | SEQ. I.D. No. 1014 | |
| S85-p5 | AATGATACGGCGACCACCGAGATCTACACTCTTTCC | CCAGGATG |
| | CTACACGACGCTCTTCCGATCTCCAGGATGATCTTGT | SEQ. I.D. No. |
| | GGAAAGGACGA | 1017 |
| | SEQ. I.D. No. 1016 | |
| S86-p5 | AATGATACGGCGACCACCGAGATCTACACTCTTTCC | GTTAATCC |
| | CTACACGACGCTCTTCCGATCTGTTAATCCGATCTTG | SEQ. I.D. No. |
| | TGGAAAGGACGA | 1019 |
| | SEQ. I.D. No. 1018 | |
| S87-p5 | AATGATACGGCGACCACCGAGATCTACACTCTTTCC | AATATGCG |
| | CTACACGACGCTCTTCCGATCTAATATGCGCGATCTT | SEQ. I.D. No. |
| | GTGGAAAGGACGA | 1021 |
| | SEQ. I.D. No. 1020 | |
| S88-p5 | AATGATACGGCGACCACCGAGATCTACACTCTTTCC | TCGAATGA |
| | CTACACGACGCTCTTCCGATCTTCGAATGAACGATCT | SEQ. I.D. No. |
| | TGTGGAAAGGACGA | 1023 |
| | SEQ. I.D. No. 1022 | |
| S89-p5 | AATGATACGGCGACCACCGAGATCTACACTCTTTCC | GATTGGAC |
| | CTACACGACGCTCTTCCGATCTGATTGGACATCTTGT | SEQ. I.D. No. |
| | GGAAAGGACGA | 1025 |
| | SEQ. I.D. No. 1024 | |
| S90-p5 | AATGATACGGCGACCACCGAGATCTACACTCTTTCC | TGACCAAG |
| | CTACACGACGCTCTTCCGATCTTGACCAAGGATCTTG | SEQ. I.D. No. |
| | TGGAAAGGACGA | 1027 |
| | SEQ. I.D. No. 1026 | |
| S91-p5 | AATGATACGGCGACCACCGAGATCTACACTCTTTCC | AGCGTTGG |
| | CTACACGACGCTCTTCCGATCTAGCGTTGGCGATCTT | SEQ. I.D. No. |
| | GTGGAAAGGACGA | 1029 |
| | SEQ. I.D. No. 1028 | |
| S92-p5 | AATGATACGGCGACCACCGAGATCTACACTCTTTCC | GAAGTGGA |
| | CTACACGACGCTCTTCCGATCTGAAGTGGAACGATC | SEQ. I.D. No. |
| | TTGTGGAAAGGACGA | 1031 |
| | SEQ. I.D. No. 1030 | |
| S93-p5 | AATGATACGGCGACCACCGAGATCTACACTCTTTCC | TGGAGATT |
| | CTACACGACGCTCTTCCGATCTTGGAGATTATCTTGT | SEQ. I.D. No. |
| | GGAAAGGACGA | 1033 |
| | SEQ. I.D. No. 1032 | |
| S94-p5 | AATGATACGGCGACCACCGAGATCTACACTCTTTCC | GTGCAGAC |
| | CTACACGACGCTCTTCCGATCTGTGCAGACGATCTTG | SEQ. I.D. No. |
| | TGGAAAGGACGA | 1035 |
| | SEQ. I.D. No. 1034 | |
| S95-p5 | AATGATACGGCGACCACCGAGATCTACACTCTTTCC | GCGCTATT |
| | CTACACGACGCTCTTCCGATCTGCGCTATTCGATCTT | SEQ. I.D. No. |
| | GTGGAAAGGACGA | 1037 |
| | SEQ. I.D. No. 1036 | |
| S96-p5 | AATGATACGGCGACCACCGAGATCTACACTCTTTCC | AAGAGATT |
| | CTACACGACGCTCTTCCGATCTAAGAGATTACGATC | SEQ. I.D. No. |
| | TTGTGGAAAGGACGA | 1039 |
| | SEQ. I.D. No. 1038 | |
| S97-p5 | AATGATACGGCGACCACCGAGATCTACACTCTTTCC | TGTGCATT |
| | CTACACGACGCTCTTCCGATCTTGTGCATTATCTTGT | SEQ. I.D. No. |
| | GGAAAGGACGA | 1041 |
| | SEQ. I.D. No. 1040 | |
| S98-p5 | AATGATACGGCGACCACCGAGATCTACACTCTTTCC | GATCGCGT |
| | CTACACGACGCTCTTCCGATCTGATCGCGTGATCTTG | SEQ. I.D. No. |
| | TGGAAAGGACGA | 1043 |
| | SEQ. I.D. No. 1042 | |
| S99-p5 | AATGATACGGCGACCACCGAGATCTACACTCTTTCC | AAGATAGT |
| | CTACACGACGCTCTTCCGATCTAAGATAGTCGATCTT | SEQ. I.D. No. |
| | GTGGAAAGGACGA | 1045 |
| | SEQ. I.D. No. 1044 | |
| S100-p5 | AATGATACGGCGACCACCGAGATCTACACTCTTTCC | CTCCCAGT |
| | CTACACGACGCTCTTCCGATCTCTCCCAGTACGATCT | SEQ. I.D. No. |
| | TGTGGAAAGGACGA | 1047 |
| | SEQ. I.D. No. 1046 | |

C. Disposable Equipment
Ink well culture bottle
Corning roller bottle (Corning, 490 cm²)
Corning 384 well clear plates Corning brand #3701
Corning 96-well clear bottom plate (3370)
Aerosol Barrier Tips
Nalgene Reservoirs, 300 mL convoluted bottom
Tupperware (6-¼″×8-⅝″×5-⅞″ h)
50 mL BD Falcon tubes
Kimtech shop towels
Eppendorf twintec 384-well PCR plates
Eppendorf twintec 96-well PCR plates

2. Strain Expansion
1. Strain pools are organized by group (Screening group 1, group 2, group 3 and group 4).
2. In the BSL3 laboratory start a growth for each strain in a separate inkwell containing 10ml 7H9+OADC supplemented with selection agents and 500 ng/ml ATC. Selective agents listed in strain table above. Inoculate with the full cryovial volume for an approximately 1:10 inoculation.
3. Incubate in 37° C. cabinet for 3-5 days until OD₆₀₀>0.3
4. Supplement AHT every 4th day by adding to 500 ng/μl final concentration.

Assay Setup
1. Prior to the day of the assay, prepare assay-ready plates by pre-aliquotting the control compounds and compound library in duplicate into clear Corning 384-well plates (#3701).
2. On the day of or before the assay, outside the BL3 lab, use the Bravo to add 20 μL of fresh 7H9-OADC-acetate (without ATC and selective agents) to each well of each 384-well assay-ready plate. For compounds that could not be prepared as assay-ready plates, instead aliquot 20 μL of 7H9 into empty plates and then pin compound into that media. Bring these plates into the BL3.
3. Take ODs of expanded strains by transferring 100 μl of each ink well culture to the wells of a Corning 96-well plate (#3370). Read the OD₆₀₀ using the Molecular Devices M5 spectrophotometer.
4. Use the “mix_calc.xlsx” spreadsheet to calculate how much of each strain to add to pool for the volume of the given assay. 8 ml of diluted culture pool at an OD of 0.005 is required for each assay plate, plus ˜50-100 mL to account for reservoir dead volume.
5. Add the calculated volume of each strain to a 50 mL conical Falcon tube. Bring to 40 mL with fresh 7H9. Wash cells with 7H9 three times (spin at 3500RPM for 10 min in Beckman Allegra Centrifuge, remove supernatant, and resuspend pellet in fresh 7H9).
6. Prepare a roller bottle containing the full calculated volume of 7H9 required for the assay. After the final wash, pipette a small volume from the roller bottle to the conical tube to resuspend the washed pellet, then transfer it back to the roller bottle. This is the diluted culture pool.
7. Use a pipettor to fill a reservoir on the Bravo deck with diluted culture pool. Delid the assay plates and place them in the BenchCel stacker. Prepare the Bravo deck with 96 LT tips and a vesphene wash reservoir.
8. Use Bravo protocol “384w inoculate” to transfer 20 μL of culture per well to assay plates.
9. Put a kimtech shop towel dampened with H₂O in the bottom of each tupperware container to guard against evaporation. Re-lid assay plates, wipe the exteriors with 1% vesphene, and seal them 8 to a tupperware.
10. Incubate in 37° C. cabinet for 14 days.

Collecting the Assay
1. Seal each plate with foil, pressing with a finger to ensure each well is thoroughly sealed. Replace the lid.
2. Double-bag plates in sets of 4, sterilizing the exteriors of the plates and bags with 1% vesphene.
3. Bake plates for 2 hours at 80° C. to heat-kill cultures. The oven holds a maximum of 64 plates simultaneously. After baking, plates are considered sterile and safe to remove from the BL3 lab.
4. Store sealed plates at −80° C. in Rm 2070 freezer.

Library Construction

PCR
1. Spin baked 384-well plates in tabletop centrifuge at 2000 rpm for 1 minute to remove condensation from seal.
2. Prepare a lysis solution of 20% DMSO with tag 8090 control plasmid:
   800 mL dH2O
   200 mL DMSO
   500 μL tag_8090 control plasmid (3.4 pg/μL)
3. Run each plate through Bravo protocol “1—mix lysis and transfer (long)”. 40 μL of lysis solution is aspirated from a reservoir and dispensed into the baked plate. The plate is mixed thoroughly, then 204, is transferred to a 384-well twintec PCR plate.
4. Heat the template aliquot in the thermocycler at 98° C. for 10 min. Store template at −80° C. when not in use.
5. Prepare PCR master mix according to table (volumes appropriate for 16 PCR plates). Dispense 510 μL per well to rows A-F, columns 1-11 of a 96-well block.

| | Volume/reaction | Volume × 4500 |
|---|---|---|
| Component | (μL) | (μL) |
| 5x Q5 buffer | 2 | 9000 |
| dNTPs (10 mM each) | 0.5 | 2250 |
| Q5 Hot Start polymerase | 0.1 | 450 |
| tag_1180 control | 0.1 | 450 |
| plasmid (150 fg/uL) | | |
| dH2O | 5.05 | 22725 |
| Total | 7.75 | 34875 |

6. Dispense 7.75 μL of master mix to wells C2-N23 of 16 384-well twintec PCR plates using Bravo protocol “2—add master mix to 384 per”. (From here forward, columns 1 & 24 and rows A, B, O, & P will be left empty to discard potential edge effects from the growth plate.)
7. Aliquot 1.25 μL of p5/p7 primer mix (5 μM each) to PCR reactions using Bravo protocol “3—add primer to 384 per”.
8. Aliquot 1 μL of boiled template to PCR reactions using Bravo protocol “4—add template to 384 per”.
9. Run PCRs on the following thermocycler protocol:

| Temperature (° C.) | Cycles | Time (s) |
|---|---|---|
| 98 | 1 | 120 |
| 98 | 22 | 10 |
| 50 | | 20 |
| 72 | | 20 |
| 72 | 1 | 120 |
| 4 | | ∞ |

10. Pool 2.8 μL from each well of PCR plates using Bravo protocol “5—pool per plates into reservoir”.

SPRI
1. Allow SPRI reagent to warm to room temperature.
2. Mix 2 mL of PCR pool with an equal volume of SPRI reagent. Pipette slowly up and down ˜10 times to thoroughly mix.
3. Incubate at room temperature for 20 min.
4. Dispense 500 μL of solution into each of two sterile Eppendorf microtubes in the magnet rack.
5. Incubate on the magnet for 3 min.
6. Aspirate and discard the supernatant, being careful not to disturb the pelleted beads.
7. Repeat steps 4-6 until all of the solution has been cleared.
8. Still on the magnet, wash each tube 3 times with 80% EtOH: add 900 μL, incubate for 30 s, then aspirate and discard the supernatant.
9. Leave the tubes open on the magnet for 15 min to dry. Pipet off any excess EtOH from the bottom of the tubes.
10. Remove the tubes from the magnet. Thoroughly resuspend the beads from the first tube in 250 μL dH2O by pipetting up and down. Transfer the resuspended solution to the second tube and resuspend those beads as well.
11. Incubate the resuspended solution off the magnet for 20 min at room temperature.
12. Return the tube to the magnet. Incubate for 3 min. Keep the supernatant and discard the beads.
13. Save 10 μL of eluent for quality control. Add equal volume of fresh SPRI beads to the remaining ˜240 μL and mix thoroughly as in step 2.
14. Repeat steps 3-9, but this time in a single Eppendorf tube. Repeat steps 10-12, this time eluting in a final volume of 75 μL.

Bioanalyzer
1. Dilute 2 μL of the purified library to 20 μL with dH2O. Perform similar 1:10 dilutions for the unpurified PCR pool and the 1×-purified sample you set aside in SPRI step 13.
2. Run an Agilent bioanalyzer chip with the diluted samples. The purified library sample will provide quantification and quality assurance. The other two samples will provide further quality control.
3. If the library looks clean (<<1% 100 bp primer vs 200 bp product) and has a good yield, prepare a 40 μL dilution at 10 nM to submit to walk-up sequencing.
4. If the library looks unclean, then repeat a cycle of SPRI and verify quality with a new bioanalyzer chip.

All publications, patents, and patent application mentioned herein are incorporated by reference to the same extent as if each individual publication, patent or patent application was specifically and individually indicated to be incorporated by reference in its entirety.

Various modifications and variations of the described methods, compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure that come within known customary practice within the art to which the invention pertains and may be applied to the features herein before set forth.

1. A recombinant hypomorph microbial cell recombinantly engineered to have reduced expression of one or more essential genes and further comprises a strain specific nucleic acid identifier that identifies the hypomorph microbial cell.

2. The recombinant hypomorph microbial cell of claim 1, wherein the strain specific nucleic acid identifier is incorporated into a genome of the hypomorph microbial cell.

3. The recombinant hypomorph microbial cell of claim 1, wherein the strain specific nucleic acid identifier comprises, in a 5′ to 3′ direction, a first primer binding site, a hypomorph specific nucleic acid sequence, and a second primer binding site, wherein the hypomorph specific nucleic acid sequence identifies the one or more essential genes having reduced expression.

4. The recombinant hypomorph microbial cell of claim 3, wherein the first primer binding site and second primer binding site are independently between 5 and 50 base pairs in length.

5. The recombinant hypomorph microbial cell of claim 1, wherein the strain specific nucleic acid identifier is between 5 and 100 base pairs in length.

6. The recombinant hypomorph microbial cell of claim 1, wherein the cell is recombinantly engineered so that the one or more essential genes are under the control of a weak promoter.

7. The recombinant hypomorph microbial cell of claim 6, wherein the weak promoter further comprises a spacer sequence between the promoter and the ribozyme binding site.

8. The recombinant hypomorph microbial cell of claim 7, wherein the spacer sequence is between 2. and 25 base pairs.

9. The recombinant hypomorph microbial cell of claim 6, wherein the weak promoter is a Sauer promoter.

10. The recombinant hypomorph microbial cell of claim 1, wherein the cell is a bacterial cell, a fungal cell, a mycological cell, a protozoal cell, a nematode cell, a trematode cell, or a cestode cell.

11-45. (canceled)

46. The recombinant hypomorph microbial cell of claim 10, wherein the bacterial cell is selected from the group consisting of Eschericia coli, Klebsiella pneumoniae, Pseudomonas aeruginosa, Staphylococcus aureus, Acinetobacter haumannii, Candida albicans, Enterobacter cloacae, Enterococcus faecalis, Enterococcus faecium, Proteus mirabalis, Streptococcus agalactiae, Stenotrophomonas maltophila, Mycobacterium tuberculosis, Mycobacterium avium-intracellulare, Mycobacterium kansasii, Mycobacterium fortuitum, Mycobacterium chelonae, Mycobacterium leprae, Mycobacterium ofricanum, Mycobacterium micron, Mycobacterium avium paratuberculosis, Mycobacterium intracellulare, Mycobacterium scrofulaceum, Mycobacterium xenopi, Alycohacterium marinum, and Mycobacterium ulceran.

47. The recombinant hypomorph microbial cell of claim 1, wherein the cell is recombinantly engineered so that the one or more essential genes encode a protein degradation tag that is appended to a gene expression product upon translation.

48. The recombinant hypomorph microbial cell of claim 47, wherein the protein degradation tag targets the gene expression product for degradation by a clp-protease.

49. The recombinant hypomorph microbial cell of claim 48, wherein the protein degradation tag is DAS-F-4.

50. The recombinant hypomorph microbial cell of claim 48, wherein the cell is further recombinantly engineered to express a protease adapter protein under the control of an inducible promoter.

51. The recombinant hypomorph microbial cell of claim 50, wherein the protease adapter protein is sspB.

52. The recombinant hypomorph microbial cell of claim 1, wherein the one or more essential genes encode proteins that are localized to the cytoplasm, cytoplasmic membrane, periplasm, outer membrane, or extracellular space.

53. The recombinant hypomorph microbial cell of claim 1, wherein the one or more essential genes are selected from the group consisting of ostA, opr86, oprL, lolB, omlA, lppL, surA, lolA, tolB, tolA, mreC, gcp, ccsX, ctaC, eno, fba, folB, gleB, marP, mdh, mshC, murG, nadE, pstP, sucD, topA, efpA, tpi, dlat, and mesJ.

54. A multiplex method for whole-cell target-based screening of microbes, comprising:
culturing a collection of recombinant hypomorph microbial cells in individual discrete volumes, wherein each individual recombinant hypomorph microbial cell of a given species is recombinantly engineered to have reduced expression of a different essential gene or combination of essential genes and further comprises a strain specific nucleic acid identifier that identifies the individual recombinant hypomorph microbial cell;
exposing each individual discrete volume, or a sub-set of individual discrete volumes, to a set of different experimental conditions; and
detecting the recombinant hypomorph microbial cells from the individual discrete volumes, wherein failure to detect one or more recombinant hypomorph microbial cells, or detection of a decreased amount of one or more recombinant hypomorph microbial cells relative to other recombinant hypomorph microbial cells or a control, indicates susceptibility of the one or more recombinant hypomorph microbial cells to the experimental condition.

55. The method of claim 54, wherein the failure to detect one or more recombinant hypomorph microbial cells, or detection of a decreased amount of one or more recombinant hypomorph microbial cells relative to other recombinant hypomorph microbial cells or a control, further indicates one or more mechanisms of action by which the one or more hypomorph cells are rendered susceptible to the experimental condition.

56. The method of claim 54, wherein detecting the recombinant hypomorph microbial cells comprises:
amplifying, using a set of nucleic acid primer pairs configured to bind to and amplify the strain specific nucleic acid identifier of the recombinant hypomorph microbial cells, the strain specific nucleic acid identifier of each hypomorph strain obtaining amplicons;
ligating a first sequencing primer and a first sequencing adapter to a first end of the amplicons resulting from the amplifying step and a second sequencing primer and a second sequencing adapter to a second end of the amplicons resulting from the amplifying step;
sequencing the amplicons resulting from the ligating step to generate a set of sequencing reads; and
determining an abundance of each hypomorph strain based on number of sequencing reads for each strain specific nucleic acid identifier.

57. The method of claim 56, wherein the nucleic acid primer pair comprises a first primer that binds to a first primer binding site in the strain specific nucleic acid identifier in the recombinant hypomorph microbial cell and a second primer that binds to a second primer binding site in the strain specific nucleic acid identifier in the recombinant hypomorph microbial cell, wherein the first and/or the second primer comprises an origin specific nucleic acid identifier that identifies individual discrete volume from which one or more hypomorph strains are detected, wherein the first and/or the second primer further comprises an experimental condition specific nucleic acid identifier that identifies experimental conditions to which the hypomorph cells were exposed.

58. The method of claim 57, wherein the nucleic acid primer pair further comprises a first sequencing primer binding site and the first sequencing adapter on the first primer, and a second sequencing primer binding site and the second sequencing adapter on the second primer.

59. The method of claim 57, wherein each sequencing read from the same individual discrete volume is identified by the origin specific nucleic acid identifier, and the experimental condition of each hypomorph is determined by the experimental condition specific nucleic acid identifier.

60. The method of claim 56, further comprising pooling all individual discrete volumes prior to amplifying the strain specific nucleic acid identifiers.

61. The method of claim 54, wherein the individual discrete volume is a well of a multi-well culture plate.

62. The method of claim 54, wherein the different experimental conditions comprise exposure to different test agents, combinations of test agents, or different concentrations of test agents or combinations of test agents.

63. The method of claim 62, wherein the test agent is a chemical agent.

64. The method of claim 62, wherein the different experimental conditions further comprise different physical growth conditions.

65. The method of claim 64, wherein the different physical growth conditions comprise different growth media, different pH, different temperatures, different atmospheric pressures, different atmospheric O₂ concentrations, different atmospheric CO₂ concentrations, or a combination thereof.


SEQUENCE LISTING (1056 sequences)

SEQ ID NO: 1 - DNA, 30 bp, Artificial Sequence
  Synthetic primer
  misc_feature (3)..(10): n is a, c, g, or t
gcnnnnnnnntatttatgcagaggccgagg

SEQ ID NO: 2 - DNA, 30 bp, Artificial Sequence
  Synthetic primer
  misc_feature (3)..(10): n is a, c, g, or t
gcnnnnnnnntgggacggtatgaataatcc

SEQ ID NO: 3 - DNA, 58 bp, Artificial Sequence
  Synthetic primer
aatgatacggcgaccaccgagatctacactctttccctacacgacgctcttccgatct

SEQ ID NO: 4 - DNA, 33 bp, Artificial Sequence
  Synthetic primer
gatcggaagagctcgtatgccgtcttctgcttg

SEQ ID NO: 5 - DNA, 20 bp, Artificial Sequence
  Synthetic primer
aatgatacggcgaccaccga

SEQ ID NO: 6 - DNA, 21 bp, Artificial Sequence
  Synthetic primer
caagcagaagacggcatacga

SEQ ID NO: 7 - DNA, 20 bp, Artificial Sequence
  Synthetic primer
aaggggcatgatgacttgac

SEQ ID NO: 8 - DNA, 20 bp, Artificial Sequence
  Synthetic primer
gagatgtcggttcccttgtg

SEQ ID NO: 9 - DNA, 19 bp, Artificial Sequence
  Synthetic primer
tcctacgggaggcagcagt

SEQ ID NO: 10 - DNA, 26 bp, Artificial Sequence
  Synthetic primer
ggactaccagggtatctaatcctgtt

SEQ ID NO: 11 - DNA, 8 bp, Artificial Sequence
  Synthetic primer
atcgactg

SEQ ID NO: 12 - DNA, 8 bp, Artificial Sequence
  Synthetic primer
gctagcag

SEQ ID NO: 13 - DNA, 8 bp, Artificial Sequence
  Synthetic primer
tactctcc

SEQ ID NO: 14 - DNA, 8 bp, Artificial Sequence
  Synthetic primer
tgacagca

SEQ ID NO: 15 - DNA, 8 bp, Artificial Sequence
  Synthetic primer
gcaggttg

SEQ ID NO: 16 - DNA, 8 bp, Artificial Sequence
  Synthetic primer
ttccagct

SEQ ID NO: 17 - DNA, 8 bp, Artificial Sequence
  Synthetic primer
tagttagc

SEQ ID NO: 18 - DNA, 8 bp, Artificial Sequence
  Synthetic primer
agcgctaa

SEQ ID NO: 19 - DNA, 8 bp, Artificial Sequence
  Synthetic primer
cggttctt

SEQ ID NO: 20 - DNA, 8 bp, Artificial Sequence
  Synthetic primer
tagcattg

SEQ ID NO: 21 - DNA, 8 bp, Artificial Sequence
  Synthetic primer
aattcaac

SEQ ID NO: 22 - DNA, 8 bp, Artificial Sequence
  Synthetic primer
ttcacaga

SEQ ID NO: 23 - DNA, 8 bp, Artificial Sequence
  Synthetic primer
gctctctt

SEQ ID NO: 24 - DNA, 8 bp, Artificial Sequence
  Synthetic primer
tgacttgg

SEQ ID NO: 25 - DNA, 8 bp, Artificial Sequence
  Synthetic primer
tatggttc

SEQ ID NO: 26 - DNA, 8 bp, Artificial Sequence
  Synthetic primer
cactagcc

SEQ ID NO: 27 - DNA, 8 bp, Artificial Sequence
  Synthetic primer
aacctctt

SEQ ID NO: 28 - DNA, 8 bp, Artificial Sequence
  Synthetic primer
ctacattg

SEQ ID NO: 29 - DNA, 8 bp, Artificial Sequence
  Synthetic primer
gcgattac

SEQ ID NO: 30 - DNA, 8 bp, Artificial Sequence
  Synthetic primer
aattggcc

SEQ ID NO: 31 - DNA, 8 bp, Artificial Sequence
  Synthetic primer
aattgctt

SEQ ID NO: 32 - DNA, 8 bp, Artificial Sequence
  Synthetic primer
ttggtctg

SEQ ID NO: 33 - DNA, 8 bp, Artificial Sequence
  Synthetic primer
catcctgg

SEQ ID NO: 34 - DNA, 8 bp, Artificial Sequence
  Synthetic primer
ggattaac

SEQ ID NO: 35 - DNA, 8 bp, Artificial Sequence
  Synthetic primer
cgcatatt

SEQ ID NO: 36 - DNA, 8 bp, Artificial Sequence
  Synthetic primer
tcattcga

SEQ ID NO: 37 - DNA, 8 bp, Artificial Sequence
  Synthetic primer
gtccaatc

SEQ ID NO: 38 - DNA, 8 bp, Artificial Sequence
  Synthetic primer
cttggtca

SEQ ID NO: 39 - DNA, 8 bp, Artificial Sequence
  Synthetic primer
ccaacgct

SEQ ID NO: 40 - DNA, 8 bp, Artificial Sequence
  Synthetic primer
tccacttc

SEQ ID NO: 41 - DNA, 8 bp, Artificial Sequence
  Synthetic primer
aatctcca

SEQ ID NO: 42 - DNA, 8 bp, Artificial Sequence
  Synthetic primer
gtctgcac

SEQ ID NO: 43 - DNA, 8 bp, Artificial Sequence
  Synthetic primer
ctgctcct

SEQ ID NO: 44 - DNA, 8 bp, Artificial Sequence
  Synthetic primer
ttagccag

SEQ ID NO: 45 - DNA, 8 bp, Artificial Sequence
  Synthetic primer
gctgattc

SEQ ID NO: 46 - DNA, 8 bp, Artificial Sequence
  Synthetic primer
gaatcgac

SEQ ID NO: 47 - DNA, 8 bp, Artificial Sequence
  Synthetic primer
agtcacct

SEQ ID NO: 48 - DNA, 8 bp, Artificial Sequence
  Synthetic primer
cacgattc

SEQ ID NO: 49 - DNA, 8 bp, Artificial Sequence
  Synthetic primer
gctccgat

SEQ ID NO: 50 - DNA, 8 bp, Artificial Sequence
  Synthetic primer
cttggctt

SEQ ID NO: 51 - DNA, 8 bp, Artificial Sequence
  Synthetic primer
gctgcact

SEQ ID NO: 52 - DNA, 8 bp, Artificial Sequence
  Synthetic primer
gaacttcg

SEQ ID NO: 53 - DNA, 8 bp, Artificial Sequence
  Synthetic primer
ctgtattc

SEQ ID NO: 54 - DNA, 8 bp, Artificial Sequence
  Synthetic primer
atatccga

SEQ ID NO: 55 - DNA, 8 bp, Artificial Sequence
  Synthetic primer
ttgtccat

SEQ ID NO: 56 - DNA, 8 bp, Artificial Sequence
  Synthetic primer
agtaagtc

SEQ ID NO: 57 - DNA, 8 bp, Artificial Sequence
  Synthetic primer
gaatatca

SEQ ID NO: 58 - DNA, 8 bp, Artificial Sequence
  Synthetic primer
caactgat

SEQ ID NO: 59 - DNA, 8 bp, Artificial Sequence
  Synthetic primer
cctgtcat

SEQ ID NO: 60 - DNA, 8 bp, Artificial Sequence
  Synthetic primer
gacggtta

SEQ ID NO: 61 - DNA, 8 bp, Artificial Sequence
  Synthetic primer
ctattagc

SEQ ID NO: 62 - DNA, 8 bp, Artificial Sequence
  Synthetic primer
tccaacca

SEQ ID NO: 63 - DNA, 8 bp, Artificial Sequence
  Synthetic primer
ctggctat

SEQ ID NO: 64 - DNA, 8 bp, Artificial Sequence
  Synthetic primer
gcggactt

SEQ ID NO: 65 - DNA, 8 bp, Artificial Sequence
  Synthetic primer
ccatcaca

SEQ ID NO: 66 - DNA, 8 bp, Artificial Sequence
  Synthetic primer
ggcaatac

SEQ ID NO: 67 - DNA, 8 bp, Artificial Sequence
  Synthetic primer
cacttcat

SEQ ID NO: 68 - DNA, 8 bp, Artificial Sequence
  Synthetic primer
caagctta

SEQ ID NO: 69 - DNA, 8 bp, Artificial Sequence
  Synthetic primer
aggtacca

SEQ ID NO: 70 - DNA, 8 bp, Artificial Sequence
  Synthetic primer
tccataac

SEQ ID NO: 71 - DNA, 8 bp, Artificial Sequence
  Synthetic primer
gtcctcat

SEQ ID NO: 72 - DNA, 8 bp, Artificial Sequence
  Synthetic primer
agtactgc

SEQ ID NO: 73 - DNA, 8 bp, Artificial Sequence
  Synthetic primer
cttgaatc

SEQ ID NO: 74 - DNA, 8 bp, Artificial Sequence
  Synthetic primer
ccaactaa

SEQ ID NO: 75 - DNA, 8 bp, Artificial Sequence
  Synthetic primer
aataccat

SEQ ID NO: 76 - DNA, 8 bp, Artificial Sequence
  Synthetic primer
gcgatatt

SEQ ID NO: 77 - DNA, 8 bp, Artificial Sequence
  Synthetic primer
gaacgcta

SEQ ID NO: 78 - DNA, 8 bp, Artificial Sequence
  Synthetic primer
ctgacatc

SEQ ID NO: 79 - DNA, 8 bp, Artificial Sequence
  Synthetic primer
gccaccat

SEQ ID NO: 80 - DNA, 8 bp, Artificial Sequence
  Synthetic primer
cgactctc

SEQ ID NO: 81 - DNA, 8 bp, Artificial Sequence
  Synthetic primer
tgctatta

SEQ ID NO: 82 - DNA, 8 bp, Artificial Sequence
  Synthetic primer
cttctggc

SEQ ID NO: 83 - DNA, 8 bp, Artificial Sequence
  Synthetic primer
atgaatta

SEQ ID NO: 84 - DNA, 8 bp, Artificial Sequence
  Synthetic primer
tactccag

SEQ ID NO: 85 - DNA, 8 bp, Artificial Sequence
  Synthetic primer
atcatacc

SEQ ID NO: 86 - DNA, 8 bp, Artificial Sequence
  Synthetic primer
cctctaac

SEQ ID NO: 87 - DNA, 8 bp, Artificial Sequence
  Synthetic primer
atcttctc

SEQ ID NO: 88 - DNA, 8 bp, Artificial Sequence
  Synthetic primer
cagctcac

SEQ ID NO: 89 - DNA, 8 bp, Artificial Sequence
  Synthetic primer
ggttatct

SEQ ID NO: 90 - DNA, 8 bp, Artificial Sequence
  Synthetic primer
tccgcata

SEQ ID NO: 91 - DNA, 8 bp, Artificial Sequence
  Synthetic primer
tgcttcac

SEQ ID NO: 92 - DNA, 8 bp, Artificial Sequence
  Synthetic primer
gcttccta

SEQ ID NO: 93 - DNA, 8 bp, Artificial Sequence
  Synthetic primer
gaccatct

SEQ ID NO: 94 - DNA, 8 bp, Artificial Sequence
  Synthetic primer
ctggtatt

SEQ ID NO: 95 - DNA, 8 bp, Artificial Sequence
  Synthetic primer
ttaatcac

SEQ ID NO: 96 - DNA, 8 bp, Artificial Sequence
  Synthetic primer
cgcgaata

SEQ ID NO: 97 - DNA, 8 bp, Artificial Sequence
  Synthetic primer
gctcacca

SEQ ID NO: 98 - DNA, 8 bp, Artificial Sequence
  Synthetic primer
tcatgtct

SEQ ID NO: 99 - DNA, 8 bp, Artificial Sequence
  Synthetic primer
atccttaa

SEQ ID NO: 100 - DNA, 8 bp, Artificial Sequence
  Synthetic primer
ttcttggc

SEQ ID NO: 101 - DNA, 8 bp, Artificial Sequence
  Synthetic primer
catcactt

SEQ ID NO: 102 - DNA, 8 bp, Artificial Sequence
  Synthetic primer
cgaacttc

SEQ ID NO: 103 - DNA, 8 bp, Artificial Sequence
  Synthetic primer
gacattaa

SEQ ID NO: 104 - DNA, 8 bp, Artificial Sequence
  Synthetic primer
ttcacctt

SEQ ID NO: 105 - DNA, 8 bp, Artificial Sequence
  Synthetic primer
ccaatctg

SEQ ID NO: 106 - DNA, 8 bp, Artificial Sequence
  Synthetic primer
cgacagtt

SEQ ID NO: 107 - DNA, 8 bp, Artificial Sequence
  Synthetic primer
aagtagag

SEQ ID NO: 108 - DNA, 8 bp, Artificial Sequence
  Synthetic primer
catgctta

SEQ ID NO: 109 - DNA, 8 bp, Artificial Sequence
  Synthetic primer
gcacatct

SEQ ID NO: 110 - DNA, 8 bp, Artificial Sequence
  Synthetic primer
tgctcgac

SEQ ID NO: 111 - DNA, 8 bp, Artificial Sequence
  Synthetic primer
agcaattc

SEQ ID NO: 112 - DNA, 8 bp, Artificial Sequence
  Synthetic primer
agttgctt

SEQ ID NO: 113 - DNA, 8 bp, Artificial Sequence
  Synthetic primer
ccagttag

SEQ ID NO: 114 - DNA, 8 bp, Artificial Sequence
  Synthetic primer
ttgagcct

SEQ ID NO: 115 - DNA, 8 bp, Artificial Sequence
  Synthetic primer
acacgatc

SEQ ID NO: 116 - DNA, 8 bp, Artificial Sequence
  Synthetic primer
ggtccaga

SEQ ID NO: 117 - DNA, 8 bp, Artificial Sequence
  Synthetic primer
gtataaca

SEQ ID NO: 118 - DNA, 8 bp, Artificial Sequence
  Synthetic primer
ttcgctga

SEQ ID NO: 119 - DNA, 8 bp, Artificial Sequence
  Synthetic primer
aacttgac

SEQ ID NO: 120 - DNA, 8 bp, Artificial Sequence
  Synthetic primer
cacatcct

SEQ ID NO: 121 - DNA, 8 bp, Artificial Sequence
  Synthetic primer
tcggaatg

SEQ ID NO: 122 - DNA, 8 bp, Artificial Sequence
  Synthetic primer
aaggatgt

SEQ ID NO: 123 - DNA, 8 bp, Artificial Sequence
  Synthetic primer
cgcgcggt

SEQ ID NO: 124 - DNA, 8 bp, Artificial Sequence
  Synthetic primer
tctggcga

SEQ ID NO: 125 - DNA, 8 bp, Artificial Sequence
  Synthetic primer
catagcga

SEQ ID NO: 126 - DNA, 8 bp, Artificial Sequence
  Synthetic primer
caggagcc

SEQ ID NO: 127 - DNA, 8 bp, Artificial Sequence
  Synthetic primer
tgtcggat

SEQ ID NO: 128 - DNA, 8 bp, Artificial Sequence
  Synthetic primer
attatgtt

SEQ ID NO: 129 - DNA, 8 bp, Artificial Sequence
  Synthetic primer
cctaccat

SEQ ID NO: 130 - DNA, 8 bp, Artificial Sequence
  Synthetic primer
tacttagc

SEQ ID NO: 131 - DNA, 8 bp, Artificial Sequence
  Synthetic primer
catgatcg

SEQ ID NO: 132 - DNA, 8 bp, Artificial Sequence
  Synthetic primer
aggatcta

SEQ ID NO: 133 - DNA, 8 bp, Artificial Sequence
  Synthetic primer
gacagtaa

SEQ ID NO: 134 - DNA, 8 bp, Artificial Sequence
  Synthetic primer
cctatgcc

SEQ ID NO: 135 - DNA, 8 bp, Artificial Sequence
  Synthetic primer
tcgccttg

SEQ ID NO: 136 - DNA, 8 bp, Artificial Sequence
  Synthetic primer
atagcgtc

SEQ ID NO: 137 - DNA, 8 bp, Artificial Sequence
  Synthetic primer
gaagaagt

SEQ ID NO: 138 - DNA, 8 bp, Artificial Sequence
  Synthetic primer
attctagg

SEQ ID NO: 139 - DNA, 8 bp, Artificial Sequence
  Synthetic primer
cgttacca

SEQ ID NO: 140 - DNA, 8 bp, Artificial Sequence
  Synthetic primer
gtctgatg

SEQ ID NO: 141 - DNA, 8 bp, Artificial Sequence
  Synthetic primer
ttacgcac

SEQ ID NO: 142 - DNA, 8 bp, Artificial Sequence
  Synthetic primer
ttgaatag

SEQ ID NO: 143 - DNA, 8 bp, Artificial Sequence
  Synthetic primer
aagacact

SEQ ID NO: 144 - DNA, 8 bp, Artificial Sequence
  Synthetic primer
cagcaagg

SEQ ID NO: 145 - DNA, 8 bp, Artificial Sequence
  Synthetic primer
tccagcaa

SEQ ID NO: 146 - DNA, 8 bp, Artificial Sequence
  Synthetic primer
ccagagct

SEQ ID NO: 147 - DNA, 8 bp, Artificial Sequence
  Synthetic primer
tccttggt

SEQ ID NO: 148 - DNA, 8 bp, Artificial Sequence
  Synthetic primer
aggttatc

SEQ ID NO: 149 - DNA, 8 bp, Artificial Sequence
  Synthetic primer
gtcatcta

SEQ ID NO: 150 - DNA, 8 bp, Artificial Sequence
  Synthetic primer
ccttcgca

SEQ ID NO: 151 - DNA, 8 bp, Artificial Sequence
  Synthetic primer
tctcggtc

SEQ ID NO: 152 - DNA, 8 bp, Artificial Sequence
  Synthetic primer
attgtctg

SEQ ID NO: 153 - DNA, 8 bp, Artificial Sequence
  Synthetic primer
gaacctag

SEQ ID NO: 154 - DNA, 8 bp, Artificial Sequence
  Synthetic primer
taatgaac

SEQ ID NO: 155 - DNA, 8 bp, Artificial Sequence
  Synthetic primer
aacgcatt

SEQ ID NO: 156 - DNA, 8 bp, Artificial Sequence
  Synthetic primer
caactctc

SEQ ID NO: 157 - DNA, 8 bp, Artificial Sequence
  Synthetic primer
ctgtaatc

SEQ ID NO: 158 - DNA, 8 bp, Artificial Sequence
  Synthetic primer
taagcaca

SEQ ID NO: 159 - DNA, 8 bp, Artificial Sequence
  Synthetic primer
aatgttct

SEQ ID NO: 160 - DNA, 8 bp, Artificial Sequence
  Synthetic primer
cagcggta

SEQ ID NO: 161 - DNA, 8 bp, Artificial Sequence
  Synthetic primer
gaccagga

SEQ ID NO: 162 - DNA, 8 bp, Artificial Sequence
  Synthetic primer
tatccagg

SEQ ID NO: 163 - DNA, 8 bp, Artificial Sequence
  Synthetic primer
acaggtat

SEQ ID NO: 164 - DNA, 8 bp, Artificial Sequence
  Synthetic primer
ccaacatt

SEQ ID NO: 165 - DNA, 8 bp, Artificial Sequence
  Synthetic primer
gccgtcga

SEQ ID NO: 166 - DNA, 8 bp, Artificial Sequence
  Synthetic primer
tatctgcc

SEQ ID NO: 167 - DNA, 8 bp, Artificial Sequence
  Synthetic primer
actaagac

SEQ ID NO: 168 - DNA, 8 bp, Artificial Sequence
  Synthetic primer
cgccttcc

SEQ ID NO: 169 - DNA, 8 bp, Artificial Sequence
  Synthetic primer
gcctagcc

SEQ ID NO: 170 - DNA, 8 bp, Artificial Sequence
  Synthetic primer
tgctgctg

SEQ ID NO: 171 - DNA, 8 bp, Artificial Sequence
  Synthetic primer
aggtaagg

SEQ ID NO: 172 - DNA, 8 bp, Artificial Sequence
  Synthetic primer
ctaactcg

SEQ ID NO: 173 - DNA, 8 bp, Artificial Sequence
  Synthetic primer
gtaacatc

SEQ ID NO: 174 - DNA, 8 bp, Artificial Sequence
  Synthetic primer
tgtaatca

SEQ ID NO: 175 - DNA, 8 bp, Artificial Sequence
  Synthetic primer
attatcaa

SEQ ID NO: 176 - DNA, 8 bp, Artificial Sequence
  Synthetic primer
ctatgcgt

SEQ ID NO: 177 - DNA, 8 bp, Artificial Sequence
  Synthetic primer
gtccacag

SEQ ID NO: 178 - DNA, 8 bp, Artificial Sequence
  Synthetic primer
ttgtctat

SEQ ID NO: 179 - DNA, 8 bp, Artificial Sequence
  Synthetic primer
aacaatgg

SEQ ID NO: 180 - DNA, 8 bp, Artificial Sequence
  Synthetic primer
attcctct

SEQ ID NO: 181 - DNA, 8 bp, Artificial Sequence
  Synthetic primer
gaaggaag

SEQ ID NO: 182 - DNA, 8 bp, Artificial Sequence
  Synthetic primer
tcgctaga

SEQ ID NO: 183 - DNA, 8 bp, Artificial Sequence
  Synthetic primer
acagttga

SEQ ID NO: 184 - DNA, 8 bp, Artificial Sequence
  Synthetic primer
caatagtc

SEQ ID NO: 185 - DNA, 8 bp, Artificial Sequence
  Synthetic primer
gaccgttg

SEQ ID NO: 186 - DNA, 8 bp, Artificial Sequence
  Synthetic primer
tctgcaag

SEQ ID NO: 187 - DNA, 8 bp, Artificial Sequence
  Synthetic primer
actgtatc

SEQ ID NO: 188 - DNA, 8 bp, Artificial Sequence
  Synthetic primer
ctaccagg

SEQ ID NO: 189 - DNA, 8 bp, Artificial Sequence
  Synthetic primer
gacctaac

SEQ ID NO: 190 - DNA, 8 bp, Artificial Sequence
  Synthetic primer
tgcaagta

SEQ ID NO: 191 - DNA, 8 bp, Artificial Sequence
  Synthetic primer
agcatgga

SEQ ID NO: 192 - DNA, 8 bp, Artificial Sequence
  Synthetic primer
cgctatgt

SEQ ID NO: 193 - DNA, 8 bp, Artificial Sequence
  Synthetic primer
gatatcca

SEQ ID NO: 194 - DNA, 8 bp, Artificial Sequence
  Synthetic primer
tgtaactc

SEQ ID NO: 195 - DNA, 8 bp, Artificial Sequence
  Synthetic primer
aggtcgca

SEQ ID NO: 196 - DNA, 8 bp, Artificial Sequence
  Synthetic primer
ctgcggat

SEQ ID NO: 197 - DNA, 8 bp, Artificial Sequence
  Synthetic primer
accaactg

SEQ ID NO: 198 - DNA, 8 bp, Artificial Sequence
  Synthetic primer
ttaatcag

SEQ ID NO: 199 - DNA, 8 bp, Artificial Sequence
  Synthetic primer
aggtgcga

SEQ ID NO: 200 - DNA, 8 bp, Artificial Sequence
  Synthetic primer
ctgtggcg

SEQ ID NO: 201 - DNA, 8 bp, Artificial Sequence
  Synthetic primer
gccgcaac

SEQ ID NO: 202 - DNA, 8 bp, Artificial Sequence
  Synthetic primer
ttatatct

SEQ ID NO: 203 - DNA, 20 bp, Artificial Sequence
  Synthetic primer
tatttatgcagaggccgagg

SEQ ID NO: 204 - DNA, 20 bp, Artificial Sequence
  Synthetic primer
ggattattcataccgtccca

SEQ ID NO: 205 - DNA, 30 bp, Artificial Sequence
  Synthetic primer
gcatcgactgtatttatgcagaggccgagg

SEQ ID NO: 206 - DNA, 30 bp, Artificial Sequence
  Synthetic primer
gcgctagcagtatttatgcagaggccgagg

SEQ ID NO: 207 - DNA, 30 bp, Artificial Sequence
  Synthetic primer
gctactctcctatttatgcagaggccgagg

SEQ ID NO: 208 - DNA, 30 bp, Artificial Sequence
  Synthetic primer
gctgacagcatatttatgcagaggccgagg

SEQ ID NO: 209 - DNA, 30 bp, Artificial Sequence
  Synthetic primer
gcgcaggttgtatttatgcagaggccgagg

SEQ ID NO: 210 - DNA, 30 bp, Artificial Sequence
  Synthetic primer
gcttccagcttatttatgcagaggccgagg

SEQ ID NO: 211 - DNA, 30 bp, Artificial Sequence
  Synthetic primer
gctagttagctatttatgcagaggccgagg

SEQ ID NO: 212 - DNA, 30 bp, Artificial Sequence
  Synthetic primer
gcagcgctaatatttatgcagaggccgagg

SEQ ID NO: 213 - DNA, 30 bp, Artificial Sequence
  Synthetic primer
gccggttctttatttatgcagaggccgagg

SEQ ID NO: 214 - DNA, 30 bp, Artificial Sequence
  Synthetic primer
gctagcattgtatttatgcagaggccgagg

SEQ ID NO: 215 - DNA, 30 bp, Artificial Sequence
  Synthetic primer
gcaattcaactatttatgcagaggccgagg

SEQ ID NO: 216 - DNA, 30 bp, Artificial Sequence
  Synthetic primer
gcttcacagatatttatgcagaggccgagg

SEQ ID NO: 217 - DNA, 30 bp, Artificial Sequence
  Synthetic primer
gcgctctctttatttatgcagaggccgagg

SEQ ID NO: 218 - DNA, 30 bp, Artificial Sequence
  Synthetic primer
gctgacttggtatttatgcagaggccgagg

SEQ ID NO: 219 - DNA, 30 bp, Artificial Sequence
  Synthetic primer
gctatggttctatttatgcagaggccgagg

SEQ ID NO: 220 - DNA, 30 bp, Artificial Sequence
  Synthetic primer
gccactagcctatttatgcagaggccgagg

SEQ ID NO: 221 - DNA, 30 bp, Artificial Sequence
  Synthetic primer
gcaacctctttatttatgcagaggccgagg

SEQ ID NO: 222 - DNA, 30 bp, Artificial Sequence
  Synthetic primer
gcctacattgtatttatgcagaggccgagg

SEQ ID NO: 223 - DNA, 30 bp, Artificial Sequence
  Synthetic primer
gcgcgattactatttatgcagaggccgagg

SEQ ID NO: 224 - DNA, 30 bp, Artificial Sequence
  Synthetic primer
gcaattggcctatttatgcagaggccgagg

SEQ ID NO: 225 - DNA, 30 bp, Artificial Sequence
  Synthetic primer
gcaattgctttatttatgcagaggccgagg

SEQ ID NO: 226 - DNA, 30 bp, Artificial Sequence
  Synthetic primer
gcttggtctgtatttatgcagaggccgagg

SEQ ID NO: 227 - DNA, 30 bp, Artificial Sequence
  Synthetic primer
gccatcctggtatttatgcagaggccgagg

SEQ ID NO: 228 - DNA, 30 bp, Artificial Sequence
  Synthetic primer
gcggattaactatttatgcagaggccgagg

SEQ ID NO: 229 - DNA, 30 bp, Artificial Sequence
  Synthetic primer
gccgcatatttatttatgcagaggccgagg

SEQ ID NO: 230 - DNA, 30 bp, Artificial Sequence
  Synthetic primer
gctcattcgatatttatgcagaggccgagg

SEQ ID NO: 231 - DNA, 30 bp, Artificial Sequence
  Synthetic primer
gcgtccaatctatttatgcagaggccgagg

SEQ ID NO: 232 - DNA, 30 bp, Artificial Sequence
  Synthetic primer
gccttggtcatatttatgcagaggccgagg

SEQ ID NO: 233 - DNA, 30 bp, Artificial Sequence
  Synthetic primer
gcccaacgcttatttatgcagaggccgagg

SEQ ID NO: 234 - DNA, 30 bp, Artificial Sequence
  Synthetic primer
gctccacttctatttatgcagaggccgagg

SEQ ID NO: 235 - DNA, 30 bp, Artificial Sequence
  Synthetic primer
gcaatctccatatttatgcagaggccgagg

SEQ ID NO: 236 - DNA, 30 bp, Artificial Sequence
  Synthetic primer
gcgtctgcactatttatgcagaggccgagg

SEQ ID NO: 237 - DNA, 30 bp, Artificial Sequence
  Synthetic primer
gcctgctccttatttatgcagaggccgagg

SEQ ID NO: 238 - DNA, 30 bp, Artificial Sequence
  Synthetic primer
gcttagccagtatttatgcagaggccgagg

SEQ ID NO: 239 - DNA, 30 bp, Artificial Sequence
  Synthetic primer
gcgctgattctatttatgcagaggccgagg

SEQ ID NO: 240 - DNA, 30 bp, Artificial Sequence
  Synthetic primer
gcgaatcgactatttatgcagaggccgagg

SEQ ID NO: 241 - DNA, 30 bp, Artificial Sequence
  Synthetic primer
gcagtcaccttatttatgcagaggccgagg

SEQ ID NO: 242 - DNA, 30 bp, Artificial Sequence
  Synthetic primer
gccacgattctatttatgcagaggccgagg

SEQ ID NO: 243 - DNA, 30 bp, Artificial Sequence
  Synthetic primer
gcgctccgattatttatgcagaggccgagg

SEQ ID NO: 244 - DNA, 30 bp, Artificial Sequence
  Synthetic primer
gccttggctttatttatgcagaggccgagg

SEQ ID NO: 245 - DNA, 30 bp, Artificial Sequence
  Synthetic primer
gcgctgcacttatttatgcagaggccgagg

SEQ ID NO: 246 - DNA, 30 bp, Artificial Sequence
  Synthetic primer
gcgaacttcgtatttatgcagaggccgagg

SEQ ID NO: 247 - DNA, 30 bp, Artificial Sequence
  Synthetic primer
gcctgtattctatttatgcagaggccgagg

SEQ ID NO: 248 - DNA, 30 bp, Artificial Sequence
  Synthetic primer
gcatatccgatatttatgcagaggccgagg

SEQ ID NO: 249 - DNA, 30 bp, Artificial Sequence
  Synthetic primer
gcttgtccattatttatgcagaggccgagg

SEQ ID NO: 250 - DNA, 30 bp, Artificial Sequence
  Synthetic primer
gcagtaagtctatttatgcagaggccgagg

SEQ ID NO: 251 - DNA, 30 bp, Artificial Sequence
  Synthetic primer
gcgaatatcatatttatgcagaggccgagg

SEQ ID NO: 252 - DNA, 30 bp, Artificial Sequence
  Synthetic primer
gccaactgattatttatgcagaggccgagg

SEQ ID NO: 253 - DNA, 30 bp, Artificial Sequence
  Synthetic primer
gccctgtcattatttatgcagaggccgagg

SEQ ID NO: 254 - DNA, 30 bp, Artificial Sequence
  Synthetic primer
gcgacggttatatttatgcagaggccgagg

SEQ ID NO: 255 - DNA, 30 bp, Artificial Sequence
  Synthetic primer
gcctattagctatttatgcagaggccgagg

SEQ ID NO: 256 - DNA, 30 bp, Artificial Sequence
  Synthetic primer
gctccaaccatatttatgcagaggccgagg

SEQ ID NO: 257 - DNA, 30 bp, Artificial Sequence
  Synthetic primer
gcctggctattatttatgcagaggccgagg

SEQ ID NO: 258 - DNA, 30 bp, Artificial Sequence
  Synthetic primer
gcgcggactttatttatgcagaggccgagg

SEQ ID NO: 259 - DNA, 30 bp, Artificial Sequence
  Synthetic primer
gcccatcacatatttatgcagaggccgagg

SEQ ID NO: 260 - DNA, 30 bp, Artificial Sequence
  Synthetic primer
gcggcaatactatttatgcagaggccgagg

SEQ ID NO: 261 - DNA, 30 bp, Artificial Sequence
  Synthetic primer
gccacttcattatttatgcagaggccgagg

SEQ ID NO: 262 - DNA, 30 bp, Artificial Sequence
  Synthetic primer
gccaagcttatatttatgcagaggccgagg

SEQ ID NO: 263 - DNA, 30 bp, Artificial Sequence
  Synthetic primer
gcaggtaccatatttatgcagaggccgagg

SEQ ID NO: 264 - DNA, 30 bp, Artificial Sequence
  Synthetic primer
gctccataactatttatgcagaggccgagg

SEQ ID NO: 265 - DNA, 30 bp, Artificial Sequence
  Synthetic primer
gcgtcctcattatttatgcagaggccgagg

SEQ ID NO: 266 - DNA, 30 bp, Artificial Sequence
  Synthetic primer
gcagtactgctatttatgcagaggccgagg

SEQ ID NO: 267 - DNA, 30 bp, Artificial Sequence
  Synthetic primer
gccttgaatctatttatgcagaggccgagg

SEQ ID NO: 268 - DNA, 30 bp, Artificial Sequence
  Synthetic primer
gcccaactaatatttatgcagaggccgagg

SEQ ID NO: 269 - DNA, 30 bp, Artificial Sequence
  Synthetic primer
gcaataccattatttatgcagaggccgagg

SEQ ID NO: 270 - DNA, 30 bp, Artificial Sequence
  Synthetic primer
gcgcgatatttatttatgcagaggccgagg

SEQ ID NO: 271 - DNA, 30 bp, Artificial Sequence
  Synthetic primer
gcgaacgctatatttatgcagaggccgagg

SEQ ID NO: 272 - DNA, 30 bp, Artificial Sequence
  Synthetic primer
gcctgacatctatttatgcagaggccgagg

SEQ ID NO: 273 - DNA, 30 bp, Artificial Sequence
  Synthetic primer
gcgccaccattatttatgcagaggccgagg

SEQ ID NO: 274 - DNA, 30 bp, Artificial Sequence
  Synthetic primer
gccgactctctatttatgcagaggccgagg

SEQ ID NO: 275 - DNA, 30 bp, Artificial Sequence
  Synthetic primer
gctgctattatatttatgcagaggccgagg

SEQ ID NO: 276 - DNA, 30 bp, Artificial Sequence
  Synthetic primer
gccttctggctatttatgcagaggccgagg

SEQ ID NO: 277 - DNA, 30 bp, Artificial Sequence
  Synthetic primer
gcatgaattatatttatgcagaggccgagg

SEQ ID NO: 278 - DNA, 30 bp, Artificial Sequence
  Synthetic primer
gctactccagtatttatgcagaggccgagg

SEQ ID NO: 279 - DNA, 30 bp, Artificial Sequence
  Synthetic primer
gcatcatacctatttatgcagaggccgagg

SEQ ID NO: 280 - DNA, 30 bp, Artificial Sequence
  Synthetic primer
gccctctaactatttatgcagaggccgagg

SEQ ID NO: 281 - DNA, 30 bp, Artificial Sequence
  Synthetic primer
gcatcttctctatttatgcagaggccgagg

SEQ ID NO: 282 - DNA, 30 bp, Artificial Sequence
  Synthetic primer
gccagctcactatttatgcagaggccgagg

SEQ ID NO: 283 - DNA, 30 bp, Artificial Sequence
  Synthetic primer
gcggttatcttatttatgcagaggccgagg

SEQ ID NO: 284 - DNA, 30 bp, Artificial Sequence
  Synthetic primer
gctccgcatatatttatgcagaggccgagg

SEQ ID NO: 285 - DNA, 30 bp, Artificial Sequence
  Synthetic primer
gctgcttcactatttatgcagaggccgagg

SEQ ID NO: 286 - DNA, 30 bp, Artificial Sequence
  Synthetic primer
gcgcttcctatatttatgcagaggccgagg

SEQ ID NO: 287 - DNA, 30 bp, Artificial Sequence
  Synthetic primer
gcgaccatcttatttatgcagaggccgagg

SEQ ID NO: 288 - DNA, 30 bp, Artificial Sequence
  Synthetic primer
gcctggtatttatttatgcagaggccgagg

SEQ ID NO: 289 - DNA, 30 bp, Artificial Sequence
  Synthetic primer
gcttaatcactatttatgcagaggccgagg

SEQ ID NO: 290 - DNA, 30 bp, Artificial Sequence
  Synthetic primer
gccgcgaatatatttatgcagaggccgagg

SEQ ID NO: 291 - DNA, 30 bp, Artificial Sequence
  Synthetic primer
gcgctcaccatatttatgcagaggccgagg

SEQ ID NO: 292 - DNA, 30 bp, Artificial Sequence
  Synthetic primer
gctcatgtcttatttatgcagaggccgagg

SEQ ID NO: 293 - DNA, 30 bp, Artificial Sequence
  Synthetic primer
gcatccttaatatttatgcagaggccgagg

SEQ ID NO: 294 - DNA, 30 bp, Artificial Sequence
  Synthetic primer
gcttcttggctatttatgcagaggccgagg

SEQ ID NO: 295 - DNA, 30 bp, Artificial Sequence
  Synthetic primer
gccatcactttatttatgcagaggccgagg

SEQ ID NO: 296 - DNA, 30 bp, Artificial Sequence
  Synthetic primer
gccgaacttctatttatgcagaggccgagg

SEQ ID NO: 297 - DNA, 30 bp, Artificial Sequence
  Synthetic primer
gcgacattaatatttatgcagaggccgagg

SEQ ID NO: 298 - DNA, 30 bp, Artificial Sequence
  Synthetic primer
gcttcacctttatttatgcagaggccgagg

SEQ ID NO: 299 - DNA, 30 bp, Artificial Sequence
  Synthetic primer
gcccaatctgtatttatgcagaggccgagg

SEQ ID NO: 300 - DNA, 30 bp, Artificial Sequence
  Synthetic primer
gccgacagtttatttatgcagaggccgagg

SEQ ID NO: 301 - DNA, 30 bp, Artificial Sequence
  Synthetic primer
gcctctactttgggacggtatgaataatcc

SEQ ID NO: 302 - DNA, 30 bp, Artificial Sequence
  Synthetic primer
gctaagcatgtgggacggtatgaataatcc

SEQ ID NO: 303 - DNA, 30 bp, Artificial Sequence
  Synthetic primer
gcagatgtgctgggacggtatgaataatcc

SEQ ID NO: 304 - DNA, 30 bp, Artificial Sequence
  Synthetic primer
gcgtcgagcatgggacggtatgaataatcc

SEQ ID NO: 305 - DNA, 30 bp, Artificial Sequence
  Synthetic primer
gcgaattgcttgggacggtatgaataatcc

SEQ ID NO: 306 - DNA, 30 bp, Artificial Sequence
  Synthetic primer
gcaagcaacttgggacggtatgaataatcc

SEQ ID NO: 307 - DNA, 30 bp, Artificial Sequence
  Synthetic primer
gcctaactggtgggacggtatgaataatcc

SEQ ID NO: 308 - DNA, 30 bp, Artificial Sequence
  Synthetic primer
gcaggctcaatgggacggtatgaataatcc

SEQ ID NO: 309 - DNA, 30 bp, Artificial Sequence
  Synthetic primer
gcgatcgtgttgggacggtatgaataatcc

SEQ ID NO: 310 - DNA, 30 bp, Artificial Sequence
  Synthetic primer
gctctggacctgggacggtatgaataatcc

SEQ ID NO: 311 - DNA, 30 bp, Artificial Sequence
  Synthetic primer
gctgttatactgggacggtatgaataatcc

SEQ ID NO: 312 - DNA, 30 bp, Artificial Sequence
  Synthetic primer
gctcagcgaatgggacggtatgaataatcc

SEQ ID NO: 313 - DNA, 30 bp, Artificial Sequence
  Synthetic primer
gcgtcaagtttgggacggtatgaataatcc

SEQ ID NO: 314 - DNA, 30 bp, Artificial Sequence
  Synthetic primer
gcaggatgtgtgggacggtatgaataatcc

SEQ ID NO: 315 - DNA, 30 bp, Artificial Sequence
  Synthetic primer
gccattccgatgggacggtatgaataatcc

SEQ ID NO: 316 - DNA, 30 bp, Artificial Sequence
  Synthetic primer
gcacatcctttgggacggtatgaataatcc

SEQ ID NO: 317 - DNA, 30 bp, Artificial Sequence
  Synthetic primer
gcaccgcgcgtgggacggtatgaataatcc

SEQ ID NO: 318 - DNA, 30 bp, Artificial Sequence
  Synthetic primer
gctcgccagatgggacggtatgaataatcc

SEQ ID NO: 319 - DNA, 30 bp, Artificial Sequence
  Synthetic primer
gctcgctatgtgggacggtatgaataatcc

SEQ ID NO: 320 - DNA, 30 bp, Artificial Sequence
  Synthetic primer
gcggctcctgtgggacggtatgaataatcc

SEQ ID NO: 321 - DNA, 30 bp, Artificial Sequence
  Synthetic primer
gcatccgacatgggacggtatgaataatcc

SEQ ID NO: 322 - DNA, 30 bp, Artificial Sequence
  Synthetic primer
gcaacataattgggacggtatgaataatcc

SEQ ID NO: 323 - DNA, 30 bp, Artificial Sequence
  Synthetic primer
gcatggtaggtgggacggtatgaataatcc

SEQ ID NO: 324 - DNA, 30 bp, Artificial Sequence
  Synthetic primer
gcgctaagtatgggacggtatgaataatcc

SEQ ID NO: 325 - DNA, 30 bp, Artificial Sequence
  Synthetic primer
gccgatcatgtgggacggtatgaataatcc

SEQ ID NO: 326 - DNA, 30 bp, Artificial Sequence
  Synthetic primer
gctagatccttgggacggtatgaataatcc

SEQ ID NO: 327 - DNA, 30 bp, Artificial Sequence
  Synthetic primer
gcttactgtctgggacggtatgaataatcc

SEQ ID NO: 328 - DNA, 30 bp, Artificial Sequence
  Synthetic primer
gcggcataggtgggacggtatgaataatcc

SEQ ID NO: 329 - DNA, 30 bp, Artificial Sequence
  Synthetic primer
gccaaggcgatgggacggtatgaataatcc

SEQ ID NO: 330 - DNA, 30 bp, Artificial Sequence
  Synthetic primer
gcgacgctattgggacggtatgaataatcc

SEQ ID NO: 331 - DNA, 30 bp, Artificial Sequence
  Synthetic primer
gcacttcttctgggacggtatgaataatcc

SEQ ID NO: 332 - DNA, 30 bp, Artificial Sequence
  Synthetic primer
gccctagaattgggacggtatgaataatcc

SEQ ID NO: 333 - DNA, 30 bp, Artificial Sequence
  Synthetic primer
gctggtaacgtgggacggtatgaataatcc

SEQ ID NO: 334 - DNA, 30 bp, Artificial Sequence
  Synthetic primer
gccatcagactgggacggtatgaataatcc

SEQ ID NO: 335 - DNA, 30 bp, Artificial Sequence
  Synthetic primer
gcgtgcgtaatgggacggtatgaataatcc

SEQ ID NO: 336 - DNA, 30 bp, Artificial Sequence
  Synthetic primer
gcctattcaatgggacggtatgaataatcc

SEQ ID NO: 337 - DNA, 30 bp, Artificial Sequence
  Synthetic primer
gcagtgtctttgggacggtatgaataatcc

SEQ ID NO: 338 - DNA, 30 bp, Artificial Sequence
  Synthetic primer
gcccttgctgtgggacggtatgaataatcc

SEQ ID NO: 339 - DNA, 30 bp, Artificial Sequence
  Synthetic primer
gcttgctggatgggacggtatgaataatcc

SEQ ID NO: 340 - DNA, 30 bp, Artificial Sequence
  Synthetic primer
gcagctctggtgggacggtatgaataatcc

SEQ ID NO: 341 - DNA, 30 bp, Artificial Sequence
  Synthetic primer
gcaccaaggatgggacggtatgaataatcc

SEQ ID NO: 342 - DNA, 30 bp, Artificial Sequence
  Synthetic primer
gcgataaccttgggacggtatgaataatcc

SEQ ID NO: 343 - DNA, 30 bp, Artificial Sequence
  Synthetic primer
gctagatgactgggacggtatgaataatcc

SEQ ID NO: 344 - DNA, 30 bp, Artificial Sequence
  Synthetic primer
gctgcgaaggtgggacggtatgaataatcc

SEQ ID NO: 345 - DNA, 30 bp, Artificial Sequence
  Synthetic primer
gcgaccgagatgggacggtatgaataatcc

SEQ ID NO: 346 - DNA, 30 bp, Artificial Sequence
  Synthetic primer
gccagacaattgggacggtatgaataatcc

SEQ ID NO: 347 - DNA, 30 bp, Artificial Sequence
  Synthetic primer
gcctaggttctgggacggtatgaataatcc

SEQ ID NO: 348 - DNA, 30 bp, Artificial Sequence
  Synthetic primer
gcgttcattatgggacggtatgaataatcc

SEQ ID NO: 349 - DNA, 30 bp, Artificial Sequence
  Synthetic primer
gcaatgcgtttgggacggtatgaataatcc

SEQ ID NO: 350 - DNA, 30 bp, Artificial Sequence
  Synthetic primer
gcgagagttgtgggacggtatgaataatcc

SEQ ID NO: 351 - DNA, 30 bp, Artificial Sequence
  Synthetic primer
gcgattacagtgggacggtatgaataatcc

SEQ ID NO: 352 - DNA, 30 bp, Artificial Sequence
  Synthetic primer
gctgtgcttatgggacggtatgaataatcc

SEQ ID NO: 353 - DNA, 30 bp, Artificial Sequence
  Synthetic primer
gcagaacatttgggacggtatgaataatcc

SEQ ID NO: 354 - DNA, 30 bp, Artificial Sequence
  Synthetic primer
gctaccgctgtgggacggtatgaataatcc

SEQ ID NO: 355 - DNA, 30 bp, Artificial Sequence
  Synthetic primer
gctcctggtctgggacggtatgaataatcc

SEQ ID NO: 356 - DNA, 30 bp, Artificial Sequence
  Synthetic primer
gccctggatatgggacggtatgaataatcc

SEQ ID NO: 357 - DNA, 30 bp, Artificial Sequence
  Synthetic primer
gcatacctgttgggacggtatgaataatcc

SEQ ID NO: 358 - DNA, 30 bp, Artificial Sequence
  Synthetic primer
gcaatgttggtgggacggtatgaataatcc

SEQ ID NO: 359 - DNA, 30 bp, Artificial Sequence
  Synthetic primer
gctcgacggctgggacggtatgaataatcc

SEQ ID NO: 360 - DNA, 30 bp, Artificial Sequence
  Synthetic primer
gcggcagatatgggacggtatgaataatcc

SEQ ID NO: 361 - DNA, 30 bp, Artificial Sequence
  Synthetic primer
gcgtcttagttgggacggtatgaataatcc

SEQ ID NO: 362 - DNA, 30 bp, Artificial Sequence
  Synthetic primer
gcggaaggcgtgggacggtatgaataatcc

SEQ ID NO: 363 - DNA, 30 bp, Artificial Sequence
  Synthetic primer
gcggctaggctgggacggtatgaataatcc

SEQ ID NO: 364 - DNA, 30 bp, Artificial Sequence
  Synthetic primer
gccagcagcatgggacggtatgaataatcc

SEQ ID NO: 365 - DNA, 30 bp, Artificial Sequence
  Synthetic primer
gcccttaccttgggacggtatgaataatcc

SEQ ID NO: 366 - DNA, 30 bp, Artificial Sequence
  Synthetic primer
gccgagttagtgggacggtatgaataatcc

SEQ ID NO: 367 - DNA, 30 bp, Artificial Sequence
  Synthetic primer
gcgatgttactgggacggtatgaataatcc

SEQ ID NO: 368 - DNA, 30 bp, Artificial Sequence
  Synthetic primer
gctgattacatgggacggtatgaataatcc

SEQ ID NO: 369 - DNA, 30 bp, Artificial Sequence
  Synthetic primer
gcttgataattgggacggtatgaataatcc

SEQ ID NO: 370 - DNA, 30 bp, Artificial Sequence
  Synthetic primer
gcacgcatagtgggacggtatgaataatcc

SEQ ID NO: 371 - DNA, 30 bp, Artificial Sequence
  Synthetic primer
gcctgtggactgggacggtatgaataatcc

SEQ ID NO: 372 - DNA, 30 bp, Artificial Sequence
  Synthetic primer
gcatagacaatgggacggtatgaataatcc

SEQ ID NO: 373 - DNA, 30 bp, Artificial Sequence
  Synthetic primer
gcccattgtttgggacggtatgaataatcc

SEQ ID NO: 374 - DNA, 30 bp, Artificial Sequence
  Synthetic primer
gcagaggaattgggacggtatgaataatcc

SEQ ID NO: 375 - DNA, 30 bp, Artificial Sequence
  Synthetic primer
gccttccttctgggacggtatgaataatcc

SEQ ID NO: 376 - DNA, 30 bp, Artificial Sequence
  Synthetic primer
gctctagcgatgggacggtatgaataatcc

SEQ ID NO: 377 - DNA, 30 bp, Artificial Sequence
  Synthetic primer
gctcaactgttgggacggtatgaataatcc

SEQ ID NO: 378 - DNA, 30 bp, Artificial Sequence
  Synthetic primer
gcgactattgtgggacggtatgaataatcc

SEQ ID NO: 379 - DNA, 30 bp, Artificial Sequence
  Synthetic primer
gccaacggtctgggacggtatgaataatcc

SEQ ID NO: 380 - DNA, 30 bp, Artificial Sequence
  Synthetic primer
gccttgcagatgggacggtatgaataatcc

SEQ ID NO: 381 - DNA, 30 bp, Artificial Sequence
  Synthetic primer
gcgatacagttgggacggtatgaataatcc

SEQ ID NO: 382 - DNA, 30 bp, Artificial Sequence
  Synthetic primer
gccctggtagtgggacggtatgaataatcc

SEQ ID NO: 383 - DNA, 30 bp, Artificial Sequence
  Synthetic primer
gcgttaggtctgggacggtatgaataatcc

SEQ ID NO: 384 - DNA, 30 bp, Artificial Sequence
  Synthetic primer
gctacttgcatgggacggtatgaataatcc

SEQ ID NO: 385 - DNA, 30 bp, Artificial Sequence
  Synthetic primer
gctccatgcttgggacggtatgaataatcc

SEQ ID NO: 386 - DNA, 30 bp, Artificial Sequence
  Synthetic primer
gcacatagcgtgggacggtatgaataatcc

SEQ ID NO: 387 - DNA, 30 bp, Artificial Sequence
  Synthetic primer
gctggatatctgggacggtatgaataatcc

SEQ ID NO: 388 - DNA, 30 bp, Artificial Sequence
  Synthetic primer
gcgagttacatgggacggtatgaataatcc

SEQ ID NO: 389 - DNA, 30 bp, Artificial Sequence
  Synthetic primer
gctgcgaccttgggacggtatgaataatcc

SEQ ID NO: 390 - DNA, 30 bp, Artificial Sequence
  Synthetic primer
gcatccgcagtgggacggtatgaataatcc

SEQ ID NO: 391 - DNA, 30 bp, Artificial Sequence
  Synthetic primer
gccagttggttgggacggtatgaataatcc

SEQ ID NO: 392 - DNA, 30 bp, Artificial Sequence
  Synthetic primer
gcctgattaatgggacggtatgaataatcc

SEQ ID NO: 393 - DNA, 30 bp, Artificial Sequence
  Synthetic primer
gctcgcaccttgggacggtatgaataatcc

SEQ ID NO: 394 - DNA, 30 bp, Artificial Sequence
  Synthetic primer
gccgccacagtgggacggtatgaataatcc

SEQ ID NO: 395 - DNA, 30 bp, Artificial Sequence
  Synthetic primer
gcgttgcggctgggacggtatgaataatcc

SEQ ID NO: 396 - DNA, 30 bp, Artificial Sequence
  Synthetic primer
gcagatataatgggacggtatgaataatcc

SEQ ID NO: 397 - DNA, 28 bp, Artificial Sequence
  Synthetic primer
atcgactgtatttatgcagaggccgagg

SEQ ID NO: 398 - DNA, 28 bp, Artificial Sequence
  Synthetic primer
gctagcagtatttatgcagaggccgagg

SEQ ID NO: 399 - DNA, 28 bp, Artificial Sequence
  Synthetic primer
tactctcctatttatgcagaggccgagg

SEQ ID NO: 400 - DNA, 28 bp, Artificial Sequence
  Synthetic primer
tgacagcatatttatgcagaggccgagg

SEQ ID NO: 401 - DNA, 28 bp, Artificial Sequence
  Synthetic primer
gcaggttgtatttatgcagaggccgagg

SEQ ID NO: 402 - DNA, 28 bp, Artificial Sequence
  Synthetic primer
ttccagcttatttatgcagaggccgagg

SEQ ID NO: 403 - DNA, 28 bp, Artificial Sequence
  Synthetic primer
tagttagctatttatgcagaggccgagg

SEQ ID NO: 404 - DNA, 28 bp, Artificial Sequence
  Synthetic primer
agcgctaatatttatgcagaggccgagg

SEQ ID NO: 405 - DNA, 28 bp, Artificial Sequence
  Synthetic primer
cggttctttatttatgcagaggccgagg

SEQ ID NO: 406 - DNA, 28 bp, Artificial Sequence
  Synthetic primer
tagcattgtatttatgcagaggccgagg

SEQ ID NO: 407 - DNA, 28 bp, Artificial Sequence
  Synthetic primer
aattcaactatttatgcagaggccgagg

SEQ ID NO: 408 - DNA, 28 bp, Artificial Sequence
  Synthetic primer
ttcacagatatttatgcagaggccgagg

SEQ ID NO: 409 - DNA, 28 bp, Artificial Sequence
  Synthetic primer
gctctctttatttatgcagaggccgagg

SEQ ID NO: 410 - DNA, 28 bp, Artificial Sequence
  Synthetic primer
tgacttggtatttatgcagaggccgagg

SEQ ID NO: 411 - DNA, 28 bp, Artificial Sequence
  Synthetic primer
tatggttctatttatgcagaggccgagg

SEQ ID NO: 412 - DNA, 28 bp, Artificial Sequence
  Synthetic primer
cactagcctatttatgcagaggccgagg

SEQ ID NO: 413 - DNA, 28 bp, Artificial Sequence
  Synthetic primer
aacctctttatttatgcagaggccgagg

SEQ ID NO: 414 - DNA, 28 bp, Artificial Sequence
  Synthetic primer
ctacattgtatttatgcagaggccgagg

SEQ ID NO: 415 - DNA, 28 bp, Artificial Sequence
  Synthetic primer
gcgattactatttatgcagaggccgagg

SEQ ID NO: 416 - DNA, 28 bp, Artificial Sequence
  Synthetic primer
aattggcctatttatgcagaggccgagg

SEQ ID NO: 417 - DNA, 28 bp, Artificial Sequence
  Synthetic primer
aattgctttatttatgcagaggccgagg

SEQ ID NO: 418 - DNA, 28 bp, Artificial Sequence
  Synthetic primer
ttggtctgtatttatgcagaggccgagg

SEQ ID NO: 419 - DNA, 28 bp, Artificial Sequence
  Synthetic primer
catcctggtatttatgcagaggccgagg

SEQ ID NO: 420 - DNA, 28 bp, Artificial Sequence
  Synthetic primer
ggattaactatttatgcagaggccgagg

SEQ ID NO: 421 - DNA, 28 bp, Artificial Sequence
  Synthetic primer
cgcatatttatttatgcagaggccgagg

SEQ ID NO: 422 - DNA, 28 bp, Artificial Sequence
  Synthetic primer
tcattcgatatttatgcagaggccgagg

SEQ ID NO: 423 - DNA, 28 bp, Artificial Sequence
  Synthetic primer
gtccaatctatttatgcagaggccgagg

SEQ ID NO: 424 - DNA, 28 bp, Artificial Sequence
  Synthetic primer
cttggtcatatttatgcagaggccgagg

SEQ ID NO: 425 - DNA, 28 bp, Artificial Sequence
  Synthetic primer
ccaacgcttatttatgcagaggccgagg

SEQ ID NO: 426 - DNA, 28 bp, Artificial Sequence
  Synthetic primer
tccacttctatttatgcagaggccgagg

SEQ ID NO: 427 - DNA, 28 bp, Artificial Sequence
  Synthetic primer
aatctccatatttatgcagaggccgagg

SEQ ID NO: 428 - DNA, 28 bp, Artificial Sequence
  Synthetic primer
gtctgcactatttatgcagaggccgagg

SEQ ID NO: 429 - DNA, 28 bp, Artificial Sequence
  Synthetic primer
ctgctccttatttatgcagaggccgagg

SEQ ID NO: 430 - DNA, 28 bp, Artificial Sequence
  Synthetic primer
ttagccagtatttatgcagaggccgagg

SEQ ID NO: 431 - DNA, 28 bp, Artificial Sequence
  Synthetic primer
gctgattctatttatgcagaggccgagg

SEQ ID NO: 432 - DNA, 28 bp, Artificial Sequence
  Synthetic primer
gaatcgactatttatgcagaggccgagg

SEQ ID NO: 433 - DNA, 28 bp, Artificial Sequence
  Synthetic primer
agtcaccttatttatgcagaggccgagg

SEQ ID NO: 434 - DNA, 28 bp, Artificial Sequence
  Synthetic primer
cacgattctatttatgcagaggccgagg

SEQ ID NO: 435 - DNA, 28 bp, Artificial Sequence
  Synthetic primer
gctccgattatttatgcagaggccgagg

SEQ ID NO: 436 - DNA, 28 bp, Artificial Sequence
  Synthetic primer
cttggctttatttatgcagaggccgagg

SEQ ID NO: 437 - DNA, 28 bp, Artificial Sequence
  Synthetic primer
gctgcacttatttatgcagaggccgagg

SEQ ID NO: 438 - DNA, 28 bp, Artificial Sequence
  Synthetic primer
gaacttcgtatttatgcagaggccgagg

SEQ ID NO: 439 - DNA, 28 bp, Artificial Sequence
  Synthetic primer
ctgtattctatttatgcagaggccgagg

SEQ ID NO: 440 - DNA, 28 bp, Artificial Sequence
  Synthetic primer
atatccgatatttatgcagaggccgagg

SEQ ID NO: 441 - DNA, 28 bp, Artificial Sequence
  Synthetic primer
ttgtccattatttatgcagaggccgagg

SEQ ID NO: 442 - DNA, 28 bp, Artificial Sequence
  Synthetic primer
agtaagtctatttatgcagaggccgagg

SEQ ID NO: 443 - DNA, 28 bp, Artificial Sequence
  Synthetic primer
gaatatcatatttatgcagaggccgagg

SEQ ID NO: 444 - DNA, 28 bp, Artificial Sequence
  Synthetic primer
caactgattatttatgcagaggccgagg

SEQ ID NO: 445 - DNA, 28 bp, Artificial Sequence
  Synthetic primer
cctgtcattatttatgcagaggccgagg

SEQ ID NO: 446 - DNA, 28 bp, Artificial Sequence
  Synthetic primer
gacggttatatttatgcagaggccgagg

SEQ ID NO: 447 - DNA, 28 bp, Artificial Sequence
  Synthetic primer
ctattagctatttatgcagaggccgagg

SEQ ID NO: 448 - DNA, 28 bp, Artificial Sequence
  Synthetic primer
tccaaccatatttatgcagaggccgagg

SEQ ID NO: 449 - DNA, 28 bp, Artificial Sequence
  Synthetic primer
ctggctattatttatgcagaggccgagg

SEQ ID NO: 450 - DNA, 28 bp, Artificial Sequence
  Synthetic primer
gcggactttatttatgcagaggccgagg

SEQ ID NO: 451 - DNA, 28 bp, Artificial Sequence
  Synthetic primer
ccatcacatatttatgcagaggccgagg

SEQ ID NO: 452 - DNA, 28 bp, Artificial Sequence
  Synthetic primer
ggcaatactatttatgcagaggccgagg

SEQ ID NO: 453 - DNA, 28 bp, Artificial Sequence
  Synthetic primer
cacttcattatttatgcagaggccgagg

SEQ ID NO: 454 - DNA, 28 bp, Artificial Sequence
  Synthetic primer
caagcttatatttatgcagaggccgagg

SEQ ID NO: 455 - DNA, 28 bp, Artificial Sequence
  Synthetic primer
aggtaccatatttatgcagaggccgagg

SEQ ID NO: 456 - DNA, 28 bp, Artificial Sequence
  Synthetic primer
tccataactatttatgcagaggccgagg

SEQ ID NO: 457 - DNA, 28 bp, Artificial Sequence
  Synthetic primer
gtcctcattatttatgcagaggccgagg

SEQ ID NO: 458 - DNA, 28 bp, Artificial Sequence
  Synthetic primer
agtactgctatttatgcagaggccgagg

SEQ ID NO: 459 - DNA, 28 bp, Artificial Sequence
  Synthetic primer
cttgaatctatttatgcagaggccgagg

SEQ ID NO: 460 - DNA, 28 bp, Artificial Sequence
  Synthetic primer
ccaactaatatttatgcagaggccgagg

SEQ ID NO: 461 - DNA, 28 bp, Artificial Sequence
  Synthetic primer
aataccattatttatgcagaggccgagg

SEQ ID NO: 462 - DNA, 28 bp, Artificial Sequence
  Synthetic primer
gcgatatttatttatgcagaggccgagg

SEQ ID NO: 463 - DNA, 28 bp, Artificial Sequence
  Synthetic primer
gaacgctatatttatgcagaggccgagg

SEQ ID NO: 464 - DNA, 28 bp, Artificial Sequence
  Synthetic primer
ctgacatctatttatgcagaggccgagg

SEQ ID NO: 465 - DNA, 28 bp, Artificial Sequence
  Synthetic primer
gccaccattatttatgcagaggccgagg

SEQ ID NO: 466 - DNA, 28 bp, Artificial Sequence
  Synthetic primer
cgactctctatttatgcagaggccgagg

SEQ ID NO: 467 - DNA, 28 bp, Artificial Sequence
  Synthetic primer
tgctattatatttatgcagaggccgagg

SEQ ID NO: 468 - DNA, 28 bp, Artificial Sequence
  Synthetic primer
cttctggctatttatgcagaggccgagg

SEQ ID NO: 469 - DNA, 28 bp, Artificial Sequence
  Synthetic primer
atgaattatatttatgcagaggccgagg

SEQ ID NO: 470 - DNA, 28 bp, Artificial Sequence
  Synthetic primer
tactccagtatttatgcagaggccgagg

SEQ ID NO: 471 - DNA, 28 bp, Artificial Sequence
  Synthetic primer
atcatacctatttatgcagaggccgagg

SEQ ID NO: 472 - DNA, 28 bp, Artificial Sequence
  Synthetic primer
cctctaactatttatgcagaggccgagg

SEQ ID NO: 473 - DNA, 28 bp, Artificial Sequence
  Synthetic primer
atcttctctatttatgcagaggccgagg

SEQ ID NO: 474 - DNA, 28 bp, Artificial Sequence
  Synthetic primer
cagctcactatttatgcagaggccgagg

SEQ ID NO: 475 - DNA, 28 bp, Artificial Sequence
  Synthetic primer
ggttatcttatttatgcagaggccgagg

SEQ ID NO: 476 - DNA, 28 bp, Artificial Sequence
  Synthetic primer
tccgcatatatttatgcagaggccgagg

SEQ ID NO: 477 - DNA, 28 bp, Artificial Sequence
  Synthetic primer
tgcttcactatttatgcagaggccgagg

SEQ ID NO: 478 - DNA, 28 bp, Artificial Sequence
  Synthetic primer
gcttcctatatttatgcagaggccgagg

SEQ ID NO: 479 - DNA, 28 bp, Artificial Sequence
  Synthetic primer
gaccatcttatttatgcagaggccgagg

SEQ ID NO: 480 - DNA, 28 bp, Artificial Sequence
  Synthetic primer
ctggtatttatttatgcagaggccgagg

SEQ ID NO: 481 - DNA, 28 bp, Artificial Sequence
  Synthetic primer
ttaatcactatttatgcagaggccgagg

SEQ ID NO: 482 - DNA, 28 bp, Artificial Sequence
  Synthetic primer
cgcgaatatatttatgcagaggccgagg

SEQ ID NO: 483 - DNA, 28 bp, Artificial Sequence
  Synthetic primer
gctcaccatatttatgcagaggccgagg

SEQ ID NO: 484 - DNA, 28 bp, Artificial Sequence
  Synthetic primer
tcatgtcttatttatgcagaggccgagg

SEQ ID NO: 485 - DNA, 28 bp, Artificial Sequence
  Synthetic primer
atccttaatatttatgcagaggccgagg

SEQ ID NO: 486 - DNA, 28 bp, Artificial Sequence
  Synthetic primer
ttcttggctatttatgcagaggccgagg

SEQ ID NO: 487 - DNA, 28 bp, Artificial Sequence
  Synthetic primer
catcactttatttatgcagaggccgagg

SEQ ID NO: 488 - DNA, 28 bp, Artificial Sequence
  Synthetic primer
cgaacttctatttatgcagaggccgagg

SEQ ID NO: 489 - DNA, 28 bp, Artificial Sequence
  Synthetic primer
gacattaatatttatgcagaggccgagg

SEQ ID NO: 490 - DNA, 28 bp, Artificial Sequence
  Synthetic primer
ttcacctttatttatgcagaggccgagg

SEQ ID NO: 491 - DNA, 28 bp, Artificial Sequence
  Synthetic primer
ccaatctgtatttatgcagaggccgagg

SEQ ID NO: 492 - DNA, 28 bp, Artificial Sequence
  Synthetic primer
cgacagtttatttatgcagaggccgagg

SEQ ID NO: 493 - DNA, 28 bp, Artificial Sequence
  Synthetic primer
ctctactttgggacggtatgaataatcc

SEQ ID NO: 494 - DNA, 28 bp, Artificial Sequence
  Synthetic primer
taagcatgtgggacggtatgaataatcc

SEQ ID NO: 495 - DNA, 28 bp, Artificial Sequence
  Synthetic primer
agatgtgctgggacggtatgaataatcc

SEQ ID NO: 496 - DNA, 28 bp, Artificial Sequence
  Synthetic primer
gtcgagcatgggacggtatgaataatcc

SEQ ID NO: 497 - DNA, 28 bp, Artificial Sequence
  Synthetic primer
gaattgcttgggacggtatgaataatcc

SEQ ID NO: 498 - DNA, 28 bp, Artificial Sequence
  Synthetic primer
aagcaacttgggacggtatgaataatcc

SEQ ID NO: 499 - DNA, 28 bp, Artificial Sequence
  Synthetic primer
ctaactggtgggacggtatgaataatcc

SEQ ID NO: 500 - DNA, 28 bp, Artificial Sequence
  Synthetic primer
aggctcaatgggacggtatgaataatcc

SEQ ID NO: 501 - DNA, 28 bp, Artificial Sequence
  Synthetic primer
gatcgtgttgggacggtatgaataatcc

SEQ ID NO: 502 - DNA, 28 bp, Artificial Sequence
  Synthetic primer
tctggacctgggacggtatgaataatcc

SEQ ID NO: 503 - DNA, 28 bp, Artificial Sequence
  Synthetic primer
tgttatactgggacggtatgaataatcc

SEQ ID NO: 504 - DNA, 28 bp, Artificial Sequence
  Synthetic primer
tcagcgaatgggacggtatgaataatcc

SEQ ID NO: 505 - DNA, 28 bp, Artificial Sequence
  Synthetic primer
gtcaagtttgggacggtatgaataatcc

SEQ ID NO: 506 - DNA, 28 bp, Artificial Sequence
  Synthetic primer
aggatgtgtgggacggtatgaataatcc

SEQ ID NO: 507 - DNA, 28 bp, Artificial Sequence
  Synthetic primer
cattccgatgggacggtatgaataatcc

SEQ ID NO: 508 - DNA, 28 bp, Artificial Sequence
  Synthetic primer
acatcctttgggacggtatgaataatcc

SEQ ID NO: 509 - DNA, 28 bp, Artificial Sequence
  Synthetic primer
accgcgcgtgggacggtatgaataatcc

SEQ ID NO: 510 - DNA, 28 bp, Artificial Sequence
  Synthetic primer
tcgccagatgggacggtatgaataatcc

SEQ ID NO: 511 - DNA, 28 bp, Artificial Sequence
  Synthetic primer
tcgctatgtgggacggtatgaataatcc

SEQ ID NO: 512 - DNA, 28 bp, Artificial Sequence
  Synthetic primer
ggctcctgtgggacggtatgaataatcc

SEQ ID NO: 513 - DNA, 28 bp, Artificial Sequence
  Synthetic primer
atccgacatgggacggtatgaataatcc

SEQ ID NO: 514 - DNA, 28 bp, Artificial Sequence
  Synthetic primer
aacataattgggacggtatgaataatcc

SEQ ID NO: 515 - DNA, 28 bp, Artificial Sequence
  Synthetic primer
atggtaggtgggacggtatgaataatcc

SEQ ID NO: 516 - DNA, 28 bp, Artificial Sequence
  Synthetic primer
gctaagtatgggacggtatgaataatcc

SEQ ID NO: 517 - DNA, 28 bp, Artificial Sequence
  Synthetic primer
cgatcatgtgggacggtatgaataatcc

SEQ ID NO: 518 - DNA, 28 bp, Artificial Sequence
  Synthetic primer
tagatccttgggacggtatgaataatcc

SEQ ID NO: 519 - DNA, 28 bp, Artificial Sequence
  Synthetic primer
ttactgtctgggacggtatgaataatcc

SEQ ID NO: 520 - DNA, 28 bp, Artificial Sequence
  Synthetic primer
ggcataggtgggacggtatgaataatcc

SEQ ID NO: 521 - DNA, 28 bp, Artificial Sequence
  Synthetic primer
caaggcgatgggacggtatgaataatcc

SEQ ID NO: 522 - DNA, 28 bp, Artificial Sequence
  Synthetic primer
gacgctattgggacggtatgaataatcc

SEQ ID NO: 523 - DNA, 28 bp, Artificial Sequence
  Synthetic primer
acttcttctgggacggtatgaataatcc

SEQ ID NO: 524 - DNA, 28 bp, Artificial Sequence
  Synthetic primer
cctagaattgggacggtatgaataatcc

SEQ ID NO: 525 - DNA, 28 bp, Artificial Sequence
  Synthetic primer
tggtaacgtgggacggtatgaataatcc

SEQ ID NO: 526 - DNA, 28 bp, Artificial Sequence
  Synthetic primer
catcagactgggacggtatgaataatcc

SEQ ID NO: 527 - DNA, 28 bp, Artificial Sequence
  Synthetic primer
gtgcgtaatgggacggtatgaataatcc

SEQ ID NO: 528 - DNA, 28 bp, Artificial Sequence
  Synthetic primer
ctattcaatgggacggtatgaataatcc

SEQ ID NO: 529 - DNA, 28 bp, Artificial Sequence
  Synthetic primer
agtgtctttgggacggtatgaataatcc

SEQ ID NO: 530 - DNA, 28 bp, Artificial Sequence
  Synthetic primer
ccttgctgtgggacggtatgaataatcc

SEQ ID NO: 531 - DNA, 28 bp, Artificial Sequence
  Synthetic primer
ttgctggatgggacggtatgaataatcc

SEQ ID NO: 532 - DNA, 28 bp, Artificial Sequence
  Synthetic primer
agctctggtgggacggtatgaataatcc

SEQ ID NO: 533 - DNA, 28 bp, Artificial Sequence
  Synthetic primer
accaaggatgggacggtatgaataatcc

SEQ ID NO: 534 - DNA, 28 bp, Artificial Sequence
  Synthetic primer
gataaccttgggacggtatgaataatcc

SEQ ID NO: 535 - DNA, 28 bp, Artificial Sequence
  Synthetic primer
tagatgactgggacggtatgaataatcc

SEQ ID NO: 536 - DNA, 28 bp, Artificial Sequence
  Synthetic primer
tgcgaaggtgggacggtatgaataatcc

SEQ ID NO: 537 - DNA, 28 bp, Artificial Sequence
  Synthetic primer
gaccgagatgggacggtatgaataatcc

SEQ ID NO: 538 - DNA, 28 bp, Artificial Sequence
  Synthetic primer
cagacaattgggacggtatgaataatcc

SEQ ID NO: 539 - DNA, 28 bp, Artificial Sequence
  Synthetic primer
ctaggttctgggacggtatgaataatcc

SEQ ID NO: 540 - DNA, 28 bp, Artificial Sequence
  Synthetic primer
gttcattatgggacggtatgaataatcc

SEQ ID NO: 541 - DNA, 28 bp, Artificial Sequence
  Synthetic primer
aatgcgtttgggacggtatgaataatcc

SEQ ID NO: 542 - DNA, 28 bp, Artificial Sequence
  Synthetic primer
gagagttgtgggacggtatgaataatcc

SEQ ID NO: 543 - DNA, 28 bp, Artificial Sequence
  Synthetic primer
gattacagtgggacggtatgaataatcc

SEQ ID NO: 544 - DNA, 28 bp, Artificial Sequence
  Synthetic primer
tgtgcttatgggacggtatgaataatcc

SEQ ID NO: 545 - DNA, 28 bp, Artificial Sequence
  Synthetic primer
agaacatttgggacggtatgaataatcc

SEQ ID NO: 546 - DNA, 28 bp, Artificial Sequence
  Synthetic primer
taccgctgtgggacggtatgaataatcc

SEQ ID NO: 547 - DNA, 28 bp, Artificial Sequence
  Synthetic primer
tcctggtctgggacggtatgaataatcc

SEQ ID NO: 548 - DNA, 28 bp, Artificial Sequence
  Synthetic primer
cctggatatgggacggtatgaataatcc

SEQ ID NO: 549 - DNA, 28 bp, Artificial Sequence
  Synthetic primer
atacctgttgggacggtatgaataatcc

SEQ ID NO: 550 - DNA, 28 bp, Artificial Sequence
  Synthetic primer
aatgttggtgggacggtatgaataatcc

SEQ ID NO: 551 - DNA, 28 bp, Artificial Sequence
  Synthetic primer
tcgacggctgggacggtatgaataatcc

SEQ ID NO: 552 - DNA, 28 bp, Artificial Sequence
  Synthetic primer
ggcagatatgggacggtatgaataatcc

SEQ ID NO: 553 - DNA, 28 bp, Artificial Sequence
  Synthetic primer
gtcttagttgggacggtatgaataatcc

SEQ ID NO: 554 - DNA, 28 bp, Artificial Sequence
  Synthetic primer
ggaaggcgtgggacggtatgaataatcc

SEQ ID NO: 555 - DNA, 28 bp, Artificial Sequence
  Synthetic primer
ggctaggctgggacggtatgaataatcc

SEQ ID NO: 556 - DNA, 28 bp, Artificial Sequence
  Synthetic primer
cagcagcatgggacggtatgaataatcc

SEQ ID NO: 557 - DNA, 28 bp, Artificial Sequence
  Synthetic primer
ccttaccttgggacggtatgaataatcc

SEQ ID NO: 558 - DNA, 28 bp, Artificial Sequence
  Synthetic primer
cgagttagtgggacggtatgaataatcc

SEQ ID NO: 559 - DNA, 28 bp, Artificial Sequence
  Synthetic primer
gatgttactgggacggtatgaataatcc

SEQ ID NO: 560 - DNA, 28 bp, Artificial Sequence
  Synthetic primer
tgattacatgggacggtatgaataatcc

SEQ ID NO: 561 - DNA, 28 bp, Artificial Sequence
  Synthetic primer
ttgataattgggacggtatgaataatcc

SEQ ID NO: 562 - DNA, 28 bp, Artificial Sequence
  Synthetic primer
acgcatagtgggacggtatgaataatcc

SEQ ID NO: 563 - DNA, 28 bp, Artificial Sequence
  Synthetic primer
ctgtggactgggacggtatgaataatcc

SEQ ID NO: 564 - DNA, 28 bp, Artificial Sequence
  Synthetic primer
atagacaatgggacggtatgaataatcc

SEQ ID NO: 565 - DNA, 28 bp, Artificial Sequence
  Synthetic primer
ccattgtttgggacggtatgaataatcc

SEQ ID NO: 566 - DNA, 28 bp, Artificial Sequence
  Synthetic primer
agaggaattgggacggtatgaataatcc

SEQ ID NO: 567 - DNA, 28 bp, Artificial Sequence
  Synthetic primer
cttccttctgggacggtatgaataatcc

SEQ ID NO: 568 - DNA, 28 bp, Artificial Sequence
  Synthetic primer
tctagcgatgggacggtatgaataatcc

SEQ ID NO: 569 - DNA, 28 bp, Artificial Sequence
  Synthetic primer
tcaactgttgggacggtatgaataatcc

SEQ ID NO: 570 - DNA, 28 bp, Artificial Sequence
  Synthetic primer
gactattgtgggacggtatgaataatcc

SEQ ID NO: 571 - DNA, 28 bp, Artificial Sequence
  Synthetic primer
caacggtctgggacggtatgaataatcc

SEQ ID NO: 572 - DNA, 28 bp, Artificial Sequence
  Synthetic primer
cttgcagatgggacggtatgaataatcc

SEQ ID NO: 573 - DNA, 28 bp, Artificial Sequence
  Synthetic primer
gatacagttgggacggtatgaataatcc

SEQ ID NO: 574 - DNA, 28 bp, Artificial Sequence
  Synthetic primer
cctggtagtgggacggtatgaataatcc

SEQ ID NO: 575 - DNA, 28 bp, Artificial Sequence
  Synthetic primer
gttaggtctgggacggtatgaataatcc

SEQ ID NO: 576 - DNA, 28 bp, Artificial Sequence
  Synthetic primer
tacttgcatgggacggtatgaataatcc

SEQ ID NO: 577 - DNA, 28 bp, Artificial Sequence
  Synthetic primer
tccatgcttgggacggtatgaataatcc

SEQ ID NO: 578 - DNA, 28 bp, Artificial Sequence
  Synthetic primer
acatagcgtgggacggtatgaataatcc

SEQ ID NO: 579 - DNA, 28 bp, Artificial Sequence
  Synthetic primer
tggatatctgggacggtatgaataatcc

SEQ ID NO: 580 - DNA, 28 bp, Artificial Sequence
  Synthetic primer
gagttacatgggacggtatgaataatcc

SEQ ID NO: 581 - DNA, 28 bp, Artificial Sequence
  Synthetic primer
tgcgaccttgggacggtatgaataatcc

SEQ ID NO: 582 - DNA, 28 bp, Artificial Sequence
  Synthetic primer
atccgcagtgggacggtatgaataatcc

SEQ ID NO: 583 - DNA, 28 bp, Artificial Sequence
  Synthetic primer
cagttggttgggacggtatgaataatcc

SEQ ID NO: 584 - DNA, 28 bp, Artificial Sequence
  Synthetic primer
ctgattaatgggacggtatgaataatcc

SEQ ID NO: 585 - DNA, 28 bp, Artificial Sequence
  Synthetic primer
tcgcaccttgggacggtatgaataatcc

SEQ ID NO: 586 - DNA, 28 bp, Artificial Sequence
  Synthetic primer
cgccacagtgggacggtatgaataatcc

SEQ ID NO: 587 - DNA, 28 bp, Artificial Sequence
  Synthetic primer
gttgcggctgggacggtatgaataatcc

SEQ ID NO: 588 - DNA, 28 bp, Artificial Sequence
  Synthetic primer
agatataatgggacggtatgaataatcc

SEQ ID NO: 589 - DNA, 20 bp, Artificial Sequence
  Synthetic primer
agagtttgatcctggctcag

SEQ ID NO: 590 - DNA, 20 bp, Artificial Sequence
  Synthetic primer
agagtttgatcmtggctcag

SEQ ID NO: 591 - DNA, 22 bp, Artificial Sequence
  Synthetic primer
cggacgggtgagtaacgcgtga

SEQ ID NO: 592 - DNA, 22 bp, Artificial Sequence
  Synthetic primer
ccagactcctacgggaggcagc

SEQ ID NO: 593 - DNA, 19 bp, Artificial Sequence
  Synthetic primer
ctcctacgggaggcagcag

SEQ ID NO: 594 - DNA, 20 bp, Artificial Sequence
  Synthetic primer
ggggaatyttccgcaatggg

SEQ ID NO: 595 - DNA, 19 bp, Artificial Sequence
  Synthetic primer
gtgccagcmgccgcggtaa

SEQ ID NO: 596 - DNA, 19 bp, Artificial Sequence
  Synthetic primer
gtgccagcagccgcggtaa

SEQ ID NO: 597 - DNA, 16 bp, Artificial Sequence
  Synthetic primer
crcctggggagtrcrg

SEQ ID NO: 598 - DNA, 16 bp, Artificial Sequence
  Synthetic primer
caacgagcgcaaccct

SEQ ID NO: 599 - DNA, 18 bp, Artificial Sequence
  Synthetic primer
gggctacacacgygcwac

SEQ ID NO: 600 - DNA, 18 bp, Artificial Sequence
  Synthetic primer
gwattaccgcggckgctg

SEQ ID NO: 601 - DNA, 25 bp, Artificial Sequence
  Synthetic primer
gactacwggggtatctaatcccwtt

SEQ ID NO: 602 - DNA, 23 bp, Artificial Sequence
  Synthetic primer
cttgtgcgggcccccgtcaattc

SEQ ID NO: 603 - DNA, 23 bp, Artificial Sequence
  Synthetic primer
  misc_feature (9)..(9): “n” at position 9 can be any nucleotide or modified nucleotide
gtcaattcntttgagtttyaryc

SEQ ID NO: 604 - DNA, 25 bp, Artificial Sequence
  Synthetic primer
  misc_feature (13)..(13): “n” at position 13 can be any nucleotide or modified nucleotide
ccccgtcaattcntttgagtttyar

SEQ ID NO: 605 - DNA, 20 bp, Artificial Sequence
  Synthetic primer
ccgtcaattcmtttragttt

SEQ ID NO: 606 - DNA, 16 bp, Artificial Sequence
  Synthetic primer
agggttgcgctcgttg

SEQ ID NO: 607 - DNA, 16 bp, Artificial Sequence
  Synthetic primer
gayttgacgtcatccm

SEQ ID NO: 608 - DNA, 16 bp, Artificial Sequence
  Synthetic primer
gayttgacgtcatcca

SEQ ID NO: 609 - DNA, 21 bp, Artificial Sequence
  Synthetic primer
cggtgtgtacaagrccygrga

SEQ ID NO: 610 - DNA, 25 bp, Artificial Sequence
  Synthetic primer
cgggcggtgtgtacaagrccygrga

SEQ ID NO: 611 - DNA, 17 bp, Artificial Sequence
  Synthetic primer
gacgggcggtgtgtrca

SEQ ID NO: 612 - DNA, 19 bp, Artificial Sequence
  Synthetic primer
ggttaccttgttacgactt

SEQ ID NO: 613 - DNA, 15 bp, Artificial Sequence
  Synthetic primer
accttgttacgactt

SEQ ID NO: 614 - DNA, 24 bp, Artificial Sequence
  Synthetic primer
acatatccaaccttatataacatt

SEQ ID NO: 615 - DNA, 24 bp, Artificial Sequence
  Synthetic primer
tctaacatacactcataataatac

SEQ ID NO: 616 - DNA, 24 bp, Artificial Sequence
  Synthetic primer
tatataattcctcataccacataa

SEQ ID NO: 617 - DNA, 24 bp, Artificial Sequence
  Synthetic primer
tcaattacactctataatacctta

SEQ ID NO: 618 - DNA, 24 bp, Artificial Sequence
  Synthetic primer
taattatacatctcatcttctaca

SEQ ID NO: 619 - DNA, 24 bp, Artificial Sequence
  Synthetic primer
ctactatacatcttactatacttt

SEQ ID NO: 620 - DNA, 24 bp, Artificial Sequence
  Synthetic primer
aacatctatctttctaactttcaa

SEQ ID NO: 621 - DNA, 24 bp, Artificial Sequence
  Synthetic primer
aacctattattctctacctataat

SEQ ID NO: 622 - DNA, 24 bp, Artificial Sequence
  Synthetic primer
ctacatctaatcattactataaca

SEQ ID NO: 623 - DNA, 24 bp, Artificial Sequence
  Synthetic primer
cattcaatacacaaatactcaaat

SEQ ID NO: 624 - DNA, 24 bp, Artificial Sequence
  Synthetic primer
cttctatctatctttcatttctat

SEQ ID NO: 625 - DNA, 24 bp, Artificial Sequence
  Synthetic primer
ttaatcttcaatataccttaccaa

SEQ ID NO: 626 - DNA, 24 bp, Artificial Sequence
  Synthetic primer
caactacacttatcattacataaa

SEQ ID NO: 627 - DNA, 24 bp, Artificial Sequence
  Synthetic primer
ttaatcttcaatataccttaccaa

SEQ ID NO: 628 - DNA, 24 bp, Artificial Sequence
  Synthetic primer
taatacataactactaactctaac

SEQ ID NO: 629 - DNA, 24 bp, Artificial Sequence
  Synthetic primer
ttcacttatctactatttcttaac

SEQ ID NO: 630 - DNA, 24 bp, Artificial Sequence
  Synthetic primer
tctataactccacttaataacata

SEQ ID NO: 631 - DNA, 24 bp, Artificial Sequence
  Synthetic primer
aacttaatctcttataactacctt

SEQ ID NO: 632 - DNA, 24 bp, Artificial Sequence
  Synthetic primer
attaattccacttaccttacaata

SEQ ID NO: 633 - DNA, 24 bp, Artificial Sequence
  Synthetic primer
attattatcattcctatctaacca

SEQ ID NO: 634 - DNA, 24 bp, Artificial Sequence
  Synthetic primer
ttaccttaactatattctacaaca

SEQ ID NO: 635 - DNA, 24 bp, Artificial Sequence
  Synthetic primer
atttacactacttacacacaataa

SEQ ID NO: 636 - DNA, 24 bp, Artificial Sequence
  Synthetic primer
tacttaaacatacaaacttactca

SEQ ID NO: 637 - DNA, 24 bp, Artificial Sequence
  Synthetic primer
tcatatactactctttaaacacta

SEQ ID NO: 638 - DNA, 24 bp, Artificial Sequence
  Synthetic primer
tctttcaaacaatacttctctaaa

SEQ ID NO: 639 - DNA, 24 bp, Artificial Sequence
  Synthetic primer
ttattacactctatactctaattc

SEQ ID NO: 640 - DNA, 24 bp, Artificial Sequence
  Synthetic primer
ctacactatatattctacacaatt

SEQ ID NO: 641 - DNA, 24 bp, Artificial Sequence
  Synthetic primer
aattcaactactctcaatttacta

SEQ ID NO: 642 - DNA, 24 bp, Artificial Sequence
  Synthetic primer
acataattctactctaactcattt

SEQ ID NO: 643 - DNA, 24 bp, Artificial Sequence
  Synthetic primer
taattatacatctcatcttctaca

SEQ ID NO: 644 - DNA, 24 bp, Artificial Sequence
  Synthetic primer
tcactaattaatcacctacatatt

SEQ ID NO: 645 - DNA, 24 bp, Artificial Sequence
  Synthetic primer
ctactatacatcttactatacttt

SEQ ID NO: 646 - DNA, 24 bp, Artificial Sequence
  Synthetic primer
aacatctatctttctaactttcaa

SEQ ID NO: 647 - DNA, 24 bp, Artificial Sequence
  Synthetic primer
atatctatcatcctactacatata

SEQ ID NO: 648 - DNA, 20 bp, Artificial Sequence
  Synthetic primer
aatgtaacgtcatgtgagcg

SEQ ID NO: 649 - DNA, 20 bp, Artificial Sequence
  Synthetic primer
atattccttgacaggccggg

SEQ ID NO: 650 - DNA, 90 bp, Artificial Sequence
  Synthetic primer
caagcagaagacggcatacgagataagatcgagtgactggagttcagacgtgtgctcttccgatcttaaagcagcgtatccacatagcgt

SEQ ID NO: 651 - DNA, 8 bp, Artificial Sequence
  Synthetic primer
aagatcga

SEQ ID NO: 652 - DNA, 8 bp, Artificial Sequence
  Synthetic primer
tcgatctt

SEQ ID NO: 653 - DNA, 90 bp, Artificial Sequence
  Synthetic primer
caagcagaagacggcatacgagataatagcgcgtgactggagttcagacgtgtgctcttccgatcttaaagcagcgtatccacatagcgt

SEQ ID NO: 654 - DNA, 8 bp, Artificial Sequence
  Synthetic primer
aatagcgc

SEQ ID NO: 655 - DNA, 8 bp, Artificial Sequence
  Synthetic primer
gcgctatt

SEQ ID NO: 656 - DNA, 90 bp, Artificial Sequence
  Synthetic primer
caagcagaagacggcatacgagataatctcttgtgactggagttcagacgtgtgctcttccgatcttaaagcagcgtatccacatagcgt

SEQ ID NO: 657 - DNA, 8 bp, Artificial Sequence
  Synthetic primer
aatctctt

SEQ ID NO: 658 - DNA, 8 bp, Artificial Sequence
  Synthetic primer
aagagatt

SEQ ID NO: 659 - DNA, 90 bp, Artificial Sequence
  Synthetic primer
caagcagaagacggcatacgagataatgcacagtgactggagttcagacgtgtgctcttccgatcttaaagcagcgtatccacatagcgt

SEQ ID NO: 660 - DNA, 8 bp, Artificial Sequence
  Synthetic primer
aatgcaca

SEQ ID NO: 661 - DNA, 8 bp, Artificial Sequence
  Synthetic primer
tgtgcatt

SEQ ID NO: 662 - DNA, 90 bp, Artificial Sequence
  Synthetic primer
caagcagaagacggcatacgagatacgcgatcgtgactggagttcagacgtgtgctcttccgatcttaaagcagcgtatccacatagcgt

SEQ ID NO: 663 - DNA, 8 bp, Artificial Sequence
  Synthetic primer
acgcgatc

SEQ ID NO: 664 - DNA, 8 bp, Artificial Sequence
  Synthetic primer
gatcgcgt

SEQ ID NO: 665 - DNA, 90 bp, Artificial Sequence
  Synthetic primer
caagcagaagacggcatacgagatactatcttgtgactggagttcagacgtgtgctcttccgatcttaaagcagcgtatccacatagcgt

SEQ ID NO: 666 - DNA, 8 bp, Artificial Sequence
  Synthetic primer
actatctt

SEQ ID NO: 667 - DNA, 8 bp, Artificial Sequence
  Synthetic primer
aagatagt

SEQ ID NO: 668 - DNA, 90 bp, Artificial Sequence
  Synthetic primer
caagcagaagacggcatacgagatactgggaggtgactggagttcagacgtgtgctcttccgatcttaaagcagcgtatccacatagcgt

SEQ ID NO: 669 - DNA, 8 bp, Artificial Sequence
  Synthetic primer
actgggag

SEQ ID NO: 670 - DNA, 8 bp, Artificial Sequence
  Synthetic primer
ctcccagt

SEQ ID NO: 671 - DNA, 90 bp, Artificial Sequence
  Synthetic primer
caagcagaagacggcatacgagatagaatcacgtgactggagttcagacgtgtgctcttccgatcttaaagcagcgtatccacatagcgt

SEQ ID NO: 672 - DNA, 8 bp, Artificial Sequence
  Synthetic primer
agaatcac

SEQ ID NO: 673 - DNA, 8 bp, Artificial Sequence
  Synthetic primer
gtgattct

SEQ ID NO: 674 - DNA, 90 bp, Artificial Sequence
  Synthetic primer
caagcagaagacggcatacgagataggattttgtgactggagttcagacgtgtgctcttccgatcttaaagcagcgtatccacatagcgt

SEQ ID NO: 675 - DNA, 8 bp, Artificial Sequence
  Synthetic primer
aggatttt

SEQ ID NO: 676 - DNA, 8 bp, Artificial Sequence
  Synthetic primer
aaaatcct

SEQ ID NO: 677 - DNA, 90 bp, Artificial Sequence
  Synthetic primer
caagcagaagacggcatacgagatagttagtcgtgactggagttcagacgtgtgctcttccgatcttaaagcagcgtatccacatagcgt

SEQ ID NO: 678 - DNA, 8 bp, Artificial Sequence
  Synthetic primer
agttagtc

SEQ ID NO: 679 - DNA, 8 bp, Artificial Sequence
  Synthetic primer
gactaact

SEQ ID NO: 680 - DNA, 90 bp, Artificial Sequence
  Synthetic primer
caagcagaagacggcatacgagatagttgagggtgactggagttcagacgtgtgctcttccgatcttaaagcagcgtatccacatagcgt

SEQ ID NO: 681 - DNA, 8 bp, Artificial Sequence
  Synthetic primer
agttgagg

SEQ ID NO: 682 - DNA, 8 bp, Artificial Sequence
  Synthetic primer
cctcaact

SEQ ID NO: 683 - DNA, 90 bp, Artificial Sequence
  Synthetic primer
caagcagaagacggcatacgagatataacgcggtgactggagttcagacgtgtgctcttccgatcttaaagcagcgtatccacatagcgt

SEQ ID NO: 684 - DNA, 8 bp, Artificial Sequence
  Synthetic primer
ataacgcg

SEQ ID NO: 685 - DNA, 8 bp, Artificial Sequence
  Synthetic primer
cgcgttat

SEQ ID NO: 686 - DNA, 90 bp, Artificial Sequence
  Synthetic primer
caagcagaagacggcatacgagatatcaaggagtgactggagttcagacgtgtgctcttccgatcttaaagcagcgtatccacatagcgt

SEQ ID NO: 687 - DNA, 8 bp, Artificial Sequence
  Synthetic primer
atcaagga

SEQ ID NO: 688 - DNA, 8 bp, Artificial Sequence
  Synthetic primer
tccttgat

SEQ ID NO: 689 - DNA, 90 bp, Artificial Sequence
  Synthetic primer
caagcagaagacggcatacgagatatcgttgggtgactggagttcagacgtgtgctcttccgatcttaaagcagcgtatccacatagcgt

SEQ ID NO: 690 - DNA, 8 bp, Artificial Sequence
  Synthetic primer
atcgttgg

SEQ ID NO: 691 - DNA, 8 bp, Artificial Sequence
  Synthetic primer
ccaacgat

SEQ ID NO: 692 - DNA, 90 bp, Artificial Sequence
  Synthetic primer
caagcagaagacggcatacgagatattggactgtgactggagttcagacgtgtgctcttccgatcttaaagcagcgtatccacatagcgt

SEQ ID NO: 693 - DNA, 8 bp, Artificial Sequence
  Synthetic primer
attggact

SEQ ID NO: 694 - DNA, 8 bp, Artificial Sequence
  Synthetic primer
agtccaat

SEQ ID NO: 695 - DNA, 90 bp, Artificial Sequence
  Synthetic primer
caagcagaagacggcatacgagatcaagcggcgtgactggagttcagacgtgtgctcttccgatcttaaagcagcgtatccacatagcgt

SEQ ID NO: 696 - DNA, 8 bp, Artificial Sequence
  Synthetic primer
caagcggc

SEQ ID NO: 697 - DNA, 8 bp, Artificial Sequence
  Synthetic primer
gccgcttg

SEQ ID NO: 698 - DNA, 90 bp, Artificial Sequence
  Synthetic primer
caagcagaagacggcatacgagatcacgctcagtgactggagttcagacgtgtgctcttccgatcttaaagcagcgtatccacatagcgt

SEQ ID NO: 699 - DNA, 8 bp, Artificial Sequence
  Synthetic primer
cacgctca

SEQ ID NO: 700 - DNA, 8 bp, Artificial Sequence
  Synthetic primer
tgagcgtg

SEQ ID NO: 701 - DNA, 90 bp, Artificial Sequence
  Synthetic primer
caagcagaagacggcatacgagatcagtttgtgtgactggagttcagacgtgtgctcttccgatcttaaagcagcgtatccacatagcgt

SEQ ID NO: 702 - DNA, 8 bp, Artificial Sequence
  Synthetic primer
cagtttgt

SEQ ID NO: 703 - DNA, 8 bp, Artificial Sequence
  Synthetic primer
acaaactg

SEQ ID NO: 704 - DNA, 90 bp, Artificial Sequence
  Synthetic primer
caagcagaagacggcatacgagatcatcgcgagtgactggagttcagacgtgtgctcttccgatcttaaagcagcgtatccacatagcgt

SEQ ID NO: 705 - DNA, 8 bp, Artificial Sequence
  Synthetic primer
catcgcga

SEQ ID NO: 706 - DNA, 8 bp, Artificial Sequence
  Synthetic primer
tcgcgatg

SEQ ID NO: 707 - DNA, 90 bp, Artificial Sequence
  Synthetic primer
caagcagaagacggcatacgagatccacaccggtgactggagttcagacgtgtgctcttccgatcttaaagcagcgtatccacatagcgt

SEQ ID NO: 708 - DNA, 8 bp, Artificial Sequence
  Synthetic primer
ccacaccg

SEQ ID NO: 709 - DNA, 8 bp, Artificial Sequence
  Synthetic primer
cggtgtgg

SEQ ID NO: 710 - DNA, 90 bp, Artificial Sequence
  Synthetic primer
caagcagaagacggcatacgagatccactgtcgtgactggagttcagacgtgtgctcttccgatcttaaagcagcgtatccacatagcgt

SEQ ID NO: 711 - DNA, 8 bp, Artificial Sequence
  Synthetic primer
ccactgtc

SEQ ID NO: 712 - DNA, 8 bp, Artificial Sequence
  Synthetic primer
gacagtgg

SEQ ID NO: 713 - DNA, 90 bp, Artificial Sequence
  Synthetic primer
caagcagaagacggcatacgagatcccacaacgtgactggagttcagacgtgtgctcttccgatcttaaagcagcgtatccacatagcgt

SEQ ID NO: 714 - DNA, 8 bp, Artificial Sequence
  Synthetic primer
cccacaac

SEQ ID NO: 715 - DNA, 8 bp, Artificial Sequence
  Synthetic primer
gttgtggg

SEQ ID NO: 716 - DNA, 90 bp, Artificial Sequence
  Synthetic primer
caagcagaagacggcatacgagatcccgtatagtgactggagttcagacgtgtgctcttccgatcttaaagcagcgtatccacatagcgt

SEQ ID NO: 717 - DNA, 8 bp, Artificial Sequence
  Synthetic primer
cccgtata

SEQ ID NO: 718 - DNA, 8 bp, Artificial Sequence
  Synthetic primer
tatacggg

SEQ ID NO: 719 - DNA, 90 bp, Artificial Sequence
  Synthetic primer
caagcagaagacggcatacgagatccctagtcgtgactggagttcagacgtgtgctcttccgatcttaaagcagcgtatccacatagcgt

SEQ ID NO: 720 - DNA, 8 bp, Artificial Sequence
  Synthetic primer
ccctagtc

SEQ ID NO: 721 - DNA, 8 bp, Artificial Sequence
  Synthetic primer
gactaggg

SEQ ID NO: 722 - DNA, 90 bp, Artificial Sequence
  Synthetic primer
caagcagaagacggcatacgagatccgtacgggtgactggagttcagacgtgtgctcttccgatcttaaagcagcgtatccacatagcgt

SEQ ID NO: 723 - DNA, 8 bp, Artificial Sequence
  Synthetic primer
ccgtacgg

SEQ ID NO: 724 - DNA, 8 bp, Artificial Sequence
  Synthetic primer
ccgtacgg

SEQ ID NO: 725 - DNA, 90 bp, Artificial Sequence
  Synthetic primer
caagcagaagacggcatacgagatcgacgaaggtgactggagttcagacgtgtgctcttccgatcttaaagcagcgtatccacatagcgt

SEQ ID NO: 726 - DNA, 8 bp, Artificial Sequence
  Synthetic primer
cgacgaag

SEQ ID NO: 727 - DNA, 8 bp, Artificial Sequence
  Synthetic primer
cttcgtcg

SEQ ID NO: 728 - DNA, 90 bp, Artificial Sequence
  Synthetic primer
caagcagaagacggcatacgagatctgaccgcgtgactggagttcagacgtgtgctcttccgatcttaaagcagcgtatccacatagcgt

SEQ ID NO: 729 - DNA, 8 bp, Artificial Sequence
  Synthetic primer
ctgaccgc

SEQ ID NO: 730 - DNA, 8 bp, Artificial Sequence
  Synthetic primer
gcggtcag

SEQ ID NO: 731 - DNA, 90 bp, Artificial Sequence
  Synthetic primer
caagcagaagacggcatacgagatgcagtgcggtgactggagttcagacgtgtgctcttccgatcttaaagcagcgtatccacatagcgt

SEQ ID NO: 732 - DNA, 8 bp, Artificial Sequence
  Synthetic primer
gcagtgcg

SEQ ID NO: 733 - DNA, 8 bp, Artificial Sequence
  Synthetic primer
cgcactgc

SEQ ID NO: 734 - DNA, 90 bp, Artificial Sequence
  Synthetic primer
caagcagaagacggcatacgagatgctaggatgtgactggagttcagacgtgtgctcttccgatcttaaagcagcgtatccacatagcgt

SEQ ID NO: 735 - DNA, 8 bp, Artificial Sequence
  Synthetic primer
gctaggat

SEQ ID NO: 736 - DNA, 8 bp, Artificial Sequence
  Synthetic primer
atcctagc

SEQ ID NO: 737 - DNA, 90 bp, Artificial Sequence
  Synthetic primer
caagcagaagacggcatacgagatgctccagagtgactggagttcagacgtgtgctcttccgatcttaaagcagcgtatccacatagcgt

SEQ ID NO: 738 - DNA, 8 bp, Artificial Sequence
  Synthetic primer
gctccaga

SEQ ID NO: 739 - DNA, 8 bp, Artificial Sequence
  Synthetic primer
tctggagc

SEQ ID NO: 740 - DNA, 90 bp, Artificial Sequence
  Synthetic primer
caagcagaagacggcatacgagatgtccgtcagtgactggagttcagacgtgtgctcttccgatcttaaagcagcgtatccacatagcgt

SEQ ID NO: 741 - DNA, 8 bp, Artificial Sequence
  Synthetic primer
gtccgtca

SEQ ID NO: 742 - DNA, 8 bp, Artificial Sequence
  Synthetic primer
tgacggac

SEQ ID NO: 743 - DNA, 90 bp, Artificial Sequence
  Synthetic primer
caagcagaagacggcatacgagatgtgggttcgtgactggagttcagacgtgtgctcttccgatcttaaagcagcgtatccacatagcgt

SEQ ID NO: 744 - DNA, 8 bp, Artificial Sequence
  Synthetic primer
gtgggttc

SEQ ID NO: 745 - DNA, 8 bp, Artificial Sequence
  Synthetic primer
gaacccac

SEQ ID NO: 746 - DNA, 90 bp, Artificial Sequence
  Synthetic primer
caagcagaagacggcatacgagatgtgtggaggtgactggagttcagacgtgtgctcttccgatcttaaagcagcgtatccacatagcgt

SEQ ID NO: 747 - DNA, 8 bp, Artificial Sequence
  Synthetic primer
gtgtggag

SEQ ID NO: 748 - DNA, 8 bp, Artificial Sequence
  Synthetic primer
ctccacac

SEQ ID NO: 749 - DNA, 90 bp, Artificial Sequence
  Synthetic primer
caagcagaagacggcatacgagatgttaagaggtgactggagttcagacgtgtgctcttccgatcttaaagcagcgtatccacatagcgt

SEQ ID NO: 750 - DNA, 8 bp, Artificial Sequence
  Synthetic primer
gttaagag

SEQ ID NO: 751 - DNA, 8 bp, Artificial Sequence
  Synthetic primer
ctcttaac

SEQ ID NO: 752 - DNA, 90 bp, Artificial Sequence
  Synthetic primer
caagcagaagacggcatacgagatgttccggggtgactggagttcagacgtgtgctcttccgatcttaaagcagcgtatccacatagcgt

SEQ ID NO: 753 - DNA, 8 bp, Artificial Sequence
  Synthetic primer
gttccggg

SEQ ID NO: 754 - DNA, 8 bp, Artificial Sequence
  Synthetic primer
cccggaac

SEQ ID NO: 755 - DNA, 90 bp, Artificial Sequence
  Synthetic primer
caagcagaagacggcatacgagatgttgggttgtgactggagttcagacgtgtgctcttccgatcttaaagcagcgtatccacatagcgt

SEQ ID NO: 756 - DNA, 8 bp, Artificial Sequence
  Synthetic primer
gttgggtt

SEQ ID NO: 757 - DNA, 8 bp, Artificial Sequence
  Synthetic primer
aacccaac

SEQ ID NO: 758 - DNA, 90 bp, Artificial Sequence
  Synthetic primer
caagcagaagacggcatacgagattaccatgtgtgactggagttcagacgtgtgctcttccgatcttaaagcagcgtatccacatagcgt

SEQ ID NO: 759 - DNA, 8 bp, Artificial Sequence
  Synthetic primer
taccatgt

SEQ ID NO: 760 - DNA, 8 bp, Artificial Sequence
  Synthetic primer
acatggta

SEQ ID NO: 761 - DNA, 90 bp, Artificial Sequence
  Synthetic primer
caagcagaagacggcatacgagattacgggcggtgactggagttcagacgtgtgctcttccgatcttaaagcagcgtatccacatagcgt

SEQ ID NO: 762 - DNA, 8 bp, Artificial Sequence
  Synthetic primer
tacgggcg

SEQ ID NO: 763 - DNA, 8 bp, Artificial Sequence
  Synthetic primer
cgcccgta

SEQ ID NO: 764 - DNA, 90 bp, Artificial Sequence
  Synthetic primer
caagcagaagacggcatacgagattcaatcacgtgactggagttcagacgtgtgctcttccgatcttaaagcagcgtatccacatagcgt

SEQ ID NO: 765 - DNA, 8 bp, Artificial Sequence
  Synthetic primer
tcaatcac

SEQ ID NO: 766 - DNA, 8 bp, Artificial Sequence
  Synthetic primer
gtgattga

SEQ ID NO: 767 - DNA, 90 bp, Artificial Sequence
  Synthetic primer
caagcagaagacggcatacgagattcataccagtgactggagttcagacgtgtgctcttccgatcttaaagcagcgtatccacatagcgt

SEQ ID NO: 768 - DNA, 8 bp, Artificial Sequence
  Synthetic primer
tcatacca

SEQ ID NO: 769 - DNA, 8 bp, Artificial Sequence
  Synthetic primer
tggtatga

SEQ ID NO: 770 - DNA, 90 bp, Artificial Sequence
  Synthetic primer
caagcagaagacggcatacgagattccggttggtgactggagttcagacgtgtgctcttccgatcttaaagcagcgtatccacatagcgt

SEQ ID NO: 771 - DNA, 8 bp, Artificial Sequence
  Synthetic primer
tccggttg

SEQ ID NO: 772 - DNA, 8 bp, Artificial Sequence
  Synthetic primer
caaccgga

SEQ ID NO: 773 - DNA, 90 bp, Artificial Sequence
  Synthetic primer
caagcagaagacggcatacgagattgacttgtgtgactggagttcagacgtgtgctcttccgatcttaaagcagcgtatccacatagcgt

SEQ ID NO: 774 - DNA, 8 bp, Artificial Sequence
  Synthetic primer
tgacttgt

SEQ ID NO: 775 - DNA, 8 bp, Artificial Sequence
  Synthetic primer
acaagtca

SEQ ID NO: 776 - DNA, 90 bp, Artificial Sequence
  Synthetic primer
caagcagaagacggcatacgagattgctgctcgtgactggagttcagacgtgtgctcttccgatcttaaagcagcgtatccacatagcgt

SEQ ID NO: 777 - DNA, 8 bp, Artificial Sequence
  Synthetic primer
tgctgctc

SEQ ID NO: 778 - DNA, 8 bp, Artificial Sequence
  Synthetic primer
gagcagca

SEQ ID NO: 779 - DNA, 90 bp, Artificial Sequence
  Synthetic primer
caagcagaagacggcatacgagattgtagaccgtgactggagttcagacgtgtgctcttccgatcttaaagcagcgtatccacatagcgt

SEQ ID NO: 780 - DNA, 8 bp, Artificial Sequence
  Synthetic primer
tgtagacc

SEQ ID NO: 781 - DNA, 8 bp, Artificial Sequence
  Synthetic primer
ggtctaca

SEQ ID NO: 782 - DNA, 90 bp, Artificial Sequence
  Synthetic primer
caagcagaagacggcatacgagatttacgttggtgactggagttcagacgtgtgctcttccgatcttaaagcagcgtatccacatagcgt

SEQ ID NO: 783 - DNA, 8 bp, Artificial Sequence
  Synthetic primer
ttacgttg

SEQ ID NO: 784 - DNA, 8 bp, Artificial Sequence
  Synthetic primer
caacgtaa

SEQ ID NO: 785 - DNA, 90 bp, Artificial Sequence
  Synthetic primer
caagcagaagacggcatacgagatttcgcggagtgactggagttcagacgtgtgctcttccgatcttaaagcagcgtatccacatagcgt

SEQ ID NO: 786 - DNA, 8 bp, Artificial Sequence
  Synthetic primer
ttcgcgga

SEQ ID NO: 787 - DNA, 8 bp, Artificial Sequence
  Synthetic primer
tccgcgaa

SEQ ID NO: 788 - DNA, 90 bp, Artificial Sequence
  Synthetic primer
caagcagaagacggcatacgagatttgatcgggtgactggagttcagacgtgtgctcttccgatcttaaagcagcgtatccacatagcgt

SEQ ID NO: 789 - DNA, 8 bp, Artificial Sequence
  Synthetic primer
ttgatcgg

SEQ ID NO: 790 - DNA, 8 bp, Artificial Sequence
  Synthetic primer
ccgatcaa

SEQ ID NO: 791 - DNA, 90 bp, Artificial Sequence
  Synthetic primer
caagcagaagacggcatacgagattttgcagtgtgactggagttcagacgtgtgctcttccgatcttaaagcagcgtatccacatagcgt

SEQ ID NO: 792 - DNA, 8 bp, Artificial Sequence
  Synthetic primer
tttgcagt

SEQ ID NO: 793 - DNA, 8 bp, Artificial Sequence
  Synthetic primer
actgcaaa

SEQ ID NO: 794 - DNA, 90 bp, Artificial Sequence
  Synthetic primer
caagcagaagacggcatacgagatcagtcgatgtgactggagttcagacgtgtgctcttccgatcttaaagcagcgtatccacatagcgt

SEQ ID NO: 795 - DNA, 8 bp, Artificial Sequence
  Synthetic primer
cagtcgat

SEQ ID NO: 796 - DNA, 8 bp, Artificial Sequence
  Synthetic primer
atcgactg

SEQ ID NO: 797 - DNA, 90 bp, Artificial Sequence
  Synthetic primer
caagcagaagacggcatacgagatctgctagcgtgactggagttcagacgtgtgctcttccgatcttaaagcagcgtatccacatagcgt

SEQ ID NO: 798 - DNA, 8 bp, Artificial Sequence
  Synthetic primer
ctgctagc

SEQ ID NO: 799 - DNA, 8 bp, Artificial Sequence
  Synthetic primer
gctagcag

SEQ ID NO: 800 - DNA, 90 bp, Artificial Sequence
  Synthetic primer
caagcagaagacggcatacgagatggagagtagtgactggagttcagacgtgtgctcttccgatcttaaagcagcgtatccacatagcgt

SEQ ID NO: 801 - DNA, 8 bp, Artificial Sequence
  Synthetic primer
ggagagta

SEQ ID NO: 802 - DNA, 8 bp, Artificial Sequence
  Synthetic primer
tactctcc

SEQ ID NO: 803 - DNA, 90 bp, Artificial Sequence
  Synthetic primer
caagcagaagacggcatacgagattgctgtcagtgactggagttcagacgtgtgctcttccgatcttaaagcagcgtatccacatagcgt

SEQ ID NO: 804 - DNA, 8 bp, Artificial Sequence
  Synthetic primer
tgctgtca

SEQ ID NO: 805 - DNA, 8 bp, Artificial Sequence
  Synthetic primer
tgacagca

SEQ ID NO: 806 - DNA, 90 bp, Artificial Sequence
  Synthetic primer
caagcagaagacggcatacgagatcaacctgcgtgactggagttcagacgtgtgctcttccgatcttaaagcagcgtatccacatagcgt

SEQ ID NO: 807 - DNA, 8 bp, Artificial Sequence
  Synthetic primer
caacctgc

SEQ ID NO: 808 - DNA, 8 bp, Artificial Sequence
  Synthetic primer
gcaggttg

SEQ ID NO: 809 - DNA, 90 bp, Artificial Sequence
  Synthetic primer
caagcagaagacggcatacgagatagctggaagtgactggagttcagacgtgtgctcttccgatcttaaagcagcgtatccacatagcgt

SEQ ID NO: 810 - DNA, 8 bp, Artificial Sequence
  Synthetic primer
agctggaa

SEQ ID NO: 811 - DNA, 8 bp, Artificial Sequence
  Synthetic primer
ttccagct

SEQ ID NO: 812 - DNA, 90 bp, Artificial Sequence
  Synthetic primer
caagcagaagacggcatacgagatgctaactagtgactggagttcagacgtgtgctcttccgatcttaaagcagcgtatccacatagcgt

SEQ ID NO: 813 - DNA, 8 bp, Artificial Sequence
  Synthetic primer
gctaacta

SEQ ID NO: 814 - DNA, 8 bp, Artificial Sequence
  Synthetic primer
tagttagc

SEQ ID NO: 815 - DNA, 90 bp, Artificial Sequence
  Synthetic primer
caagcagaagacggcatacgagatttagcgctgtgactggagttcagacgtgtgctcttccgatcttaaagcagcgtatccacatagcgt

SEQ ID NO: 816 - DNA, 8 bp, Artificial Sequence
  Synthetic primer
ttagcgct

SEQ ID NO: 817 - DNA, 8 bp, Artificial Sequence
  Synthetic primer
agcgctaa

SEQ ID NO: 818 - DNA, 90 bp, Artificial Sequence
  Synthetic primer
caagcagaagacggcatacgagataagaaccggtgactggagttcagacgtgtgctcttccgatcttaaagcagcgtatccacatagcgt

SEQ ID NO: 819 - DNA, 8 bp, Artificial Sequence
  Synthetic primer
aagaaccg

SEQ ID NO: 820 - DNA, 8 bp, Artificial Sequence
  Synthetic primer
cggttctt

SEQ ID NO: 821 - DNA, 90 bp, Artificial Sequence
  Synthetic primer
caagcagaagacggcatacgagatcaatgctagtgactggagttcagacgtgtgctcttccgatcttaaagcagcgtatccacatagcgt

SEQ ID NO: 822 - DNA, 8 bp, Artificial Sequence
  Synthetic primer
caatgcta

SEQ ID NO: 823 - DNA, 8 bp, Artificial Sequence
  Synthetic primer
tagcattg

SEQ ID NO: 824 - DNA, 90 bp, Artificial Sequence
  Synthetic primer
caagcagaagacggcatacgagatgttgaattgtgactggagttcagacgtgtgctcttccgatcttaaagcagcgtatccacatagcgt

SEQ ID NO: 825 - DNA, 8 bp, Artificial Sequence
  Synthetic primer
gttgaatt

SEQ ID NO: 826 - DNA, 8 bp, Artificial Sequence
  Synthetic primer
aattcaac

SEQ ID NO: 827 - DNA, 90 bp, Artificial Sequence
  Synthetic primer
caagcagaagacggcatacgagattctgtgaagtgactggagttcagacgtgtgctcttccgatcttaaagcagcgtatccacatagcgt

SEQ ID NO: 828 - DNA, 8 bp, Artificial Sequence
  Synthetic primer
tctgtgaa

SEQ ID NO: 829 - DNA, 8 bp, Artificial Sequence
  Synthetic primer
ttcacaga

SEQ ID NO: 830 - DNA, 90 bp, Artificial Sequence
  Synthetic primer
caagcagaagacggcatacgagataagagagcgtgactggagttcagacgtgtgctcttccgatcttaaagcagcgtatccacatagcgt

SEQ ID NO: 831 - DNA, 8 bp, Artificial Sequence
  Synthetic primer
aagagagc

SEQ ID NO: 832 - DNA, 8 bp, Artificial Sequence
  Synthetic primer
gctctctt

SEQ ID NO: 833 - DNA, 98 bp, Artificial Sequence
  Synthetic primer
caagcagaagacggcatacgagatccaagtcagtgactggagttcagacgtgtgctcttccgatcttaaagcagcgtatccacatagcgtaagagagc

SEQ ID NO: 834 - DNA, 16 bp, Artificial Sequence
  Synthetic primer
ccaagtcaaagagagc

SEQ ID NO: 835 - DNA, 16 bp, Artificial Sequence
  Synthetic primer
tgacttggaagagagc

SEQ ID NO: 836 - DNA, 98 bp, Artificial Sequence
  Synthetic primer
caagcagaagacggcatacgagatgaaccatagtgactggagttcagacgtgtgctcttccgatcttaaagcagcgtatccacatagcgtaagagagc

SEQ ID NO: 837 - DNA, 16 bp, Artificial Sequence
  Synthetic primer
gaaccataaagagagc

SEQ ID NO: 838 - DNA, 16 bp, Artificial Sequence
  Synthetic primer
tatggttcaagagagc

SEQ ID NO: 839 - DNA, 90 bp, Artificial Sequence
  Synthetic primer
caagcagaagacggcatacgagatggctagtggtgactggagttcagacgtgtgctcttccgatcttaaagcagcgtatccacatagcgt

SEQ ID NO: 840 - DNA, 8 bp, Artificial Sequence
  Synthetic primer
ggctagtg

SEQ ID NO: 841 - DNA, 8 bp, Artificial Sequence
  Synthetic primer
cactagcc

SEQ ID NO: 842 - DNA, 90 bp, Artificial Sequence
  Synthetic primer
caagcagaagacggcatacgagataagaggttgtgactggagttcagacgtgtgctcttccgatcttaaagcagcgtatccacatagcgt

SEQ ID NO: 843 - DNA, 8 bp, Artificial Sequence
  Synthetic primer
aagaggtt

SEQ ID NO: 844 - DNA, 8 bp, Artificial Sequence
  Synthetic primer
aacctctt

SEQ ID NO: 845 - DNA, 90 bp, Artificial Sequence
  Synthetic primer
caagcagaagacggcatacgagatcaatgtaggtgactggagttcagacgtgtgctcttccgatcttaaagcagcgtatccacatagcgt

SEQ ID NO: 846 - DNA, 8 bp, Artificial Sequence
  Synthetic primer
caatgtag

SEQ ID NO: 847 - DNA, 8 bp, Artificial Sequence
  Synthetic primer
ctacattg

SEQ ID NO: 848 - DNA, 84 bp, Artificial Sequence
  Synthetic primer
aatgatacggcgaccaccgagatctacactctttccctacacgacgctcttccgatctatcgtacgatcttgtggaaaggacga

SEQ ID NO: 849 - DNA, 8 bp, Artificial Sequence
  Synthetic primer
atcgtacg

SEQ ID NO: 850 - DNA, 85 bp, Artificial Sequence
  Synthetic primer
aatgatacggcgaccaccgagatctacactctttccctacacgacgctcttccgatctactatctggatcttgtggaaaggacga

SEQ ID NO: 851 - DNA, 8 bp, Artificial Sequence
  Synthetic primer
actatctg

SEQ ID NO: 852 - DNA, 86 bp, Artificial Sequence
  Synthetic primer
aatgatacggcgaccaccgagatctacactctttccctacacgacgctcttccgatcttagcgagtcgatcttgtggaaaggacga

SEQ ID NO: 853 - DNA, 8 bp, Artificial Sequence
  Synthetic primer
tagcgagt

SEQ ID NO: 854 - DNA, 87 bp, Artificial Sequence
  Synthetic primer
aatgatacggcgaccaccgagatctacactctttccctacacgacgctcttccgatctctgcgtgtacgatcttgtggaaaggacga

SEQ ID NO: 855 - DNA, 8 bp, Artificial Sequence
  Synthetic primer
ctgcgtgt

SEQ ID NO: 856 - DNA, 84 bp, Artificial Sequence
  Synthetic primer
aatgatacggcgaccaccgagatctacactctttccctacacgacgctcttccgatcttcatcgagatcttgtggaaaggacga

SEQ ID NO: 857 - DNA, 8 bp, Artificial Sequence
  Synthetic primer
tcatcgag

SEQ ID NO: 858 - DNA, 85 bp, Artificial Sequence
  Synthetic primer
aatgatacggcgaccaccgagatctacactctttccctacacgacgctcttccgatctcgtgagtggatcttgtggaaaggacga

SEQ ID NO: 859 - DNA, 8 bp, Artificial Sequence
  Synthetic primer
cgtgagtg

SEQ ID NO: 860 - DNA, 86 bp, Artificial Sequence
  Synthetic primer
aatgatacggcgaccaccgagatctacactctttccctacacgacgctcttccgatctggatatctcgatcttgtggaaaggacga

SEQ ID NO: 861 - DNA, 8 bp, Artificial Sequence
  Synthetic primer
ggatatct

SEQ ID NO: 862 - DNA, 87 bp, Artificial Sequence
  Synthetic primer
aatgatacggcgaccaccgagatctacactctttccctacacgacgctcttccgatctgacaccgtacgatcttgtggaaaggacga

SEQ ID NO: 863 - DNA, 8 bp, Artificial Sequence
  Synthetic primer
gacaccgt

SEQ ID NO: 864 - DNA, 84 bp, Artificial Sequence
  Synthetic primer
aatgatacggcgaccaccgagatctacactctttccctacacgacgctcttccgatctctactataatcttgtggaaaggacga

SEQ ID NO: 865 - DNA, 8 bp, Artificial Sequence
  Synthetic primer
ctactata

SEQ ID NO: 866 - DNA, 85 bp, Artificial Sequence
  Synthetic primer
aatgatacggcgaccaccgagatctacactctttccctacacgacgctcttccgatctcgttactagatcttgtggaaaggacga

SEQ ID NO: 867 - DNA, 8 bp, Artificial Sequence
  Synthetic primer
cgttacta

SEQ ID NO: 868 - DNA, 86 bp, Artificial Sequence
  Synthetic primer
aatgatacggcgaccaccgagatctacactctttccctacacgacgctcttccgatctagagtcaccgatcttgtggaaaggacga

SEQ ID NO: 869 - DNA, 8 bp, Artificial Sequence
  Synthetic primer
agagtcac

SEQ ID NO: 870 - DNA, 87 bp, Artificial Sequence
  Synthetic primer
aatgatacggcgaccaccgagatctacactctttccctacacgacgctcttccgatcttacgagacacgatcttgtggaaaggacga

SEQ ID NO: 871 - DNA, 8 bp, Artificial Sequence
  Synthetic primer
tacgagac

SEQ ID NO: 872 - DNA, 84 bp, Artificial Sequence
  Synthetic primer
aatgatacggcgaccaccgagatctacactctttccctacacgacgctcttccgatctacgtctcgatcttgtggaaaggacga

SEQ ID NO: 873 - DNA, 8 bp, Artificial Sequence
  Synthetic primer
acgtctcg

SEQ ID NO: 874 - DNA, 85 bp, Artificial Sequence
  Synthetic primer
aatgatacggcgaccaccgagatctacactctttccctacacgacgctcttccgatcttcgacgaggatcttgtggaaaggacga

SEQ ID NO: 875 - DNA, 8 bp, Artificial Sequence
  Synthetic primer
tcgacgag

SEQ ID NO: 876 - DNA, 86 bp, Artificial Sequence
  Synthetic primer
aatgatacggcgaccaccgagatctacactctttccctacacgacgctcttccgatctgatcgtgtcgatcttgtggaaaggacga

SEQ ID NO: 877 - DNA, 8 bp, Artificial Sequence
  Synthetic primer
gatcgtgt

SEQ ID NO: 878 - DNA, 87 bp, Artificial Sequence
  Synthetic primer
aatgatacggcgaccaccgagatctacactctttccctacacgacgctcttccgatctgtcagataacgatcttgtggaaaggacga

SEQ ID NO: 879 - DNA, 8 bp, Artificial Sequence
  Synthetic primer
gtcagata

SEQ ID NO: 880 - DNA, 84 bp, Artificial Sequence
  Synthetic primer
aatgatacggcgaccaccgagatctacactctttccctacacgacgctcttccgatctacgacgtgatcttgtggaaaggacga

SEQ ID NO: 881 - DNA, 8 bp, Artificial Sequence
  Synthetic primer
acgacgtg

SEQ ID NO: 882 - DNA, 85 bp, Artificial Sequence
  Synthetic primer
aatgatacggcgaccaccgagatctacactctttccctacacgacgctcttccgatctatatacacgatcttgtggaaaggacga

SEQ ID NO: 883 - DNA, 8 bp, Artificial Sequence
  Synthetic primer
atatacac

SEQ ID NO: 884 - DNA, 86 bp, Artificial Sequence
  Synthetic primer
aatgatacggcgaccaccgagatctacactctttccctacacgacgctcttccgatctcgtcgctacgatcttgtggaaaggacga

SEQ ID NO: 885 - DNA, 8 bp, Artificial Sequence
  Synthetic primer
cgtcgcta

SEQ ID NO: 886 - DNA, 87 bp, Artificial Sequence
  Synthetic primer
aatgatacggcgaccaccgagatctacactctttccctacacgacgctcttccgatctctagagctacgatcttgtggaaaggacga

SEQ ID NO: 887 - DNA, 8 bp, Artificial Sequence
  Synthetic primer
ctagagct

SEQ ID NO: 888 - DNA, 84 bp, Artificial Sequence
  Synthetic primer
aatgatacggcgaccaccgagatctacactctttccctacacgacgctcttccgatctgctctagtatcttgtggaaaggacga

SEQ ID NO: 889 - DNA, 8 bp, Artificial Sequence
  Synthetic primer
gctctagt

SEQ ID NO: 890 - DNA, 25 bp, Artificial Sequence
  Synthetic primer
cactgagatcttgtggaaaggacga

SEQ ID NO: 891 - DNA, 8 bp, Artificial Sequence
  Synthetic primer
gacactga

SEQ ID NO: 892 - DNA, 86 bp, Artificial Sequence
  Synthetic primer
aatgatacggcgaccaccgagatctacactctttccctacacgacgctcttccgatcttgcgtacgcgatcttgtggaaaggacga

SEQ ID NO: 893 - DNA, 8 bp, Artificial Sequence
  Synthetic primer
tgcgtacg

SEQ ID NO: 894 - DNA, 87 bp, Artificial Sequence
  Synthetic primer
aatgatacggcgaccaccgagatctacactctttccctacacgacgctcttccgatcttagtgtagacgatcttgtggaaaggacga

SEQ ID NO: 895 - DNA, 8 bp, Artificial Sequence
  Synthetic primer
tagtgtag

SEQ ID NO: 896 - DNA, 84 bp, Artificial Sequence
  Synthetic primer
aatgatacggcgaccaccgagatctacactctttccctacacgacgctcttccgatctaagcagcaatcttgtggaaaggacga

SEQ ID NO: 897 - DNA, 8 bp, Artificial Sequence
  Synthetic primer
aagcagca

SEQ ID NO: 898 - DNA, 85 bp, Artificial Sequence
  Synthetic primer
aatgatacggcgaccaccgagatctacactctttccctacacgacgctcttccgatctacgcgtgagatcttgtggaaaggacga

SEQ ID NO: 899 - DNA, 8 bp, Artificial Sequence
  Synthetic primer
acgcgtga

SEQ ID NO: 900 - DNA, 86 bp, Artificial Sequence
  Synthetic primer
aatgatacggcgaccaccgagatctacactctttccctacacgacgctcttccgatctcgatctaccgatcttgtggaaaggacga

SEQ ID NO: 901 - DNA, 8 bp, Artificial Sequence
  Synthetic primer
cgatctac

SEQ ID NO: 902 - DNA, 87 bp, Artificial Sequence
  Synthetic primer
aatgatacggcgaccaccgagatctacactctttccctacacgacgctcttccgatcttgcgtcacacgatcttgtggaaaggacga

SEQ ID NO: 903 - DNA, 8 bp, Artificial Sequence
  Synthetic primer
tgcgtcac

SEQ ID NO: 904 - DNA, 84 bp, Artificial Sequence
  Synthetic primer
aatgatacggcgaccaccgagatctacactctttccctacacgacgctcttccgatctgtctagtgatcttgtggaaaggacga

SEQ ID NO: 905 - DNA, 8 bp, Artificial Sequence
  Synthetic primer
gtctagtg

SEQ ID NO: 906 - DNA, 85 bp, Artificial Sequence
  Synthetic primer
aatgatacggcgaccaccgagatctacactctttccctacacgacgctcttccgatctctagtatggatcttgtggaaaggacga

SEQ ID NO: 907 - DNA, 8 bp, Artificial Sequence
  Synthetic primer
ctagtatg

SEQ ID NO: 908 - DNA, 86 bp, Artificial Sequence
  Synthetic primer
aatgatacggcgaccaccgagatctacactctttccctacacgacgctcttccgatctgatagcgtcgatcttgtggaaaggacga

SEQ ID NO: 909 - DNA, 8 bp, Artificial Sequence
  Synthetic primer
gatagcgt

SEQ ID NO: 910 - DNA, 87 bp, Artificial Sequence
  Synthetic primer
aatgatacggcgaccaccgagatctacactctttccctacacgacgctcttccgatcttctacactacgatcttgtggaaaggacga

SEQ ID NO: 911 - DNA, 8 bp, Artificial Sequence
  Synthetic primer
tctacact

SEQ ID NO: 912 - DNA, 84 bp, Artificial Sequence
  Synthetic primer
aatgatacggcgaccaccgagatctacactctttccctacacgacgctcttccgatctaactctcgatcttgtggaaaggacga

SEQ ID NO: 913 - DNA, 8 bp, Artificial Sequence
  Synthetic primer
aactctcg

SEQ ID NO: 914 - DNA, 85 bp, Artificial Sequence
  Synthetic primer
aatgatacggcgaccaccgagatctacactctttccctacacgacgctcttccgatctactatgtcgatcttgtggaaaggacga

SEQ ID NO: 915 - DNA, 8 bp, Artificial Sequence
  Synthetic primer
actatgtc

SEQ ID NO: 916 - DNA, 86 bp, Artificial Sequence
  Synthetic primer
aatgatacggcgaccaccgagatctacactctttccctacacgacgctcttccgatctagtagcgtcgatcttgtggaaaggacga

SEQ ID NO: 917 - DNA, 8 bp, Artificial Sequence
  Synthetic primer
agtagcgt

SEQ ID NO: 918 - DNA, 87 bp, Artificial Sequence
  Synthetic primer
aatgatacggcgaccaccgagatctacactctttccctacacgacgctcttccgatctcagtgagtacgatcttgtggaaaggacga

SEQ ID NO: 919 - DNA, 8 bp, Artificial Sequence
  Synthetic primer
cagtgagt

SEQ ID NO: 920 - DNA, 84 bp, Artificial Sequence
  Synthetic primer
aatgatacggcgaccaccgagatctacactctttccctacacgacgctcttccgatctcgtactcaatcttgtggaaaggacga

SEQ ID NO: 921 - DNA, 8 bp, Artificial Sequence
  Synthetic primer
cgtactca

SEQ ID NO: 922 - DNA, 85 bp, Artificial Sequence
  Synthetic primer
aatgatacggcgaccaccgagatctacactctttccctacacgacgctcttccgatctctacgcaggatcttgtggaaaggacga

SEQ ID NO: 923 - DNA, 8 bp, Artificial Sequence
  Synthetic primer
ctacgcag

SEQ ID NO: 924 - DNA, 86 bp, Artificial Sequence
  Synthetic primer
aatgatacggcgaccaccgagatctacactctttccctacacgacgctcttccgatctggagactacgatcttgtggaaaggacga

SEQ ID NO: 925 - DNA, 8 bp, Artificial Sequence
  Synthetic primer
ggagacta

SEQ ID NO: 926 - DNA, 87 bp, Artificial Sequence
  Synthetic primer
aatgatacggcgaccaccgagatctacactctttccctacacgacgctcttccgatctgtcgctcgacgatcttgtggaaaggacga

SEQ ID NO: 927 - DNA, 8 bp, Artificial Sequence
  Synthetic primer
gtcgctcg

SEQ ID NO: 928 - DNA, 84 bp, Artificial Sequence
  Synthetic primer
aatgatacggcgaccaccgagatctacactctttccctacacgacgctcttccgatctgtcgtagtatcttgtggaaaggacga

SEQ ID NO: 929 - DNA, 8 bp, Artificial Sequence
  Synthetic primer
gtcgtagt

SEQ ID NO: 930 - DNA, 85 bp, Artificial Sequence
  Synthetic primer
aatgatacggcgaccaccgagatctacactctttccctacacgacgctcttccgatcttagcagacgatcttgtggaaaggacga

SEQ ID NO: 931 - DNA, 8 bp, Artificial Sequence
  Synthetic primer
tagcagac

SEQ ID NO: 932 - DNA, 86 bp, Artificial Sequence
  Synthetic primer
aatgatacggcgaccaccgagatctacactctttccctacacgacgctcttccgatcttcatagaccgatcttgtggaaaggacga

SEQ ID NO: 933 - DNA, 8 bp, Artificial Sequence
  Synthetic primer
tcatagac

SEQ ID NO: 934 - DNA, 87 bp, Artificial Sequence
  Synthetic primer
aatgatacggcgaccaccgagatctacactctttccctacacgacgctcttccgatcttcgctataacgatcttgtggaaaggacga

SEQ ID NO: 935 - DNA, 8 bp, Artificial Sequence
  Synthetic primer
tcgctata

SEQ ID NO: 936 - DNA, 84 bp, Artificial Sequence
  Synthetic primer
aatgatacggcgaccaccgagatctacactctttccctacacgacgctcttccgatctaagtcgagatcttgtggaaaggacga

SEQ ID NO: 937 - DNA, 8 bp, Artificial Sequence
  Synthetic primer
aagtcgag

SEQ ID NO: 938 - DNA, 85 bp, Artificial Sequence
  Synthetic primer
aatgatacggcgaccaccgagatctacactctttccctacacgacgctcttccgatctatacttcggatcttgtggaaaggacga

SEQ ID NO: 939 - DNA, 8 bp, Artificial Sequence
  Synthetic primer
atacttcg

SEQ ID NO: 940 - DNA, 86 bp, Artificial Sequence
  Synthetic primer
aatgatacggcgaccaccgagatctacactctttccctacacgacgctcttccgatctagctgctacgatcttgtggaaaggacga

SEQ ID NO: 941 - DNA, 8 bp, Artificial Sequence
  Synthetic primer
agctgcta

SEQ ID NO: 942 - DNA, 87 bp, Artificial Sequence
  Synthetic primer
aatgatacggcgaccaccgagatctacactctttccctacacgacgctcttccgatctcatagagaacgatcttgtggaaaggacga

SEQ ID NO: 943 - DNA, 8 bp, Artificial Sequence
  Synthetic primer
catagaga

SEQ ID NO: 944 - DNA, 84 bp, Artificial Sequence
  Synthetic primer
aatgatacggcgaccaccgagatctacactctttccctacacgacgctcttccgatctgctaatagatcttgtggaaaggacga

SEQ ID NO: 945 - DNA, 8 bp, Artificial Sequence
  Synthetic primer
gctaatag

SEQ ID NO: 946 - DNA, 85 bp, Artificial Sequence
  Synthetic primer
aatgatacggcgaccaccgagatctacactctttccctacacgacgctcttccgatcttggttggagatcttgtggaaaggacga

SEQ ID NO: 947 - DNA, 8 bp, Artificial Sequence
  Synthetic primer
tggttgga

SEQ ID NO: 948 - DNA, 86 bp, Artificial Sequence
  Synthetic primer
aatgatacggcgaccaccgagatctacactctttccctacacgacgctcttccgatctatagccagcgatcttgtggaaaggacga

SEQ ID NO: 949 - DNA, 8 bp, Artificial Sequence
  Synthetic primer
atagccag

SEQ ID NO: 950 - DNA, 87 bp, Artificial Sequence
  Synthetic primer
aatgatacggcgaccaccgagatctacactctttccctacacgacgctcttccgatctgagccagtacgatcttgtggaaaggacga

SEQ ID NO: 951 - DNA, 8 bp, Artificial Sequence
  Synthetic primer
gagccagt

SEQ ID NO: 952 - DNA, 84 bp, Artificial Sequence
  Synthetic primer
aatgatacggcgaccaccgagatctacactctttccctacacgacgctcttccgatcttgtgatggatcttgtggaaaggacga

SEQ ID NO: 953 - DNA, 8 bp, Artificial Sequence
  Synthetic primer
tgtgatgg

SEQ ID NO: 954 - DNA, 85 bp, Artificial Sequence
  Synthetic primer
aatgatacggcgaccaccgagatctacactctttccctacacgacgctcttccgatctgtattgccgatcttgtggaaaggacga

SEQ ID NO: 955 - DNA, 8 bp, Artificial Sequence
  Synthetic primer
gtattgcc

SEQ ID NO: 956 - DNA, 86 bp, Artificial Sequence
  Synthetic primer
aatgatacggcgaccaccgagatctacactctttccctacacgacgctcttccgatctatgaagtgcgatcttgtggaaaggacga

SEQ ID NO: 957 - DNA, 8 bp, Artificial Sequence
  Synthetic primer
atgaagtg

SEQ ID NO: 958 - DNA, 87 bp, Artificial Sequence
  Synthetic primer
aatgatacggcgaccaccgagatctacactctttccctacacgacgctcttccgatcttaagcttgacgatcttgtggaaaggacga

SEQ ID NO: 959 - DNA, 8 bp, Artificial Sequence
  Synthetic primer
taagcttg

SEQ ID NO: 960 - DNA, 84 bp, Artificial Sequence
  Synthetic primer
aatgatacggcgaccaccgagatctacactctttccctacacgacgctcttccgatcttggtacctatcttgtggaaaggacga

SEQ ID NO: 961 - DNA, 8 bp, Artificial Sequence
  Synthetic primer
tggtacct

SEQ ID NO: 962 - DNA, 85 bp, Artificial Sequence
  Synthetic primer
aatgatacggcgaccaccgagatctacactctttccctacacgacgctcttccgatctgttatggagatcttgtggaaaggacga

SEQ ID NO: 963 - DNA, 8 bp, Artificial Sequence
  Synthetic primer
gttatgga

SEQ ID NO: 964 - DNA, 86 bp, Artificial Sequence
  Synthetic primer
aatgatacggcgaccaccgagatctacactctttccctacacgacgctcttccgatctatgaggaccgatcttgtggaaaggacga

SEQ ID NO: 965 - DNA, 8 bp, Artificial Sequence
  Synthetic primer
atgaggac

SEQ ID NO: 966 - DNA, 87 bp, Artificial Sequence
  Synthetic primer
aatgatacggcgaccaccgagatctacactctttccctacacgacgctcttccgatctgcagtactacgatcttgtggaaaggacga

SEQ ID NO: 967 - DNA, 8 bp, Artificial Sequence
  Synthetic primer
gcagtact

SEQ ID NO: 968 - DNA, 84 bp, Artificial Sequence
  Synthetic primer
aatgatacggcgaccaccgagatctacactctttccctacacgacgctcttccgatctcttgaatcatcttgtggaaaggacga

SEQ ID NO: 969 - DNA, 8 bp, Artificial Sequence
  Synthetic primer
cttgaatc

SEQ ID NO: 970 - DNA, 85 bp, Artificial Sequence
  Synthetic primer
aatgatacggcgaccaccgagatctacactctttccctacacgacgctcttccgatctccaactaagatcttgtggaaaggacga

SEQ ID NO: 971 - DNA, 8 bp, Artificial Sequence
  Synthetic primer
ccaactaa

SEQ ID NO: 972 - DNA, 86 bp, Artificial Sequence
  Synthetic primer
aatgatacggcgaccaccgagatctacactctttccctacacgacgctcttccgatctaataccatcgatcttgtggaaaggacga

SEQ ID NO: 973 - DNA, 8 bp, Artificial Sequence
  Synthetic primer
aataccat

SEQ ID NO: 974 - DNA, 87 bp, Artificial Sequence
  Synthetic primer
aatgatacggcgaccaccgagatctacactctttccctacacgacgctcttccgatctacctatgcacgatcttgtggaaaggacga

SEQ ID NO: 975 - DNA, 8 bp, Artificial Sequence
  Synthetic primer
acctatgc

SEQ ID NO: 976 - DNA, 84 bp, Artificial Sequence
  Synthetic primer
aatgatacggcgaccaccgagatctacactctttccctacacgacgctcttccgatctgaacgctaatcttgtggaaaggacga

SEQ ID NO: 977 - DNA, 8 bp, Artificial Sequence
  Synthetic primer
gaacgcta

SEQ ID NO: 978 - DNA, 85 bp, Artificial Sequence
  Synthetic primer
aatgatacggcgaccaccgagatctacactctttccctacacgacgctcttccgatctctgacatcgatcttgtggaaaggacga

SEQ ID NO: 979 - DNA, 8 bp, Artificial Sequence
  Synthetic primer
ctgacatc

SEQ ID NO: 980 - DNA, 86 bp, Artificial Sequence
  Synthetic primer
aatgatacggcgaccaccgagatctacactctttccctacacgacgctcttccgatctgccaccatcgatcttgtggaaaggacga

SEQ ID NO: 981 - DNA, 8 bp, Artificial Sequence
  Synthetic primer
gccaccat

SEQ ID NO: 982 - DNA, 87 bp, Artificial Sequence
  Synthetic primer
aatgatacggcgaccaccgagatctacactctttccctacacgacgctcttccgatctcgactctcacgatcttgtggaaaggacga

SEQ ID NO: 983 - DNA, 8 bp, Artificial Sequence
  Synthetic primer
cgactctc

SEQ ID NO: 984 - DNA, 84 bp, Artificial Sequence
  Synthetic primer
aatgatacggcgaccaccgagatctacactctttccctacacgacgctcttccgatcttgctattaatcttgtggaaaggacga

SEQ ID NO: 985 - DNA, 8 bp, Artificial Sequence
  Synthetic primer
tgctatta

SEQ ID NO: 986 - DNA, 85 bp, Artificial Sequence
  Synthetic primer
aatgatacggcgaccaccgagatctacactctttccctacacgacgctcttccgatctcttctggcgatcttgtggaaaggacga

SEQ ID NO: 987 - DNA, 8 bp, Artificial Sequence
  Synthetic primer
cttctggc

SEQ ID NO: 988 - DNA, 86 bp, Artificial Sequence
  Synthetic primer
aatgatacggcgaccaccgagatctacactctttccctacacgacgctcttccgatctatgaattacgatcttgtggaaaggacga

SEQ ID NO: 989 - DNA, 8 bp, Artificial Sequence
  Synthetic primer
atgaatta

SEQ ID NO: 990 - DNA, 87 bp, Artificial Sequence
  Synthetic primer
aatgatacggcgaccaccgagatctacactctttccctacacgacgctcttccgatcttactccagacgatcttgtggaaaggacga

SEQ ID NO: 991 - DNA, 8 bp, Artificial Sequence
  Synthetic primer
tactccag

SEQ ID NO: 992 - DNA, 84 bp, Artificial Sequence
  Synthetic primer
aatgatacggcgaccaccgagatctacactctttccctacacgacgctcttccgatctatcataccatcttgtggaaaggacga

SEQ ID NO: 993 - DNA, 8 bp, Artificial Sequence
  Synthetic primer
atcatacc

SEQ ID NO: 994 - DNA, 85 bp, Artificial Sequence
  Synthetic primer
aatgatacggcgaccaccgagatctacactctttccctacacgacgctcttccgatctcctctaacgatcttgtggaaaggacga

SEQ ID NO: 995 - DNA, 8 bp, Artificial Sequence
  Synthetic primer
cctctaac

SEQ ID NO: 996 - DNA, 86 bp, Artificial Sequence
  Synthetic primer
aatgatacggcgaccaccgagatctacactctttccctacacgacgctcttccgatctatcttctccgatcttgtggaaaggacga

SEQ ID NO: 997 - DNA, 8 bp, Artificial Sequence
  Synthetic primer
atcttctc

SEQ ID NO: 998 - DNA, 87 bp, Artificial Sequence
  Synthetic primer
aatgatacggcgaccaccgagatctacactctttccctacacgacgctcttccgatctcagctcacacgatcttgtggaaaggacga

SEQ ID NO: 999 - DNA, 8 bp, Artificial Sequence
  Synthetic primer
cagctcac

SEQ ID NO: 1000 - DNA, 84 bp, Artificial Sequence
  Synthetic primer
aatgatacggcgaccaccgagatctacactctttccctacacgacgctcttccgatctggttatctatcttgtggaaaggacga

SEQ ID NO: 1001 - DNA, 8 bp, Artificial Sequence
  Synthetic primer
ggttatct

SEQ ID NO: 1002 - DNA, 85 bp, Artificial Sequence
  Synthetic primer
aatgatacggcgaccaccgagatctacactctttccctacacgacgctcttccgatcttccgcatagatcttgtggaaaggacga

SEQ ID NO: 1003 - DNA, 8 bp, Artificial Sequence
  Synthetic primer
tccgcata

SEQ ID NO: 1004 - DNA, 86 bp, Artificial Sequence
  Synthetic primer
aatgatacggcgaccaccgagatctacactctttccctacacgacgctcttccgatcttgcttcaccgatcttgtggaaaggacga

SEQ ID NO: 1005 - DNA, 8 bp, Artificial Sequence
  Synthetic primer
tgcttcac

SEQ ID NO: 1006 - DNA, 87 bp, Artificial Sequence
  Synthetic primer
aatgatacggcgaccaccgagatctacactctttccctacacgacgctcttccgatctgcttcctaacgatcttgtggaaaggacga

SEQ ID NO: 1007 - DNA, 8 bp, Artificial Sequence
  Synthetic primer
gcttccta

SEQ ID NO: 1008 - DNA, 84 bp, Artificial Sequence
  Synthetic primer
aatgatacggcgaccaccgagatctacactctttccctacacgacgctcttccgatctgtaatcgcatcttgtggaaaggacga

SEQ ID NO: 1009 - DNA, 8 bp, Artificial Sequence
  Synthetic primer
gtaatcgc

SEQ ID NO: 1010 - DNA, 85 bp, Artificial Sequence
  Synthetic primer
aatgatacggcgaccaccgagatctacactctttccctacacgacgctcttccgatctggccaattgatcttgtggaaaggacga

SEQ ID NO: 1011 - DNA, 8 bp, Artificial Sequence
  Synthetic primer
ggccaatt

SEQ ID NO: 1012 - DNA, 86 bp, Artificial Sequence
  Synthetic primer
aatgatacggcgaccaccgagatctacactctttccctacacgacgctcttccgatctaagcaattcgatcttgtggaaaggacga

SEQ ID NO: 1013 - DNA, 8 bp, Artificial Sequence
  Synthetic primer
aagcaatt

SEQ ID NO: 1014 - DNA, 87 bp, Artificial Sequence
  Synthetic primer
aatgatacggcgaccaccgagatctacactctttccctacacgacgctcttccgatctcagaccaaacgatcttgtggaaaggacga

SEQ ID NO: 1015 - DNA, 8 bp, Artificial Sequence
  Synthetic primer
cagaccaa

SEQ ID NO: 1016 - DNA, 84 bp, Artificial Sequence
  Synthetic primer
aatgatacggcgaccaccgagatctacactctttccctacacgacgctcttccgatctccaggatgatcttgtggaaaggacga

SEQ ID NO: 1017 - DNA, 8 bp, Artificial Sequence
  Synthetic primer
ccaggatg

SEQ ID NO: 1018 - DNA, 85 bp, Artificial Sequence
  Synthetic primer
aatgatacggcgaccaccgagatctacactctttccctacacgacgctcttccgatctgttaatccgatcttgtggaaaggacga

SEQ ID NO: 1019 - DNA, 8 bp, Artificial Sequence
  Synthetic primer
gttaatcc

SEQ ID NO: 1020 - DNA, 86 bp, Artificial Sequence
  Synthetic primer
aatgatacggcgaccaccgagatctacactctttccctacacgacgctcttccgatctaatatgcgcgatcttgtggaaaggacga

SEQ ID NO: 1021 - DNA, 8 bp, Artificial Sequence
  Synthetic primer
aatatgcg

SEQ ID NO: 1022 - DNA, 87 bp, Artificial Sequence
  Synthetic primer
aatgatacggcgaccaccgagatctacactctttccctacacgacgctcttccgatcttcgaatgaacgatcttgtggaaaggacga

SEQ ID NO: 1023 - DNA, 8 bp, Artificial Sequence
  Synthetic primer
tcgaatga

SEQ ID NO: 1024 - DNA, 84 bp, Artificial Sequence
  Synthetic primer
aatgatacggcgaccaccgagatctacactctttccctacacgacgctcttccgatctgattggacatcttgtggaaaggacga

SEQ ID NO: 1025 - DNA, 8 bp, Artificial Sequence
  Synthetic primer
gattggac

SEQ ID NO: 1026 - DNA, 85 bp, Artificial Sequence
  Synthetic primer
aatgatacggcgaccaccgagatctacactctttccctacacgacgctcttccgatcttgaccaaggatcttgtggaaaggacga

SEQ ID NO: 1027 - DNA, 8 bp, Artificial Sequence
  Synthetic primer
tgaccaag

SEQ ID NO: 1028 - DNA, 86 bp, Artificial Sequence
  Synthetic primer
aatgatacggcgaccaccgagatctacactctttccctacacgacgctcttccgatctagcgttggcgatcttgtggaaaggacga

SEQ ID NO: 1029 - DNA, 8 bp, Artificial Sequence
  Synthetic primer
agcgttgg

SEQ ID NO: 1030 - DNA, 87 bp, Artificial Sequence
  Synthetic primer
aatgatacggcgaccaccgagatctacactctttccctacacgacgctcttccgatctgaagtggaacgatcttgtggaaaggacga

SEQ ID NO: 1031 - DNA, 8 bp, Artificial Sequence
  Synthetic primer
gaagtgga

SEQ ID NO: 1032 - DNA, 84 bp, Artificial Sequence
  Synthetic primer
aatgatacggcgaccaccgagatctacactctttccctacacgacgctcttccgatcttggagattatcttgtggaaaggacga

SEQ ID NO: 1033 - DNA, 8 bp, Artificial Sequence
  Synthetic primer
tggagatt

SEQ ID NO: 1034 - DNA, 85 bp, Artificial Sequence
  Synthetic primer
aatgatacggcgaccaccgagatctacactctttccctacacgacgctcttccgatctgtgcagacgatcttgtggaaaggacga

SEQ ID NO: 1035 - DNA, 8 bp, Artificial Sequence
  Synthetic primer
gtgcagac

SEQ ID NO: 1036 - DNA, 86 bp, Artificial Sequence
  Synthetic primer
aatgatacggcgaccaccgagatctacactctttccctacacgacgctcttccgatctgcgctattcgatcttgtggaaaggacga

SEQ ID NO: 1037 - DNA, 8 bp, Artificial Sequence
  Synthetic primer
gcgctatt

SEQ ID NO: 1038 - DNA, 87 bp, Artificial Sequence
  Synthetic primer
aatgatacggcgaccaccgagatctacactctttccctacacgacgctcttccgatctaagagattacgatcttgtggaaaggacga

SEQ ID NO: 1039 - DNA, 8 bp, Artificial Sequence
  Synthetic primer
aagagatt

SEQ ID NO: 1040 - DNA, 84 bp, Artificial Sequence
  Synthetic primer
aatgatacggcgaccaccgagatctacactctttccctacacgacgctcttccgatcttgtgcattatcttgtggaaaggacga

SEQ ID NO: 1041 - DNA, 8 bp, Artificial Sequence
  Synthetic primer
tgtgcatt

SEQ ID NO: 1042 - DNA, 85 bp, Artificial Sequence
  Synthetic primer
aatgatacggcgaccaccgagatctacactctttccctacacgacgctcttccgatctgatcgcgtgatcttgtggaaaggacga

SEQ ID NO: 1043 - DNA, 8 bp, Artificial Sequence
  Synthetic primer
gatcgcgt

SEQ ID NO: 1044 - DNA, 86 bp, Artificial Sequence
  Synthetic primer
aatgatacggcgaccaccgagatctacactctttccctacacgacgctcttccgatctaagatagtcgatcttgtggaaaggacga

SEQ ID NO: 1045 - DNA, 8 bp, Artificial Sequence
  Synthetic primer
aagatagt

SEQ ID NO: 1046 - DNA, 87 bp, Artificial Sequence
  Synthetic primer
aatgatacggcgaccaccgagatctacactctttccctacacgacgctcttccgatctctcccagtacgatcttgtggaaaggacga

SEQ ID NO: 1047 - DNA, 8 bp, Artificial Sequence
  Synthetic primer
ctcccagt

SEQ ID NO: 1048 - DNA, 177 bp, Artificial Sequence
  Synthetic promoter
ttctagagcacagctaacaccacgtcgtccctatctgctgccctaggtctatgagtggttgctggataactttacgcatgcataaggctcggtatctatattcagggagaccacaacggtttccctctacaaataattttgtttaacttttactagagtcacacaggaaagtactag

SEQ ID NO: 1049 - DNA, 176 bp, Artificial Sequence
  Synthetic promoter
ttctagagcacagctaacaccacgtcgtccctatctgctgccctaggtctatgagtggttgctggataactttacggtgcataaggctcggtatctatattcagggagaccacaacggtttccctctacaaataattttgtttaacttttactagagtcacacaggaaagtactag

SEQ ID NO: 1050 - DNA, 179 bp, Artificial Sequence
  Synthetic promoter
ttctagagcacagctaacaccacgtcgtccctatctgctgccctaggtctatgagtggttgctggataacgcggtgggcatgcataaggctcgtataatatattcagggagaccacaacggtttccctctacaaataattttgtttaacttttactagagtcacacaggaaagtactag

SEQ ID NO: 1051 - DNA, 179 bp, Artificial Sequence
  Synthetic promoter
ttctagagcacagctaacaccacgtcgtccctatctgctgccctaggtctatgagtggttgctggataactttacgggcatgcataaggctcggtatctatattcagggagaccacaacggtttccctctacaaataattttgtttaacttttactagagtcacacaggaaagtactag

SEQ ID NO: 1052 - DNA, 179 bp, Artificial Sequence
  Synthetic promoter
ttctagagcacagctaacaccacgtcgtccctatctgctgccctaggtctatgagtggttgctggataactttacgggcatgcataaggctcgtaggatatattcagggagaccacaacggtttccctctacaaataattttgtttaacttttactagagtcacacaggaaagtactag

SEQ ID NO: 1053 - DNA, 179 bp, Artificial Sequence
  Synthetic promoter
ttctagagcacagctaacaccacgtcgtccctatctgctgccctaggtctatgagtggttgctggataactttacgggcatgcataaggctcgtaatatatattcagggagaccacaacggtttccctctacaaataattttgtttaacttttactagagtcacacaggaaagtactag

SEQ ID NO: 1054 - DNA, 179 bp, Artificial Sequence
  Synthetic promoter
ttctagagcacagctaacaccacgtcgtccctatctgctgccctaggtctatgagtggttgctggataactttacgggcatgcataaggctcgtaaaatatattcagggagaccacaacggtttccctctacaaataattttgtttaacttttactagagtcacacaggaaagtactag

SEQ ID NO: 1055 - DNA, 179 bp, Artificial Sequence
  Synthetic promoter
ttctagagcacagctaacaccacgtcgtccctatctgctgccctaggtctatgagtggttgctggataactttacgggcatgcataaggctcgtatgatatattcagggagaccacaacggtttccctctacaaataattttgtttaacttttactagagtcacacaggaaagtactag

SEQ ID NO: 1056 - DNA, 179 bp, Artificial Sequence
  Synthetic promoter
ttctagagcacagctaacaccacgtcgtccctatctgctgccctaggtctatgagtggttgctggataactttacgggcatgcataaggctcgtataatatattcagggagaccacaacggtttccctctacaaataattttgtttaacttttactagagtcacacaggaaagtactag